(19) United States
(12) Patent Application Publication
(10) Pub. No.: US 2026/0146253 A1
(43) Pub. Date: May 28, 2026

(54) COMPOSITIONS AND METHODS FOR PROGRAMMED DEATH LIGAND RECEPTOR (PD-L1) EXPRESSION

(71) Applicant: NOVO NORDISK A/S, Bagsværd (DK)

(72) Inventors: Shanthi Ganesh, Shrewsbury, MA (US); Marc Abrams, Natick, MA (US); Henryk T. Dudek, Belmont, MA (US); Harini Sivagurunatha Krishnan, Lexington, MA (US)

(21) Appl. No.: 19/460,949

(22) Filed: Jan. 27, 2026

Related U.S. Application Data
(63) Continuation of application No. PCT/US24/39733, filed on Jul. 26, 2024.
(60) Provisional application No. 63/516,270, filed on Jul. 28, 2023.

(51) Int. Cl. C12N 15/113; A61K 31/712; A61P 35/00

(52) U.S. Cl. CPC C12N 15/1138; A61K 31/712; A61P 35/00; C12N 2310/11; C12N 2310/321; C12N 2310/3515

(57) ABSTRACT

Oligonucleotides are provided herein that inhibit CD274 expression. Also provided are compositions including the same and uses thereof, particularly uses relating to treating diseases, disorders and/or conditions associated with aberrant CD274 expression.

### CROSS-RELATED APPLICATIONS

This application is a Continuation of Application No. PCT/US24/39733 filed on Jul. 26, 2024, which claims the benefit of U.S. Provisional Application No. 63/516,270 filed Jul. 28, 2023. The entire contents of these applications are incorporated herein by reference in their entirety.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 22, 2024, is named “DCY-10825.xml” and is 3,044,713 bytes in size. The sequence listing contained in this .XML file is part of the specification and is hereby incorporated by reference herein in its entirety.

### BACKGROUND

Currently, chemotherapy is the leading cancer therapy worldwide, often combined with surgery, or surgery and radiotherapy, depending on tumor type and stage (Abbas et al., An Overview of Cancer Treatment Modalities/IntechOpen, 2018). Since the discovery of several important mutations that contribute to carcinogenesis (e.g., adaptive immune resistance) these mutations and the proteins they represent have been extensively used as targets for the development of more selective drugs and drug combinations to treat cancer patients. Despite the effectiveness of these drugs, multidrug resistance (MDR) is often seen in patients, which often results in tumor relapse, limited therapeutic options and low quality of life for patients. In addition, cancer research has often been focused on tumor cells even though the effect of the tumor microenvironment and the ‘normal’ or non-cancerous cells within it that have been shown to play a key role in tumor progression, development and MDR (Klemm et al., TRENDS CELL BIOL (2015) 25(4): 198-213). Novel therapies that target different facets of the TME that contribute to tumor growth are needed.

### SUMMARY OF DISCLOSURE

The disclosure is based, in part, on the discovery of oligonucleotides that target PD-L1 mRNA and reduce expression. The disclosure is further based on the discovery that a combination of PD-L1 oligonucleotide and a CTLA-4 inhibitor provides synergistic anti-tumor efficacy for tumors having varying tumor microenvironments. Specifically, as demonstrated herein, a PD-L1 oligonucleotide conjugated to a lipid (e.g., a C18 hydrocarbon chain conjugated to the 5′ terminal nucleotide), when delivered alone or in combination with a CTLA-4 antibody, reduced tumor volume in vivo. Further, as shown herein, treatment with the PD-L1 oligonucleotide reduced tumor burden in an inflamed tumor microenvironment. In addition, the efficacy of the PD-L1 was dependent on the presence of CD8+ T cells.

Accordingly, in some aspects, the disclosure provides an oligonucleotide comprising an antisense strand comprising the nucleotide sequence of SEQ ID NO: 728 and a sense strand comprising the nucleotide sequence of SEQ ID NO: 487, wherein the sense strand comprises a saturated C18 hydrocarbon chain conjugated to the 5′ terminal nucleotide of the sense strand, and wherein each of the antisense and sense strands comprise at least one 2′-modified nucleotide and at least one modified internucleotide linkage.

In some aspects, the disclosure provides an oligonucleotide comprising an antisense strand comprising the nucleotide sequence of SEQ ID NO: 728 and a sense strand comprising the nucleotide sequence of SEQ ID NO:487, wherein the sense strand comprises a saturated C18 hydrocarbon chain conjugated to the 2′ carbon of the ribose ring of the 5′ terminal nucleotide of the sense strand via a linker, and wherein each of the antisense and sense strands comprise at least one 2′-modified nucleotide and at least one modified internucleotide linkage.

In some aspects, the disclosure provides an oligonucleotide comprising an antisense strand comprising the nucleotide sequence of SEQ ID NO: 725 and a sense strand comprising the nucleotide sequence of SEQ ID NO: 484, wherein the sense strand comprises a saturated C18 hydrocarbon chain conjugated to the 5′ terminal nucleotide of the sense strand, and wherein each of the antisense and sense strands comprise at least one 2′-modified nucleotide and at least one modified internucleotide linkage.

In some aspects, the disclosure provides an oligonucleotide comprising an antisense strand comprising the nucleotide sequence of SEQ ID NO: 725 and a sense strand comprising the nucleotide sequence of SEQ ID NO: 484, wherein the sense strand comprises a saturated C18 hydrocarbon chain conjugated to the 2′ carbon of the ribose ring of the 5′ terminal nucleotide of the sense strand via a linker, and wherein each of the antisense and sense strands comprise at least one 2′-modified nucleotide and at least one modified internucleotide linkage.

In some aspects, the disclosure provides an oligonucleotide comprising an antisense strand comprising the nucleotide sequence of SEQ ID NO: 732 and a sense strand comprising the nucleotide sequence of SEQ ID NO: 491, wherein the sense strand comprises a saturated C18 hydrocarbon chain conjugated to the 5′ terminal nucleotide of the sense strand, and wherein each of the antisense and sense strands comprise at least one 2′-modified nucleotide and at least one modified internucleotide linkage.

In some aspects, the disclosure provides an oligonucleotide comprising an antisense strand comprising the nucleotide sequence of SEQ ID NO: 732 and a sense strand comprising the nucleotide sequence of SEQ ID NO: 491, wherein the sense strand comprises a saturated C18 hydrocarbon chain conjugated to the 2′ carbon of the ribose ring of the 5′ terminal nucleotide of the sense strand via a linker, and wherein each of the antisense and sense strands comprise at least one 2′-modified nucleotide and at least one modified internucleotide linkage.

In some embodiments of any of the foregoing or related aspects, the 2′-modified nucleotide comprises a 2′-modification selected from 2′-aminoethyl, 2′-fluoro, 2′-O-methyl, 2′-O-methoxyethyl, and 2′-deoxy-2′-fluoro-β-d-arabinonucleic acid.

In some embodiments of any of the foregoing or related aspects, about 10-15%, 10%, 11%, 12%, 13%, 14% or 15% of the nucleotides of the sense strand comprise a 2′-fluoro modification.

In some embodiments, about 25-35%, 25%, 26%, 27%, 28%, 29%, 30%, 31%, 32%, 33%, 34% or 35% of the nucleotides of the antisense strand comprise a 2′-fluoro modification. In some embodiments, about 25-35%, 25%, 26%, 27%, 28%, 29%, 30%, 31%, 32%, 33%, 34% or 35% of the nucleotides of the oligonucleotide comprise a 2′-fluoro modification.

In some embodiments of any of the foregoing or related aspects, positions 8-11 of the sense strand each comprise a 2′-fluoro modification. In some embodiments, positions 2, 3, 4, 5, 7, 10 and 14 of the antisense strand each comprise a 2′-fluoro modification. In some embodiments, the remaining nucleotides comprise a 2′-O-methyl modification, provided the 5′ terminal nucleotide of the sense strand conjugated to the saturated C18 hydrocarbon chain does not comprise a 2′-O-methyl modification.

In some embodiments of any of the foregoing or related aspects, the at least one modified internucleotide linkage is a phosphorothioate linkage.

In some embodiments of any of the foregoing or related aspects, the sense strand comprises a phosphorothioate linkage between positions 1 and 2 of the sense strand. In some embodiments, the sense strand comprises a phosphorothioate linkage between positions 1 and 2, 2 and 3, and 3 and 4 of the sense strand. In some embodiments, the antisense strand comprises a phosphorothioate linkage between positions 1 and 2, 2 and 3, 20 and 21, and 21 and 22. In some embodiments, the antisense strand comprises a phosphorothioate linkage between positions 1 and 2, 2 and 3, 3 and 4, 20 and 21, and 21 and 22.

In some embodiments of any of the foregoing or related aspects, the 4′-carbon of the sugar of the 5′-nucleotide of the antisense strand comprises a phosphate analog. In some embodiments, the phosphate analog is oxymethyl phosphonate, vinyl phosphonate or malonyl phosphonate.

In some aspects, the disclosure provides an oligonucleotide comprising an antisense strand and a sense strand, wherein the antisense strand is 20 to 30 nucleotides in length and has a region of complementarity of 19 to 29 nucleotides to a target sequence of CD274 as set forth in any one of SEQ ID NOs: 2, 5, and 9, wherein the sense strand is 28 to 40 nucleotides in length and comprises at its 3′ end a stem-loop set forth as: S1-L-S2, wherein S1 is complementary to S2, and wherein L forms a loop between S1 and S2 of 3 to 5 nucleotides in length, wherein the antisense strand and the sense strand form a duplex region of at least 19 nucleotides in length, and wherein the sense strand comprises a C18 hydrocarbon chain conjugated to the 5′ terminal nucleotide of the sense strand.

In some aspects, the disclosure provides an oligonucleotide comprising an antisense strand of about 20 to 22 nucleotides in length and a sense strand of about 28 to 40 nucleotides in length, wherein the antisense and sense strands from an asymmetric duplex region of about 20 to 22 base pairs comprising a 3′ terminal overhang of at least 1 nucleotide of the antisense strand, wherein the antisense strand comprises a region of complementarity of 19 to 21 nucleotides to a target sequence of CD274 as set forth in any one of SEQ ID NOs: 2, 5, and 9, wherein the sense strand comprises: (i) a stem-loop at the 3′ end of the sense strand, wherein the stem-loop comprises a nucleotide sequence represented by the formula: 5′-S1-L-S2-3′, wherein S1 is complementary to S2, and wherein L forms a loop between S1 and S2 of 3 to 5 nucleotides in length, and (ii) at least one C18 hydrocarbon chain conjugated to the 5′ terminal nucleotide of the sense strand, and wherein each of the antisense and sense strands comprise at least one 2′-modified nucleotide and at least one modified internucleotide linkage.

In some embodiments of any of the foregoing or related aspects, the antisense strand comprises a sequence as set forth in any one of SEQ ID NOs: 725, 728, and 732.

In some embodiments of any of the foregoing or related aspects, the sense strand comprises a sequence as set forth in any one of SEQ ID NOs: 966, 969, and 973.

In some aspects, the disclosure provides a CD274-targeting oligonucleotide for reducing CD274 expression comprising a sense strand comprising the nucleotide sequence as set forth in SEQ ID NO: 1050 and an antisense strand comprising the nucleotide sequence as set forth in SEQ ID NO: 1005.

In some embodiments of any of the foregoing or related aspects, L is a tetraloop. In some embodiments, L is 4 nucleotides in length. In some embodiments, L comprises a sequence set forth as GAAA.

In some embodiments of any of the foregoing or related aspects, the antisense strand comprises a 3′ terminal overhang of one or more nucleotides in length. In some embodiments, the 3′ terminal overhang is 2 nucleotides in length, optionally wherein the 3′ terminal overhang sequence is GG.

In some embodiments of any of the foregoing or related aspects, the oligonucleotide comprises at least one modified nucleotide. In some embodiments, the modified nucleotide is a 2′-modified nucleotide. In some embodiments, the 2′-modified nucleotide comprises a 2′-modification selected from 2′-aminoethyl, 2′-fluoro, 2′-O-methyl, 2′-O-methoxyethyl, and 2′-deoxy-2′-fluoro-β-d-arabinonucleic acid.

In some embodiments of any of the foregoing or related aspects, about 10-15%, 10%, 11%, 12%, 13%, 14% or 15% of the nucleotides of the sense strand comprise a 2′-fluoro modification.

In some embodiments, about 25-35%, 25%, 26%, 27%, 28%, 29%, 30%, 31%, 32%, 33%, 34% or 35% of the nucleotides of the antisense strand comprise a 2′-fluoro modification. In some embodiments, about 25-35%, 25%, 26%, 27%, 28%, 29%, 30%, 31%, 32%, 33%, 34% or 35% of the nucleotides of the oligonucleotide comprise a 2′-fluoro modification.

In some embodiments of any of the foregoing or related aspects, the sense strand comprises 36 nucleotides with positions 1-36 from 5′ to 3′, wherein positions 8-11 comprise a 2′-fluoro modification. In some embodiments, the antisense strand comprises 22 nucleotides with positions 1-22 from 3′ to 5′, and wherein positions 2, 3, 4, 5, 7, 10 and 14 comprise a 2′-fluoro modification.

In some embodiments of any of the foregoing or related aspects, the remaining nucleotides comprise a 2′-O-methyl modification, provided the 5′ terminal nucleotide of the sense strand conjugated to the saturated C18 hydrocarbon chain does not comprise a 2′-O-methyl modification.

In some embodiments of any of the foregoing or related aspects, the oligonucleotide comprises at least one modified internucleotide linkage. In some embodiments, the at least one modified internucleotide linkage is a phosphorothioate linkage.

In some embodiments of any of the foregoing or related aspects, the sense strand comprises a phosphorothioate linkage between positions 1 and 2 of the sense strand. In some embodiments, the sense strand comprises a phosphorothioate linkage between positions 1 and 2, 2 and 3, and 3 and 4 of the sense strand. In some embodiments, the antisense strand comprises 22 nucleotides with positions 1-22 from 3′ to 5′, wherein the antisense strand comprises a phosphorothioate linkage between positions 1 and 2, 2 and 3, 20 and 21, and 21 and 22. In some embodiments, the antisense strand comprises 22 nucleotides with positions 1-22 from 3′ to 5′, wherein the antisense strand comprises a phosphorothioate linkage between positions 1 and 2, 2 and 3, 3 and 4, 20 and 21, and 21 and 22.

In some embodiments of any of the foregoing or related aspects, the 4′-carbon of the sugar of the 5′-nucleotide of the antisense strand comprises a phosphate analog. In some embodiments, the phosphate analog is oxymethyl phosphonate, vinyl phosphonate or malonyl phosphonate.

In some aspects, the disclosure provides a pharmaceutical composition comprising an oligonucleotide of any embodiments of the foregoing or related aspects, and a pharmaceutically acceptable carrier, delivery agent or excipient.

In some aspects, the disclosure provides a method of treating cancer in a subject, the method comprising administering to the subject an effective amount of an oligonucleotide or pharmaceutical composition of any embodiments of the foregoing or related aspects.

In some aspects, the disclosure provides a method of treating a disease, disorder or condition associated with activated CD274 expression, comprising administering to a subject in need thereof an oligonucleotide or pharmaceutical composition of any embodiments of the foregoing or related aspects.

In some aspects, the disclosure provides a method of treating cancer in a subject, the method comprising administering to the subject an effective amount of an oligonucleotide or pharmaceutical composition of any embodiments of the foregoing or related aspects, in combination with a CTLA4 inhibitor.

In some aspects, the disclosure provides a method of treating a treating a disease, disorder or condition associated with activated CD274 expression, comprising administering to a subject in need thereof an oligonucleotide or pharmaceutical composition of any embodiments of the foregoing or related aspects, in combination with a CTLA4 inhibitor.

In some embodiments of any of the foregoing or related aspects, the disease, disorder, or condition associated with activated CD274 expression is a cancer. In some embodiments, the cancer is selected from carcinoma, sarcoma, melanoma, lymphoma, and leukemia, prostate cancer, breast cancer, hepatocellular carcinoma (HCC), colorectal cancer, pancreatic cancer and glioblastoma.

In some embodiments of any of the foregoing or related aspects, the cancer comprises an immunosuppressive tumor microenvironment. In some embodiments, the cancer comprises an inflamed tumor microenvironment. In some embodiments, the inflamed tumor microenvironment comprises infiltrating T cells.

In some embodiments of any of the foregoing or related aspects, the CTLA-4 inhibitor is an antibody. In some embodiments, the antibody is an anti-CTLA-4 antibody. In some embodiments, the anti-CTLA-4 antibody is selected from Ipilimumab and Tremelimumab.

In some embodiments, the disclosure provides a method for delivering a CD274 targeting oligonucleotide to a lymph node of a subject, comprising administering an oligonucleotide of any embodiments of the foregoing or related aspects.

In some embodiments of any of the foregoing or related aspects, the lymph node is a tumor draining lymph node

In some embodiments, the disclosure provides for use of an oligonucleotide of any embodiments of the foregoing or related aspects in the manufacture of a medicament for the treatment of a disease, disorder, or condition associated with CD274 expression, optionally for the treatment of cancer.

In some aspects, the disclosure provides an oligonucleotide of any embodiments of the foregoing or related aspects, for use, or adaptable for use, in the treatment of a disease, disorder or condition associated with CD274 expression, optionally for the treatment of cancer.

In some aspects, the disclosure provides a kit comprising an oligonucleotide of any embodiments of the foregoing or related aspects, an optional pharmaceutically acceptable carrier, and a package insert comprising instructions for administration to a subject having a disease, disorder or condition associated with CD274 expression.

In some embodiments of any of the foregoing or related aspects, the disease, disorder or condition associated with CD274 expression is cancer.

In some aspects, the disclosure provides for use of an oligonucleotide of any embodiments of the foregoing or related aspects in the manufacture of a medicament for the treatment of a disease, disorder, or condition associated with CD274 expression, in combination with a CTLA4 inhibitor.

In some aspects, the disclosure provides an oligonucleotide of any embodiments of the foregoing or related aspects for use, or adaptable for use, in the treatment of a disease, disorder or condition associated with CD274 expression, in combination with a CTLA4 inhibitor.

In some aspects, the disclosure provides a kit an oligonucleotide of any embodiments of the foregoing or related aspects, an optional pharmaceutically acceptable carrier, and a package insert comprising instructions for administration of the RNAi oligonucleotide in combination with a CTLA4 inhibitor to a subject having a disease, disorder or condition associated with CD274 expression.

In some embodiments of any of the foregoing or related aspects, the disease, disorder or condition associated with CD274 expression is cancer.

### BRIEF DESCRIPTION OF THE DRAWINGS

FIG. 1A provides the structure of an exemplary RNAi oligonucleotide molecule having chemical modifications with a C18 lipid conjugated to the stem-loop, referred to as “GalXC-CD274-C18”. A GalXC-CD274-C18 oligonucleotide specific for mouse Cd274 is indicated as “GalXC-mCD274-C18” throughout.

FIG. 1B provides structures of lipid tails suitable for conjugation to RNAi oligonucleotide molecules.

FIGS. 2A-2F are graphs demonstrating remaining mouse Cd274 mRNA in tumor microenvironment (TME) and tumor draining lymph node (TDLN) following subcutaneous treatment with 25 mg/kg of GalXC-mCD274-C18 RNAi oligonucleotide at Day 1 and Day 4 (q3dx2) in checkpoint resistant Pan02 murine pancreatic tumor (FIGS. 2A-2B), checkpoint inhibitor resistant 4T1 murine triple negative breast tumor (FIGS. 2C-2D), a checkpoint inhibitor partially sensitive MC-38 murine colorectal tumor (FIGS. 2E-2F), and checkpoint inhibitor sensitive Hepa1-6 hepatoma tumor (FIG. 2G) bearing mice. Tissue from the TME and TDLN was collected 7 days after the final administration of the RNAi oligonucleotide. Control mice were administered PBS.

FIG. 3 is a graph representing remaining mouse Aldh2 mRNA levels in Cd11b and Cd11c cells isolated from tumor draining lymph nodes of Pan02 tumor bearing mice following treatment with an RNAi oligonucleotide targeting ALDH2 (i.e, GalXC-ALDH2-C18) subcutaneously at 25 mg/kg. Tissue from the TDLN was collected 3 days after the administration of the RNAi oligonucleotide. Control mice were administered PBS.

FIGS. 4A-4B are images showing the treatment regimen and GalXC-Placebo-C18 molecule (FIG. 4A) used to treat mice bearing 4T1 tumors with GalXC-mCD274-C18 RNAi oligonucleotide or GalXC-Placebo-C18 subcutaneously at 25 mg/kg or anti-PD-L1 monoclonal antibody (mAb) intraperitoneally at 10 mg/kg. Mice were treated at Day 1 and Day 4, and 7 days after the final administration of the RNAi oligonucleotide or placebo TDLN was collected and processed for immunohistochemistry of CD11c and PD-L1 expression (FIG. 4B).

FIGS. 5A-5C are graphs demonstrating remaining mouse Cd274, Ifng, and Gzmb mRNA following treatment with GalXC-mCD274-C18 RNAi oligonucleotide or anti-PD-L1 mAb in TDLNs of Pan02 (FIG. 5A), 4T1 (FIG. 5B), or MC-38 (FIG. 5C) tumor bearing mice. Mice were administered either PBS or the RNAi oligonucleotide subcutaneously at 25 mg/kg for q3dx2 (administered Day 1 and Day 4), or anti-PD-L1 mAb intraperitoneally at 10 mg/kg for q3dx2. Tissue was collected 7 days following administration of the RNAi oligonucleotide or mAb.

FIG. 6 provides images of CD8 immunohistochemistry in tumor microenvironment (TME) of MC-38 xenograft tumors treated with GalXC-mCD274-C18 RNAi oligonucleotide or anti-PD-L1 mAb. Mice were administered either PBS or the RNAi oligonucleotide subcutaneously at 25 mg/kg at[q3dx2]×2 (administered Day 1, 4, 8 and 12), or anti-PD-L1 mAb intraperitoneally at 10 mg/kg for q3dx2. Tissue was collected 7 days following administration of the RNAi oligonucleotide or mAb.

FIG. 7 provides images of CD8 immunohistochemistry in tumor microenvironment (TME) of 4T1 xenograft tumors treated with GalXC-mCD274-C18 RNAi oligonucleotide or anti-PD-L1 monoclonal antibody (mAb). Mice were administered either PBS or the RNAi oligonucleotide subcutaneously at 25 mg/kg for q3dx2 (administered Day 1 and Day 4), or anti-PD-L1 mAb intraperitoneally at 10 mg/kg for q3dx2. Tissue was collected 7 days following administration of the RNAi oligonucleotide or mAb.

FIGS. 8A-8B are graphs showing the anti-tumor effect of subcutaneous treatment with GalXC-mCD274-C18 RNAi oligonucleotide or anti-PD-L1 mAb. Tumor volume was measured in immunocompetent mice bearing 4T1 (FIG. 8A) and Pan02 (FIG. 8B) tumors. Mice with Pan02 tumors were treated with four 25 mg/kg (q3dx2 per cycle per week) of the GalXC-CD274 conjugate or PBS or mice with 4T1 tumors were treated with three 50 mg/kg doses of GalXC-CD274 or GalXC-Placebo conjugate (q3dx3) and both of the tumors were treated with the same frequency but at 10 mg/kg of mAb.

FIGS. 9A-9B provide a graph measuring tumor volume (FIG. 9A) and lung metastasis tumor images (FIG. 9B) following treatment with i) GalXC-placebo-C18; or ii) anti-PD-L1 mAb; or, iii) GalXC-mCD274-C18. Immunocompromised mice with 4T1 xenograft tumors were administered GalXC-mCD274-C18 RNAi oligonucleotide subcutaneously at 25 mg/kg or anti-PDL1 mAb intraperitoneally at 10 mg/kg on day 14, 17, and 20. On day 24, the lungs of the mice were photographed to capture the lung metastasis

FIGS. 10A-10B provide a graph measuring tumor volume (FIG. 10A) and lung metastasis tumor images (FIG. 10B) following treatment with i) GalXC-placebo-C18; or ii) anti-PD-L1 mAb; or, iii) GalXC-mCD274-C18. Immunocompetent mice with 4T1 xenograft tumors were administered GalXC-mCD274-C18 RNAi oligonucleotide subcutaneously at 25 mg/kg or anti-PD-L1 mAb intraperitoneally at 10 mg/kg for on day 14, 17, and 20. On day 24, the lungs of the mice were photographed to capture the lung metastasis.

FIGS. 11A-11B are graphs showing the anti-tumor effect of subcutaneous treatment with GalXC-mCD274-C18 RNAi oligonucleotide or anti-PD-L1 mAb. Tumor volume was measured in immunocompetent mice bearing MC-38 murine colorectal tumors (FIG. 11A) and Hepa1-6 murine hepatocellular tumors (FIG. 11B). Mice were treated with four 25 mg/kg doses of conjugate at q3dx2 per cycle per week for 2 weeks and with the same dosing frequency but at 10 mg/kg of mAb. Doses were given on Day 11, 14, 18 and 21.

FIG. 12A is a graph showing tumor volume following combination of GalXC-mCD274-C18 RNAi oligonucleotide and anti-PD-L1 mAb. Mice with checkpoint inhibitor partially sensitive MC-38 tumors were administered GalXC-Placebo-C18, GalXC-Placebo-C18 with anti-PD-L1 antibody, GalXC-mCD274-C18, or GalXC-mCD274-C18 in combination with anti-PD-L1 mAb. GalXC-mCD274-C18 was administered subcutaneously at 25 mg/kg or anti-PD-L1 mAb intraperitoneally at 10 mg/kg on Days 8, 11, 15, and 18.

FIG. 12B provides images of perforin immunohistochemistry in tumors of mice with checkpoint inhibitor partially sensitive MC-38 tumors administered GalXC-Placebo-C18, GalXC-Placebo-C18 with anti-PD-L1 antibody, GalXC-mCD274-C18, or GalXC-mCD274-C18 in combination with anti-PD-L1 mAb. GalXC-mCD274-C18 was administered subcutaneously at 25 mg/kg or anti-PD-L1 mAb intraperitoneally at 10 mg/kg on Days 8, 11, 15, and 18.

FIG. 13A is a graph showing tumor volume following treatment with i) GalXC-placebo-C18; or ii) anti-PD-L1 mAb; or, iii) GalXC-mCD274-C18. Mice with checkpoint inhibitor resistant 4T1 tumors were administered GalXC-mCD274-C18 RNAi oligonucleotide subcutaneously at 25 mg/kg or anti-PD-L1 mAb intraperitoneally at 10 mg/kg for on Days 6, 9, and 12.

FIG. 13B is a graph showing tumor volume following combination of GalXC-CD274 RNAi oligonucleotide and anti-CTLA-4 mAb. Mice with checkpoint inhibitor resistant 4T1 tumors were administered GalXC-Placebo-C18, GalXC-Placebo-C18 with anti-CTLA-4 mAb, GalXC-mCD274-C18, or GalXC-mCD274-C18 in combination with anti-CTLA-4 mAb. GalXC-mCD274-C18 was administered subcutaneously at 25 mg/kg and anti-CTLA-4 intraperitoneally at 10 mg/kg on Days 8, 11, and 14.

FIG. 13C provides images of CD8⁺ immunohistochemistry in tumors of mice with checkpoint inhibitor resistant 4T1 tumors administered GalXC-Placebo-C18, GalXC-Placebo-C18 with anti-CTLA-4 mAb, GalXC-mCD274-C18, or GalXC-mCD274-C18 in combination with anti-CTLA-4 mAb. GalXC-mCD274-C18 was administered subcutaneously at 25 mg/kg and anti-CTLA-4 intraperitoneally at 10 mg/kg on Days 8, 11, and 14.

FIG. 14 is a graph depicting the percent (%) of human CD274 mRNA remaining in RKO (human colon carcinoma) cells endogenously expressing human CD274, after 28-hour treatment with 1 nM of GalNAc-CD274 oligonucleotides targeting various regions of the CD274 gene.

FIG. 15 is a graph depicting the percent (%) of human CD274 mRNA remaining in RKO cells endogenously expressing human CD274, after 28-hour treatment with 0.3 nM, 1 nM, or 3 nM of GalNAc-CD274 oligonucleotides targeting various regions of the CD274 gene. % mRNA remaining was normalized to HPRT and SFRS9 housekeeping genes and mock transfection control.

FIGS. 16A-16B provide graphs depicting the percent (%) of human CD274 mRNA remaining in liver of mice exogenously expressing human CD274 (hydrodynamic injection model) after treatment with GalNAc-conjugated CD274 oligonucleotides. Mice were dosed subcutaneously with 2 mg/kg of the indicated GalNAc-CD274 oligonucleotides formulated in PBS. Three days post-dose mice were hydrodynamically injected (HDI) with 50 μg/mouse of a DNA ORF plasmid encoding human CD274. The level of human CD274 mRNA was determined from livers collected 20 hours after injection.

FIG. 17 provides structures of RNAi oligonucleotide molecules having chemical modifications with GalNAc conjugated to the stem-loop.

FIG. 18 provides a graph depicting the dose response of GalNAc-conjugated CD274 oligonucleotides of FIG. 17. The percent (%) of human CD274 mRNA remaining in liver of mice exogenously expressing CD274 (HDI model) after subcutaneous treatment with human GalNAc-conjugated CD274 oligonucleotides at 2 doses (0.3 mg/kg or 1 mg/kg) was measured. Three days post-dose mice were hydrodynamically injected (HDI) with 50 μg/mouse of a DNA ORF plasmid encoding CD274. The level of human CD274 mRNA was determined from livers collected 24 hours later.

FIG. 19 provides a graph depicting the dose response of GalNAc-conjugated CD274 oligonucleotides of FIG. 17. The percent (%) of human CD274 mRNA remaining in liver of mice exogenously expressing CD274 (HDI model) after subcutaneous treatment with human GalNAc-conjugated CD274 oligonucleotides at 2 doses (0.1 mg/kg or 0.3 mg/kg) was measured. Three days post-dose mice were hydrodynamically injected (HDI) with 50 μg/mouse of a DNA ORF plasmid encoding CD274. The level of human CD274 mRNA was determined from livers collected 24 hours later.

FIG. 20 provides graphs depicting the percent (%) remaining human CD274 mRNA in H460 lung carcinoma cells endogenously expressing CD274 (reverse transfection for CD274 expression was performed overnight using lipofectamine RNAiMAX) treated with GalNAc-CD274-094 or GalNAc-CD274-098. H640 cells were treated for 24 hours with a series of dose levels (0.0032 nM, 0.16 nM, 0.08 nM, 0.04 nM, 2 nM, 10 nM, and 50 nM) of oligonucleotide to generate IC₅₀ curves.

FIG. 21 provides graphs depicting the percent (%) remaining human CD274 mRNA in human primary macrophages endogenously expressing CD274 (reverse transfection for CD274 expression was performed overnight using lipofectamine RNAiMAX) treated with GalNAc-CD274-094 or GalNAc-CD274-098. Human primary macrophages were polarized to M2 immunosuppressive phase with IL-10 cytokine and stimulated with lipopolysaccharide. Macrophages were treated for 72 hours with a series of dose levels (0.0032 nM, 0.16 nM, 0.08 nM, 0.04 nM, 2 nM, 10 nM, and 50 nM) of oligonucleotide to generate IC₅₀ curves.

FIG. 22 provides a graph depicting the percent (%) remaining human CD274 mRNA in DC immune cell culture expressing CD274 (reverse transfection for CD274 expression was performed overnight using lipofectamine RNAiMAX) treated with GalNAc-CD274-094 or GalNAc-CD274-098. Cells were first treated with suppressive cytokines IL-10 and treated for 72 hours with a series of doses (0.2 nM, 1 nM, and 5 nM) of oligonucleotide and percent (%) remaining of CD274 mRNA was plotted.

FIG. 23 provides a graph depicting the cytokine production associated with CD274 mRNA downregulation described in FIG. 22. Supernatants were collected from the plate and proinflammatory cytokine IFN-7 level was measured using MSD V-plex assay kit.

FIGS. 24A and 24B are graphs demonstrating remaining mouse Aldh2 mRNA from bulk tumor (FIG. 24A), and liver (FIG. 24B) of Pan02 xenografts. Mice were treated with 25 mg/kg of the specified GalXC-ALDH2-lipid conjugate and mRNA was measured on day 3.

FIGS. 24C and 24D are graphs demonstrating remaining mouse Aldh2 mRNA from bulk tumor (FIG. 24C) and tumor draining lymph node (TdLN) from mice with Pan02 xenografts on day 7 and day 14 after treatment with 25 mg/kg of the specified GalXC-ALDH2-lipid conjugate.

FIG. 25 provides the structure of an the CD274-0098 RNAi oligonucleotide molecule having chemical modifications with a C18 lipid conjugated to 5′ terminal nucleotide.

### DETAILED DESCRIPTION

Programmed death-ligand 1 (cluster of differentiation 274, CD274, or PD-L1) is a type I transmembrane inhibitory receptor ligand expressed on immune cells and some tumor cells. Interaction of the ligand with the PD-1 receptor inhibits T-cell activation and subsequent cytokine production. Expression in tumor cells provides an ability to evade the tumor response by repressing cytotoxic T-cell activation. Although tumoral PD-L1 has been widely used to identify patients most likely to respond to therapy, recent evidence suggests that PD-L1 expressed by immune cells, especially antigen presenting dendritic cells (APCs or CD11c expressing DCs), is a better biomarker to predict clinical response than PD-L1 expressed by tumor cells. Additionally, most research associated with PD-L1 and PD-1 has been focused on the extrinsic role of inhibiting the immune system, but more recently a tumor intrinsic role of PD-L1 was shown to be involved in certain cancer types (Wu, Y et al, Front. Immunol. 10:2022, 2019, Hudson, K et al, Front. Immunol. 11:568931, 2020). Intracellular PD-L1 expressed by APCs demonstrated a role in regulating DC migration from tumor to tumor draining lymph nodes. Lack of silencing of intracellular PD-L1 on DCs may impair the antigen presentation machinery in tumors and facilitate resistance to immunotherapy. Monoclonal antibodies (mAbs) are designed to mainly target extracellular/membranous PD-L1 and are unlikely to reach the intracellular version of PD-L1. Without wishing to be bound by theory, PD-L1 RNAi oligonucleotides conjugated with GalNAc or lipid moieties have the ability to inhibit both extracellular and intracellular PD-L1 and are effective to reduce PD-L1 expression for therapy. Thus, cells with membranous or extracellular PD-L1 are targetable by mAbs, but cells with intracellular and extracellular PD-L1 require therapies including the PD-L1 RNAi oligonucleotides described herein for inhibition of PD-L1.

According to some aspects, the disclosure provides oligonucleotides (e.g., RNAi oligonucleotides) that reduce CD274 expression in the tumor microenvironment. In some embodiments, the oligonucleotides provided herein are designed to treat diseases associated with CD274 expression in tumors. In some respects, the disclosure provides methods of treating a disease associated with overall CD274 expression by reducing CD274 expression in specific cells (e.g., tumor cells) or in organs.

### Oligonucleotide Inhibitors of CD274 Expression

### CD274 Target Sequences

In some embodiments, an oligonucleotide herein (e.g., an RNAi oligonucleotide) is targeted to a target sequence comprising a CD274 mRNA. In some embodiments, an oligonucleotide described herein is targeted to a target sequence within a CD274 mRNA sequence. In some embodiments, the oligonucleotide described herein corresponds to a target sequence within a CD274 mRNA sequence. In some embodiments, the oligonucleotide, or a portion, fragment, or strand thereof (e.g., an antisense strand or a guide strand of a double-stranded (ds) RNAi oligonucleotide) binds or anneals to a target sequence comprising CD274 mRNA, thereby inhibiting CD274 expression.

In some embodiments, the oligonucleotide is targeted to a CD274 target sequence for the purpose of inhibiting CD274 expression in vivo. In some embodiments, the amount or extent of inhibition of CD274 expression by an oligonucleotide targeted to a CD274 target sequence correlates with the potency of the oligonucleotide. In some embodiments, the amount or extent of inhibition of CD274 expression by an oligonucleotide targeted to a CD274 target sequence correlates with the amount or extent of therapeutic benefit in a subject or patient having a disease, disorder or condition associated with CD274 expression treated with the oligonucleotide.

Through examination of the nucleotide sequence of mRNAs encoding CD274, including mRNAs of multiple different species (e.g., human, cynomolgus monkey, and mouse; see, e.g., Example 7) and as a result of in vitro and in vivo testing (see, e.g., Examples 2-7), it has been discovered that certain nucleotide sequences of CD274 mRNA are more amenable than others to oligonucleotide-based inhibition and are thus useful as target sequences for the oligonucleotides herein. In some embodiments, a sense strand of an oligonucleotide (e.g., an RNAi oligonucleotide) described herein comprises a CD274 target sequence. In some embodiments, a portion or region of the sense strand of an oligonucleotide described herein (e.g., an RNAi oligonucleotide) comprises a CD274 target sequence. In some embodiments, a CD274 target sequence comprises, or consists of, a sequence of any one of SEQ ID NOs: 1-2 and 4-241. In some embodiments, a CD274 target sequence comprises, or consists of, the sequence set forth in SEQ ID NO: 2, 4, 5, 6, 7, 9, or 20. In some embodiments, a CD274 target sequence comprises, or consists of, the sequence set forth in SEQ ID NO: 2. In some embodiments, a CD274 target sequence comprises, or consists of, the sequence set forth in SEQ ID NO: 4. In some embodiments, a CD274 target sequence comprises, or consists of, the sequence set forth in SEQ ID NO: 5. In some embodiments, a CD274 target sequence comprises, or consists of, the sequence set forth in SEQ ID NO: 6. In some embodiments, a CD274 target sequence comprises, or consists of, the sequence set forth in SEQ ID NO: 7. In some embodiments, a CD274 target sequence comprises, or consists of, the sequence set forth in SEQ ID NO: 9. In some embodiments, a CD274 target sequence comprises, or consists of, the sequence set forth in SEQ ID NO: 20.

### CD274 Targeting Sequences

In some embodiments, the oligonucleotides herein (e.g., RNAi oligonucleotides) have regions of complementarity to CD274 mRNA (e.g., within a target sequence of CD274 mRNA) for purposes of targeting the CD274 mRNA in cells and inhibiting and/or reducing CD274 expression. In some embodiments, the oligonucleotides herein comprise a CD274 targeting sequence (e.g., an antisense strand or a guide strand of an RNAi oligonucleotide) having a region of complementarity that binds or anneals to a CD274 target sequence by complementary (Watson-Crick) base pairing. The targeting sequence or region of complementarity is generally of a suitable length and base content to enable binding or annealing of the oligonucleotide (or a strand thereof) to a CD274 mRNA for purposes of inhibiting and/or reducing CD274 expression. In some embodiments, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence or region of complementarity is about 12 to about 30 (e.g., 12 to 30, 12 to 22, 15 to 25, 17 to 21, 18 to 27, 19 to 27, or 15 to 30) nucleotides in length. In some embodiments, the targeting sequence or region of complementarity is about 12, 13, 14, 15, 16, 17, 18, 19, 20, 21, 22, 23, 24, 25, 26, 27, 28, 29 or 30 nucleotides in length. In some embodiments, the targeting sequence or region of complementarity is 18 nucleotides in length. In some embodiments, the targeting sequence or region of complementarity is 19 nucleotides in length. In some embodiments, the targeting sequence or region of complementarity is 20 nucleotides in length. In some embodiments, the targeting sequence or region of complementarity is 21 nucleotides in length. In some embodiments, the targeting sequence or region of complementarity is 22 nucleotides in length. In some embodiments, the targeting sequence or region of complementarity is 23 nucleotides in length. In some embodiments, the targeting sequence or region of complementarity is 24 nucleotides in length. In some embodiments, an oligonucleotide comprises a target sequence or region of complementarity complementary to a sequence of any one of SEQ ID NOs: 1-2 and 4-241, and the targeting sequence or region of complementarity is 18 nucleotides in length. In some embodiments, an oligonucleotide comprises a target sequence or region of complementarity complementary to a sequence of any one of SEQ ID NOs: 1-2 and 4-241, and the targeting sequence or region of complementarity is 19 nucleotides in length. In some embodiments, an oligonucleotide comprises a target sequence or region of complementarity complementary to a sequence of SEQ ID NO: 1037, and the targeting sequence or region of complementarity is 20 nucleotides in length. In some embodiments, an oligonucleotide comprises a target sequence or region of complementarity complementary to a sequence of SEQ ID NO: 1037, and the targeting sequence or region of complementarity is 21 nucleotides in length. In some embodiments, an oligonucleotide comprises a target sequence or region of complementarity complementary to a sequence of SEQ ID NO: 1037, and the targeting sequence or region of complementarity is 22 nucleotides in length. In some embodiments, an oligonucleotide comprises a target sequence or region of complementarity complementary to a sequence of SEQ ID NO: 1037, and the targeting sequence or region of complementarity is 23 nucleotides in length. In some embodiments, an oligonucleotide comprises a target sequence or region of complementarity complementary to a sequence of SEQ ID NO: 1037 and the targeting sequence or region of complementarity is 24 nucleotides in length.

In some embodiments, an oligonucleotide herein (e.g., an RNAi oligonucleotide) comprises a targeting sequence or a region of complementarity (e.g., an antisense strand or a guide strand of a double-stranded oligonucleotide) that is fully complementary to a CD274 target sequence. In some embodiments, the targeting sequence or region of complementarity is partially complementary to a CD274 target sequence. In some embodiments, the oligonucleotide comprises a targeting sequence or region of complementarity that is fully complementary to a CD274 target sequence. In some embodiments, the oligonucleotide comprises a targeting sequence or region of complementarity that is partially complementary to a CD274 target sequence.

In some embodiments, the oligonucleotide comprises a targeting sequence or region of complementarity that is fully complementary to a sequence of any one of SEQ ID NOs: 1-2 and 4-241. In some embodiments, the oligonucleotide comprises a targeting sequence or region of complementarity that is fully complementary to the sequence set forth in SEQ ID NOs: 2, 4, 5, 6, 7, 9, or 20. In some embodiments, the oligonucleotide comprises a targeting sequence or region of complementarity that is partially complementary to a sequence of any one of SEQ ID NOs: 1-2 and 4-241. In some embodiments, the oligonucleotide comprises a targeting sequence or region of complementarity that is partially complementary to the sequence set forth in SEQ ID NOs: 2, 4, 5, 6, 7, 9, or 20. Some embodiments, the oligonucleotide comprises a targeting sequence or region of complementarity that is partially complementary to the sequence set forth in SEQ ID NO: 2. In some embodiments, the oligonucleotide comprises a targeting sequence or region of complementarity that is partially complementary to the sequence set forth in SEQ ID NO: 5. In some embodiments, the oligonucleotide comprises a targeting sequence or region of complementarity that is partially complementary to the sequence set forth in SEQ ID NO: 9.

In some embodiments, an oligonucleotide herein (e.g., an RNAi oligonucleotide) comprises a targeting sequence or region of complementarity that is complementary to a contiguous sequence of nucleotides within a CD274 mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the oligonucleotide comprises a targeting sequence or region of complementarity that is complementary to a contiguous sequence of nucleotides within a CD274 mRNA, wherein the contiguous sequence of nucleotides is 10, 11, 12, 13, 14, 15, 16, 17, 18, 19 or 20 nucleotides in length. In some embodiments, the oligonucleotide comprises a targeting sequence or region of complementarity that is complementary to a contiguous sequence of nucleotides within a CD274 mRNA, wherein the contiguous sequence of nucleotides is 19 nucleotides in length. In some embodiments, the oligonucleotide comprises a targeting sequence or region of complementarity that is complementary to a contiguous sequence of nucleotides within a CD274 mRNA, wherein the contiguous sequence of nucleotides is 20 nucleotides in length.

In some embodiments, an oligonucleotide herein (e.g., an RNAi oligonucleotide) comprises a targeting sequence or a region of complementary that is complementary to a contiguous sequence of nucleotides of any one of SEQ ID NOs: 2, 4, 5, 6, 7, 9, or 20, optionally wherein the contiguous sequence of nucleotides is 19 nucleotides in length. In some embodiments, an oligonucleotide herein (e.g., an RNAi oligonucleotide) comprises a targeting sequence or a region of complementary that is complementary to a contiguous sequence of nucleotides of SEQ ID NO: 2, optionally wherein the contiguous sequence of nucleotides is 19 nucleotides in length. In some embodiments, an oligonucleotide herein (e.g., an RNAi oligonucleotide) comprises a targeting sequence or a region of complementary that is complementary to a contiguous sequence of nucleotides of SEQ ID NO: 5, optionally wherein the contiguous sequence of nucleotides is 19 nucleotides in length. In some embodiments, an oligonucleotide herein (e.g., an RNAi oligonucleotide) comprises a targeting sequence or a region of complementary that is complementary to a contiguous sequence of nucleotides of SEQ ID NO: 9, optionally wherein the contiguous sequence of nucleotides is 19 nucleotides in length. In some embodiments, the oligonucleotide comprises a targeting sequence or a region of complementary that is complementary to a contiguous sequence of nucleotides of any one of SEQ ID NOs: 2, 4, 5, 6, 7, 9, or 20, wherein the contiguous sequence of nucleotides is 19 nucleotides in length. In some embodiments, the oligonucleotide comprises a targeting sequence or a region of complementary that is complementary to a contiguous sequence of nucleotides of SEQ ID NO: 2, wherein the contiguous sequence of nucleotides is 19 nucleotides in length. In some embodiments, the oligonucleotide comprises a targeting sequence or a region of complementary that is complementary to a contiguous sequence of nucleotides of SEQ ID NO: 5, wherein the contiguous sequence of nucleotides is 19 nucleotides in length. In some embodiments, the oligonucleotide comprises a targeting sequence or a region of complementary that is complementary to a contiguous sequence of nucleotides of SEQ ID NO: 9, wherein the contiguous sequence of nucleotides is 19 nucleotides in length. In some embodiments, the oligonucleotide comprises a targeting sequence or a region of complementary that is complementary to a contiguous sequence of nucleotides of SEQ ID NO: 1037, wherein the contiguous sequence of nucleotides is 20 nucleotides in length.

In some embodiments, an oligonucleotide herein (e.g., an RNAi oligonucleotide) comprises a targeting sequence or region of complementarity having one or more base pair (bp) mismatches with the corresponding CD274 target sequence. In some embodiments, the targeting sequence or region of complementarity may have up to about 1, up to about 2, up to about 3, up to about 4, up to about 5, etc. mismatches with the corresponding CD274 target sequence provided that the ability of the targeting sequence or region of complementarity to bind or anneal to the CD274 mRNA under appropriate hybridization conditions and/or the ability of the oligonucleotide to inhibit CD274 expression is maintained. Alternatively, the targeting sequence or region of complementarity may have no more than 1, no more than 2, no more than 3, no more than 4, or no more than 5 mismatches with the corresponding CD274 target sequence provided that the ability of the targeting sequence or region of complementarity to bind or anneal to the CD274 mRNA under appropriate hybridization conditions and/or the ability of the oligonucleotide to inhibit CD274 expression is maintained. In some embodiments, the oligonucleotide comprises a targeting sequence or region of complementarity having 1 mismatch with the corresponding target sequence. In some embodiments, the oligonucleotide comprises a targeting sequence or region of complementarity having 2 mismatches with the corresponding target sequence. In some embodiments, the oligonucleotide comprises a targeting sequence or region of complementarity having 3 mismatches with the corresponding target sequence. In some embodiments, the oligonucleotide comprises a targeting sequence or region of complementarity having 4 mismatches with the corresponding target sequence. In some embodiments, the oligonucleotide comprises a targeting sequence or region of complementarity having 5 mismatches with the corresponding target sequence. In some embodiments, the oligonucleotide comprises a targeting sequence or region of complementarity having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed throughout the targeting sequence or region of complementarity. In some embodiments, the oligonucleotide comprises a targeting sequence or region of complementarity having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof. In some embodiments, the oligonucleotide comprises a targeting sequence or a region of complementary that is complementary to a contiguous sequence of nucleotides of any one of SEQ ID NOs: 1-2 and 4-241, wherein the targeting sequence or region of complementarity may have up to about 1, up to about 2, up to about 3, up to about 4, up to about 5, etc. mismatches with the corresponding CD274 target sequence. In some embodiments, the oligonucleotide comprises a targeting sequence or a region of complementary that is complementary to a contiguous sequence of nucleotides of any one of SEQ ID NOs: 1-2 and 4-241, wherein the targeting sequence or region of complementarity may have no more than 1, no more than 2, no more than 3, no more than 4, or no more than 5 mismatches with the corresponding CD274 target sequence. In some embodiments, the oligonucleotide comprises a targeting sequence or a region of complementary that is complementary to a contiguous sequence of nucleotides of any one of SEQ ID NOs: 1-2 and 4-241, wherein the targeting sequence or region of complementarity may have up to about 1, up to about 2, up to about 3, up to about 4, up to about 5, etc. mismatches with the corresponding CD274 target sequence. In some embodiments, the oligonucleotide comprises a targeting sequence or a region of complementary that is complementary to a contiguous sequence of nucleotides of any one of SEQ ID NOs: 2, 4, 5, 6, 7, 9, or 20, wherein the targeting sequence or region of complementarity may have no more than 1, no more than 2, no more than 3, no more than 4, or no more than 5 mismatches with the corresponding CD274 target sequence.

### Types of Oligonucleotides

A variety of oligonucleotide types and/or structures are useful for targeting CD274 in the methods herein including, but not limited to, RNAi oligonucleotides, antisense oligonucleotides (ASOs), miRNAs, etc. Any of the oligonucleotide types described herein or elsewhere are contemplated for use as a framework to incorporate a CD274 targeting sequence herein for the purposes of inhibiting CD274 expression.

In some embodiments, the oligonucleotides herein inhibit CD274 expression by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended dsRNAs where at least one end of at least one strand is extended beyond a duplex targeting region, including structures where one of the strands includes a thermodynamically stabilizing tetraloop structure (see, e.g., U.S. Pat. Nos. 8,513,207 and 8,927,705, as well as Intl. Patent Application Publication No. WO 2010/033225). Such structures may include single-stranded (ss) extensions (on one or both sides of the molecule) as well as double-stranded (ds) extensions.

In some embodiments, the oligonucleotides herein engage with the RNAi pathway downstream of the involvement of Dicer (e.g., Dicer cleavage). In some embodiments, the oligonucleotides described herein are Dicer substrates. In some embodiments, upon endogenous Dicer processing, double-stranded nucleic acids of 19-23 nucleotides in length capable of reducing CD274 expression are produced. In some embodiments, the oligonucleotide has an overhang (e.g., of 1, 2, or 3 nucleotides in length) in the 3′ end of the antisense strand. In some embodiments, the oligonucleotide (e.g., siRNA) comprises a 21-nucleotide guide strand that is antisense to a target RNA and a complementary passenger strand, in which both strands anneal to form a 19-bp duplex and 2 nucleotide overhangs at either or both 3′ ends. Longer oligonucleotide designs also are available including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621 and 9,193,753.

In some embodiments, the oligonucleotides herein comprise sense and antisense strands that are both in the range of about 17 to 36 (e.g., 17 to 36, 20 to 25 or 21-23) nucleotides in length. In some embodiments, the oligonucleotides described herein comprise an antisense strand of 19-30 nucleotides in length and a sense strand of 19-50 nucleotides in length, wherein the antisense and sense strands are separate strands which form an asymmetric duplex region having an overhang of 1-4 nucleotides at the 3′ terminus of the antisense strand. In some embodiments, an oligonucleotide herein comprises a sense and antisense strand that are both in the range of about 19-22 nucleotides in length. In some embodiments, the sense and antisense strands are of equal length. In some embodiments, an oligonucleotide comprises sense and antisense strands, such that there is a 3′-overhang on either the sense strand or the antisense strand, or both the sense and antisense strand. In some embodiments, for oligonucleotides that have sense and antisense strands that are both in the range of about 21-23 nucleotides in length, a 3′ overhang on the sense, antisense, or both sense and antisense strands is 1 or 2 nucleotides in length. In some embodiments, the oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other oligonucleotide designs for use with the compositions and methods herein include: 16-mer siRNAs (see, e.g., NUCLEIC ACIDS IN CHEMISTRY AND BIOLOGY, Blackburn (ed.), ROYAL SOCIETY OF CHEMISTRY, 2006), shRNAs (e.g., having 19 bp or shorter stems; see, e.g., Moore et al. (2010) METHODS MOL. BIOL. 629:141-158), blunt siRNAs (e.g., of 19 bps in length; see, e.g., Kraynack & Baker (2006) RNA 12:163-176), asymmetrical siRNAs (aiRNA; see, e.g., Sun et al. (2008) NAT. BIOTECHNOL. 26:1379-82), asymmetric shorter-duplex siRNA (see, e.g., Chang et al. (2009) MOL. THER. 17:725-32), fork siRNAs (see, e.g., Hohjoh (2004) FEBS LETT. 557:193-98), ss siRNAs (Elsner (2012) NAT. BIOTECHNOL. 30:1063), dumbbell-shaped circular siRNAs (see, e.g., Abe et al. (2007) J. AM. CHEM. SOC. 129:15108-09), and small internally segmented interfering RNA (siRNA; see, e.g., Bramsen et al. (2007) NUCLEIC ACIDS RES. 35:5886-97). Further non-limiting examples of an oligonucleotide structures that may be used in some embodiments to reduce or inhibit the expression of CD274 are microRNA (miRNA), short hairpin RNA (shRNA) and short siRNA (see, e.g., Hamilton et al. (2002) EMBO J. 21:4671-79; see also, US Patent Application Publication No. 2009/0099115).

Still, in some embodiments, an oligonucleotide for reducing or inhibiting CD274 expression herein is single-stranded (ss). Such structures may include but are not limited to single-stranded RNAi molecules. Recent efforts have demonstrated the activity of ss RNAi molecules (see, e.g., Matsui et al. (2016) MOL. THER. 24:946-55). However, in some embodiments, oligonucleotides herein are antisense oligonucleotides (ASOs). An antisense oligonucleotide is a single-stranded oligonucleotide that has a nucleobase sequence which, when written in the 5′ to 3′ direction, comprises the reverse complement of a targeted segment of a particular nucleic acid and is suitably modified (e.g., as a gapmer) to induce RnaseH-mediated cleavage of its target RNA in cells or (e.g., as a mixmer) so as to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes (see, e.g., Bennett et al. (2017) ANNU. REV. PHARMACOL. 57:81-105).

In some embodiments, the antisense oligonucleotide shares a region of complementarity with CD274 mRNA. In some embodiments, the antisense oligonucleotide targets various areas of the human CD274 gene identified as NM_014143.4. In some embodiments, the antisense oligonucleotide targets various areas of the cynomolgus monkey CD274 gene identified as XM_005581779.2. In some embodiments, the antisense oligonucleotide targets various areas of the mouse CD274 gene identified as NM_021893.3. In some embodiments, the antisense oligonucleotide is 15-50 nucleotides in length. In some embodiments, the antisense oligonucleotide is 15-25 nucleotides in length. In some embodiments, the antisense oligonucleotide is 22 nucleotides in length. In some embodiments, the antisense oligonucleotide is complementary to any one of SEQ ID NOs: 1-2 and 4-241. In some embodiments, the antisense oligonucleotide is at least 15 contiguous nucleotides in length. In some embodiments, the antisense oligonucleotide is at least 19 contiguous nucleotides in length. In some embodiments, the antisense oligonucleotide is at least 20 contiguous nucleotides in length. In some embodiments, the antisense oligonucleotide differs by 1, 2, or 3 nucleotides from the target sequence.

### Double-Stranded Oligonucleotides

In some aspects, the disclosure provides double-stranded (ds) RNAi oligonucleotides for targeting CD274 mRNA and inhibiting CD274 expression (e.g., via the RNAi pathway) comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked. In some embodiments, the sense strand and antisense strand form a duplex region, wherein the sense strand and antisense strand, or a portion thereof, binds with one another in a complementary fashion (e.g., by Watson-Crick base pairing).

In some embodiments, the sense strand has a first region (R1) and a second region (R2), wherein R2 comprises a first subregion (S1), a tetraloop or triloop (L), and a second subregion (S2), wherein L is located between S1 and S2, and wherein S1 and S2 form a second duplex (D2). D2 may have various length. In some embodiments, D2 is about 1-6 bp in length. In some embodiments, D2 is 2-6, 3-6, 4-6, 5-6, 1-5, 2-5, 3-5 or 4-5 bp in length. In some embodiments, D2 is 1, 2, 3, 4, 5 or 6 bp in length. In some embodiments, D2 is 6 bp in length.

In some embodiments, R1 of the sense strand and the antisense strand form a first duplex (D1). In some embodiments, D1 is at least about 15 (e.g., at least 15, at least 16, at least 17, at least 18, at least 19, at least 20 or at least 21) nucleotides in length. In some embodiments, D1 is in the range of about 12 to 30 nucleotides in length (e.g., 12 to 30, 12 to 27, 15 to 22, 18 to 22, 18 to 25, 18 to 27, 18 to 30 or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29 or 30 nucleotides in length. In some embodiments, D1 is 20 nucleotides in length. In some embodiments, D1 comprising sense strand and antisense strand does not span the entire length of the sense strand and/or antisense strand. In some embodiments, D1 comprising the sense strand and antisense strand spans the entire length of either the sense strand or antisense strand or both. In certain embodiments, D1 comprising the sense strand and antisense strand spans the entire length of both the sense strand and the antisense strand.

In some embodiments, an oligonucleotide provided herein comprises a sense strand having a sequence of any one of SEQ ID NOs: 1-2 and 4-241 and an antisense strand comprising a complementary sequence selected from SEQ ID NOs: 242-243 and 245-482. In some embodiments, an oligonucleotide provided herein comprises a sense strand having a sequence of SEQ ID NO: 2, and an antisense strand comprising a complementary sequence of SEQ ID NO: 245. In some embodiments, an oligonucleotide provided herein comprises a sense strand having a sequence of SEQ ID NO: 5, and an antisense strand comprising a complementary sequence of SEQ ID NO: 246. In some embodiments, an oligonucleotide provided herein comprises a sense strand having a sequence of SEQ ID NO: 9, and an antisense strand comprising a complementary sequence of SEQ ID NO: 250.

In some embodiments, an oligonucleotide provided herein (e.g., an RNAi oligonucleotide) comprises a sense strand having a sequence of any one of SEQ ID NOs: 483-484 and 486-723 and an antisense strand comprising a complementary sequence selected from SEQ ID NOs: 724-725 and 727-964.

In some embodiments, an oligonucleotide provided herein (e.g., an RNAi oligonucleotide) comprises a sense strand and an antisense strand comprising nucleotide sequences selected from:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively.

In some embodiments, an oligonucleotide provided herein (e.g., an RNAi oligonucleotide) comprises a sense strand and an antisense strand comprising nucleotide sequences selected from:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively.

In some embodiments, the sense strand comprises the sequence of SEQ ID NO: 484 and the antisense strand comprises the sequence of SEQ ID NO: 725. In some embodiments, the sense strand comprises the sequence of SEQ ID NO: 486 and the antisense strand comprises the sequence of SEQ ID NO: 727. In some embodiments, the sense strand comprises the sequence of SEQ ID NO: 487 and the antisense strand comprises the sequence of SEQ ID NO: 728. In some embodiments, the sense strand comprises the sequence of SEQ ID NO: 488 and the antisense strand comprises the sequence of SEQ ID NO: 729. In some embodiments, the sense strand comprises the sequence of SEQ ID NO: 489 and the antisense strand comprises the sequence of SEQ ID NO: 730. In some embodiments, the sense strand comprises the sequence of SEQ ID NO: 491 and the antisense strand comprises the sequence of SEQ ID NO: 732. In some embodiments, the sense strand comprises the sequence of SEQ ID NO: 502 and the antisense strand comprises the sequence of SEQ ID NO: 743.

It should be appreciated that, in some embodiments, sequences presented in the Sequence Listing may be referred to in describing the structure of an oligonucleotide (e.g., an RNAi oligonucleotide) or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, an oligonucleotide herein (e.g., an RNAi oligonucleotide) comprises a 25-nucleotide sense strand and a 27-nucleotide antisense strand that when acted upon by a Dicer enzyme results in an antisense strand that is incorporated into the mature RISC. In some embodiments, the 25-nucleotide sense strand comprises a sequence selected from SEQ ID NOs: 1-2 and 4-241. In some embodiments, the 27-nucleotide antisense strand comprises a sequence selected from SEQ ID NOs: 242-243 and 245-482. In some embodiments, the sense strand of the oligonucleotide is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the oligonucleotide is longer than 25 nucleotides (e.g., 26, 27, 28, 29 or 30 nucleotides). In some embodiments, the sense strand of the oligonucleotide comprises a nucleotide sequence selected from SEQ ID NOs: 483-484 and 486-723, wherein the nucleotide sequence is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the oligonucleotide comprises a nucleotide sequence selected from SEQ ID NOs: 483-484 and 486-723, wherein the nucleotide sequence is longer than 25 nucleotides (e.g., 26, 27, 28, 29 or 30 nucleotides).

In some embodiments, oligonucleotides herein (e.g., RNAi oligonucleotides) have one 5′ end that is thermodynamically less stable when compared to the other 5′ end. In some embodiments, an asymmetric oligonucleotide is provided that includes a blunt end at the 3′ end of a sense strand and a 3′-overhang at the 3′ end of an antisense strand. In some embodiments, the 3′-overhang on the antisense strand is about 1-8 nucleotides in length (e.g., 1, 2, 3, 4, 5, 6, 7 or 8 nucleotides in length). In some embodiments, the oligonucleotide has an overhang comprising two (2) nucleotides on the 3′ end of the antisense (guide) strand. However, other overhangs are possible. In some embodiments, an overhang is a 3′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. In some embodiments, the oligonucleotide comprises a targeting sequence or a region of complementary that is complementary to a contiguous sequence of nucleotides of any one of SEQ ID NOs: 1-2 and 4-241, and a 5′-overhang comprising a length of between 1 and 6 nucleotides. In some embodiments, the oligonucleotide comprises a sense strand comprising a nucleotide sequence selected from SEQ ID NOs: 483-484 and 486-723, wherein the oligonucleotide comprises a 5′-overhang comprising a length of between 1 and 6 nucleotides. In some embodiments, the oligonucleotide comprises an antisense strand comprising a nucleotide sequence selected from SEQ ID NOs: 724-725 and 727-964, wherein the oligonucleotide comprises a 5′-overhang comprising a length of between 1 and 6 nucleotides. In some embodiments, the oligonucleotide comprises a sense strand comprising a nucleotide sequence selected from SEQ ID NOs: 483-484 and 486-723 and antisense strand comprising a nucleotide sequence selected from SEQ ID NOs: 724-725 and 727-964, wherein the oligonucleotide comprises a 5′-overhang comprising a length of between 1 and 6 nucleotides.

In some embodiments, two (2) terminal nucleotides on the 3′ end of an antisense strand are modified. In some embodiments, the two (2) terminal nucleotides on the 3′ end of the antisense strand are complementary with the target mRNA (e.g., CD274 mRNA). In some embodiments, the two (2) terminal nucleotides on the 3′ end of the antisense strand are not complementary with the target mRNA. In some embodiments, the two (2) terminal nucleotides on the 3′ end of the antisense strand of an oligonucleotide herein are unpaired. In some embodiments, the two (2) terminal nucleotides on the 3′ end of the antisense strand of an oligonucleotide herein comprise unpaired purines or pyrimidines. In some embodiments, the two (2) terminal nucleotides on the 3′ end of the antisense strand of an oligonucleotide herein comprise unpaired purines. In some embodiments, the two (2) terminal nucleotides on the 3′ end of the antisense strand of an oligonucleotide herein comprise unpaired GG, AA, AG, or GA. In some embodiments, the two (2) terminal nucleotides on the 3′ end of the antisense strand of an oligonucleotide herein comprise an unpaired GG. In some embodiments, one or both of the two (2) terminal GG nucleotides on each 3′ end of an oligonucleotide herein is not complementary with the target mRNA. In some embodiments, the oligonucleotide comprises a targeting sequence or a region of complementary that is complementary to a contiguous sequence of nucleotides of any one of SEQ ID NOs: 1-2 and 4-241, wherein the two (2) terminal nucleotides on the 3′ end of the antisense strand of the oligonucleotide herein comprises an unpaired GG. In some embodiments, the oligonucleotide comprises an antisense strand comprising a nucleotide sequence selected from SEQ ID NOs: 242-243 and 245-482, wherein the two (2) terminal nucleotides on the 3′ end of the antisense strand of the oligonucleotide comprises an unpaired GG. In some embodiments, the oligonucleotide comprises a sense strand comprising a nucleotide sequence selected from SEQ ID NOs: 483-484 and 486-723 and antisense strand comprising a nucleotide sequence selected from SEQ ID NOs: 724-725 and 727-964, wherein the two (2) terminal nucleotides on the 3′ end of the antisense strand of the oligonucleotide comprises an unpaired GG.

In some embodiments, there is one or more (e.g., 1, 2, 3, 4 or 5) mismatch(s) between a sense and antisense strand comprising an oligonucleotide herein (e.g., an RNAi oligonucleotide). If there is more than one mismatch between a sense and antisense strand, they may be positioned consecutively (e.g., 2, 3 or more in a row), or interspersed throughout the region of complementarity. In some embodiments, the 3′ end of the sense strand comprises one or more mismatches. In some embodiments, two (2) mismatches are incorporated at the 3′ end of the sense strand. In some embodiments, base mismatches, or destabilization of segments at the 3′ end of the sense strand of an oligonucleotide herein improves or increases the potency of the oligonucleotide. In some embodiments, the sense and antisense strands of an oligonucleotide herein comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein there is one or more (e.g., 1, 2, 3, 4 or 5) mismatch(s) between the sense and antisense strands.

In some embodiments, an oligonucleotide provided herein (e.g., an RNAi oligonucleotide) comprises a sense strand and an antisense strand comprising nucleotide sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
wherein there is one or more (e.g., 1, 2, 3, 4 or 5) mismatch(s) between the sense and antisense strands.

In some embodiments, an oligonucleotide provided herein (e.g., an RNAi oligonucleotide) comprises a sense strand and an antisense strand comprising nucleotide sequences of:
(a) SEQ ID NOs: 484 and 725, respectively;
(b) SEQ ID NOs: 487 and 728, respectively; and,
(b) SEQ ID NOs: 491 and 732, respectively,
wherein there is one or more (e.g., 1, 2, 3, 4 or 5) mismatch(s) between the sense and antisense strands.

### Antisense Strands

In some embodiments, an antisense strand of an oligonucleotide herein (e.g., an RNAi oligonucleotide) is referred to as a “guide strand”. For example, an antisense strand that engages with RNA-induced silencing complex (RISC) and binds to an Argonaute protein such as Ago2, or engages with or binds to one or more similar factors, and directs silencing of a target gene, as the antisense strand is referred to as a guide strand. In some embodiments, a sense strand comprising a region of complementary to a guide strand is referred to herein as a “passenger strand.”

In some embodiments, an oligonucleotide herein (e.g., an RNAi oligonucleotide) comprises an antisense strand of up to about 50 nucleotides in length (e.g., up to 50, up to 40, up to 35, up to 30, up to 27, up to 25, up to 21, up to 19, up to 17 or up to 12 nucleotides in length).

In some embodiments, an oligonucleotide comprises an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, an oligonucleotide comprises an antisense strand in a range of about 12 to about 40 (e.g., 12 to 40, 12 to 36, 12 to 32, 12 to 28, 15 to 40, 15 to 36, 15 to 32, 15 to 28, 17 to 22, 17 to 25, 19 to 27, 19 to 30, 20 to 40, 22 to 40, 25 to 40 or 32 to 40) nucleotides in length. In some embodiments, an oligonucleotide comprises antisense strand of 15 to 30 nucleotides in length. In some embodiments, an antisense strand of any one of the oligonucleotides disclosed herein is of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, an oligonucleotide comprises an antisense strand of 22 nucleotides in length.

In some embodiments, an oligonucleotide disclosed herein (e.g., an RNAi oligonucleotide) for targeting CD274 comprises an antisense strand comprising or consisting of a sequence as set forth in any one of SEQ ID NOs: 242-243 and 245-482. In some embodiments, an oligonucleotide disclosed herein (e.g., an RNAi oligonucleotide) for targeting CD274 comprises an antisense strand comprising or consisting of a sequence as set forth in SEQ ID NO: 243. In some embodiments, an oligonucleotide disclosed herein (e.g., an RNAi oligonucleotide) for targeting CD274 comprises an antisense strand comprising or consisting of a sequence as set forth in SEQ ID NO: 246. In some embodiments, an oligonucleotide disclosed herein (e.g., an RNAi oligonucleotide) for targeting CD274 comprises an antisense strand comprising or consisting of a sequence as set forth in SEQ ID NO: 250. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 242-243 and 245-482. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in SEQ ID NO: 243. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in SEQ ID NO: 246. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in SEQ ID NO: 250. In some embodiments, an oligonucleotide disclosed herein for targeting CD274 comprises an antisense strand comprising or consisting of a sequence as set forth in any one of SEQ ID NOs: 724-725 and 727-964. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 724-725 and 727-964. In some embodiments, an oligonucleotide disclosed herein for targeting CD274 comprises an antisense strand comprising or consisting of a sequence as set forth in any one of SEQ ID NOs: 724-725 and 727-964. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 725, 727, 728, 729, 730, 732, and 743. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in SEQ ID NO: 725. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in SEQ ID NO: 728. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in SEQ ID NO: 732. In some embodiments, an oligonucleotide disclosed herein for targeting CD274 comprises an antisense strand comprising or consisting of a sequence as set forth in any one of SEQ ID NOs: 725, 727, 728, 729, 730, 732, and 743. In some embodiments, an oligonucleotide disclosed herein for targeting CD274 comprises an antisense strand comprising or consisting of a sequence as set forth in SEQ ID NO: 725. In some embodiments, an oligonucleotide disclosed herein for targeting CD274 comprises an antisense strand comprising or consisting of a sequence as set forth in SEQ ID NO: 728. In some embodiments, an oligonucleotide disclosed herein for targeting CD274 comprises an antisense strand comprising or consisting of a sequence as set forth in SEQ ID NO: 732.

In some embodiments, an oligonucleotide herein comprises an antisense strand comprising a nucleotide sequence selected from SEQ ID NOs: 242-243 and 245-482. In some embodiments, an oligonucleotide herein comprises an antisense strand comprising a nucleotide sequence selected from SEQ ID NOs: 243, 245, 246, 247, 248, 250, or 261. In some embodiments, an oligonucleotide herein comprises an antisense strand comprising a nucleotide sequence of SEQ ID NO: 243. In some embodiments, an oligonucleotide herein comprises an antisense strand comprising a nucleotide sequence of SEQ ID NO: 246. In some embodiments, an oligonucleotide herein comprises an antisense strand comprising a nucleotide sequence of SEQ ID NO: 250.

### Sense Strands

In some embodiments, an oligonucleotide disclosed herein (e.g., an RNAi oligonucleotide) for targeting CD274 mRNA and inhibiting CD274 expression comprises a sense strand sequence as set forth in any one of SEQ ID NOs: 1-2 and 4-241. In some embodiments, an oligonucleotide herein has a sense strand comprised of at least about 12 (e.g., at least 13, at least 14, at least 15, at least 16, at least 17, at least 18, or at least 19) contiguous nucleotides of a sequence as set forth in in any one of SEQ ID NOs: 1-2 and 4-241. In some embodiments, an oligonucleotide disclosed herein for targeting CD274 mRNA and inhibiting CD274 expression comprises a sense strand sequence as set forth in any one of SEQ ID NOs: 483-484 and 486-723. In some embodiments, an oligonucleotide herein has a sense strand comprised of least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 483-484 and 486-723. In some embodiments, an oligonucleotide disclosed herein for targeting CD274 mRNA and inhibiting CD274 expression comprises a sense strand sequence as set forth in any one of SEQ ID NOs: 484, 486, 487, 488, 489, 491, and 502. In some embodiments, an oligonucleotide disclosed herein for targeting CD274 mRNA and inhibiting CD274 expression comprises a sense strand sequence as set forth in SEQ ID NO: 484. In some embodiments, an oligonucleotide disclosed herein for targeting CD274 mRNA and inhibiting CD274 expression comprises a sense strand sequence as set forth in SEQ ID NO: 487. In some embodiments, an oligonucleotide disclosed herein for targeting CD274 mRNA and inhibiting CD274 expression comprises a sense strand sequence as set forth in SEQ ID NO: 491. In some embodiments, an oligonucleotide herein has a sense strand that comprise at least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 484, 486, 487, 488, 489, 491, and 502. In some embodiments, an oligonucleotide herein has a sense strand that comprise at least about 12 (e.g., at least 13, at least 14, at least 15, at least 16, at least 17, at least 18, at least 19, at least 20, at least 21, at least 22 or at least 23) contiguous nucleotides of a sequence as set forth in SEQ ID NO: 484. In some embodiments, an oligonucleotide herein has a sense strand that comprise at least about 12 (e.g., at least 13, at least 14, at least 15, at least 16, at least 17, at least 18, at least 19, at least 20, at least 21, at least 22 or at least 23) contiguous nucleotides of a sequence as set forth in SEQ ID NO: 487. In some embodiments, an oligonucleotide herein has a sense strand that comprise at least about 12 (e.g., at least 13, at least 14, at least 15, at least 16, at least 17, at least 18, at least 19, at least 20, at least 21, at least 22 or at least 23) contiguous nucleotides of a sequence as set forth in SEQ ID NO: 491. In some embodiments, an oligonucleotide herein has a sense strand that comprise at least about 12 (e.g., at least 13, at least 14, at least 15, at least 16, at least 17, at least 18, or at least 19) contiguous nucleotides of a sequence as set forth in any one of SEQ ID NOs: 2, 4, 5, 6, 7, 9, or 20.

In some embodiments, an oligonucleotide provided herein (e.g., an RNAi oligonucleotide) comprises a sense strand (or passenger strand) of up to about 50 nucleotides in length (e.g., up to 50, up to 40, up to 36, up to 30, up to 27, up to 25, up to 21, up to 19, up to 17 or up to 12 nucleotides in length). In some embodiments, an oligonucleotide herein comprises a sense strand of at least about 12 nucleotides in length (e.g., at least 12, at least 15, at least 19, at least 21, at least 25, at least 27, at least 30, at least 36 or at least 38 nucleotides in length). In some embodiments, an oligonucleotide herein comprises a sense strand in a range of about 12 to about 50 (e.g., 12 to 50, 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, an oligonucleotide herein comprises a sense strand of 15 to 50 nucleotides in length. In some embodiments, an oligonucleotide herein comprises a sense strand of 18 to 36 nucleotides in length. In some embodiments, an oligonucleotide herein comprises a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, an oligonucleotide herein comprises a sense strand of 36 nucleotides in length.

In some embodiments, an oligonucleotide provided herein (e.g., an RNAi oligonucleotide) comprises a sense strand comprising a stem-loop structure at the 3′ end of the sense strand. In some embodiments, the stem-loop is formed by intrastrand base pairing. In some embodiments, a sense strand comprises a stem-loop structure at its 5′ end. In some embodiments, the stem of the stem-loop comprises a duplex of 2, 3, 4, 5, 6, 7, 8, 9, 10, 11, 12, 13 or 14 nucleotides in length. In some embodiments, the stem of the stem-loop comprises a duplex of 2 nucleotides in length. In some embodiments, the stem of the stem-loop comprises a duplex of 3 nucleotides in length. In some embodiments, the stem of the stem-loop comprises a duplex of 4 nucleotides in length. In some embodiments, the stem of the stem-loop comprises a duplex of 5 nucleotides in length. In some embodiments, the stem of the stem-loop comprises a duplex of 6 nucleotides in length. In some embodiments, the stem of the stem-loop comprises a duplex of 7 nucleotides in length. In some embodiments, the stem of the stem-loop comprises a duplex of 8 nucleotides in length. In some embodiments, the stem of the stem-loop comprises a duplex of 9 nucleotides in length. In some embodiments, the stem of the stem-loop comprises a duplex of 10 nucleotides in length. In some embodiments, the stem of the stem-loop comprises a duplex of 11 nucleotides in length. In some embodiments, the stem of the stem-loop comprises a duplex of 12 nucleotides in length. In some embodiments, the stem of the stem-loop comprises a duplex of 13 nucleotides in length. In some embodiments, the stem of the stem-loop comprises a duplex of 14 nucleotides in length.

In some embodiments, a stem-loop provides the oligonucleotide protection against degradation (e.g., enzymatic degradation), facilitates or improves targeting and/or delivery to a target cell, tissue, or organ, or both. For example, in some embodiments, the loop of a stem-loop is comprised of nucleotides comprising one or more modifications that facilitate, improve, or increase targeting to a target mRNA (e.g., a CD274 mRNA), inhibition of target gene expression (e.g., CD274 expression), and/or delivery, uptake, and/or penetrance into a target cell, tissue, or organ, or a combination thereof. In some embodiments, the stem-loop itself or modification(s) to the stem-loop do not affect or do not substantially affect the inherent gene expression inhibition activity of the oligonucleotide, but facilitates, improves, or increases stability (e.g., provides protection against degradation) and/or delivery, uptake, and/or penetrance of the oligonucleotide to a target cell, tissue, or organ. In certain embodiments, an oligonucleotide herein comprises a sense strand comprising (e.g., at its 3′ end) a stem-loop set forth as: S1-L-S2, in which S1 is complementary to S2, and in which L forms a single-stranded loop of linked nucleotides between S1 and S2 of up to about 10 nucleotides in length (e.g., 3, 4, 5, 6, 7, 8, 9 or 10 nucleotides in length). In some embodiments, the loop (L) is 3 nucleotides in length. In some embodiments, the loop (L) is 4 nucleotides in length. In some embodiments, the loop (L) is 5 nucleotides in length. In some embodiments, the loop (L) is 6 nucleotides in length. In some embodiments, the loop (L) is 7 nucleotides in length. In some embodiments, the loop (L) is 8 nucleotides in length. In some embodiments, the loop (L) is 9 nucleotides in length. In some embodiments, the loop (L) is 10 nucleotides in length.

In some embodiments, the tetraloop comprises the sequence 5′-GAAA-3′. In some embodiments, the stem loop comprises the sequence 5′-GCAGCCGAAAGGCUGC-3′ (SEQ ID NO: 856).

In some embodiments, an oligonucleotide provided herein (e.g., an RNAi oligonucleotide) comprises a targeting sequence or a region of complementary that is complementary to a contiguous sequence of nucleotides of any one of SEQ ID NOs: 1-2 and 4-241, and the oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the oligonucleotide comprises a targeting sequence or a region of complementary that is complementary to a contiguous sequence of nucleotides of any one of SEQ ID NOs: 1-2 and 4-241, and the oligonucleotide comprises a sense strand comprising (e.g., at its 3′ end) a stem-loop set forth as: S1-L-S2, in which S1 is complementary to S2, and in which L forms a single-stranded loop between S1 and S2 of 4 nucleotides in length.

In some embodiments, an oligonucleotide provided herein (e.g., an RNAi oligonucleotide) comprises a targeting sequence or a region of complementary that is complementary to a contiguous sequence of nucleotides of SEQ ID NO: 2, and the oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the oligonucleotide comprises a targeting sequence or a region of complementary that is complementary to a contiguous sequence of nucleotides of SEQ ID NO: 2, and the oligonucleotide comprises a sense strand comprising (e.g., at its 3′ end) a stem-loop set forth as: S1-L-S2, in which S1 is complementary to S2, and in which L forms a single-stranded loop between S1 and S2 of 4 nucleotides in length. In some embodiments, an oligonucleotide provided herein (e.g., an RNAi oligonucleotide) comprises a targeting sequence or a region of complementary that is complementary to a contiguous sequence of nucleotides of SEQ ID NO: 5, and the oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the oligonucleotide comprises a targeting sequence or a region of complementary that is complementary to a contiguous sequence of nucleotides of SEQ ID NO: 5, and the oligonucleotide comprises a sense strand comprising (e.g., at its 3′ end) a stem-loop set forth as: S1-L-S2, in which S1 is complementary to S2, and in which L forms a single-stranded loop between S1 and S2 of 4 nucleotides in length. In some embodiments, an oligonucleotide provided herein (e.g., an RNAi oligonucleotide) comprises a targeting sequence or a region of complementary that is complementary to a contiguous sequence of nucleotides of SEQ ID NO: 9, and the oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the oligonucleotide comprises a targeting sequence or a region of complementary that is complementary to a contiguous sequence of nucleotides of SEQ ID NO: 9, and the oligonucleotide comprises a sense strand comprising (e.g., at its 3′ end) a stem-loop set forth as: S1-L-S2, in which S1 is complementary to S2, and in which L forms a single-stranded loop between S1 and S2 of 4 nucleotides in length.

In some embodiments, a loop (L) of a stem-loop having the structure S1-L-S2 as described herein is a triloop. In some embodiments, the oligonucleotide comprises a targeting sequence or a region of complementary that is complementary to a contiguous sequence of nucleotides of any one of SEQ ID NOs: 1-2 and 4-241 and a triloop. In some embodiments, the triloop comprises ribonucleotides, deoxyribonucleotides, modified nucleotides, ligands (e.g., delivery ligands), and combinations thereof.

In some embodiments, a loop (L) of a stem-loop having the structure S1-L-S2 as described above is a tetraloop as describe in U.S. Pat. No. 10,131,912, incorporated herein by reference. In some embodiments, an oligonucleotide herein comprises a targeting sequence or a region of complementary that is complementary to a contiguous sequence of nucleotides of any one of SEQ ID NOs: 1-2 and 4-241 and a tetraloop. In some embodiments, the tetraloop comprises ribonucleotides, deoxyribonucleotides, modified nucleotides, ligands (e.g., delivery ligands), and combinations thereof.

### Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 29, 21, 22, 23, 24, 25, 26, 27, 28, 29 or 30 nucleotides in length. In some embodiments, a duplex formed between a sense and antisense strand is 12 nucleotides in length. In some embodiments, a duplex formed between a sense and antisense strand is 13 nucleotides in length. In some embodiments, a duplex formed between a sense and antisense strand is 14 nucleotides in length. In some embodiments, a duplex formed between a sense and antisense strand is 15 nucleotides in length. In some embodiments, a duplex formed between a sense and antisense strand is 16 nucleotides in length. In some embodiments, a duplex formed between a sense and antisense strand is 17 nucleotides in length. In some embodiments, a duplex formed between a sense and antisense strand is 18 nucleotides in length. In some embodiments, a duplex formed between a sense and antisense strand is 19 nucleotides in length. In some embodiments, a duplex formed between a sense and antisense strand is 20 nucleotides in length. In some embodiments, a duplex formed between a sense and antisense strand is 21 nucleotides in length. In some embodiments, a duplex formed between a sense and antisense strand is 22 nucleotides in length. In some embodiments, a duplex formed between a sense and antisense strand is 23 nucleotides in length. In some embodiments, a duplex formed between a sense and antisense strand is 24 nucleotides in length. In some embodiments, a duplex formed between a sense and antisense strand is 25 nucleotides in length. In some embodiments, a duplex formed between a sense and antisense strand is 26 nucleotides in length. In some embodiments, a duplex formed between a sense and antisense strand is 27 nucleotides in length. In some embodiments, a duplex formed between a sense and antisense strand is 28 nucleotides in length. In some embodiments, a duplex formed between a sense and antisense strand is 29 nucleotides in length. In some embodiments, a duplex formed between a sense and antisense strand is 30 nucleotides in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand. In some embodiments, the sense and antisense strands of an oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length)

In some embodiments, a duplex between a sense and antisense strand spans the entire length of both the sense strand and the antisense strand. In some embodiments, the sense and antisense strands of an oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743,
   respectively wherein a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length).

In some embodiments, a duplex between a sense and antisense strand spans the entire length of both the sense strand and the antisense strand. In some embodiments, the sense and antisense strands of an oligonucleotide comprise nucleotides sequences selected from:
(a) SEQ ID NOs: 484 and 725, respectively;
(b) SEQ ID NOs: 487 and 728, respectively; and,
(c) SEQ ID NOs: 491 and 732, respectively,
   respectively wherein a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length).

### Oligonucleotide Termini

In some embodiments, an oligonucleotide disclosed herein (e.g., an RNAi oligonucleotide) comprises a sense strand and an antisense strand, wherein the termini of either or both strands comprise a blunt end. In some embodiments, an oligonucleotide herein comprises sense and antisense strands that are separate strands which form an asymmetric duplex region having an overhang at the 3′ terminus of the antisense strand. In some embodiments, an oligonucleotide herein comprises a sense strand and an antisense strand, wherein the termini of either or both strands comprise an overhang comprising one or more nucleotides. In some embodiments, the one or more nucleotides comprising the overhang are unpaired nucleotides. In some embodiments, an oligonucleotide herein comprises a sense strand and an antisense strand, wherein the 3′ termini of the sense strand and the 5′ termini of the antisense strand comprise a blunt end. In some embodiments, an oligonucleotide herein comprises a sense strand and an antisense strand, wherein the 5′ termini of the sense strand and the 3′ termini of the antisense strand comprise a blunt end.

In some embodiments, an oligonucleotide herein comprises a sense strand and an antisense strand, wherein the 3′ terminus of either or both strands comprise a 3′-overhang comprising one or more nucleotides. In some embodiments, an oligonucleotide herein comprises a sense strand and an antisense strand, wherein the sense strand comprises a 3′-overhang comprising one or more nucleotides. In some embodiments, an oligonucleotide herein comprises a sense strand and an antisense strand, wherein the antisense strand comprises a 3′-overhang comprising one or more nucleotides. In some embodiments, an oligonucleotide herein comprises a sense strand and an antisense strand, wherein both the sense strand and the antisense strand comprises a 3′-overhang comprising one or more nucleotides.

In some embodiments, the 3′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 3′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 3′-overhang is (1) nucleotide in length. In some embodiments, the 3′-overhang is two (2) nucleotides in length. In some embodiments, the 3′-overhang is three (3) nucleotides in length. In some embodiments, the 3′-overhang is four (4) nucleotides in length. In some embodiments, the 3′-overhang is five (5) nucleotides in length. In some embodiments, the 3′-overhang is six (6) nucleotides in length. In some embodiments, the 3′-overhang is seven (7) nucleotides in length. In some embodiments, the 3′-overhang is eight (8) nucleotides in length. In some embodiments, the 3′-overhang is nine (9) nucleotides in length. In some embodiments, the 3′-overhang is ten (10) nucleotides in length. In some embodiments, the 3′-overhang is eleven (11) nucleotides in length. In some embodiments, the 3′-overhang is twelve (12) nucleotides in length. In some embodiments, the 3′-overhang is thirteen (13) nucleotides in length. In some embodiments, the 3′-overhang is fourteen (14) nucleotides in length. In some embodiments, the 3′-overhang is fifteen (15) nucleotides in length. In some embodiments, the 3′-overhang is sixteen (16) nucleotides in length. In some embodiments, the 3′-overhang is seventeen (17) nucleotides in length. In some embodiments, the 3′-overhang is eighteen (18) nucleotides in length. In some embodiments, the 3′-overhang is nineteen (19) nucleotides in length. In some embodiments, the 3′-overhang is twenty (20) nucleotides in length.

In some embodiments, an oligonucleotide disclosed herein (e.g., an RNAi oligonucleotide) comprises a sense strand and an antisense strand, wherein the antisense strand comprises a 3′-overhang, wherein the sense and antisense strands of the oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   and wherein the antisense strand comprises a 3′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 3′-overhang is two (2) nucleotides in length.

In some embodiments, an oligonucleotide disclosed herein (e.g., an RNAi oligonucleotide) comprises a sense strand and an antisense strand, wherein the antisense strand comprises a 3′-overhang, wherein the sense and antisense strands of the oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   and wherein the antisense strand comprises a 3′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 3′-overhang is two (2) nucleotides in length.

In some embodiments, an oligonucleotide disclosed herein (e.g., an RNAi oligonucleotide) comprises a sense strand and an antisense strand, wherein the antisense strand comprises a 3′-overhang, wherein the sense and antisense strands of the oligonucleotide comprise nucleotides sequences of:
(a) SEQ ID NOs: 484 and 725, respectively;
(b) SEQ ID NOs: 487 and 728, respectively; and,
(c) SEQ ID NOs: 491 and 732, respectively,
   and wherein the antisense strand comprises a 3′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 3′-overhang is two (2) nucleotides in length.

In some embodiments, an oligonucleotide herein comprises a sense strand and an antisense strand, wherein the 5′ terminus of either or both strands comprise a 5′-overhang comprising one or more nucleotides. In some embodiments, an oligonucleotide herein comprises a sense strand and an antisense strand, wherein the sense strand comprises a 5′-overhang comprising one or more nucleotides. In some embodiments, an oligonucleotide herein comprises a sense strand and an antisense strand, wherein the antisense strand comprises a 5′-overhang comprising one or more nucleotides. In some embodiments, an oligonucleotide herein comprises a sense strand and an antisense strand, wherein both the sense strand and the antisense strand comprises a 5′-overhang comprising one or more nucleotides.

In some embodiments, the 5′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 5′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 5′-overhang is (1) nucleotide in length. In some embodiments, the 5′-overhang is two (2) nucleotides in length. In some embodiments, the 5′-overhang is three (3) nucleotides in length. In some embodiments, the 5′-overhang is four (4) nucleotides in length. In some embodiments, the 5′-overhang is five (5) nucleotides in length. In some embodiments, the 5′-overhang is six (6) nucleotides in length. In some embodiments, the 5′-overhang is seven (7) nucleotides in length. In some embodiments, the 5′-overhang is eight (8) nucleotides in length. In some embodiments, the 5′-overhang is nine (9) nucleotides in length. In some embodiments, the 5′-overhang is ten (10) nucleotides in length. In some embodiments, the 5′-overhang is eleven (11) nucleotides in length. In some embodiments, the 5′-overhang is twelve (12) nucleotides in length. In some embodiments, the 5′-overhang is thirteen (13) nucleotides in length. In some embodiments, the 5′-overhang is fourteen (14) nucleotides in length. In some embodiments, the 5′-overhang is fifteen (15) nucleotides in length. In some embodiments, the 5′-overhang is sixteen (16) nucleotides in length. In some embodiments, the 5′-overhang is seventeen (17) nucleotides in length. In some embodiments, the 5′-overhang is eighteen (18) nucleotides in length. In some embodiments, the 5′-overhang is nineteen (19) nucleotides in length. In some embodiments, the 5′-overhang is twenty (20) nucleotides in length.

In some embodiments, an oligonucleotide disclosed herein (e.g., an RNAi oligonucleotide) comprises a sense strand and an antisense strand, wherein the antisense strand comprises a 5′-overhang, wherein the sense and antisense strands of the oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   and wherein the antisense strand comprises a 3′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 3′-overhang is two (2) nucleotides in length.

In some embodiments, an oligonucleotide disclosed herein (e.g., an RNAi oligonucleotide) comprises a sense strand and an antisense strand, wherein the antisense strand comprises a 5′-overhang, wherein the sense and antisense strands of the oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   and wherein the antisense strand comprises a 5′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 5′-overhang is two (2) nucleotides in length.

In some embodiments, an oligonucleotide disclosed herein (e.g., an RNAi oligonucleotide) comprises a sense strand and an antisense strand, wherein the antisense strand comprises a 5′-overhang, wherein the sense and antisense strands of the oligonucleotide comprise nucleotides sequences of:
(a) SEQ ID NOs: 484 and 725, respectively;
(b) SEQ ID NOs: 487 and 728, respectively; and,
(c) SEQ ID NOs: 491 and 732, respectively,
   and wherein the antisense strand comprises a 5′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 5′-overhang is two (2) nucleotides in length.

In some embodiments, one or more (e.g., 2, 3, 4, 5, or more) nucleotides comprising the 3′ terminus or 5′ terminus of a sense and/or antisense strand are modified. For example, in some embodiments, one or two terminal nucleotides of the 3′ terminus of the antisense strand are modified. In some embodiments, the last nucleotide at the 3′ terminus of an antisense strand is modified, such that it comprises 2′ modification, or it comprises, a 2′-O-methoxyethyl. In some embodiments, the last one or two terminal nucleotides at the 3′ terminus of an antisense strand are complementary with the target. In some embodiments, the last one or two nucleotides at the 3′ terminus of the antisense strand are not complementary with the target.

In some embodiments, an oligonucleotide disclosed herein (e.g., an RNAi oligonucleotide) comprises a sense strand and an antisense strand, wherein the 3′ terminus of the sense strand comprises a step-loop described herein and the 3′ terminus of the antisense strand comprises a 3′-overhang described herein. In some embodiments, an oligonucleotide herein (e.g., an RNAi oligonucleotide) comprises a sense strand and an antisense strand that form a nicked tetraloop structure described herein, wherein the 3′ terminus of the sense strand comprises a stem-loop, wherein the loop is a tetraloop described herein, and wherein the 3′ terminus of the antisense strand comprises a 3′-overhang described herein. In some embodiments, the 3′-overhang is two (2) nucleotides in length. In some embodiments, the two (2) nucleotides comprising the 3′-overhang both comprise guanine (G) nucleobases. Typically, one or both of the nucleotides comprising the 3′-overhang of the antisense strand are not complementary with the target mRNA.

### Oligonucleotide Modifications

In some embodiments, an oligonucleotide described herein (e.g., an RNAi oligonucleotide) comprises a modification. Oligonucleotides (e.g., RNAi oligonucleotides) may be modified in various ways to improve or control specificity, stability, delivery, bioavailability, resistance from nuclease degradation, immunogenicity, base-pairing properties, RNA distribution and cellular uptake and other features relevant to therapeutic or research use.

In some embodiments, the modification is a modified sugar. In some embodiments, the modification is a 5′-terminal phosphate group. In some embodiments, the modification is a modified internucleotide linkage. In some embodiments, the modification is a modified base. In some embodiments, an oligonucleotide described herein can comprise any one of the modifications described herein or any combination thereof. For example, in some embodiments, an oligonucleotide described herein comprises at least one modified sugar, a 5′-terminal phosphate group, at least one modified internucleotide linkage, and at least one modified base. In some embodiments, the sense and antisense strands of an oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein the oligonucleotide comprises at least one modified sugar, a 5′-terminal phosphate group, at least one modified internucleotide linkage, and at least one modified base.

In some embodiments, an oligonucleotide described herein comprises at least one modified sugar, a 5′-terminal phosphate group, at least one modified internucleotide linkage, and at least one modified base. In some embodiments, the sense and antisense strands of an oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   wherein the oligonucleotide comprises at least one modified sugar, a 5′-terminal phosphate group, at least one modified internucleotide linkage, and at least one modified base.
   In some embodiments, an oligonucleotide described herein comprises at least one modified sugar, a 5′-terminal phosphate group, at least one modified internucleotide linkage, and at least one modified base. In some embodiments, the sense and antisense strands of an oligonucleotide comprise nucleotides sequences selected from the group consisting of:
(a) SEQ ID NOs: 484 and 725, respectively;
(b) SEQ ID NOs: 487 and 728, respectively; and,
(c) SEQ ID NOs: 491 and 732, respectively,
   wherein the oligonucleotide comprises at least one modified sugar, a 5′-terminal phosphate group, at least one modified internucleotide linkage, and at least one modified base.

The number of modifications on an oligonucleotide (e.g., an RNAi oligonucleotide) and the position of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in some embodiments, all or substantially all the nucleotides of an oligonucleotide are modified. In some embodiments, more than half of the nucleotides are modified. In some embodiments, less than half of the nucleotides are modified. In some embodiments, the sugar moiety of all nucleotides comprising the oligonucleotide is modified at the 2′ position. The modifications may be reversible or irreversible. In some embodiments, an oligonucleotide as disclosed herein has a number and type of modified nucleotides sufficient to cause the desired characteristics (e.g., protection from enzymatic degradation, capacity to target a desired cell after in vivo administration, and/or thermodynamic stability).

### Sugar Modifications

In some embodiments, an oligonucleotide described herein (e.g., an RNAi oligonucleotide) comprises a modified sugar. In some embodiments, a modified sugar (also referred herein to a sugar analog) includes a modified deoxyribose or ribose moiety in which, for example, one or more modifications occur at the 2′, 3′, 4′ and/or 5′ carbon position of the sugar. In some embodiments, a modified sugar may also include non-natural alternative carbon structures such as those present in locked nucleic acids (“LNA”; see, e.g., Koshkin et al. (1998) TETRAHEDRON 54:3607-30), unlocked nucleic acids (“UNA”; see, e.g., Snead et al. (2013) MOL. THER-NUCL. ACIDS 2:e103) and bridged nucleic acids (“BNA”; see, e.g., Imanishi & Obika (2002) CHEM COMMUN. (CAMB) 21:1653-59).

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl](2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, an oligonucleotide (e.g., an RNAi oligonucleotide) described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the oligonucleotide comprises at least about 1 modified nucleotide (e.g., at least 1, at least 5, at least 10, at least 15, at least 20, at least 25, at least 30, at least 35, or more). In some embodiments, the antisense strand of the oligonucleotide comprises at least about 1 modified nucleotide (e.g., at least 1, at least 5, at least 10, at least 15, at least 20, or more).

In some embodiments, all the nucleotides of the sense strand of the oligonucleotide are modified. In some embodiments, all the nucleotides of the antisense strand of the oligonucleotide are modified. In some embodiments, all the nucleotides of the oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid).

In some embodiments, the disclosure provides oligonucleotides having different modification patterns. In some embodiments, an oligonucleotide herein comprises a sense strand having a modification pattern as set forth in the Examples and Sequence Listing and an antisense strand having a modification pattern as set forth in the Examples and Sequence Listing.

In some embodiments, an oligonucleotide disclosed herein (e.g., an RNAi oligonucleotide) comprises an antisense strand having nucleotides that are modified with 2′-F. In some embodiments, an oligonucleotide herein comprises an antisense strand comprising nucleotides that are modified with 2′-F and 2′-OMe. In some embodiments, an oligonucleotide disclosed herein comprises a sense strand having nucleotides that are modified with 2′-F. In some embodiments, an oligonucleotide disclosed herein comprises a sense strand comprises nucleotides that are modified with 2′-F and 2′-OMe.

In some embodiments, an oligonucleotide described herein comprises a sense strand with about 10-15%, 10%, 11%, 12%, 13%, 14% or 15% of the nucleotides of the sense strand comprising a 2′-fluoro modification. In some embodiments, about 11% of the nucleotides of the sense strand comprise a 2-fluoro modification. In some embodiments, an oligonucleotide described herein comprises an antisense strand with about 25-35%, 25%, 26%, 27%, 28%, 29%, 30%, 31%, 32%, 33%, 34% or 35% of the nucleotides of the antisense strand comprising a 2′-fluoro modification. In some embodiments, about 32% of the nucleotides of the antisense strand comprise a 2′-fluoro modification. In some embodiments, the oligonucleotide has about 15-25%, 15%, 16%, 17%, 18%, 19%, 20%, 21%, 22%, 23%, 24%, or 25% of its nucleotides comprising a 2′-fluoro modification. In some embodiments, about 19% of the nucleotides in the oligonucleotide comprise a 2′-fluoro modification.

In some embodiments, one or more of positions 8, 9, 10 or 11 of the sense strand is modified with a 2′-F group. In some embodiments, one or more of positions 3, 8, 9, 10, 12, 13 and 17 of the sense strand is modified with a 2′-F group. In some embodiments, one or more of positions 2, 3, 4, 5, 7, 10 and 14 of the antisense strand is modified with a 2′-F group. In some embodiments, one or more of positions 2, 3, 4, 5, 7, 8, 10, 14, 16 and 19 is modified with a 2′-F group. In some embodiments, the sugar moiety at each of nucleotides at positions 1-7 and 12-20 in the sense strand is modified with a 2′-OMe. In some embodiments, the sugar moiety at each of nucleotides at positions 1-7, 12-27 and 31-36 in the sense strand is modified with a 2′-OMe. In some embodiments, the sugar moiety at each of nucleotides at positions 6, 9, 11-13, 15, 17, 18 and 20-22 in the sense strand is modified with a 2′-OMe.

In some embodiments, the sense and antisense strands of an oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein one or more of positions 8, 9, 10 or 11 of the sense strand is modified with a 2′-F group.

In some embodiments, the sense and antisense strands of an oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   wherein one or more of positions 8, 9, 10 or 11 of the sense strand is modified with a 2′-F group.

In some embodiments, the sense and antisense strands of an oligonucleotide comprise nucleotides sequences selected from the group consisting of:
(a) SEQ ID NOs: 484 and 725, respectively;
(b) SEQ ID NOs: 487 and 728, respectively; and,
(c) SEQ ID NOs: 491 and 732, respectively,
   wherein one or more of positions 8, 9, 10 or 11 of the sense strand is modified with a 2′-F group.

In some embodiments, an oligonucleotide provided herein comprises an antisense strand having the sugar moiety of each of the nucleotides at positions 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 4, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 3, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 3, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, 8, 10, 14, 16 and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-F.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-OMe.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises a sense strand having the sugar moiety at positions 8-11 modified with 2′-F. In some embodiments, an oligonucleotide provided herein comprises a sense strand having the sugar moiety at positions 3, 8, 9, 10, 12, 13 and 17 modified with 2′-F. In some embodiments, an oligonucleotide provided herein comprises a sense strand having the sugar moiety at positions 1-7 and 12-17 or 12-20 modified with 2′OMe. In some embodiments, an oligonucleotide provided herein comprises a sense strand having the sugar moiety at positions 1-7, 12-27 and 31-36 modified with 2′OMe. In some embodiments, an oligonucleotide provided herein comprises a sense strand having the sugar moiety of each of the nucleotides at positions 1-7 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA). In some embodiments, an oligonucleotide provided herein comprises a sense strand having the sugar moiety at positions 1-2, 4-7, 11, 14-16 and 18-20 modified with 2′OMe. In some embodiments, an oligonucleotide provided herein comprises a sense strand having the sugar moiety of each of the nucleotides at positions 1-2, 4-7, 11, 14-16 and 18-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-F.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-OMe.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

### 5′-Terminal Phosphate

In some embodiments, an oligonucleotide described herein (e.g., an RNAi oligonucleotide) comprises a sense strand and an antisense strand, wherein the antisense strand comprises a 5′-terminal phosphate. In some embodiments, 5′-terminal phosphate groups of an RNAi oligonucleotide enhance the interaction with Ago2. However, oligonucleotides comprising a 5′-phosphate group may be susceptible to degradation via phosphatases or other enzymes, which can limit their performance and/or bioavailability in vivo. In some embodiments, an oligonucleotide herein includes analogs of 5′ phosphates that are resistant to such degradation. In some embodiments, the phosphate analog is oxymethyl phosphonate, vinylphosphonate or malonylphosphonate, or a combination thereof. In certain embodiments, the 5′ terminus of an oligonucleotide strand is attached to chemical moiety that mimics the electrostatic and steric properties of a natural 5′-phosphate group (“phosphate mimic”). In some embodiments, the sense and antisense strands of an oligonucleotide comprise nucleotide sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein the antisense strand comprises a 5′-terminal phosphate, optionally a 5′-terminal phosphate analog.

In some embodiments, the sense and antisense strands of an oligonucleotide comprise nucleotide sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   wherein the antisense strand comprises a 5′-terminal phosphate, optionally a 5′-terminal phosphate analog.

In some embodiments, the sense and antisense strands of an oligonucleotide comprise nucleotide sequences from the group consisting of: (a) SEQ ID NOs: 484 and 725, respectively;
(a) SEQ ID NOs: 484 and 725, respectively;
(b) SEQ ID NOs: 487 and 728, respectively; and,
(c) SEQ ID NOs: 491 and 732, respectively,
   wherein the antisense strand comprises a 5′-terminal phosphate, optionally a 5′-terminal phosphate analog.

In some embodiments, an oligonucleotide herein (e.g., an RNAi oligonucleotide) has a phosphate analog at a 4′-carbon position of the sugar (referred to as a “4′-phosphate analog”). See, e.g., Intl. Patent Application Publication No. WO 2018/045317. In some embodiments, an oligonucleotide herein comprises a 4′-phosphate analog at a 5′-terminal nucleotide. In some embodiments, a phosphate analog is an oxymethyl phosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the amino methyl group is bound to the 4′-carbon of the sugar moiety or analog thereof. In certain embodiments, a 4′-phosphate analog is an oxymethyl phosphonate. In some embodiments, an oxymethyl phosphonate is represented by the formula —O—CH₂—PO(OH)₂, —O—CH₂—PO(OR)₂, or —O—CH2-POOH(R), in which R is independently selected from H, CH₃, an alkyl group, CH₂CH₂CN, CH₂OCOC(CH₃)₃, CH₂OCH₂CH₂Si(CH₃)₃ or a protecting group. In certain embodiments, the alkyl group is CH₂CH₃. More typically, R is independently selected from H, CH₃ or CH₂CH₃. In some embodiment, R is CH3. In some embodiments, the 4′-phosphate analog is 4′-oxymethyl phosphonate.

In some embodiments, an oligonucleotide provided herein comprises an antisense strand comprising a 4′-phosphate analog at the 5′-terminal nucleotide, wherein 5′-terminal nucleotide comprises the following structure:

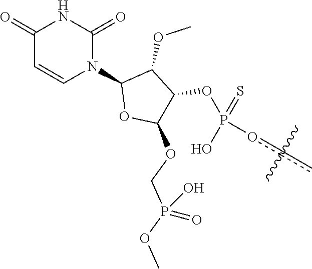

4′-O-monomethylphosphonate-2′-O-methyluridine phosphorothioate [MePhosphonate-4O-mUs].

### Modified Internucleotide Linkage

In some embodiments, an oligonucleotide provided herein (e.g., a RNAi oligonucleotide) comprises a modified internucleotide linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, an oligonucleotide provided herein (e.g., a RNAi oligonucleotide) has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the sense and antisense strands of an oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein the oligonucleotide comprises a modified internucleotide linkage.

In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the sense and antisense strands of an oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   wherein the oligonucleotide comprises a modified internucleotide linkage.

In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the sense and antisense strands of an oligonucleotide comprise nucleotides sequences selected from the group consisting of:
(a) SEQ ID NOs: 484 and 725, respectively;
(b) SEQ ID NOs: 487 and 728, respectively; and,
(c) SEQ ID NOs: 491 and 732, respectively,
   wherein the oligonucleotide comprises a modified internucleotide linkage.

### Base Modifications

In some embodiments, an oligonucleotide provided herein (e.g., an RNAi oligonucleotides) comprises one or more modified nucleobases. In some embodiments, modified nucleobases (also referred to herein as base analogs) are linked at the 1′ position of a nucleotide sugar moiety. In certain embodiments, a modified nucleobase is a nitrogenous base. In some embodiments, a modified nucleobase does not contain nitrogen atom. See, e.g., US Patent Application Publication No. 2008/0274462. In some embodiments, a modified nucleotide comprises a universal base. In some embodiments, a modified nucleotide does not contain a nucleobase (abasic). In some embodiments, the sense and antisense strands of an oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively.
   wherein the oligonucleotide comprises one or more modified nucleobases.

In some embodiments, a modified nucleotide comprises a universal base. In some embodiments, a modified nucleotide does not contain a nucleobase (abasic). In some embodiments, the sense and antisense strands of an oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   wherein the oligonucleotide comprises one or more modified nucleobases.

In some embodiments, a modified nucleotide comprises a universal base. In some embodiments, a modified nucleotide does not contain a nucleobase (abasic). In some embodiments, the sense and antisense strands of an oligonucleotide comprise nucleotides sequences selected from the group consisting of:
(a) SEQ ID NOs: 484 and 725, respectively;
(b) SEQ ID NOs: 487 and 728, respectively; and,
(c) SEQ ID NOs: 491 and 732, respectively,
   wherein the oligonucleotide comprises one or more modified nucleobases.

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid (e.g., a CD274 mRNA), a single-stranded nucleic acid containing a universal base forms a duplex with the target nucleic acid that has a lower Tₘ than a duplex formed with the complementary nucleic acid. In some embodiments, when compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al. (1995) NUCLEIC ACIDS RES. 23:4363-4370; Loakes et al. (1995) NUCLEIC ACIDS RES. 23:2361-66; and Loakes & Brown (1994) NUCLEIC ACIDS RES. 22:4039-43).

### Targeting Ligands

In some embodiments, it is desirable to target an oligonucleotide provided herein (e.g., an RNAi oligonucleotide) to one or more cells or cell type, tissues, organs, or anatomical regions or compartments. Such a strategy may help to avoid undesirable effects to the organism treated and/or to avoid undue loss of the oligonucleotide to cells, tissues, organs, or anatomical regions or compartments that would not benefit from the oligonucleotide or its effects (e.g., inhibition or reduction of CD274 expression). Accordingly, in some embodiments, oligonucleotides disclosed herein (e.g., RNAi oligonucleotides) are modified to facilitate targeting and/or delivery to particular cells or cell types, tissues, organs, or anatomical regions or compartments (e.g., to facilitate delivery of the oligonucleotide to tumor). In some embodiments, an oligonucleotide comprises at least one nucleotide (e.g., 1, 2, 3, 4, 5, 6 or more nucleotides) conjugated to one or more targeting ligand(s). In some embodiments, the sense and antisense strands of an oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein the oligonucleotide comprises a targeting ligand conjugated to at least one nucleotide.

In some embodiments, an oligonucleotide comprises at least one nucleotide (e.g., 1, 2, 3, 4, 5, 6 or more nucleotides) conjugated to one or more targeting ligand(s). In some embodiments, the sense and antisense strands of an oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   wherein the oligonucleotide comprises a targeting ligand conjugated to at least one nucleotide.

In some embodiments, the sense and antisense strands of an oligonucleotide comprise nucleotides sequences of:
(a) SEQ ID NOs: 484 and 725, respectively;
(b) SEQ ID NOs: 487 and 728, respectively; and,
(c) SEQ ID NOs: 491 and 732, respectively,
   wherein the oligonucleotide comprises a targeting ligand conjugated to at least one nucleotide.

In some embodiments, the targeting ligand comprises a carbohydrate, amino sugar, cholesterol, peptide, polypeptide, protein, or part of a protein (e.g., an antibody or antibody fragment), or lipid. In certain embodiments, the targeting ligand is a carbohydrate comprising at least one GalNAc moiety.

In some embodiments, 1 or more (e.g., 1, 2, 3, 4, 5 or 6) nucleotides of an oligonucleotide provided herein (e.g., an RNAi oligonucleotide) are each conjugated to a separate targeting ligand (e.g., a GalNAc moiety). In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ terminus of the sense or antisense strand) such that the targeting ligands resemble bristles of a toothbrush and the oligonucleotide resembles a toothbrush. For example, an oligonucleotide may comprise a stem-loop at either the 5′ or 3′ terminus of the sense strand and 1, 2, 3 or 4 nucleotides of the loop of the stem may be individually conjugated to a targeting ligand. In some embodiments, an oligonucleotide provided by the disclosure (e.g., a RNAi oligonucleotide) comprises a stem-loop at the 3′ terminus of the sense strand, wherein the loop of the stem-loop comprises a triloop or a tetraloop, and wherein the 3 or 4 nucleotides comprising the triloop or tetraloop, respectively, are individually conjugated to a targeting ligand. In some embodiments, an oligonucleotide provided by the disclosure (e.g., a RNAi oligonucleotide) comprises a stem-loop at the 3′ terminus of the sense strand, wherein the loop of the stem-loop comprises a tetraloop, and wherein 3 nucleotides of the tetraloop are individually conjugated to a targeting ligand.

a. GalNAc Targeting Ligands

GalNAc is a high affinity carbohydrate ligand for the asialoglycoprotein receptor (ASGPR), which is primarily expressed on the surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotides of the instant disclosure can be used to target these oligonucleotides to the ASGPR expressed on cells. In some embodiments, an oligonucleotide of the instant disclosure (e.g., an RNAi oligonucleotide) is conjugated to at least one or more GalNAc moieties, wherein the GalNAc moieties target the oligonucleotide to an ASGPR expressed on human liver cells (e.g., human hepatocytes). In some embodiments, the GalNAc moiety target the oligonucleotide to the liver.

In some embodiments, an oligonucleotide of the instant disclosure (e.g., an RNAi oligonucleotide) is conjugated directly or indirectly to a monovalent GalNAc moiety. In some embodiments, the oligonucleotide is conjugated directly or indirectly to more than one monovalent GalNAc (i.e., is conjugated to 2, 3 or 4 monovalent GalNAc moieties and is typically conjugated to 3 or 4 monovalent GalNAc moieties). In some embodiments, an oligonucleotide is conjugated to one or more bivalent GalNAc, trivalent GalNAc or tetravalent GalNAc moieties. In some embodiments, a bivalent, trivalent or tetravalent GalNAc moiety is conjugated to an oligonucleotide via a branched linker. In some embodiments, a monovalent GalNAc moiety is conjugated to a first nucleotide and a bivalent, trivalent, or tetravalent GalNAc moiety is conjugated to a second nucleotide via a branched linker.

In some embodiments, one (1) or more (e.g., 1, 2, 3, 4, 5 or 6) nucleotides of an oligonucleotide described herein (e.g., an RNAi oligonucleotide) are each conjugated to a GalNAc moiety. In some embodiments, two (2) to four (4) nucleotides of a tetraloop are each conjugated to a separate GalNAc moiety. In some embodiments, one (1) to three (3) nucleotides of a triloop are each conjugated to a separate GalNAc moiety. In some embodiments, targeting ligands are conjugated to two (2) to four (4) nucleotides at either ends of the sense or antisense strand (e.g., ligands are conjugated to a two (2) to four (4) nucleotide overhang or extension on the 5′ or 3′ terminus of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, three (3) or four (4) GalNAc moieties can be conjugated to nucleotides in the tetraloop of the sense strand where each GalNAc moiety is conjugated to one (1) nucleotide.

In some embodiments, an oligonucleotide described herein (e.g., an RNAi oligonucleotide) comprises a tetraloop, wherein the tetraloop (L) is any combination of adenine (A) and guanine (G) nucleotides. In some embodiments, the tetraloop (L) comprises a monovalent GalNAc moiety attached to any one or more guanine (G) nucleotides of the tetraloop via any linker described herein, as depicted below (X=heteroatom):

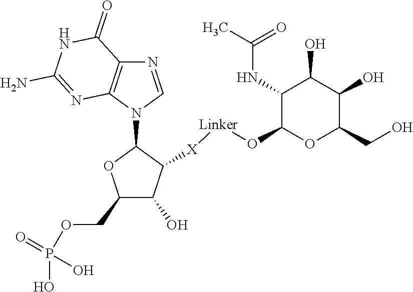

In some embodiments, the tetraloop (L) has a monovalent GalNAc attached to any one or more adenine nucleotides of the tetraloop via any linker described herein, as depicted below (X=heteroatom):

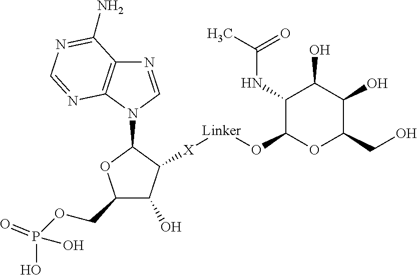

In some embodiments, an oligonucleotide herein (e.g., an RNAi oligonucleotide) comprises a monovalent GalNAc moiety attached to a guanine (G) nucleotide referred to as [ademG-GalNAc] or 2′-aminodiethoxymethanol-Guanine-GalNAc, as depicted below:

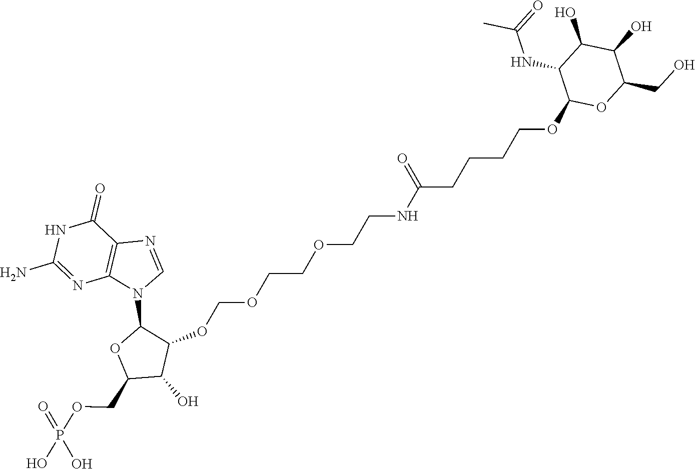 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an oligonucleotide herein comprises a monovalent GalNAc moiety attached to an adenine nucleotide, referred to as [ademA-GalNAc] or 2′-aminodiethoxymethanol-Adenine-GalNAc, as depicted below:

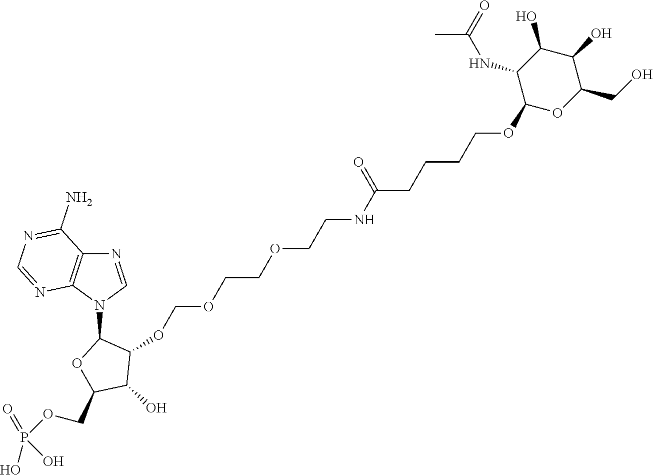 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

An example of such conjugation is shown below for a loop comprising from 5′ to 3′ the nucleotide sequence GAAA (L=linker, X=heteroatom). Such a loop may be present, for example, at positions 27-30 of a sense strand provided herein. In the chemical formula,

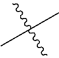

is used to describe an attachment point to the oligonucleotide strand.

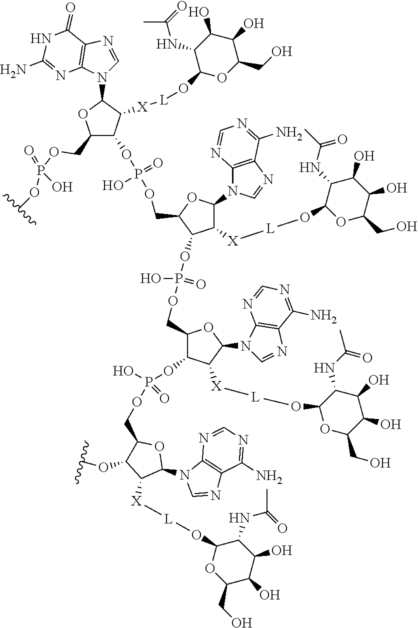

Appropriate methods or chemistry (e.g., click chemistry) can be used to link a targeting ligand to a nucleotide. In some embodiments, a targeting ligand is conjugated to a nucleotide comprising an oligonucleotide herein (e.g., an RNAi oligonucleotide) using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO2016/100401. In some embodiments, the linker is a labile linker. However, in other embodiments, the linker is stable. An example is shown below for a loop comprising from 5′ to 3′ the nucleotides GAAA, in which GalNAc moieties are attached to nucleotides of the loop using an acetal linker. Such a loop may be present, for example, at positions 27-30 of the any one of the sense strands. In the chemical formula,

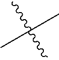

is an attachment point to the oligonucleotide strand.

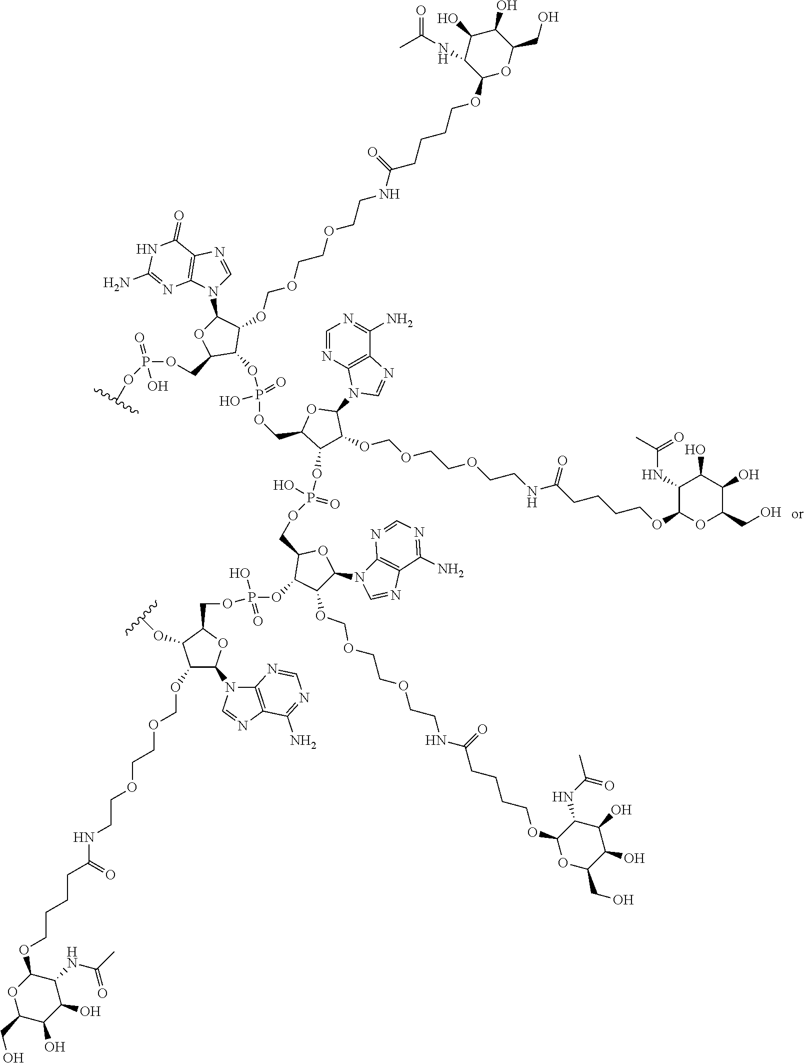 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

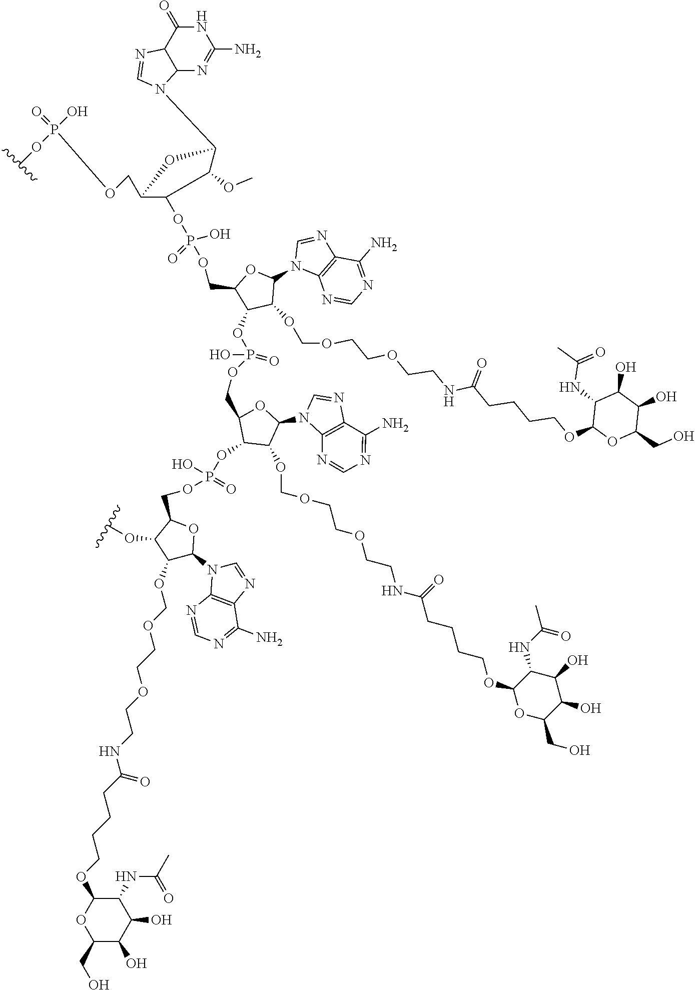

As mentioned various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and the oligonucleotide. In some embodiments, the oligonucleotides herein (e.g., RNAi oligonucleotides) do not have a GalNAc conjugated thereto.

In some embodiments, the sense and antisense strands of an oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein the oligonucleotide comprises at least one GalNAc moiety conjugated to a nucleotide.

In some embodiments, the sense and antisense strands of an oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   wherein the oligonucleotide comprises at least one GalNAc moiety conjugated to a nucleotide.
   b. Lipid Targeting Ligands

In some embodiments, the disclosure provides an oligonucleotide-ligand conjugate comprising an oligonucleotide comprising nucleotide sequence for inhibiting expression of a target mRNA expressed in tumor (e.g., CD274) and one or more targeting ligands conjugated to the oligonucleotide. In some embodiments, an oligonucleotide-ligand conjugate described herein comprises a nucleotide sequence and one or more targeting ligands, wherein the nucleotide sequence comprises one or more nucleosides (nucleic acids) conjugated with one or more targeting ligands represented by formula I-a:

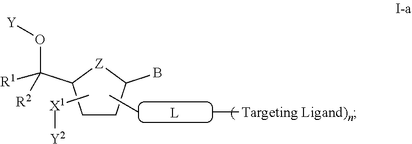

or a pharmaceutically acceptable salt thereof,
wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃; or
R¹ and R² on the same carbon are taken together with their intervening atoms to form a 3-7 membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur; or
two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
each targeting ligand is a lipid conjugate moiety (LC); and wherein each LC is independently a lipid conjugate moiety comprising a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, —V¹CR²W¹—, or

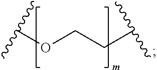

m is 1-50;
X¹, V¹ and W¹ are independently —C(R)₂—, —OR, —O—, —S—, —Se—, or —NR—;
Y is hydrogen, a suitable hydroxyl protecting group,

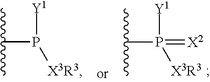

R³ is hydrogen, a suitable protecting group, a suitable prodrug, or an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, S, or NR;
X³ is —O—, —S—, —BH₂—, or a covalent bond;
Y¹ is a linking group attaching to the 2′- or 3′-terminal of a nucleoside, a nucleotide, or an oligonucleotide;
Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support; and
Z is —O—, —S—, —NR—, or —CR₂—.

In some embodiments, the oligonucleotide-ligand conjugate comprises one or more nucleic acids conjugated with targeting ligands represented by formula II-a:

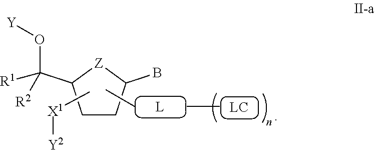

or a pharmaceutically acceptable salt thereof.

In some embodiments, the oligonucleotide-ligand conjugate comprises one or more nucleic acids conjugated with targeting ligands represented by formula II-b or II-c:

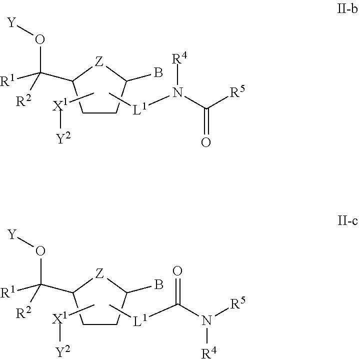

or a pharmaceutically acceptable salt thereof, wherein:
L¹ is a covalent bond, a monovalent or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or

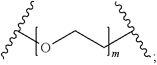

R⁴ is hydrogen, R^{A}, or a suitable amine protection group; and
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR.

In some embodiments, R⁵ is selected from

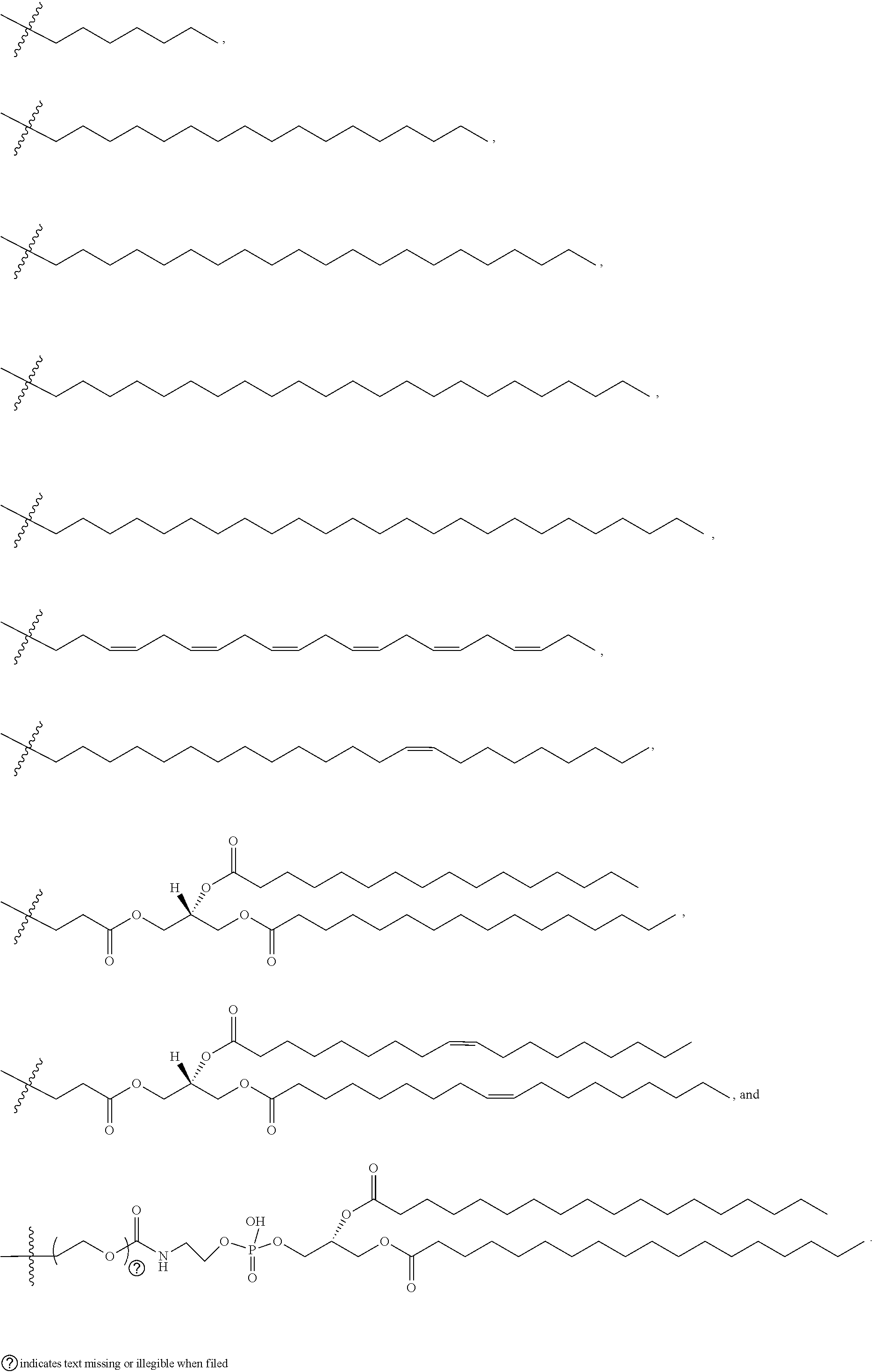

In some embodiments, R⁵ is selected from:

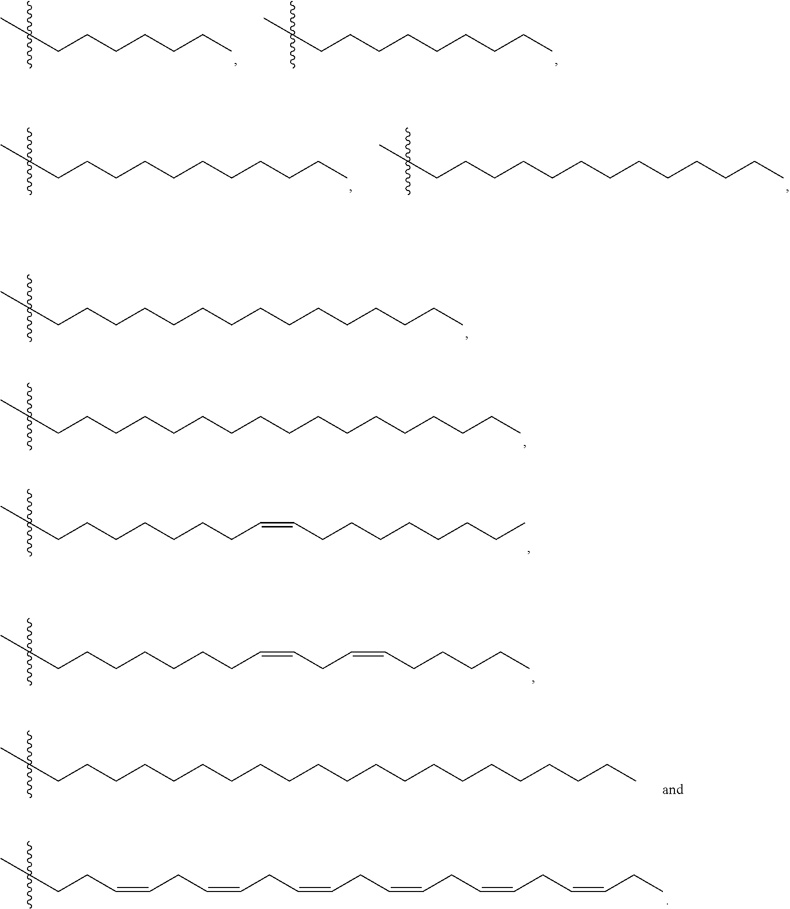

In some embodiments, R⁵ is

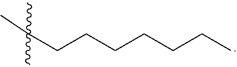 [SMILES: *CCCCCCC]

In some embodiments, R⁵ is

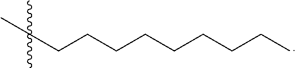 [SMILES: *CCCCCCCCC]

In some embodiments, R⁵ is

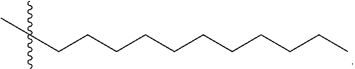 [SMILES: *CCCCCCCCCCC]

In some embodiments, R⁵ is

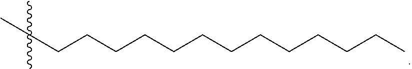 [SMILES: *CCCCCCCCCCCCC]

In some embodiments, R⁵ is

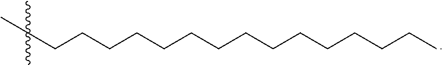 [SMILES: *CCCCCCCCCCCCCCC]

In some embodiments, R⁵ is

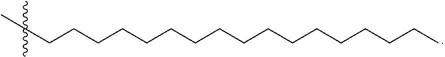 [SMILES: *CCCCCCCCCCCCCCCCC]

In some embodiments, R5 is

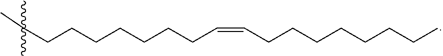 [SMILES: *CCCCCCC/C=C\CCCCCCCC]

In some embodiments, R⁵ is

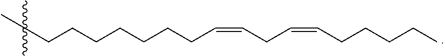 [SMILES: *CCCCCCC/C=C\C/C=C\CCCCC]

In some embodiments, R⁵ is

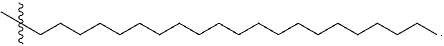 [SMILES: *CCCCCCCCCCCCCCCCCCCCC]

In some embodiments, R⁵ is

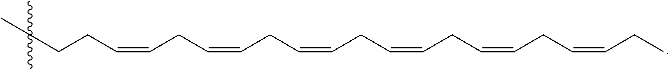 [SMILES: *CC/C=C\C/C=C\C/C=C\C/C=C\C/C=C\C/C=C\CC]

In some embodiments, R⁵ is

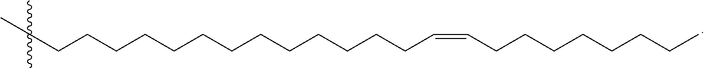 [SMILES: *CCCCCCCCCCCCC/C=C\CCCCCCCC]

In some embodiments, R⁵ is

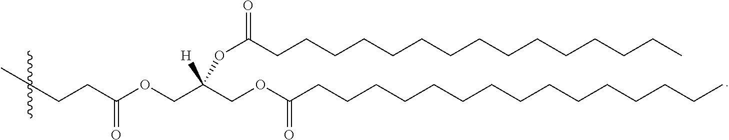 [SMILES: *CCC(=O)OC[C@@H](COC(=O)CCCCCCCCCCCCCCC)OC(=O)CCCCCCCCCCCCCCC]

In some embodiments, R⁵ is

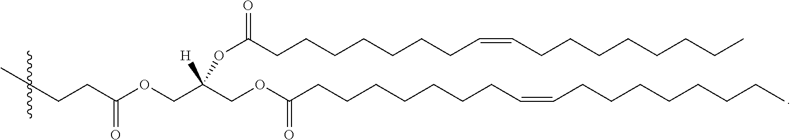 [SMILES: *CCC(=O)OC[C@@H](COC(=O)CCCCCCC/C=C\CCCCCCCC)OC(=O)CCCCCCC/C=C\CCCCCCCC]

In some embodiments, R⁵ is

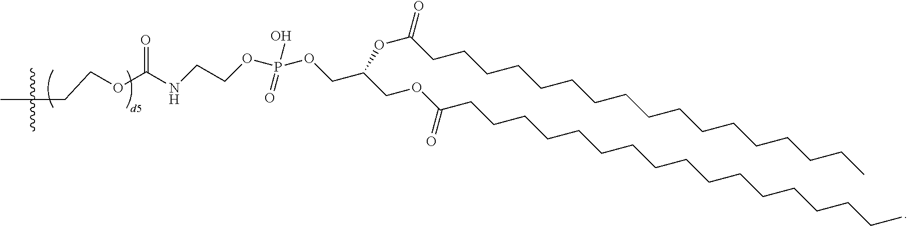

In some embodiments, the oligonucleotide-ligand conjugate comprises one or more nucleic acids conjugated with targeting ligands represented by formula II-Ib or II-Ic:

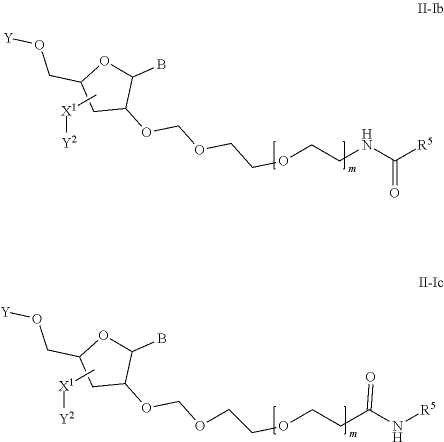

or a pharmaceutically acceptable salt thereof; wherein
B is a nucleobase or hydrogen;
m is 1-50;
X¹ is —O—, or —S—;
Y is hydrogen,

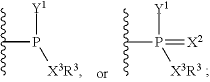

R³ is hydrogen, or a suitable protecting group;
X² is O, or S;
X³ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support;
R⁵ is adamantyl, or a saturated or unsaturated, straight, or branched C1-50 hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—; and
R is hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁵ is selected from

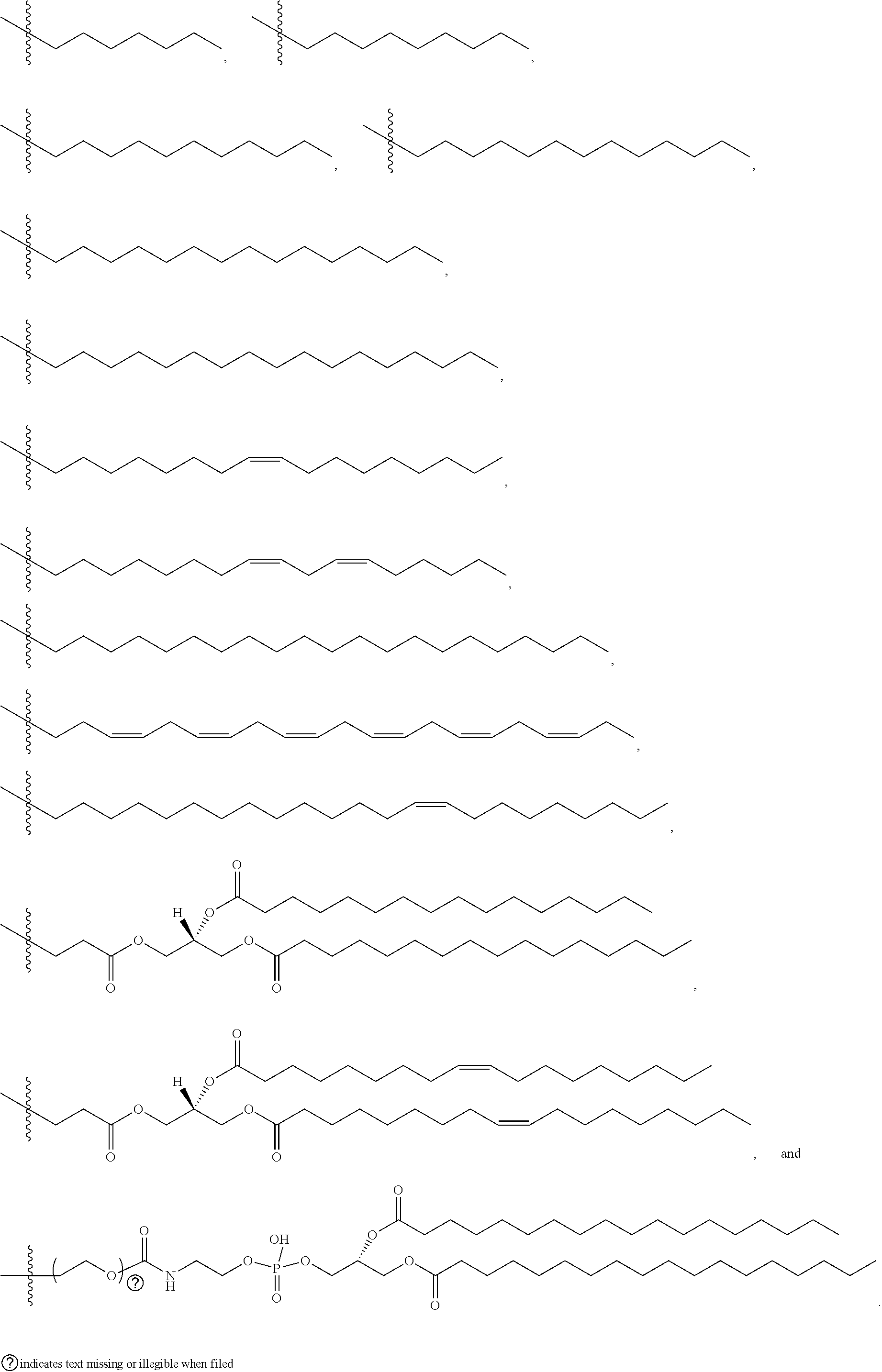

In some embodiments, R⁵ is

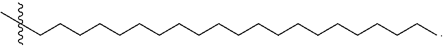 [SMILES: *CCCCCCCCCCCCCCCCCCCCC]

In some embodiments, the nucleotide sequence of the oligonucleotide comprises 1-10 targeting ligands. In some embodiments, the nucleotide sequence comprises 1, 2 or 3 targeting ligands. In some embodiments, the nucleotide sequence comprises 1 targeting ligand.

In some embodiments, the oligonucleotide of the oligonucleotide-ligand conjugate is a double-stranded molecule. In some embodiments, the oligonucleotide is an RNAi molecule. In some embodiments, the double stranded oligonucleotide comprises a stem loop. In some embodiments, the ligand is conjugated to any of the nucleotides in the stem loop. In some embodiments, the ligand is conjugated to the first nucleotide from 5′ to 3′, in the stem loop. In some embodiments, the ligand is conjugated to the second nucleotide from 5′ to 3′ in the stem loop. In some embodiments, the ligand is conjugated to the third nucleotide from 5′ to 3′ in the stem loop. In some embodiments, the ligand is conjugated to the fourth nucleotide from 5′ to 3′ in the stem loop. In some embodiments, the ligand is conjugated to one, two, three, or four of the nucleotides in the stem loop. In some embodiments, the ligand is conjugated to three of the nucleotides in the stem loop.

In some embodiments, the oligonucleotide-ligand conjugate comprises a sense strand of 36 nucleotides with positions numbered 1-36 from 5′ to 3′. In some embodiments, the oligonucleotide-ligand conjugate comprises a lipid conjugated to position 1 of a 36-nucleotide sense strand. In some embodiments, the oligonucleotide-ligand conjugate comprises a lipid conjugated to the 5′ terminal nucleotide of the sense strand. In some embodiments, the oligonucleotide-ligand conjugate comprises a lipid conjugated to the 5′ terminal nucleotide of a 36-nucleotide sense strand. In some embodiments, the oligonucleotide-ligand conjugate comprises a lipid conjugated to position 27 of a 36-nucleotide sense strand. In some embodiments, the oligonucleotide-ligand conjugate comprises a lipid conjugated to position 28 of a 36-nucleotide sense strand. In some embodiments, the oligonucleotide conjugate comprises a lipid conjugated to position 29 of a 36-nucleotide sense strand. In some embodiments, the oligonucleotide conjugate comprises a lipid conjugated to position 30 of a 36-nucleotide sense strand.

In some embodiments, the oligonucleotide-ligand conjugate comprises a C8-C30 hydrocarbon chain conjugated to position 1 of a 36-nucleotide sense strand. In some embodiments, the oligonucleotide-ligand conjugate comprises a C22 hydrocarbon chain conjugated to position 1 of a 36-nucleotide sense strand.

In some embodiments, the oligonucleotide-ligand conjugate comprises a lipid conjugated to the 5′ terminal nucleotide of the sense strand. In some embodiments, the oligonucleotide-ligand conjugate comprises a C8-C30 hydrocarbon chain conjugated to the 5′terminal nucleotide of the sense strand. In some embodiments, the oligonucleotide-ligand conjugate comprises a C22 hydrocarbon chain conjugated to the 5′ terminal nucleotide of the sense strand. In some embodiments, the oligonucleotide-ligand conjugate comprises a C18 hydrocarbon chain conjugated to the 5′ terminal nucleotide of the sense strand.

In some embodiments, the oligonucleotide-ligand conjugate comprises a hydrocarbon chain conjugated to the 2′ carbon of the ribose ring of the 5′ terminal nucleotide of the sense strand. In some embodiments, the oligonucleotide-ligand conjugate comprises a C8-C30 hydrocarbon chain conjugated to the 2′ carbon of the ribose ring of the 5′ terminal nucleotide of the sense strand. In some embodiments, the oligonucleotide-ligand conjugate comprises a C22 hydrocarbon chain conjugated to the 2′ carbon of the ribose ring of the 5′ terminal nucleotide of the sense strand. In some embodiments, the oligonucleotide-ligand conjugate comprises a C18 hydrocarbon chain conjugated to the 2′ carbon of the ribose ring of the 5′ terminal nucleotide of the sense strand.

In some embodiments, the oligonucleotide-ligand conjugate comprises a lipid conjugated to the 5′ terminal nucleotide of the sense strand via a linker. In some embodiments, the oligonucleotide-ligand conjugate comprises a C8-C30 hydrocarbon chain conjugated to the 5′terminal nucleotide of the sense strand via a linker. In some embodiments, the oligonucleotide-ligand conjugate comprises a C22 hydrocarbon chain conjugated to the 5′ terminal nucleotide of the sense strand via a linker. In some embodiments, the oligonucleotide-ligand conjugate comprises a C18 hydrocarbon chain conjugated to the 5′ terminal nucleotide of the sense strand via a linker.

In some embodiments, the oligonucleotide-ligand conjugate comprises a hydrocarbon chain conjugated to the 2′ carbon of the ribose ring of the 5′ terminal nucleotide of the sense strand via a linker. In some embodiments, the oligonucleotide-ligand conjugate comprises a C8-C30 hydrocarbon chain conjugated to the 2′ carbon of the ribose ring of the 5′ terminal nucleotide of the sense strand via a linker. In some embodiments, the oligonucleotide-ligand conjugate comprises a C22 hydrocarbon chain conjugated to the 2′ carbon of the ribose ring of the 5′ terminal nucleotide of the sense strand via a linker. In some embodiments, the oligonucleotide-ligand conjugate comprises a C18 hydrocarbon chain conjugated to the 2′ carbon of the ribose ring of the 5′ terminal nucleotide of the sense strand via a linker.

In some embodiments, an oligonucleotide-ligand conjugate comprises an antisense strand of 15 to 30 nucleotides and a sense strand of 15 to 40 nucleotide, wherein the sense and antisense strands form a duplex region, wherein the antisense strand comprises a region of complementarity to a target sequence expressed in the adrenal gland or adrenal cortex, wherein the sense strand comprises at its 3′ end a stem-loop comprising a tetraloop comprising 4 nucleosides, wherein one or more of the 4 nucleosides is represented by formula II-Ib:

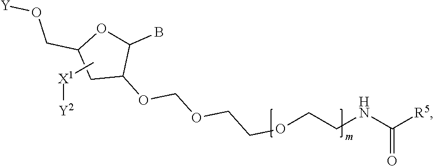

wherein B is selected from an adenine and a guanine nucleobase, and wherein R⁵ is a hydrocarbon chain. In some embodiments, m is 1, X1 is O, Y2 is an internucleotide linking group attaching to the 5′ terminal of a nucleoside,

Y is represented by

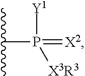

Y1 is a linking group attaching to the 2′ or 3′ terminal of a nucleotide, X2 is O, X3 is O, and R3 is H. In some embodiments, the hydrocarbon chain is a C8-C30 hydrocarbon chain. In some embodiments, the hydrocarbon chain is a C22 hydrocarbon chain. In some embodiments, the C22 hydrocarbon chain is represented by

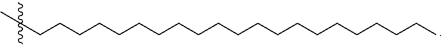 [SMILES: *CCCCCCCCCCCCCCCCCCCCC]

In some embodiments, the 4 nucleosides of the tetraloop are numbered 1-4 from 5′ to 3′ and position 1 is represented by formula II-Ib. In some embodiments, position 2 is represented by formula II-Ib. In some embodiments, position 3 is represented by formula II-Ib. In some embodiments, position 4 is represented by formula II-Ib. In some embodiments, the sense strand is 36 nucleotides with positions numbered 1-36 from 5′ to 3′, wherein the stem-loop comprises nucleotides at positions 21-36, and wherein one or more nucleosides at positions 27-30 are represented by formula II-Ib. In some embodiments, the antisense strand is 22 nucleotides.

In some aspects, the disclosure provides oligonucleotide-ligand conjugates for targeting a target mRNA (e.g., a target mRNA regulating immune suppression) and inhibiting or reducing target gene expression (e.g., via the RNAi pathway), wherein the oligonucleotide-ligand conjugate is a double-stranded (ds) nucleic acid molecule comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked. In some embodiments, the sense strand and antisense strand form a duplex region, wherein the sense strand and antisense strand, or a portion thereof, binds or anneals to one another in a complementary manner (e.g., by Watson-Crick base pairing).

In some embodiments, an oligonucleotide-ligand conjugate comprises an antisense strand of 15 to 30 nucleotides and a sense strand of 15 to 40 nucleotide, wherein the sense and antisense strands form a duplex region, wherein the antisense strand comprises a region of complementarity to a target sequence expressed in the adrenal gland or adrenal cortex, and wherein the 5′ terminal nucleotide of the sense strand comprises a nucleoside represented by formula II-Ib:

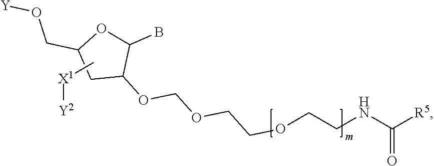

wherein B is selected from an adenine and a guanine nucleobase, and wherein R⁵ is a hydrocarbon chain. In some embodiments, m is 1, X1 is O, Y2 is an internucleotide linking group attaching to the 5′ terminal of a nucleoside,

Y is represented by

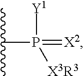

Y1 is a linking group attaching to the 2′ or 3′ terminal of a nucleotide, X2 is O, X3 is O, and R3 is H. In some embodiments, the hydrocarbon chain is a C8-C30 hydrocarbon chain. In some embodiments, the hydrocarbon chain is a C22 hydrocarbon chain. In some embodiments, the C22 hydrocarbon chain is represented by

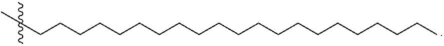 [SMILES: *CCCCCCCCCCCCCCCCCCCCC]

In some embodiments, the sense and antisense strands of an oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein the oligonucleotide comprises at least one lipid moiety conjugated to a nucleotide.

In some embodiments, the sense and antisense strands of an oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   wherein the oligonucleotide comprises at least one lipid moiety conjugated to a nucleotide.

In some embodiments, the sense and antisense strands of an oligonucleotide comprise nucleotides sequences selected from the group consisting of:
(a) SEQ ID NOs: 484 and 725, respectively;
(b) SEQ ID NOs: 487 and 728, respectively; and,
(c) SEQ ID NOs: 491 and 732, respectively,
   wherein the oligonucleotide comprises at least one lipid moiety conjugated to a nucleotide.

In some embodiments, the sense and antisense strands of an oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein the oligonucleotide comprises at least one hydrocarbon chain conjugated to a nucleotide.

In some embodiments, the sense and antisense strands of an oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   wherein the oligonucleotide comprises at least one hydrocarbon conjugated to a nucleotide.

In some embodiments, the sense and antisense strands of an oligonucleotide comprise nucleotides sequences selected from the group consisting of:
(a) SEQ ID NOs: 484 and 725, respectively;
(b) SEQ ID NOs: 487 and 728, respectively; and,
(c) SEQ ID NOs: 491 and 732, respectively,
   wherein the oligonucleotide comprises at least on hydrocarbon conjugated to a nucleotide.

In some embodiments, the sense and antisense strands of an oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein the oligonucleotide comprises at least one C18 hydrocarbon chain conjugated to a nucleotide.

In some embodiments, the sense and antisense strands of an oligonucleotide comprise nucleotides sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   wherein the oligonucleotide comprises at least one C18 hydrocarbon conjugated to a nucleotide.

In some embodiments, the sense and antisense strands of an oligonucleotide comprise nucleotides sequences selected from the group consisting of:
(a) SEQ ID NOs: 484 and 725, respectively;
(b) SEQ ID NOs: 487 and 728, respectively; and,
(c) SEQ ID NOs: 491 and 732, respectively,
   wherein the oligonucleotide comprises at least one C18 hydrocarbon conjugated to a nucleotide.

In some embodiments, the sense and antisense strands of an oligonucleotide comprise nucleotides sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively,
   wherein the oligonucleotide comprises at least one C22 hydrocarbon chain conjugated to a nucleotide.

In some embodiments, the sense and antisense strands of an oligonucleotide comprise nucleotides sequences selected from the group consisting of:

(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively,
   wherein the oligonucleotide comprises at least one C22 hydrocarbon conjugated to a nucleotide.

### Exemplary Oligonucleotides for Reducing CD274 Expression

In some embodiments, the CD274-targeting oligonucleotide (e.g., an RNAi oligonucleotide) for reducing CD274 expression provided by the disclosure comprise a sense strand and an antisense strand, wherein all nucleotides comprising the sense strand and antisense strand are modified, wherein the antisense strand comprises a region of complementarity to a CD274 mRNA target sequence of any one of SEQ ID NOs: 1-2 and 4-241 and wherein the region of complementarity is at least 15 contiguous nucleotides in length. In some embodiments, the 5′-terminal nucleotide of the antisense strand comprises 4′-O-monomethylphosphonate-2′-O-methyluridine [MePhosphonate-4O-mU], as described herein. In some embodiments, the 5′-terminal nucleotide of the antisense strand comprises a phosphorothioate linkage. In some embodiments, the antisense strand and the sense strand comprise one or more 2′-fluoro (2′-F) and 2′-O-methyl (2′-OMe) modified nucleotides and at least one phosphorothioate linkage. In some embodiments, the antisense strand comprises four (4) phosphorothioate linkages and the sense strand comprises one (1) phosphorothioate linkage. In some embodiments, the antisense strand comprises five (5) phosphorothioate linkages and the sense strand comprises one (1) phosphorothioate linkage.

In some embodiments, an oligonucleotide provided herein (e.g., an RNAi oligonucleotide) comprises a sense strand having a sequence of any one of SEQ ID NOs: 483-484 and 486-723 and an antisense strand comprising a complementary sequence selected from SEQ ID NOs: 724-725 and 727-964.

In some embodiments, an oligonucleotide provided herein (e.g., an RNAi oligonucleotide) comprises a sense strand having a sequence of any one of SEQ ID NOs: 965-1000 and an antisense strand comprising a complementary sequence selected from SEQ ID NOs: 1001-1036.

In some embodiments, an oligonucleotide provided herein (e.g., and RNAi oligonucleotide) for reducing CD274 expression comprises:
a sense strand comprising a 2′-F modified nucleotide at positions 8-11, a 2′-OMe modified nucleotide at positions 1-7, 12-27, and 31-36, a GalNAc-conjugated nucleotide at position 28, 29 and 30; and a phosphorothioate linkage between positions 1 and 2;
an antisense strand comprising a 2′-F modified nucleotide at positions 2, 3, 4, 5, 7, 10 and 14, a 2′-OMe at positions 1, 6, 8, 9, 11-13, and 15-22, a phosphorothioate linkage between positions 1 and 2, positions 2 and 3, positions 3 and 4, positions 20 and 21, and positions 21 and 22, and a 5′-terminal nucleotide at position 1 comprising a 4′-phosphate analog, optionally wherein the 5′-terminal nucleotide comprises 4′-O-monomethylphosphonate-2′-O-methyluridine [MePhosphonate-4O-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively.

In some embodiments, the CD274-targeting oligonucleotide (e.g., an RNAi oligonucleotide) for reducing CD274 expression comprise:
a sense strand comprising a 2′-F modified nucleotide at positions 8-11, a 2′-OMe modified nucleotide at positions 1-7, 12-27, and 31-36, a GalNAc-conjugated nucleotide at position 28, 29 and 30; and a phosphorothioate linkage between positions 1 and 2;
an antisense strand comprising a 2′-F modified nucleotide at positions 2, 3, 4, 5, 7, 10 and 14, a 2′-OMe at positions 1, 6, 8, 9, 11-13, and 15-22, a phosphorothioate linkage between positions 1 and 2, positions 2 and 3, positions 3 and 4, positions 20 and 21, and positions 21 and 22, and a 5′-terminal nucleotide at position 1 comprising a 4′-phosphate analog, optionally wherein the 5′-terminal nucleotide comprises 4′-O-monomethylphosphonate-2′-O-methyluridine [MePhosphonate-4O-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
(a) SEQ ID NOs: 484 and 725, respectively;
(b) SEQ ID NOs: 486 and 727, respectively;
(c) SEQ ID NOs: 487 and 728, respectively;
(d) SEQ ID NOs: 488 and 729, respectively;
(e) SEQ ID NOs: 489 and 730, respectively;
(f) SEQ ID NOs: 491 and 732, respectively; and,
(g) SEQ ID NOs: 502 and 743, respectively.

In some embodiments, a CD274-targeting oligonucleotide for reducing CD274 expression provided by the disclosure comprises a sense strand comprising the nucleotide sequence as set forth in SEQ ID NO: 484 and an antisense strand comprising the nucleotide sequence as set forth in SEQ ID NO: 725. In some embodiments, a CD274-targeting oligonucleotide for reducing CD274 expression provided by the disclosure comprises a sense strand comprising the nucleotide sequence as set forth in SEQ ID NO: 486 and an antisense strand comprising the nucleotide sequence as set forth in SEQ ID NO: 727. In some embodiments, a CD274-targeting oligonucleotide for reducing CD274 expression provided by the disclosure comprises a sense strand comprising the nucleotide sequence as set forth in SEQ ID NO: 487 and an antisense strand comprising the nucleotide sequence as set forth in SEQ ID NO: 728. In some embodiments, a CD274-targeting oligonucleotide for reducing CD274 expression provided by the disclosure comprises a sense strand comprising the nucleotide sequence as set forth in SEQ ID NO: 488 and an antisense strand comprising the nucleotide sequence as set forth in SEQ ID NO: 729. In some embodiments, a CD274-targeting oligonucleotide for reducing CD274 expression provided by the disclosure comprises a sense strand comprising the nucleotide sequence as set forth in SEQ ID NO: 489 and an antisense strand comprising the nucleotide sequence as set forth in SEQ ID NO: 730. In some embodiments, a CD274-targeting oligonucleotide for reducing CD274 expression provided by the disclosure comprises a sense strand comprising the nucleotide sequence as set forth in SEQ ID NO: 491 and an antisense strand comprising the nucleotide sequence as set forth in SEQ ID NO: 732. In some embodiments, a CD274-targeting oligonucleotide for reducing CD274 expression provided by the disclosure comprises a sense strand comprising the nucleotide sequence as set forth in SEQ ID NO: 502 and an antisense strand comprising the nucleotide sequence as set forth in SEQ ID NO: 743.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 243; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 245; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 246; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 247; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 248; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 250; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 261; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 243; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 245; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 246; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 247; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 248; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 250; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 261; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 243; and (ii) a sense strand of 19-50 nucleotides in length comprising a region of complementarity to the antisense strand, wherein the region of complementarity to the antisense strand is set forth in SEQ ID NO: 2, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 245; and (ii) a sense strand of 19-50 nucleotides in length comprising a region of complementarity to the antisense strand, wherein the region of complementarity to the antisense strand is set forth in SEQ ID NO: 4, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 246; and (ii) a sense strand of 19-50 nucleotides in length comprising a region of complementarity to the antisense strand, wherein the region of complementarity to the antisense strand is set forth in SEQ ID NO: 5, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 247; and (ii) a sense strand of 19-50 nucleotides in length comprising a region of complementarity to the antisense strand, wherein the region of complementarity to the antisense strand is set forth in SEQ ID NO:6, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 248; and (ii) a sense strand of 19-50 nucleotides in length comprising a region of complementarity to the antisense strand, wherein the region of complementarity to the antisense strand is set forth in SEQ ID NO: 7, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 250; and (ii) a sense strand of 19-50 nucleotides in length comprising a region of complementarity to the antisense strand, wherein the region of complementarity to the antisense strand is set forth in SEQ ID NO: 9, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 261; and (ii) a sense strand of 19-50 nucleotides in length comprising a region of complementarity to the antisense strand, wherein the region of complementarity to the antisense strand is set forth in SEQ ID NO: 20, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 243; and (ii) a sense strand of 19-50 nucleotides in length comprising a region of complementarity to the antisense strand and a stem-loop at the 3′terminus, wherein the region of complementarity to the antisense strand is set forth in SEQ ID NO: 2, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 245; and (ii) a sense strand of 19-50 nucleotides in length comprising a region of complementarity to the antisense strand and a stem-loop at the 3′terminus, wherein the region of complementarity to the antisense strand is set forth in SEQ ID NO: 4, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 246; and (ii) a sense strand of 19-50 nucleotides in length comprising a region of complementarity to the antisense strand and a stem-loop at the 3′terminus, wherein the region of complementarity to the antisense strand is set forth in SEQ ID NO: 5, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 247; and (ii) a sense strand of 19-50 nucleotides in length comprising a region of complementarity to the antisense strand and a stem-loop at the 3′terminus, wherein the region of complementarity to the antisense strand is set forth in SEQ ID NO: 6, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 248; and (ii) a sense strand of 19-50 nucleotides in length comprising a region of complementarity to the antisense strand and a stem-loop at the 3′terminus, wherein the region of complementarity to the antisense strand is set forth in SEQ ID NO: 7, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 250; and (ii) a sense strand of 19-50 nucleotides in length comprising a region of complementarity to the antisense strand and a stem-loop at the 3′terminus, wherein the region of complementarity to the antisense strand is set forth in SEQ ID NO: 9, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a CD274-targeting oligonucleotide (e.g., an RNAi oligonucleotide) for reducing CD274 expression comprises (i) an antisense strand of 19-30 nucleotides in length, wherein the antisense strand comprises a nucleotide sequence comprising a region of complementarity to a CD274 mRNA target sequence, wherein the region of complementarity is set forth in SEQ ID NO: 261; and (ii) a sense strand of 19-50 nucleotides in length comprising a region of complementarity to the antisense strand and a stem-loop at the 3′terminus, wherein the region of complementarity to the antisense strand is set forth in SEQ ID NO: 20, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, the disclosure provides an oligonucleotide (e.g., an RNAi oligonucleotide) for reducing CD274 expression, wherein the oligonucleotide comprises a sense strand and an antisense strand according to:

Sense Strand:
5′-mX-S-mX-mX-mX-mX-mX-mX-fX-fX-fX-fX-mX-mX-mX-
mX-mX-mX-mX-mX-mX-mX-mX-mX-mX-mX-mX-mX-[ademX-
L]-mX-mX-mX-mX-mX-mX-mX-mX-3′;

hybridized to:

Antisense Strand:
5′-[MePhosphonate-40-mX]-S-fX-S-fX-fX-fX-mX-fX-
mX-mX-fX-mX-mX-mX-fX-mX-mX-mX-mX-mX-mX-S-mX-S-
mX-3′;

wherein mX=2′-O-methyl modified nucleotide, fX=2′-fluoro modified nucleotide, —S—=phosphorothioate linkage, -=phosphodiester linkage, [MePhosphonate-4O-mX]=4′-O-monomethylphosphonate-2′-O-methyl modified nucleotide, and AdemX-L=Lipid molecule (e.g., C18) attached to a nucleotide.
In some embodiments, the disclosure provides an oligonucleotide (e.g., an RNAi oligonucleotide) for reducing CD274 expression, wherein the oligonucleotide comprises a sense strand and an antisense strand according to:

Sense Strand:
5′-mX-S-mX-mX-mX-mX-mX-mX-fX-fX-fX-fX-mX-mX-mX-
mX-mX-mX-mX-mX-mX-mX-mX-mX-mX-mX-mX-mX-[ademX-
GalNAc]-[ademX-GalNAc]-[ademX-GalNAc]-mX-mX-mX-
mX-mX-mX-3′;

hybridized to:

Antisense Strand:
5′-[MePhosphonate-40-mX]-S-fX-S-fX-S-fX-fX-mX-
fX-mX-mX-fX-mX-mX-mX-fX-mX-mX-mX-mX-mX-mX-S-mX-
S-mX-3′;

wherein mX=2′-O-methyl modified nucleotide, fX=2′-fluoro modified nucleotide, —S—=phosphorothioate linkage, -=phosphodiester linkage, [MePhosphonate-4O-mX]=4′-O-monomethylphosphonate-2′-O-methyl modified nucleotide, and ademX-GalNAc=GalNAc attached to a nucleotide.

In some embodiments, the disclosure provides an RNAi oligonucleotide for reducing CD274 expression, the oligonucleotide comprising a sense strand and an antisense strand, wherein the sense strand comprises the sequence and all of the modifications of 5′ [ademGs-C18][mG][mA][mU][mA][mU][mU][fU][fG][fC][fU][mG][mU][mC][mU][mU][mU][mA][mU][mA][mG][mC][mA][mG][mC][mC][mG][mA][mA][mA][mG][mG][mC][mU][mG][mC]-3′ (SEQ ID NO: 1050), and wherein the antisense strand comprises the sequence and all of the modifications of 5′ [MePhosphonate-4O-mUs][fAs][fUs][fA][fA][mA][fG][mA][mC][fA][mG][mC][mA][fA][mA][mU][mA][mU][mC][mCs][mGs][mG]-3′ (SEQ ID NO: 1005), wherein mC, mA, mG, mU=2′-OMe ribonucleosides; fA, fC, fG, fU=2′F ribonucleosides; s=phosphorothioate, and wherein ademA-GalNAc=GalNAc modified adenine nucleotide.

In some embodiments, the disclosure provides an RNAi oligonucleotide for reducing CD274 expression, the oligonucleotide comprising a sense strand and an antisense strand, wherein the sense strand comprises the sequence and all of the modifications of 5′ [mAs][mU][mG][mA][mG][mG][mA][fU][fA][fU][fU][mU][mG][mC][mU][mG][mU][mC][m U][mA][mG][mC][mA][mG][mC][mC][mG][ademA-GalNAc][ademA-GalNAc][ademA-GalNAc][mG][mG][mC][mU][mG][mC]-3′ (SEQ ID NO: 966), and wherein the antisense strand comprises the sequence and all of the modifications of 5′ [MePhosphonate-4O-mUs][fAs][fGs][fA][fC][mA][fG][mC][mA][fA][mA][mU][mA][fU][mC][mC][mU][mC][mA][mUs][mGs][mG]3′ (SEQ ID NO: 1002), wherein mC, mA, mG, mU=2′-OMe ribonucleosides; fA, fC, fG, fU=2′F ribonucleosides; s=phosphorothioate, and wherein ademA-GalNAc=GalNAc modified adenine nucleotide.

In some embodiments, the disclosure provides an RNAi oligonucleotide for reducing CD274 expression, the oligonucleotide comprising a sense strand and an antisense strand, wherein the sense strand comprises the sequence and all of the modifications of 5′ [mAs][mG][mG][mA][mU][mA][mU][fU][fU][fG][fC][mU][mG][mU][mC][mU][mU][mU][mA][mA][mG][mC][mA][mG][mC][mC][mG][ademA-GalNAc][ademA-GalNAc][ademA-GalNAc][mG][mG][mC][mU][mG][mC]-3′ (SEQ ID NO: 968), and wherein the antisense strand comprises the sequence and all of the modifications of 5′ [MePhosphonate-4O-mUs][fUs][fAs][fA][fA][mG][fA][mC][mA][fG][mC][mA][mA][fA][mU][mA][mU][mC][mC][mUs][mGs][mG]-3′ (SEQ ID NO: 1004), wherein mC, mA, mG, mU=2′-OMe ribonucleosides; fA, fC, fG, fU=2′F ribonucleosides; s=phosphorothioate, and wherein ademA-GalNAc=GalNAc modified adenine nucleotide.

In some embodiments, the disclosure provides an RNAi oligonucleotide for reducing CD274 expression, the oligonucleotide comprising a sense strand and an antisense strand, wherein the sense strand comprises the sequence and all of the modifications of 5′-[mGs][mG][mA][mU][mA][mU][mU][fU][fG][fC][fU][mG][mU][mC][mU][mU][mU][mA][m U][mA][mG][mC][mA][mG][mC][mC][mG][ademA-GalNAc][ademA-GalNAc][ademA-GalNAc][mG][mG][mC][mU][mG][mC]-3′ (SEQ ID NO: 969), and wherein the antisense strand comprises the sequence and all of the modifications of 5′-[MePhosphonate-4O-mUs][fAs][fUs][fA][fA][mA][fG][mA][mC][fA][mG][mC][mA][fA][mA][mU][mA][mU][mC][mCs][mGs][mG]-3′ (SEQ ID NO: 1005), wherein mC, mA, mG, mU=2′-OMe ribonucleosides; fA, fC, fG, fU=2′F ribonucleosides; s=phosphorothioate, and wherein ademA-GalNAc=GalNAc modified adenine nucleotide.

In some embodiments, the disclosure provides, an RNAi oligonucleotide for reducing CD274 expression, the oligonucleotide comprising a sense strand and an antisense strand, wherein the sense strand comprises the sequence and all of the modifications of 5′-[mAs][mU][mA][mU][mU][mU][mG][fC][fU][fG][fU][mC][mU][mU][mU][mA][mU][mA][m U][mA][mG][mC][mA][mG][mC][mC][mG][ademA-GalNAc][ademA-GalNAc][ademA-GalNAc][mG][mG][mC][mU][mG][mC]-3′ (SEQ ID NO: 970), and wherein the antisense strand comprises the sequence and all of the modifications of 5′-[MePhosphonate-4O-mUs][fAs][fUs][fA][fU][mA][fA][mA][mG][fA][mC][mA][mG][fC][mA][mA][mA][mU][mA][mUs][mGs][mG]-3′ (SEQ ID NO: 1006), wherein mC, mA, mG, mU=2′-OMe ribonucleosides; fA, fC, fG, fU=2′F ribonucleosides; s=phosphorothioate, and wherein ademA-GalNAc=GalNAc modified adenine nucleotide.

In some embodiments, the disclosure provides, an RNAi oligonucleotide for reducing CD274 expression, the oligonucleotide comprising a sense strand and an antisense strand, wherein the sense strand comprises the sequence and all of the modifications of 5′-[mAs][mU][mU][mU][mG][mC][mU][fG][fU][fC][fU][mU][mU][mA][mU][mA][mU][mU][m C][mA][mG][mC][mA][mG][mC][mC][mG][ademA-GalNAc][ademA-GalNAc][ademA-GalNAc][mG][mG][mC][mU][mG][mC]-3′ (SEQ ID NO: 971), and wherein the antisense strand comprises the sequence and all of the modifications of 5′-[MePhosphonate-4O-mUs][fGs][fAs][fA][fU][mA][fU][mA][mA][fA][mG][mA][mC][fA][mG][mC][mA][mA][mA][mUs][mGs][mG]-3′ (SEQ ID NO: 1007), wherein mC, mA, mG, mU=2′-OMe ribonucleosides; fA, fC, fG, fU=2′F ribonucleosides; s=phosphorothioate, and wherein ademA-GalNAc=GalNAc modified adenine nucleotide.

In some embodiments, the disclosure provides, an RNAi oligonucleotide for reducing CD274 expression, the oligonucleotide comprising a sense strand and an antisense strand, wherein the sense strand comprises the sequence and all of the modifications of 5′ [mGs][mC][mA][mA][mU][mA][mU][fG][fA][fC][fA][mA][mU][mU][mG][mA][mA][mU][m G][mA][mG][mC][mA][mG][mC][mC][mG][ademA-GalNAc][ademA-GalNAc][ademA-GalNAc][mG][mG][mC][mU][mG][mC]-3′ (SEQ ID NO: 973), and wherein the antisense strand comprises the sequence and all of the modifications of 5′-[MePhosphonate-4O-mUs][fCs][fAs][fU][fU][mC][fA][mA][mU][fU][mG][mU][mC][fA][mU][mA][mU][mU][mG][mCs][mGs][mG]-3′ (SEQ ID NO: 1009), wherein mC, mA, mG, mU=2′-OMe ribonucleosides; fA, fC, fG, fU=2′F ribonucleosides; s=phosphorothioate, and wherein ademA-GalNAc=GalNAc modified adenine nucleotide.

In some embodiments, the disclosure provides, an RNAi oligonucleotide for reducing CD274 expression, the oligonucleotide comprising a sense strand and an antisense strand, wherein the sense strand comprises the sequence and all of the modifications of 5′-[mGs][mA][mU][mA][mA][mG][mA][fA][fC][fA][fU][mU][mA][mU][mU][mC][mA][mA][m U][mA][mG][mC][mA][mG][mC][mC][mG][ademA-GalNAc][ademA-GalNAc][ademA-GalNAc][mG][mG][mC][mU][mG][mC]-3′ (SEQ ID NO: 984), and wherein the antisense strand comprises the sequence and all of the modifications of 5′-[MePhosphonate-4O-mUs][fAs][fUs][fU][fG][mA][fA][mU][mA][fA][mU][mG][mU][fU][mC][mU][mU][mA][mU][mCs][mGs][mG]-3′ (SEQ ID NO: 1020), wherein mC, mA, mG, mU=2′-OMe ribonucleosides; fA, fC, fG, fU=2′F ribonucleosides; s=phosphorothioate, and wherein ademA-GalNAc=GalNAc modified adenine nucleotide.

In some embodiments, the disclosure provides an oligonucleotide (e.g., an RNAi oligonucleotide) for reducing CD274 expression, wherein the oligonucleotide comprises a sense strand and an antisense strand according to:

Sense Strand:
5′-[ademXs-L][mX][mX][X][mX][mX][mX][fX][fX][fX]
[X][mX][mX][mX][mX][mX][mX][mX][mX][mX][mX][mX]
[mX][mX][mX][mX][mX][mX][mX][mX][mX][mX][mX][mX]
[mX][X]-3′

### Hybridized to:

Antisense Strand:
5′-[MePhosphonate-40-mXs][fXs][fXs][fX][fX][mX]
[fX][X][mX][fX][X][mX][mX][fX][mX][X][mX][mX]
[mX][mXs][mXs][mX]-3′;

wherein mX=2′-O-methyl modified nucleotide, fX=2′-fluoro modified nucleotide, s=phosphorothioate linkage,][=phosphodiester linkage, [MePhosphonate-4O-mX]=4′-O-monomethylphosphonate-2′-O-methyl modified nucleotide, and AdemX-L=Lipid molecule (e.g., C18) attached to a nucleotide.

In some embodiments, the disclosure provides an oligonucleotide (e.g., an RNAi oligonucleotide) for reducing CD274 expression, wherein the oligonucleotide comprises a sense strand and an antisense strand according to:

Sense Strand:
5′-[ademXs-C18][mX][X][mX][mX][mX][mX][X][fX]
[fX][fX][mX][mX][mX][mX][mX][mX][X][mX][mX][mX]
[mX][X][mX][mX][mX][mX][mX][mX][mX][mX][X][mX]
[mX][mX][X]-3′

### Hybridized to:

Antisense Strand:
5′-[MePhosphonate-40-mXs][fXs][fXs][fX][fX][mX]
[X][mX][mX][fX][mX][mX][mX][fX][mX][mX][mX][mX]
[mX][mXs][mXs][mX]-3′;

wherein mX=2′-O-methyl modified nucleotide, fX=2′-fluoro modified nucleotide, s=phosphorothioate linkage,][=phosphodiester linkage, [MePhosphonate-4O-mX]=4′-O-monomethylphosphonate-2′-O-methyl modified nucleotide, and AdemX-C18=C18 hydrocarbon chain attached to a nucleotide.

In some embodiments, an oligonucleotide for reducing expression of CD274 mRNA comprises a sense strand and an antisense strand comprising nucleotide sequences selected from:
(a) SEQ ID NOs: 484 and 725, respectively;
(b) SEQ ID NOs: 487 and 728, respectively; and,
(c) SEQ ID NOs: 491 and 732, respectively,
   wherein the sense and antisense strands are modified based on the pattern below

Sense Strand:
5′-[ademXs-C18][mX][mX][mX][mX][X][mX][fX][fX]
[X][fX][mX][mX][mX][mX][mX][mX][mX][mX][mX][mX]
[X][mX][mX][mX][X][X][mX][mX][mX][X][mX][mX]
[mX][X][mX]-3′

### Hybridized to:

Antisense Strand:
5′-[MePhosphonate-40-mXs][fXs][fXs][fX][fX][mX]
[fX][mX][mX][fX][mX][mX][mX][fX][mX][mX][mX][mX]
[mX][mXs][mXs][mX]-3′;

wherein mX=2′-O-methyl modified nucleotide, fX=2′-fluoro modified nucleotide, s=phosphorothioate linkage,][=phosphodiester linkage, [MePhosphonate-4O-mX]=4′-O-monomethylphosphonate-2′-O-methyl modified nucleotide, and AdemX-C18=C18 hydrocarbon chain attached to a nucleotide.

In some embodiments, the disclosure provides an oligonucleotide (e.g., an RNAi oligonucleotide) for reducing CD274 expression, wherein the oligonucleotide comprises a sense strand and an antisense strand according to:

Sense Strand:
5′-[ademXs-L][mX][mX][mX][mX][mX][mX][fX][X][fX]
[fX][mX][mX][mX][mX][mX][mX][mX][mX][mX][mX][mX]
[mX][mX][mX][mX][mX][mX][mX][X][mX][mX][mX][X]
[mX][mX]-3′

### Hybridized to:

Antisense Strand:
5′-[MePhosphonate-40-mXs][fXs][fX][fX][fX][mX]
[fX][mX][mX][fX][mX][mX][mX][X][mX][mX][X][mX]
[X][mXs][mXs][mX]-3′;

wherein mX=2′-O-methyl modified nucleotide, fX=2′-fluoro modified nucleotide, s=phosphorothioate linkage,][=phosphodiester linkage, [MePhosphonate-4O-mX]=4′-O-monomethylphosphonate-2′-O-methyl modified nucleotide, and AdemX-L=Lipid molecule (e.g., C18) attached to a nucleotide.

In some embodiments, the disclosure provides an oligonucleotide (e.g., an RNAi oligonucleotide) for reducing CD274 expression, wherein the oligonucleotide comprises a sense strand and an antisense strand according to:

Sense Strand:
5′-[ademXs-C18][mX][mX][mX][mX][X][mX][X][X]
[X][fX][X][mX][mX][mX][mX][mX][mX][mX][mX][X]
[mX][mX][mX][mX][mX][mX][mX][mX][mX][mX][mX]
[mX][mX][mX][mX]-3′

### Hybridized to:

Antisense Strand:
5′-[MePhosphonate-4O-mXs][fXs][fX][fX][fX][mX][fX]
[mX][mX][fX][mX][mX][mX][fX][mX][mX][mX][mX][mX]
[mXs][mXs][mX]-3′;

wherein mX=2′-O-methyl modified nucleotide, fX=2′-fluoro modified nucleotide, s=phosphorothioate linkage,][=phosphodiester linkage, [MePhosphonate-4O-mX]=4′-O-monomethylphosphonate-2′-O-methyl modified nucleotide, and AdemX-C18=C18 hydrocarbon chain attached to a nucleotide.

In some embodiments, an oligonucleotide for reducing expression of CD274 mRNA comprises a sense strand and an antisense strand comprising nucleotide sequences selected from:
(a) SEQ ID NOs: 484 and 725, respectively;
(b) SEQ ID NOs: 487 and 728, respectively; and,
(c) SEQ ID NOs: 491 and 732, respectively,
   wherein the sense and antisense strands are modified based on the pattern below

Sense Strand:
5′-[ademXs-C18][mX][mX][mX][mX][mX][mX][fX][fX]
[fX][fX][mX][mX][mX][mX][mX][mX][mX][mX][mX][mX]
[mX][mX][mX][mX][mX][mX][mX][mX][mX][mX][mX][mX]
[mX][mX][mX]-3′

### Hybridized to:

Antisense Strand:
5′-[MePhosphonate-4O-mXs][fXs][fX][fX][fX][mX][fX]
[mX][mX][fX][mX][mX][mX][fX][mX][mX][mX][mX][mX]
[mXs][mXs][mX]-3′;

wherein mX=2′-O-methyl modified nucleotide, fX=2′-fluoro modified nucleotide, s=phosphorothioate linkage,][=phosphodiester linkage, [MePhosphonate-4O-mX]=4′-O-monomethylphosphonate-2′-O-methyl modified nucleotide, and AdemX-C18=C18 hydrocarbon chain attached to a nucleotide.

In some embodiments, the disclosure provides an oligonucleotide (e.g., an RNAi oligonucleotide) for reducing CD274 expression, wherein the oligonucleotide comprises a sense strand and an antisense strand comprising nucleotide sequences selected from the group consisting of:
(a) SEQ ID NOs: 965 and 1001, respectively;
(b) SEQ ID NOs: 966 and 1002, respectively;
(c) SEQ ID NOs: 968 and 1004, respectively;
(d) SEQ ID NOs: 969 and 1005, respectively;
(e) SEQ ID NOs: 970 and 1006, respectively;
(f) SEQ ID NOs: 971 and 1007, respectively;
(g) SEQ ID NOs: 972 and 1008, respectively;
(h) SEQ ID NOs: 974 and 1010, respectively;
(i) SEQ ID NOs: 975 and 1011, respectively;
(j) SEQ ID NOs: 976 and 1012, respectively;
(k) SEQ ID NOs: 977 and 1013, respectively;
(l) SEQ ID NOs: 978 and 1014, respectively;
(m) SEQ ID NOs: 979 and 1015, respectively;
(n) SEQ ID NOs: 980 and 1016, respectively;
(o) SEQ ID NOs: 981 and 1017, respectively;
(p) SEQ ID NOs: 982 and 1018, respectively;
(q) SEQ ID NOs: 983 and 1019, respectively;
(r) SEQ ID NOs: 984 and 1020, respectively;
(s) SEQ ID NOs: 985 and 1021, respectively;
(t) SEQ ID NOs: 986 and 1022, respectively;
(u) SEQ ID NOs: 987 and 1023, respectively;
(v) SEQ ID NOs: 988 and 1024, respectively;
(w) SEQ ID NOs: 989 and 1025, respectively;
(x) SEQ ID NOs: 990 and 1026, respectively;
(y) SEQ ID NOs: 991 and 1027, respectively;
(z) SEQ ID NOs: 992 and 1028, respectively;
(aa) SEQ ID NOs: 993 and 1029, respectively;
(bb) SEQ ID NOs: 994 and 1030, respectively;
(cc) SEQ ID NOs: 995 and 1031, respectively;
(dd) SEQ ID NOs: 996 and 1032, respectively;
(ee) SEQ ID NOs: 997 and 1033, respectively;
(ff) SEQ ID NOs: 998 and 1034, respectively;
(gg) SEQ ID NOs: 999 and 1035, respectively;
(hh) SEQ ID NOs: 1000 and 1036, respectively; and,
(ii) SEQ ID NOs: 973 and 1009, respectively.

In some embodiments, a CD274-targeting oligonucleotide for reducing CD274 expression provided by the disclosure comprises a sense strand comprising the nucleotide sequence as set forth in SEQ ID NO: 966 and an antisense strand comprising the nucleotide sequence as set forth in SEQ ID NO: 1002. In some embodiments, a CD274-targeting oligonucleotide for reducing CD274 expression provided by the disclosure comprises a sense strand comprising the nucleotide sequence as set forth in SEQ ID NO: 968 and an antisense strand comprising the nucleotide sequence as set forth in SEQ ID NO: 1004. In some embodiments, a CD274-targeting oligonucleotide for reducing CD274 expression provided by the disclosure comprises a sense strand comprising the nucleotide sequence as set forth in SEQ ID NO: 969 and an antisense strand comprising the nucleotide sequence as set forth in SEQ ID NO: 1005. In some embodiments, a CD274-targeting oligonucleotide for reducing CD274 expression provided by the disclosure comprises a sense strand comprising the nucleotide sequence as set forth in SEQ ID NO: 1050 and an antisense strand comprising the nucleotide sequence as set forth in SEQ ID NO: 1005. In some embodiments, a CD274-targeting oligonucleotide for reducing CD274 expression provided by the disclosure comprises a sense strand comprising the nucleotide sequence as set forth in SEQ ID NO: 970 and an antisense strand comprising the nucleotide sequence as set forth in SEQ ID NO: 1006. In some embodiments, a CD274-targeting oligonucleotide for reducing CD274 expression provided by the disclosure comprises a sense strand comprising the nucleotide sequence as set forth in SEQ ID NO: 971 and an antisense strand comprising the nucleotide sequence as set forth in SEQ ID NO: 1007. In some embodiments, a CD274-targeting oligonucleotide for reducing CD274 expression provided by the disclosure comprises a sense strand comprising the nucleotide sequence as set forth in SEQ ID NO: 973 and an antisense strand comprising the nucleotide sequence as set forth in SEQ ID NO: 1009. In some embodiments, a CD274-targeting oligonucleotide for reducing CD274 expression provided by the disclosure comprises a sense strand comprising the nucleotide sequence as set forth in SEQ ID NO: 984 and an antisense strand comprising the nucleotide sequence as set forth in SEQ ID NO: 1020.

In some embodiments, a CD274-targeting oligonucleotide for reducing CD274 expression provided by the disclosure comprises an antisense strand comprising the nucleotide sequence of SEQ ID NO: 728 and a sense strand comprising the nucleotide sequence of SEQ ID NO: 487, wherein the sense strand comprises a saturated C18 hydrocarbon chain conjugated to the 5′ terminal nucleotide of the sense strand, and wherein each of the antisense and sense strands comprise at least one 2′-modified nucleotide and at least one modified internucleotide linkage.

In some embodiments, a CD274-targeting oligonucleotide for reducing CD274 expression provided by the disclosure comprises an antisense strand comprising the nucleotide sequence of SEQ ID NO: 728 and a sense strand comprising the nucleotide sequence of SEQ ID NO:487, wherein the sense strand comprises a saturated C18 hydrocarbon chain conjugated to the 2′ carbon of the ribose ring of the 5′ terminal nucleotide of the sense strand via a linker, and wherein each of the antisense and sense strands comprise at least one 2′-modified nucleotide and at least one modified internucleotide linkage.

In some embodiments, a CD274-targeting oligonucleotide for reducing CD274 expression provided by the disclosure comprises an antisense strand comprising the nucleotide sequence of SEQ ID NO: 725 and a sense strand comprising the nucleotide sequence of SEQ ID NO: 484, wherein the sense strand comprises a saturated C18 hydrocarbon chain conjugated to the 5′ terminal nucleotide of the sense strand, and wherein each of the antisense and sense strands comprise at least one 2′-modified nucleotide and at least one modified internucleotide linkage.

In some embodiments, a CD274-targeting oligonucleotide for reducing CD274 expression provided by the disclosure comprises an antisense strand comprising the nucleotide sequence of SEQ ID NO: 725 and a sense strand comprising the nucleotide sequence of SEQ ID NO: 484, wherein the sense strand comprises a saturated C18 hydrocarbon chain conjugated to the 2′ carbon of the ribose ring of the 5′ terminal nucleotide of the sense strand via a linker, and wherein each of the antisense and sense strands comprise at least one 2′-modified nucleotide and at least one modified internucleotide linkage.

In some embodiments, a CD274-targeting oligonucleotide for reducing CD274 expression provided by the disclosure comprises an antisense strand comprising the nucleotide sequence of SEQ ID NO: 732 and a sense strand comprising the nucleotide sequence of SEQ ID NO: 491, wherein the sense strand comprises a saturated C18 hydrocarbon chain conjugated to the 5′ terminal nucleotide of the sense strand, and wherein each of the antisense and sense strands comprise at least one 2′-modified nucleotide and at least one modified internucleotide linkage.

In some embodiments, a CD274-targeting oligonucleotide for reducing CD274 expression provided by the disclosure comprises an antisense strand comprising the nucleotide sequence of SEQ ID NO: 732 and a sense strand comprising the nucleotide sequence of SEQ ID NO: 491, wherein the sense strand comprises a saturated C18 hydrocarbon chain conjugated to the 2′ carbon of the ribose ring of the 5′ terminal nucleotide of the sense strand via a linker, and wherein each of the antisense and sense strands comprise at least one 2′-modified nucleotide and at least one modified internucleotide linkage.

In some embodiments, a CD274-targeting oligonucleotide for reducing CD274 expression provided by the disclosure comprises an antisense strand and a sense strand, wherein the antisense strand is 20 to 30 nucleotides in length and has a region of complementarity of 19 to 29 nucleotides to a target sequence of CD274 as set forth in any one of SEQ ID NOs: 2, 5, and 9, wherein the sense strand is 28 to 40 nucleotides in length and comprises at its 3′ end a stem-loop set forth as: S1-L-S2, wherein S1 is complementary to S2, and wherein L forms a loop between S1 and S2 of 3 to 5 nucleotides in length, wherein the antisense strand and the sense strand form a duplex region of at least 19 nucleotides in length, and wherein the sense strand comprises a C18 hydrocarbon chain conjugated to the 5′ terminal nucleotide of the sense strand.

In some embodiments, a CD274-targeting oligonucleotide for reducing CD274 expression provided by the disclosure comprises an antisense strand of about 20 to 22 nucleotides in length and a sense strand of about 28 to 40 nucleotides in length, wherein the antisense and sense strands from an asymmetric duplex region of about 20 to 22 base pairs comprising a 3′ terminal overhang of at least 1 nucleotide of the antisense strand, wherein the antisense strand comprises a region of complementarity of 19 to 21 nucleotides to a target sequence of CD274 as set forth in any one of SEQ ID NOs: 2, 5, and 9, wherein the sense strand comprises: (i) a stem-loop at the 3′ end of the sense strand, wherein the stem-loop comprises a nucleotide sequence represented by the formula: 5′-S1-L-S2-3′, wherein S1 is complementary to S2, and wherein L forms a loop between S1 and S2 of 3 to 5 nucleotides in length, and (ii) at least one C18 hydrocarbon chain conjugated to the 5′ terminal nucleotide of the sense strand, and wherein each of the antisense and sense strands comprise at least one 2′-modified nucleotide and at least one modified internucleotide linkage.

### Formulations

Various formulations (e.g., pharmaceutical formulations) have been developed for oligonucleotide use. For example, oligonucleotides (e.g., RNAi oligonucleotides) can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., RNAi oligonucleotides) reduce the expression of CD274. Such compositions can be suitably formulated such that when administered to a subject, either into the immediate environment of a target cell or systemically, a sufficient portion of the oligonucleotides enter the cell to reduce CD274 expression. Any variety of suitable oligonucleotide formulations can be used to deliver oligonucleotides for the reduction of CD274 as disclosed herein. In some embodiments, an oligonucleotide is formulated in buffer solutions such as phosphate buffered saline solutions, liposomes, micellar structures, and capsids. Any of the oligonucleotides described herein may be provided not only as nucleic acids, but also in the form of a pharmaceutically acceptable salt.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin).

In some embodiments, a pharmaceutical composition is formulated to be compatible with its intended route of administration. Examples of routes of administration include parenteral (e.g., intravenous, intramuscular, intraperitoneal, intradermal, subcutaneous), oral (e.g., inhalation), transdermal (e.g., topical), transmucosal and rectal administration.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., a RNAi oligonucleotide for reducing CD274 expression) or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Cytotoxic T Lymphocyte-Associated Antigen (CTLA4) Inhibitors

In some embodiments, the disclosure provides a CTLA4 inhibitor for use in combination with an oligonucleotide described herein. In some embodiments, the CTLA4 inhibitor inhibits association of CTLA4 with its ligand B7.1 or B7.2. In some embodiments, the CTLA4 inhibitor is specific for CTLA4.

In some embodiments, the CTLA4 inhibitor is an anti-CTLA4 antibody. In some embodiments, the antibody is a full-length antibody. In some embodiments, the antibody is an antibody fragment. In some embodiments, the CTLA4 inhibitor is a small molecule.

In some embodiments, the anti-CTLA4 antibody is Ipilimumab (MDX-010). In some embodiments, the anti-CTLA4 antibody is Tremelimumab.

In some embodiments, the anti-CTLA4 antibody is any anti-CTLA4 antibody known in the art, including, but not limited to, the anti-CTLA4 antibodies disclosed in Ascierto et al. “Anti-CTLA4 monoclonal antibodies: the past and the future in clinical application” J. of Translational Medicine. 9(196): 2011.

In some embodiments, the anti-CTLA4 antibody described herein binds to CTLA4 with an affinity of about 30 nM to about 100 nM. In some embodiments, the anti-CTLA4 antibody described herein binds to CTLA4 with an affinity of about 30 nM. In some embodiments, the anti-PD-L1 antibody described herein binds to CTLA4 with an affinity of about 40 nM. In some embodiments, the anti-CTLA4 antibody described herein binds to CTLA4 with an affinity of about 50 nM. In some embodiments, the anti-CTLA4 antibody described herein binds to CTLA4 with an affinity of about 60 nM. In some embodiments, the anti-CTLA4 antibody described herein binds to CTLA4 with an affinity of about 70 nM. In some embodiments, the anti-CTLA4 antibody described herein binds to CTLA4 with an affinity of about 80 nM. In some embodiments, the anti-CTLA4 antibody described herein binds to CTLA4 with an affinity of about 90 nM. In some embodiments, the anti-CTLA4 antibody described herein binds to CTLA4 with an affinity of about 100 nM.

In some embodiments, the antibody is generated using display technologies. Display technologies used to generate antibody polypeptides include any of the display techniques (e.g. display library screening techniques). In some embodiments, synthetic antibodies are designed, selected, or optimized by screening target antigens using display technologies (e.g. phage display technologies). Phage display libraries may comprise millions to billions of phage vectors, each expressing unique antibody fragments on their viral coats. Such libraries may provide richly diverse resources that are used to select potentially hundreds of antibody fragments with diverse levels of affinity for one or more antigens of interest (McCafferty, et al., 1990. Nature. 348:552-4; Edwards, B. M. et al., 2003. JMB. 334: 103-18; Schofield, D. et al., 2007. Genome Biol. 8, R254 and Pershad, K. et al., 2010. Protein Engineering Design and Selection. 23:279-88; the contents of each of which are herein incorporated by reference in their entirety). Often, the antibody fragments present in such libraries comprise scFv antibody fragments, comprising a fusion protein of V_{H} and V_{L} antibody domains joined by a flexible linker. In some cases, scFvs may contain the same sequence with the exception of unique sequences encoding variable loops of the CDRs. In some cases, scFvs are expressed as fusion proteins, linked to viral coat proteins (e.g. the N-terminus of the viral pill coat protein). VL chains may be expressed separately for assembly with VH chains in the periplasm prior to complex incorporation into viral coats. Precipitated library members may be sequenced from the bound phage to obtain cDNA encoding desired scFvs. Antibody variable domains or CDRs from such sequences may be directly incorporated into antibody sequences for recombinant antibody production or mutated and utilized for further optimization through in vitro affinity maturation.

In some embodiments, the sequences of the polypeptides to be encoded in the viral genomes are produced using yeast surface display technology. In some embodiments, recombinant antibodies are developed by displaying the antibody fragment of interest as a fusion to on the surface of the yeast, where the protein interacts with proteins and small molecules in a solution. scFvs with affinity toward desired receptors may be isolated from the yeast surface using magnetic separation and flow cytometry. Several cycles of yeast surface display and isolation may be done to attain scFvs with desired properties through directed evolution.

Methods for determining the affinity of an antibody for its antigen are known in the art. An exemplary method for determining binding affinity employs surface plasmon resonance. Surface plasmon resonance is an optical phenomenon that allows for the analysis of realtime biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51: 19-26; Jonsson, U., i (1991) Biotechniques 11:620-627; Johnsson, B., et al. (1995) J. Mol. Recognit. 8: 125-131; and Johnsson, B., et al. (1991) Anal. Biochem. 198:268-277.

### Methods of Use

### Reducing CD274 Expression

In some embodiments, the disclosure provides methods for contacting or delivering to a cell or population of cells an effective amount of oligonucleotides provided herein (e.g., RNAi oligonucleotides) to reduce CD274 expression. In some embodiments, a reduction of CD274 expression is determined by measuring a reduction in the amount or level of CD274 mRNA, PD-L1 protein, PD-L1 activity in a cell. The methods include those described herein and known to one of ordinary skill in the art.

Methods provided herein are useful in any appropriate cell type. In some embodiments, a cell is any cell that expresses CD274 mRNA (e.g., hepatocytes). In some embodiments, the cell is a primary cell obtained from a subject. In some embodiments, the primary cell has undergone a limited number of passages such that the cell substantially maintains its natural phenotypic properties. In some embodiments, a cell to which the oligonucleotide is delivered is ex vivo or in vitro (i.e., can be delivered to a cell in culture or to an organism in which the cell resides).

In some embodiments, the oligonucleotides herein (e.g., RNAi oligonucleotides) are delivered to a cell or population of cells using a nucleic acid delivery method known in the art including, but not limited to, injection of a solution containing the oligonucleotides, bombardment by particles covered by the oligonucleotides, exposing the cell or population of cells to a solution containing the oligonucleotides, or electroporation of cell membranes in the presence of the oligonucleotides. Other methods known in the art for delivering oligonucleotides to cells may be used, such as lipid-mediated carrier transport, chemical-mediated transport, and cationic liposome transfection such as calcium phosphate, and others.

In some embodiments, reduction of CD274 expression is determined by an assay or technique that evaluates one or more molecules, properties, or characteristics of a cell or population of cells associated with CD274 expression, or by an assay or technique that evaluates molecules that are directly indicative of CD274 expression in a cell or population of cells (e.g., CD274 mRNA or PD-L1 protein). In some embodiments, the extent to which an oligonucleotide provided herein reduces CD274 expression is evaluated by comparing CD274 expression in a cell or population of cells contacted with the oligonucleotide to an appropriate control (e.g., an appropriate cell or population of cells not contacted with the oligonucleotide or contacted with a control oligonucleotide). In some embodiments, a control amount or level of CD274 expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering an oligonucleotide described herein (e.g., an RNAi oligonucleotide) to a cell or a population of cells results in a reduction in CD274 expression in a cell or population of cells not contacted with the oligonucleotide or contacted with a control oligonucleotide. In some embodiments, the reduction in CD274 expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of CD274 expression. In some embodiments, the control amount or level of CD274 expression is an amount or level of CD274 mRNA and/or PD-L1 protein in a cell or population of cells that has not been contacted with an oligonucleotide herein. In some embodiments, the effect of delivery of an oligonucleotide herein to a cell or population of cells according to a method herein is assessed after any finite period or amount of time (e.g., minutes, hours, days, weeks, months). For example, in some embodiments, CD274 expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotide to the cell or population of cells. In some embodiments, CD274 expression is determined in a cell or population of cells at least about 1 month, about 2 months, about 3 months, about 4 months, about 5 months, or about 6 months or more after contacting or delivering the oligonucleotide to the cell or population of cells.

In some embodiments, an oligonucleotide provided herein (e.g., an RNAi oligonucleotide) is delivered in the form of a transgene that is engineered to express in a cell the oligonucleotide or strands comprising the oligonucleotide (e.g., its sense and antisense strands). In some embodiments, an oligonucleotide herein is delivered using a transgene engineered to express any oligonucleotide disclosed herein. Transgenes may be delivered using viral vectors (e.g., adenovirus, retrovirus, vaccinia virus, poxvirus, adeno-associated virus, or herpes simplex virus) or non-viral vectors (e.g., plasmids or synthetic mRNAs). In some embodiments, transgenes can be injected directly to a subject.

In some embodiments, contacting or delivering an oligonucleotide described herein (e.g., an RNAi oligonucleotide) to the tumor draining lymph node results in a reduction in CD274 expression in a tumor draining lymph node not contacted with the oligonucleotide or contacted with a control oligonucleotide. In some embodiments, the reduction in CD274 expression in the tumor draining lymph node is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of CD274 expression in a tumor draining lymph node not treated with a CD274 oligonucleotide. In some embodiments, the control amount or level of CD274 expression is an amount or level of CD274 mRNA and/or PD-L1 protein in a cell or population of cells that has not been contacted with an oligonucleotide herein.

In some embodiments, contacting or delivering an oligonucleotide described herein (e.g., an RNAi oligonucleotide) to the tumor microenvironment results in a reduction in CD274 expression in a tumor microenvironment not contacted with the oligonucleotide or contacted with a control oligonucleotide. In some embodiments, the reduction in CD274 expression in the tumor microenvironment is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of CD274 expression in a tumor microenvironment not treated with a CD274 oligonucleotide. In some embodiments, the control amount or level of CD274 expression is an amount or level of CD274 mRNA and/or PD-L1 protein in a cell or population of cells that has not been contacted with an oligonucleotide herein.

### Combination of CD274 Oligonucleotide and CTLA4 Inhibitors

In some embodiments, the disclosure provides CD274 oligonucleotides for use, or adaptable for use, to treat a subject (e.g., a human having a disease, disorder or condition associated with CD274 expression) that has received or is receiving a CTLA4 inhibitor.

In some embodiments, methods described herein comprise selecting a subject having a disease, disorder or condition associated with CD274 expression and/or CTLA4 expression or is predisposed to the same. In some instances, the methods can include selecting an individual having a marker for a disease associated with CD274 expression and/or CTLA4 expression such as cancer or other chronic lymphoproliferative disorders.

Likewise, and as detailed herein, the methods also may include steps such as measuring or obtaining a baseline value for a marker of CD274 expression and/or CTLA4 expression, and then comparing such obtained value to one or more other baseline values or values obtained after being administered the oligonucleotide to assess the effectiveness of treatment.

In some embodiments, the disclosure provides methods of treating a subject having, suspected of having, or at risk of developing a disease, disorder, or condition with a CD274 oligonucleotide herein, wherein the subject has received or is receiving a CTLA4 inhibitor. In some embodiments, the disclosure provides methods of treating a subject having, suspected of having, or at risk of developing a disease, disorder, or condition with a CTLA4 inhibitor described herein, wherein the subject has received or is receiving a CD274 oligonucleotide described herein.

In some aspects, the disclosure provides methods of treating or attenuating the onset or progression of a disease, disorder or condition associated with CD274 expression using a CD274 oligonucleotide herein in combination with a CTLA4 inhibitor. In other aspects, the disclosure provides methods to achieve one or more therapeutic benefits in a subject having a disease, disorder or condition associated with CD274 expression using a CD274 oligonucleotide herein in combination with a CTLA4 inhibitor. In some embodiments of the methods herein, the subject is treated by administering a therapeutically effective amount of a CD274 oligonucleotide herein in combination with a CTLA4 inhibitor. In some embodiments of the methods herein, the subject is treated by administering a therapeutically effective amount of a CD274 oligonucleotide herein to a subject that has received or is receiving a CTLA4 inhibitor. In some embodiments of the methods herein, the subject is treated by administering a therapeutically effective amount of a CTLA4 inhibitor to a subject that has received or is receiving a CD274 oligonucleotide herein. In some embodiments, the subject is treated therapeutically. In some embodiments, the subject is treated prophylactically.

In some embodiments of the methods herein, one or more CD274 oligonucleotides herein, or a pharmaceutical composition comprising one or more CD274 oligonucleotides, is administered to a subject having a disease, disorder or condition associated with CD274 expression that has received or is receiving a CTLA4 inhibitor, such that CD274 expression is reduced in the subject, thereby treating the subject. In some embodiments, an amount or level of CD274 mRNA is reduced in the subject. In some embodiments, an amount or level of CD274 and/or protein is reduced in the subject. In some embodiments of the methods herein, one or more CD274 oligonucleotides herein, or a pharmaceutical composition comprising one or more CD274 oligonucleotides, is administered to a subject having a disease, disorder or condition associated with CD274 expression that has received or is receiving a CTLA4 inhibitor such that CD274 expression and CTLA4 signaling is reduced in the subject, thereby treating the subject. In some embodiments, an amount or level of CD274 mRNA and CTLA4 signaling is reduced in the subject. In some embodiments, an amount or level of CD274 and/or protein is reduced in the subject and CTLA4 signaling is reduced in the subject.

In some embodiments, a therapeutically effective amount of a CD274 oligonucleotide and/or CTLA4 inhibitor is administered to a subject. A therapeutically acceptable amount may be an amount that can therapeutically treat a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ. Typically, oligonucleotides herein are administered intravenously or subcutaneously.

As a non-limiting set of examples, the oligonucleotides herein would typically be administered quarterly (once every three months), bi-monthly (once every two months), monthly or weekly. For example, the oligonucleotides may be administered every week or at intervals of two, or three weeks. Alternatively, the oligonucleotides may be administered daily. In some embodiments, a subject is administered one or more loading doses of the oligonucleotide followed by one or more maintenance doses of the oligonucleotide.

In some embodiments, a CTLA4 inhibitor (e.g., an anti-CTLA4 antibody) herein is administered quarterly (once every three months), bi-monthly (once every two months), monthly or weekly. For example, the inhibitor is administered every week or at intervals of two, or three weeks. Alternatively, the inhibitor is administered daily.

In some embodiments the oligonucleotides herein are administered in combination with a CTLA4 inhibitor. In some embodiments the oligonucleotide and inhibitor are administered in combination concurrently, sequentially (in any order), or intermittently. For example, the oligonucleotide and inhibitor may be co-administered concurrently. Alternatively, the oligonucleotide may be administered and followed any amount of time later (e.g., one hour, one day, one week or one month) by the administration of the inhibitor, or vice versa.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

In some embodiments, the disclosure provides a method of treating cancer in a subject, the method comprising (i) administering an oligonucleotide comprising an antisense strand of 15 to 30 nucleotides in length and a sense strand of 15 to 40 nucleotides in length, wherein the antisense and sense strands form a duplex region, wherein the antisense strand comprises a region of complementarity to a CD274 mRNA target sequence, and wherein the region of complementarity is at least 15 contiguous nucleotides in length, and (ii) administering a CTLA-4 inhibitor.

In some embodiments, the disclosure provides a method of treating a disease, disorder or condition associated with activated CD274 expression, comprising administering to a subject in need thereof an RNAi oligonucleotide, and a CTLA-4 inhibitor, wherein the oligonucleotide comprises an antisense strand of 15 to 30 nucleotides in length and a sense strand of 15 to 40 nucleotides in length, wherein the antisense and sense strands form a duplex region, wherein the antisense strand comprises a region of complementarity to a CD274 mRNA target sequence, and wherein the region of complementarity is at least 15 contiguous nucleotides in length.

### Combination of CD274 Oligonucleotide and Cancer Therapy

In some embodiments, the disclosure provides CD274 oligonucleotides for use, or adaptable for use, to treat a subject (e.g., a human having a disease, disorder or condition associated with CD274 expression) that has received or is receiving a cancer therapy. In some embodiments, the cancer therapy is chemotherapy, immunotherapy, radiation therapy, resection surgery, targeted therapy, transplant (solid tissue or stem cell) or a combination thereof.

In some embodiments, methods described herein comprise selecting a subject having a disease, disorder or condition associated with CD274 expression or is predisposed to the same. In some embodiments, the methods comprise selecting an individual having a marker for a disease associated with CD274 expression such as cancer.

In some embodiments, the disclosure provides methods of treating a subject having, suspected of having, or at risk of developing a disease, disorder, or condition with a CD274 oligonucleotide herein, wherein the subject has received or is receiving a cancer therapy.

In some embodiments, the disclosure provides methods of treating or attenuating the onset or progression of a disease, disorder or condition associated with CD274 expression using a CD274 oligonucleotide herein in combination with a cancer therapy. In some embodiments, the disclosure provides methods of treating or attenuating the onset or progression of a disease, disorder or condition associated with CD274 expression using a CD274 oligonucleotide herein in combination with one or more of chemotherapy, immunotherapy, radiation therapy, resection surgery, targeted therapy, transplant (solid tissue or stem cell). In some embodiments, the disclosure provides methods to achieve one or more therapeutic benefits in a subject having a disease, disorder, or condition associated with CD274 expression using a CD274 oligonucleotide herein in combination with a cancer therapy. In some embodiments of the methods herein, the subject is treated by administering a therapeutically effective amount of a CD274 oligonucleotide herein in combination with a cancer therapy. In some embodiments of the methods herein, the subject is treated by administering a therapeutically effective amount of a CD274 oligonucleotide herein to a subject that has received or is receiving a cancer therapy. In some embodiments of the methods herein, the subject is treated by administering a therapeutically effective amount of a cancer therapy to a subject that has received or is receiving a CD274 oligonucleotide herein.

In some embodiments of the methods herein, one or more CD274 oligonucleotides herein, or a pharmaceutical composition comprising one or more CD274 oligonucleotides, is administered to a subject having a disease, disorder or condition associated with CD274 expression that has received or is receiving a cancer therapy, such that CD274 expression is reduced in the subject, thereby treating the subject. In some embodiments, an amount or level of CD274 mRNA is reduced in the subject. In some embodiments, an amount or level of CD274 and/or protein is reduced in the subject.

In some embodiments, a therapeutically effective amount of a CD274 oligonucleotide and/or cancer therapy is administered to a subject. A therapeutically acceptable amount may be an amount that can therapeutically treat a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, the disclosure provides a method of treating cancer in a subject, the method comprising (i) administering an oligonucleotide comprising an antisense strand of 15 to 30 nucleotides in length and a sense strand of 15 to 40 nucleotides in length, wherein the antisense and sense strands form a duplex region, wherein the antisense strand comprises a region of complementarity to a CD274 mRNA target sequence, and wherein the region of complementarity is at least 15 contiguous nucleotides in length, and (ii) administering a cancer therapy.

In some embodiments, the disclosure provides a method of treating a disease, disorder or condition associated with activated CD274 expression, comprising administering to a subject in need thereof an RNAi oligonucleotide, and a cancer therapy, wherein the oligonucleotide comprises an antisense strand of 15 to 30 nucleotides in length and a sense strand of 15 to 40 nucleotides in length, wherein the antisense and sense strands form a duplex region, wherein the antisense strand comprises a region of complementarity to a CD274 mRNA target sequence, and wherein the region of complementarity is at least 15 contiguous nucleotides in length.

In some embodiments, the disclosure provides a method of treating a disease, disorder or condition associated with activated CD274 expression, comprising administering to a subject in need thereof an RNAi oligonucleotide, and a cancer therapy, wherein the oligonucleotide comprises a sense strand comprising SEQ ID NO: 1050 and an antisense strand comprising SEQ ID NO: 1005.

In some embodiments, the disclosure provides a method of treating a disease, disorder or condition associated with activated CD274 expression, comprising administering to a subject in need thereof an RNAi oligonucleotide, and a cancer therapy, wherein the oligonucleotide comprises a sense strand and an antisense strand comprising nucleotide sequences selected from:
(a) SEQ ID NOs: 484 and 725, respectively;
(b) SEQ ID NOs: 487 and 728, respectively; and,
(c) SEQ ID NOs: 491 and 732, respectively,
   wherein the sense and antisense strands are modified based on the pattern below

Sense Strand:
5′-[ademXs-C18][mX][mX][mX][mX][mX][mX][fX][fX]
[fX][fX][mX][mX][mX][mX][mX][mX][mX][mX][mX][mX]
[mX][mX][mX][mX][mX][mX][mX][mX][mX][mX][mX][mX]
[mX][mX][mX]-3′

### Hybridized to:

Antisense Strand:
5′-[MePhosphonate-4O-mXs][fXs][fXs][fX][fX][mX][fX]
[mX][mX][fX][mX][mX][mX][fX][mX][mX][mX][mX][mX]
[mXs][mXs][mX]-3′;

wherein mX=2′-O-methyl modified nucleotide, fX=2′-fluoro modified nucleotide, s=phosphorothioate linkage,][=phosphodiester linkage, [MePhosphonate-4O-mX]=4′-O-monomethylphosphonate-2′-O-methyl modified nucleotide, and AdemX-C18=C18 hydrocarbon chain attached to a nucleotide.

In some embodiments, the disclosure provides a method of treating a disease, disorder or condition associated with activated CD274 expression, comprising administering to a subject in need thereof an RNAi oligonucleotide, and a cancer therapy, wherein the oligonucleotide comprises a sense strand and an antisense strand comprising nucleotide sequences selected from:
(a) SEQ ID NOs: 484 and 725, respectively;
(b) SEQ ID NOs: 487 and 728, respectively; and,
(c) SEQ ID NOs: 491 and 732, respectively,
   wherein the sense and antisense strands are modified based on the pattern below

Sense Strand:
5′-[ademXs-C18][mX][mX][mX][mX][mX][mX[fX][fX][fX]
[fX][mX][mX][mX][mX][mX][mX][mX][mX][mX][mX][mX]
[mX][mX][mX][mX][mX][mX][mX][mX][mX][mX][mX][mX]
[mX][mX]-3′

### Hybridized to:

Antisense Strand:
5′-[MePhosphonate-4O-mXs][fXs][fX][fX][fX][mX][fX]
[mX][mX][fX][mX][mX][mX][fX][mX][mX][mX][mX][mX]
[mXs][mXs][mX]-3′ ;

wherein mX=2′-O-methyl modified nucleotide, fX=2′-fluoro modified nucleotide, s=phosphorothioate linkage,][=phosphodiester linkage, [MePhosphonate-4O-mX]=4′-O-monomethylphosphonate-2′-O-methyl modified nucleotide, and AdemX-C18=C18 hydrocarbon chain attached to a nucleotide.

### Cancers

In some embodiments, the CD274 oligonucleotide, alone or in combination with a CTLA4 inhibitor are used to treat a cancer or a tumor. In some embodiments, the tumor is a primary tumor. In some embodiments, the tumor is a metastatic tumor. In some embodiments, the tumor is a refractory tumor. In some embodiments, the tumor is a Stage I, Stage II, Stage III, or Stage IV tumor. In some embodiments, the tumor is a solid-tumor. Solid-tumors refer to conditions where the cancer forms a mass.

In some embodiments, the cancer is a thyroid cancer, papillary thyroid carcinoma, head and neck cancer, liver cancer, colorectal cancer, pancreatic cancer, breast cancer, ovarian cancer, lung cancer, carcinoma, blastoma, medulloblastoma, retinoblastoma, sarcoma, liposarcoma, synovial cell sarcoma, neuroendocrine tumors, carcinoid tumors, gastrinoma, islet cell cancer, mesothelioma, schwannoma, acoustic neuroma, meningioma, adenocarcinoma, lymphoid malignancies, squamous cell cancer, epithelial squamous cell cancer, small-cell lung cancer (SCLC), non-small cell lung cancer (NSCLC), adenocarcinoma of the lung, squamous carcinoma of the lung, cancer of the peritoneum, hepatocellular cancer, gastric or stomach cancer, gastrointestinal cancer, glioblastoma, cervical cancer, bladder cancer, hepatoma, metastatic breast cancer, colon cancer, rectal cancer, endometrial or uterine carcinoma, salivary gland carcinoma, kidney or renal cancer, prostate cancer, vulval cancer, hepatic carcinoma, anal carcinoma, penile carcinoma, Merkel cell cancer, testicular cancer, esophageal cancer, or tumors of the biliary tract. In some embodiments, the cancer is refractory to anti-PD1, anti-PDL1 and/or anti-CTLA4 therapy. In some embodiments, the cancer is a pancreatic cancer or lung cancer. In some embodiments, the cancer comprises tumors with immunosuppressive tumor microenvironments. In some embodiments, the cancer is resistant to immune checkpoint therapy. In some embodiments, the cancer is partially resistant to immune checkpoint therapy. In some embodiments, the cancer is sensitive to immune checkpoint therapy.

In some embodiments, the CD274 oligonucleotide, alone or in combination with a CLTA4 inhibitor reduces tumor volume. Tumor volume is measured using methods know to one of skill in the art. For example, extracted tumors are measured manually using calipers. Other methods include imagine methods such as ultrasound and MRI. In some embodiments, the oligonucleotide conjugate reduces tumor volume by at least 5%, at least 10%, at least 20%, at least 30%, at least 40%, at least 50%, at least 60%, at least 70%, at least 80%, or at least 90% compared to an untreated tumor.

### Treatment Methods

The disclosure provides oligonucleotides (e.g., RNAi oligonucleotides) for use as a medicament, in particular for use in a method for the treatment of diseases, disorders, and conditions associated with expression of CD274. The disclosure also provides oligonucleotides for use, or adaptable for use, to treat a subject (e.g., a human having a disease, disorder or condition associated with CD274 expression) that would benefit from reducing CD274 expression. In some respects, the disclosure provides oligonucleotides for use, or adapted for use, to treat a subject having a disease, disorder or condition associated with expression of CD274. The disclosure also provides oligonucleotides for use, or adaptable for use, in the manufacture of a medicament or pharmaceutical composition for treating a disease, disorder or condition associated with CD274 expression. In some embodiments, the oligonucleotides for use, or adaptable for use, target CD274 mRNA and reduce CD274 expression (e.g., via the RNAi pathway). In some embodiments, the oligonucleotides for use, or adaptable for use, target CD274 mRNA and reduce the amount or level of CD274 mRNA, PD-L1 protein and/or CD274 activity.

In addition, in some embodiments of the methods herein, a subject having a disease, disorder, or condition associated with CD274 expression or is predisposed to the same is selected for treatment with an oligonucleotide provided herein (e.g., an RNAi oligonucleotide). In some embodiments, the method comprises selecting an individual having a marker (e.g., a biomarker) for a disease, disorder, or condition associated with CD274 expression or predisposed to the same, such as, but not limited to, CD274 mRNA, PD-L1 protein, or a combination thereof. Likewise, and as detailed below, some embodiments of the methods provided by the disclosure include steps such as measuring or obtaining a baseline value for a marker of CD274 expression (e.g., CD274 mRNA), and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder or condition associated with a CD274 expression with an oligonucleotide provided herein. In some aspects, the disclosure provides methods of treating or attenuating the onset or progression of a disease, disorder or condition associated with CD274 expression using the oligonucleotides herein. In other aspects, the disclosure provides methods to achieve one or more therapeutic benefits in a subject having a disease, disorder, or condition associated with CD274 expression using the oligonucleotides provided herein. In some embodiments of the methods herein, the subject is treated by administering a therapeutically effective amount of any one or more of the oligonucleotides provided herein. In some embodiments, treatment comprises reducing CD274 expression. In some embodiments, the subject is treated therapeutically. In some embodiments, the subject is treated prophylactically.

In some embodiments of the methods herein, one or more oligonucleotides herein (e.g., RNAi oligonucleotides), or a pharmaceutical composition comprising one or more oligonucleotides, is administered to a subject having a disease, disorder or condition associated with CD274 expression such that CD274 expression is reduced in the subject, thereby treating the subject. In some embodiments, an amount or level of CD274 mRNA is reduced in the subject. In some embodiments, an amount or level of PD-L1 protein is reduced in the subject. In some embodiments, an amount or level of PD-L1 activity is reduced in the subject.

In some embodiments of the methods herein, an oligonucleotide provided herein (e.g., an RNAi oligonucleotide), or a pharmaceutical composition comprising the oligonucleotide, is administered to a subject having a disease, disorder or condition associated with CD274 such that CD274 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to CD274 expression prior to administration of one or more oligonucleotides or pharmaceutical composition. In some embodiments, CD274 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to CD274 expression in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein (e.g., RNAi oligonucleotides), or a pharmaceutical composition comprising the oligonucleotide or oligonucleotides, is administered to a subject having a disease, disorder or condition associated with CD274 expression such that an amount or level of CD274 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of CD274 mRNA prior to administration of the oligonucleotide or pharmaceutical composition. In some embodiments, an amount or level of CD274 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of CD274 mRNA in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein, or a pharmaceutical composition comprising the oligonucleotide or oligonucleotides, is administered to a subject having a disease, disorder or condition associated with CD274 expression such that an amount or level of PD-L1 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of PD-L1 protein prior to administration of the oligonucleotide or pharmaceutical composition. In some embodiments, an amount or level of PD-L1 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of PD-L1 protein in a subject (e.g., a reference or control subject) not receiving the oligonucleotide or oligonucleotides or pharmaceutical composition or receiving a control oligonucleotide, oligonucleotides or pharmaceutical composition or treatment.

In some embodiments of the methods herein, an oligonucleotide or oligonucleotides (e.g., RNAi oligonucleotides) herein, or a pharmaceutical composition comprising the oligonucleotide or oligonucleotides, is administered to a subject having a disease, disorder or condition associated with CD274 such that an amount or level of CD274 gene activity/expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of CD274 activity prior to administration of the oligonucleotide or pharmaceutical composition. In some embodiments, an amount or level of CD274 activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of CD274 activity in a subject (e.g., a reference or control subject) not receiving the oligonucleotide or pharmaceutical composition or receiving a control oligonucleotide, pharmaceutical composition or treatment.

In some embodiments of the methods herein, an oligonucleotide provided herein (e.g., an RNAi oligonucleotide), or a pharmaceutical composition comprising the oligonucleotide, is administered to a subject having a disease, disorder or condition associated with CD274 such that CD274 expression is reduced in macrophages by at least about 30%, about 35%, about 40%, about 45%, about 50%, about 55%, about 60%, about 65%, about 70%, about 75%, about 80%, about 85%, about 90%, about 95%, about 99% or greater than 99% when compared to CD274 expression prior to administration of one or more oligonucleotides or pharmaceutical composition. In some embodiments, CD274 expression is reduced in macrophages by at least about 30%, about 35%, about 40%, about 45%, about 50%, about 55%, about 60%, about 65%, about 70%, about 75%, about 80%, about 85%, about 90%, about 95%, about 99% or greater than 99% when compared to CD274 expression in macrophages (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide provided herein (e.g., an RNAi oligonucleotide), or a pharmaceutical composition comprising the oligonucleotide, is administered to a subject having a disease, disorder or condition associated with CD274 such that CD274 expression is reduced in dendritic cells by at least about 30%, about 35%, about 40%, about 45%, about 50%, about 55%, about 60%, about 65%, about 70%, about 75%, about 80%, about 85%, about 90%, about 95%, about 99% or greater than 99% when compared to CD274 expression prior to administration of one or more oligonucleotides or pharmaceutical composition. In some embodiments, CD274 expression is reduced in dendritic cells by at least about 30%, about 35%, about 40%, about 45%, about 50%, about 55%, about 60%, about 65%, about 70%, about 75%, about 80%, about 85%, about 90%, about 95%, about 99% or greater than 99% when compared to CD274 expression in dendritic cells (e.g., a reference or control subject) not receiving the oligonucleotide or oligonucleotides or pharmaceutical composition or receiving a control oligonucleotide or oligonucleotides, pharmaceutical composition or treatment.

Suitable methods for determining CD274 expression, the amount or level of CD274 mRNA, PD-L1 protein, PD-L1 activity, or a biomarker related to or affected by modulation of CD274 expression (e.g., a plasma biomarker), in the subject, or in a sample from the subject, are known in the art. Further, the Examples set forth herein illustrate methods for determining CD274 expression.

In some embodiments, CD274 expression, the amount or level of CD274 mRNA, PD-L1 protein, PD-L1 activity, or a biomarker related to or affected by modulation of CD274 expression, or any combination thereof, is reduced in a cell (e.g., a hepatocyte), a population or a group of cells (e.g., an organoid), an organ, blood or a fraction thereof (e.g., plasma), a tissue, a sample (e.g., a biopsy sample), or any other appropriate biological material obtained or isolated from the subject. In some embodiments, CD274 expression, the amount or level of CD274 mRNA, PD-L1 protein, PD-L1 activity, or a biomarker related to or affected by modulation of CD274 expression, or any combination thereof, is reduced in more than one type of cell (e.g., a hepatocyte and one or more other type(s) of cell), more than one groups of cells, more than one organ (e.g., liver and one or more other organ(s)), more than one fraction of blood (e.g., plasma and one or more other blood fraction(s)), more than one type of tissue (e.g., liver tissue and one or more other type(s) of tissue), or more than one type of sample (e.g., a liver biopsy sample and one or more other type(s) of biopsy sample).

Because of their high specificity, the oligonucleotides provided herein (e.g., RNAi oligonucleotides) specifically target mRNA of target genes (e.g., CD274 mRNA) of cells and tissue(s), or organs(s). In preventing disease, the target gene may be one which is required for initiation or maintenance of the disease or which has been identified as being associated with a higher risk of contracting the disease. In treating disease, the oligonucleotide can be brought into contact with the cells, tissue(s), or organ(s) exhibiting or responsible for mediating the disease. For example, an oligonucleotide (e.g., an RNAi oligonucleotide) substantially identical to all or part of a wild-type (i.e., native) or mutated gene associated with a disorder or condition associated with CD274 expression may be brought into contact with or introduced into a cell or tissue type of interest such as a tumor cells or tumor tissue.

Examples of a disease, disorder or condition associated with CD274 expression include, but are not limited to cancer, hepatitis B virus (HBV) infection, hepatitis C virus (HCV) infection, and human immunodeficiency virus (HIV) infection.

In some embodiments, the target gene may be a target gene from any mammal, such as a human target. Any target gene may be silenced according to the method described herein.

Methods described herein typically involve administering to a subject an effective amount of an oligonucleotide herein (e.g., a RNAi oligonucleotide), that is, an amount that produces or generates a desirable therapeutic result. A therapeutically acceptable amount may be an amount that therapeutically treats a disease or disorder. The appropriate dosage for any one subject will depend on certain factors, including the subject's size, body surface area, age, the composition to be administered, the active ingredient(s) in the composition, time and route of administration, general health, and other drugs being administered concurrently.

In some embodiments, a subject is administered any one of the compositions herein (e.g., a composition comprising an RNAi oligonucleotide described herein) either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ. Typically, oligonucleotides herein are administered intravenously or subcutaneously.

In some embodiments, an oligonucleotide herein (e.g., an RNAi oligonucleotide), or a pharmaceutical composition comprising the oligonucleotide, is administered alone or in combination. In some embodiments, the oligonucleotides herein are administered in combination concurrently, sequentially (in any order), or intermittently. For example, two oligonucleotides may be co-administered concurrently. Alternatively, one oligonucleotide may be administered and followed any amount of time later (e.g., one hour, one day, one week or one month) by the administration of a second oligonucleotide.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Kits

In some embodiments, the disclosure provides a kit comprising an oligonucleotide herein (e.g., an RNAi oligonucleotide), and instructions for use. In some embodiments, the kit comprises an oligonucleotide herein, and a package insert containing instructions for use of the kit and/or any component thereof. In some embodiments, the kit comprises, in a suitable container, an oligonucleotide herein, one or more controls, and various buffers, reagents, enzymes and other standard ingredients well known in the art. In some embodiments, the container comprises at least one vial, well, test tube, flask, bottle, syringe, or other container means, into which the oligonucleotide is placed, and in some instances, suitably aliquoted. In some embodiments where an additional component is provided, the kit contains additional containers into which this component is placed. The kits can also include a means for containing the oligonucleotide and any other reagent in close confinement for commercial sale. Such containers may include injection or blow-molded plastic containers into which the desired vials are retained. Containers and/or kits can include labeling with instructions for use and/or warnings.

In some embodiments, a kit comprises an oligonucleotide herein (e.g., an RNAi oligonucleotide), and a pharmaceutically acceptable carrier, or a pharmaceutical composition comprising the oligonucleotide and instructions for treating or delaying progression of a disease, disorder or condition associated with CD274 expression in a subject in need thereof.

### Definitions

As used herein, the term “antisense oligonucleotide” encompasses a nucleic acid-based molecule which has a sequence complementary to all or part of the target mRNA, in particular seed sequence thereby capable of forming a duplex with a mRNA. Thus, the term “antisense oligonucleotide”, as used herein, may be referred to as “complementary nucleic acid-based inhibitor”.

As used herein, “approximately” or “about,” as applied to one or more values of interest, refers to a value that is similar to a stated reference value. In certain embodiments, “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

As used herein, “administer,” “administering,” “administration” and the like refers to providing a substance (e.g., an oligonucleotide) to a subject in a manner that is pharmacologically useful (e.g., to treat a disease, disorder, or condition in the subject).

As used herein, “attenuate,” “attenuating,” “attenuation” and the like refers to reducing or effectively halting. As a non-limiting example, one or more of the treatments herein may reduce or effectively halt the progression of cancer in a subject. This attenuation may be exemplified by, for example, a decrease in one or more aspects (e.g., symptoms, tissue characteristics, and cellular, inflammatory, or immunological activity, etc.) of cancer, no detectable progression (worsening) of one or more aspects of cancer, or no detectable aspects of cancer in a subject when they might otherwise be expected. In some embodiments, one or more of the treatments herein may reduce or effectively halt hepatitis B virus (HBV) infection, hepatitis C virus (HCV) infection, or human immunodeficiency virus (HIV) infection. This attenuation may be exemplified by, for example, a decrease in one or more aspects (e.g., symptoms, tissue characteristics, and cellular, inflammatory, or immunological activity, etc.) of viral infection, no detectable progression (worsening) of one or more aspects of viral infection, or no detectable aspects of viral infection in a subject when they might otherwise be expected.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded oligonucleotide” or “ds oligonucleotide” refers to an oligonucleotide that is substantially in a duplex form. In some embodiments, the complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed (e.g., having overhangs at one or both ends). In some embodiments, a double-stranded oligonucleotide comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect.

As used herein, the term “CD274” or “PD-L1” refers to cluster of differentiation 274 or programmed death-ligand 1. The CD274 gene encodes a type I transmembrane protein which interacts with the PD-1 receptor to inhibit T-cell activation.

As used herein, “labile linker” refers to a linker that can be cleaved (e.g., by acidic pH). A “fairly stable linker” refers to a linker that cannot be cleaved.

As used herein, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cell), the two antiparallel regions, which flank the unpaired region, hybridize to form a duplex (referred to as a “stem”).

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified internucleotide linkage may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single-stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (DsiRNA), antisense oligonucleotide, short siRNA or ss siRNA. In some embodiments, a double-stranded (dsRNA) is an RNAi oligonucleotide.

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of an oligonucleotide. In certain embodiments, the overhang is a 3′ or 5′ overhang on the antisense strand or sense strand of an oligonucleotide.

As used herein, “phosphate analog” refers to a chemical moiety that mimics the electrostatic and/or steric properties of a phosphate group. In some embodiments, the phosphate analog mimics the electrostatic and/or steric properties of a phosphate group in biologic systems. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylene phosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethyl phosphonate, in which the oxygen atom of the oxymethyl group is bound to the sugar moiety (e.g., at its 4′-carbon) or analog thereof. See, e.g., US Patent Publication No. 2019-0177729. Other modifications have been developed for the 5′ end of oligonucleotides (see, e.g., Intl. Patent Application No. WO 2011/133871; U.S. Pat. No. 8,927,513; and Prakash et al. (2015) NUCLEIC ACIDS RES. 43:2993-3011).

As used herein, “reduced expression” of a gene (e.g., CD274) refers to a decrease in the amount or level of RNA transcript (e.g., CD274 mRNA) or protein encoded by the gene and/or a decrease in the amount or level of activity of the gene in a cell, a population of cells, a sample, or a subject, when compared to an appropriate reference (e.g., a reference cell, population of cells, sample or subject). For example, the act of contacting a cell with an oligonucleotide herein (e.g., an oligonucleotide comprising an antisense strand having a nucleotide sequence that is complementary to a nucleotide sequence comprising CD274 mRNA) may result in a decrease in the amount or level of CD274 mRNA, PD-L1 protein and/or activity (e.g., via degradation of CD274 mRNA by the RNAi pathway) when compared to a cell that is not treated with the oligonucleotide. Similarly, and as used herein, “reducing expression” refers to an act that results in reduced expression of a gene (e.g., CD274).

As used herein, “reduction of CD274 expression” refers to a decrease in the amount or level of CD274 mRNA, PD-L1 protein, PD-L1 activity in a cell, a population of cells, a sample or a subject when compared to an appropriate reference (e.g., a reference cell, population of cells, sample, or subject).

As used herein, “region of complementarity” refers to a sequence of nucleotides of a nucleic acid (e.g., an oligonucleotide) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a double-stranded oligonucleotide having a sense strand (passenger) and antisense strand (guide), in which the antisense strand or part of the antisense strand is used by the Argonaute 2 (Ago2) endonuclease in the cleavage of a target mRNA (e.g., CD274 mRNA) or (b) a single-stranded oligonucleotide having a single antisense strand, where that antisense strand (or part of that antisense strand) is used by the Ago2 endonuclease in the cleavage of a target mRNA (e.g., CD274 mRNA).

As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits, and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule (e.g., a carbohydrate, amino sugar, cholesterol, polypeptide, or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a Tₘ of at least about 50° C., at least about 55° C., at least about 56° C., at least about 58° C., at least about 60° C., at least about 65° C. or at least about 75° C. in 10 mM Na₂HPO₄ to a hairpin comprising a duplex of at least 2 base pairs (bp) in length. In some embodiments, a tetraloop can confer a Tm of at least about 50° C., at least about 55° C., at least about 56° C., at least about 58° C., at least about 60° C., at least about 65° C. or at least about 75° C. in 10 mM NaH₂PO₄ to a hairpin comprising a duplex of at least 2 base pairs (bp) in length. In some embodiments, a tetraloop may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al. (1990) NATURE 346:680-82; Heus & Pardi (1991) SCIENCE 253:191-94). In some embodiments, a tetraloop comprises or consists of 3 to 6 nucleotides and is typically 4 to 5 nucleotides. In certain embodiments, a tetraloop comprises or consists of 3, 4, 5 or 6 nucleotides, which may or may not be modified (e.g., which may or may not be conjugated to a targeting moiety). In one embodiment, a tetraloop consists of 4 nucleotides. Any nucleotide may be used in the tetraloop and standard IUPAC-IUB symbols for such nucleotides may be used as described in Cornish-Bowden (1985) NUCLEIC ACIDS RES. 13:3021-30. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al. (1990) PROC. NATL. ACAD. SCI. USA 87:8467-71; Antao et al. (1991) NUCLEIC ACIDS RES. 19:5901-05). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). See, e.g., Nakano et al. (2002) BIOCHEM. 41:14281-92; Shinji et al. (2000) NIPPON KAGAKKAI KOEN YOKOSHU 78:731. In some embodiments, the tetraloop is contained within a nicked tetraloop structure.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### OTHER EMBODIMENTS

E1. An oligonucleotide for reducing CD274 expression, the oligonucleotide comprising an antisense strand of 15 to 30 nucleotides in length and a sense strand of 15 to 40 nucleotides in length, wherein the sense strand and antisense strand form a duplex region, wherein the antisense strand has a region of complementarity to a target sequence of CD274 as set forth in any one of SEQ ID NOs: 1-2 and 4-241.
E2. The oligonucleotide of embodiment E1, wherein the region of complementarity is fully complementary to the target sequence of CD274.
E3. The oligonucleotide of any one of embodiments E1 or E2, wherein the antisense strand is 19 to 27 nucleotides in length.
E4. The oligonucleotide of any one of embodiments E1 to E3, wherein the antisense strand is 21 to 27 nucleotides in length, optionally wherein the antisense strand is 22 nucleotides in length.
E5. The oligonucleotide of embodiment E1 wherein the sense strand is 19 to 40 nucleotides in length, optionally wherein the sense strand is 36 nucleotides in length.
E6. The oligonucleotide of any one of embodiments E1-E5, wherein the duplex region is at least 19 nucleotides in length.
E7. The oligonucleotide of any one of embodiments E1-E6, wherein the duplex region is at least 20 nucleotides in length, optionally wherein the duplex region is 21 nucleotides in length.
E8. The oligonucleotide of any one of embodiments E1-E7, wherein the region of complementarity is at least 19 contiguous nucleotides in length.
E9. The oligonucleotide of any one of embodiments E1-E8, wherein the region of complementarity is at least 21 contiguous nucleotides in length.
E10. The oligonucleotide of any one of embodiments E1-E9, wherein the antisense strand comprises a sequence as set forth in any one of SEQ ID NOs: 242-243 and 245-482.
E11. The oligonucleotide of any one of embodiments E1-E10, wherein the sense strand comprises a sequence as set forth in any one of SEQ ID NOs: 1-2 and 4-241.
E12. The oligonucleotide of any one of embodiments E1 to E11, wherein the sense strand comprises at its 3′ end a stem-loop set forth as: S1-L-S2, wherein S1 is complementary to S2, and wherein L forms a loop between S1 and S2 of 3 to 5 nucleotides in length.
E13. An oligonucleotide for reducing CD274 expression, the oligonucleotide comprising an antisense strand and a sense strand, wherein the antisense strand is 21 to 27 nucleotides in length and has a region of complementarity to a target sequence of CD274 as set forth in any one of SEQ ID NOs: 1-2 and 4-241, wherein the sense strand comprises at its 3′ end a stem-loop set forth as: S1-L-S2, wherein S1 is complementary to S2, and wherein L forms a loop between S1 and S2 of 3 to 5 nucleotides in length, and wherein the antisense strand and the sense strand form a duplex region of at least 19 nucleotides in length.
E14. The oligonucleotide of embodiment E13, wherein the region of complementarity is at least 19 nucleotides in length and is fully complementary to the target sequence.
E15. The oligonucleotide of any one of embodiments E12-E14, wherein L is a tetraloop.
E16. The oligonucleotide of any one of embodiments E12-E15, wherein L is 4 nucleotides in length.
E17. The oligonucleotide of any one of embodiments E12-E16, wherein L comprises a sequence set forth as GAAA.
E18. The oligonucleotide of any one of embodiments E1-E17, wherein (i) the antisense strand is 27 nucleotides in length and the sense strand is 25 nucleotides in length, or (ii) the antisense strand is 22 nucleotides in length and the sense strand is 36 nucleotides in length.
E19. The oligonucleotide of embodiment E18, wherein the antisense strand and sense strand form a duplex region of 25 nucleotides in length or 20 nucleotides in length.
E20. The oligonucleotide of any one of embodiments E1-E19, wherein the antisense strand comprises a 3′ overhang sequence of one or more nucleotides in length, optionally wherein the 3′ overhang sequence is 2 nucleotides in length, optionally wherein the 3′ overhang sequence is GG.
E21. The oligonucleotide of any one of the preceding embodiments, wherein the oligonucleotide comprises at least one modified nucleotide.
E22. The oligonucleotide of embodiment E21, wherein the modified nucleotide comprises a 2′-modification.
E23. The oligonucleotide of embodiment E22, wherein the 2′-modification is a modification selected from 2′-aminoethyl, 2′-fluoro, 2′-O-methyl, 2′-O-methoxyethyl, and 2′-deoxy-2′-fluoro-β-d-arabinonucleic acid.
E24. The oligonucleotide of any one of embodiments E20-E23, wherein about 10-15%, 10%, 11%, 12%, 13%, 14% or 15% of the nucleotides of the sense strand comprise a 2′-fluoro modification.
E25. The oligonucleotide of any one of embodiments E20-E24, wherein about 25-35%, 25%, 26%, 27%, 28%, 29%, 30%, 31%, 32%, 33%, 34% or 35% of the nucleotides of the antisense strand comprise a 2′-fluoro modification.
E26. The oligonucleotide of any one of embodiments E20-E25, wherein about 25-35%, 25%, 26%, 27%, 28%, 29%, 30%, 31%, 32%, 33%, 34% or 35% of the nucleotides of the oligonucleotide comprise a 2′-fluoro modification.
E27. The oligonucleotide of any one of embodiments E20-E26, wherein the sense strand comprises 36 nucleotides with positions 1-36 from 5′ to 3′, wherein positions 8-11 comprise a 2′-fluoro modification.
E28. The oligonucleotide of any one of embodiments E20-E27, wherein the antisense strand comprises 22 nucleotides with positions 1-22 from 3′ to 5′, and wherein positions 2, 3, 4, 5, 7, 10 and 14 comprise a 2′-fluoro modification.
E29. The oligonucleotide of any one of embodiments E23-E28, wherein the remaining nucleotides comprise a 2′-O-methyl modification.
E30. The oligonucleotide of embodiment E27, wherein positions 1-7, 12-27 and 31-36 of the sense strand comprise a 2′-O-methyl modification.
E31. The oligonucleotide of embodiment E28 or E30, wherein positions 1, 6, 8, 9, 11-13 and 15-22 of the antisense strand comprise a 2′-O-methyl modification.
E32. The oligonucleotide of any one of embodiments E20-E31, wherein all of the nucleotides of the oligonucleotide are modified.
E33. The oligonucleotide of any one of the preceding embodiments, wherein the oligonucleotide comprises at least one modified internucleotide linkage.
E34. The oligonucleotide of embodiment E33, wherein the at least one modified internucleotide linkage is a phosphorothioate linkage.
E35. The oligonucleotide of embodiment E34, wherein the sense strand comprises a phosphorothioate linkage between positions 1 and 2 of the sense strand.
E36. The oligonucleotide of embodiment E34, wherein the sense strand comprises a phosphorothioate linkage between positions 1 and 2, 2 and 3, and 3 and 4 of the sense strand.
E37. The oligonucleotide of any one of embodiments E34-E36, wherein the antisense strand comprises 22 nucleotides with positions 1-22 from 3′ to 5′, wherein the antisense strand comprises a phosphorothioate linkage between positions 1 and 2, 2 and 3, 20 and 21, and 21 and 22.
E38. The oligonucleotide of any one of the preceding embodiments, wherein the 4′-carbon of the sugar of the 5′-nucleotide of the antisense strand comprises a phosphate analog.
E39. The oligonucleotide of embodiment E38, wherein the phosphate analog is oxymethyl phosphonate, vinyl phosphonate or malonyl phosphonate.
E40. The oligonucleotide of any one of the preceding embodiments, wherein at least one nucleotide of the oligonucleotide is conjugated to one or more targeting ligands.
E41. The oligonucleotide of embodiment E40, wherein the nucleotide is conjugated to more than one targeting ligands, wherein the targeting ligands are the same or are different.
E42. The oligonucleotide of embodiment E40 or E41, wherein the one or more targeting ligands is selected from carbohydrate, amino sugar, cholesterol, polypeptide, or lipid.
E43. The oligonucleotide of E40 or E41, wherein the targeting ligand comprises a N-acetylgalactosamine (GalNAc) moiety.
E44. The oligonucleotide of embodiment E43, wherein the GalNAc moiety is a monovalent GalNAc moiety, a bivalent GalNAc moiety, a trivalent GalNAc moiety or a tetravalent GalNAc moiety.
E45. The oligonucleotide of any one of embodiments E12-E44, wherein up to 4 nucleotides of L of the stem-loop are each conjugated to a monovalent GalNAc moiety.
E46. The oligonucleotide of any one of embodiments E1-E45, wherein the sense strand and antisense strand comprise nucleotide sequences selected from the group consisting of:
(a) SEQ ID NOs: 483 and 724, respectively;
(b) SEQ ID NOs: 484 and 725, respectively;
(c) SEQ ID NOs: 486 and 727, respectively;
(d) SEQ ID NOs: 487 and 728, respectively;
(e) SEQ ID NOs: 488 and 729, respectively;
(f) SEQ ID NOs: 489 and 730, respectively;
(g) SEQ ID NOs: 490 and 731, respectively;
(h) SEQ ID NOs: 492 and 733, respectively;
(i) SEQ ID NOs: 493 and 734, respectively;
(j) SEQ ID NOs: 494 and 735, respectively;
(k) SEQ ID NOs: 495 and 736, respectively;
(l) SEQ ID NOs: 496 and 737, respectively;
(m) SEQ ID NOs: 497 and 738, respectively;
(n) SEQ ID NOs: 498 and 739, respectively;
(o) SEQ ID NOs: 499 and 740, respectively;
(p) SEQ ID NOs: 500 and 741, respectively;
(q) SEQ ID NOs: 501 and 742, respectively;
(r) SEQ ID NOs: 502 and 743, respectively;
(s) SEQ ID NOs: 503 and 744, respectively;
(t) SEQ ID NOs: 504 and 745, respectively;
(u) SEQ ID NOs: 505 and 746, respectively;
(v) SEQ ID NOs: 506 and 747, respectively;
(w) SEQ ID NOs: 507 and 748, respectively;
(x) SEQ ID NOs: 508 and 749, respectively;
(y) SEQ ID NOs: 509 and 750, respectively;
(z) SEQ ID NOs: 510 and 751, respectively;
(aa) SEQ ID NOs: 511 and 752, respectively;
(bb) SEQ ID NOs: 512 and 753, respectively;
(cc) SEQ ID NOs: 513 and 754, respectively;
(dd) SEQ ID NOs: 514 and 755, respectively;
(ee) SEQ ID NOs: 515 and 756, respectively;
(ff) SEQ ID NOs: 516 and 757, respectively;
(gg) SEQ ID NOs: 517 and 758, respectively;
(hh) SEQ ID NOs: 518 and 758, respectively; and,
(ii) SEQ ID NOs: 491 and 732, respectively.
   E47. The oligonucleotide of any one of embodiments E1-E45, wherein the sense strand comprises the nucleotide sequence of SEQ ID NO: 484 and the antisense strand comprises the nucleotides sequence of SEQ ID NO: 725.
   E48. The oligonucleotide of any one of embodiments E1-E45, wherein the sense strand comprises the nucleotide sequence of SEQ ID NO: 486 and the antisense strand comprises the nucleotides sequence of SEQ ID NO: 727.
   E49. The oligonucleotide of any one of embodiments E1-E45, wherein the sense strand comprises the nucleotide sequence of SEQ ID NO: 487 and the antisense strand comprises the nucleotides sequence of SEQ ID NO: 728.
   E50. The oligonucleotide of any one of embodiments E1-E45, wherein the sense strand comprises the nucleotide sequence of SEQ ID NO: 488 and the antisense strand comprises the nucleotides sequence of SEQ ID NO: 729.
   E51. The oligonucleotide of any one of embodiments E1-E45, wherein the sense strand comprises the nucleotide sequence of SEQ ID NO: 489 and the antisense strand comprises the nucleotides sequence of SEQ ID NO: 730.
   E52. The oligonucleotide of any one of embodiments E1-E45, wherein the sense strand comprises the nucleotide sequence of SEQ ID NO: 491 and the antisense strand comprises the nucleotides sequence of SEQ ID NO: 732.
   E53. The oligonucleotide of any one of embodiments E1-E45, wherein the sense strand comprises the nucleotide sequence of SEQ ID NO: 502 and the antisense strand comprises the nucleotides sequence of SEQ ID NO: 743.
   E54. The oligonucleotide of any one of embodiments E1-E45, wherein the sense strand comprises a nucleotide sequence of any one of SEQ ID NOs: 965-1000.
   E55. The oligonucleotide of any one of embodiments E1-E45, wherein the antisense strand comprises a nucleotide sequence of any one of SEQ ID NOs: 1001-1036.
   E56. The oligonucleotide of any one of embodiments E1-E45, wherein the sense strand and antisense strand comprise nucleotide sequences selected from the consisting of:
(a) SEQ ID NOs: 965 and 1001, respectively;
(b) SEQ ID NOs: 966 and 1002, respectively;
(c) SEQ ID NOs: 968 and 1004, respectively;
(d) SEQ ID NOs: 969 and 1005, respectively;
(e) SEQ ID NOs: 970 and 1006, respectively;
(f) SEQ ID NOs: 971 and 1007, respectively;
(g) SEQ ID NOs: 972 and 1008, respectively;
(h) SEQ ID NOs: 974 and 1010, respectively;
(i) SEQ ID NOs: 975 and 1011, respectively;
(j) SEQ ID NOs: 976 and 1012, respectively;
(k) SEQ ID NOs: 977 and 1013, respectively;
(l) SEQ ID NOs: 978 and 1014, respectively;
(m) SEQ ID NOs: 979 and 1015, respectively;
(n) SEQ ID NOs: 980 and 1016, respectively;
(o) SEQ ID NOs: 981 and 1017, respectively;
(p) SEQ ID NOs: 982 and 1018, respectively;
(q) SEQ ID NOs: 983 and 1019, respectively;
(r) SEQ ID NOs: 984 and 1020, respectively;
(s) SEQ ID NOs: 985 and 1021, respectively;
(t) SEQ ID NOs: 986 and 1022, respectively;
(u) SEQ ID NOs: 987 and 1023, respectively;
(v) SEQ ID NOs: 988 and 1024, respectively;
(w) SEQ ID NOs: 989 and 1025, respectively;
(x) SEQ ID NOs: 990 and 1026, respectively;
(y) SEQ ID NOs: 991 and 1027, respectively;
(z) SEQ ID NOs: 992 and 1028, respectively;
(aa) SEQ ID NOs: 993 and 1029, respectively;
(bb) SEQ ID NOs: 994 and 1030, respectively;
(cc) SEQ ID NOs: 995 and 1031, respectively;
(dd) SEQ ID NOs: 996 and 1032, respectively;
(ee) SEQ ID NOs: 997 and 1033, respectively;
(ff) SEQ ID NOs: 998 and 1034, respectively;
(gg) SEQ ID NOs: 999 and 1035, respectively;
(hh) SEQ ID NOs: 1000 and 1036, respectively; and,
(ii) SEQ ID NOs: 973 and 1009, respectively.
   E57. The oligonucleotide of any one of embodiments E1-E45, wherein the sense strand comprises the nucleotide sequence of SEQ ID NO: 966 and the antisense strand comprises the nucleotides sequence of SEQ ID NO: 1002.
   E58. The oligonucleotide of any one of embodiments E1-E45, wherein the sense strand comprises the nucleotide sequence of SEQ ID NO: 968 and the antisense strand comprises the nucleotides sequence of SEQ ID NO: 1004.
   E59. The oligonucleotide of any one of embodiments E1-E45, wherein the sense strand comprises the nucleotide sequence of SEQ ID NO: 969 and the antisense strand comprises the nucleotides sequence of SEQ ID NO: 1005.
   E60. The oligonucleotide of any one of embodiments E1-E45, wherein the sense strand comprises the nucleotide sequence of SEQ ID NO: 970 and the antisense strand comprises the nucleotides sequence of SEQ ID NO: 1006.
   E61. The oligonucleotide of any one of embodiments E1-E45, wherein the sense strand comprises the nucleotide sequence of SEQ ID NO: 971 and the antisense strand comprises the nucleotides sequence of SEQ ID NO: 1007.
   E62. The oligonucleotide of any one of embodiments E1-E45, wherein the sense strand comprises the nucleotide sequence of SEQ ID NO: 973 and the antisense strand comprises the nucleotides sequence of SEQ ID NO: 1009.
   E63. The oligonucleotide of any one of embodiments E1-E45, wherein the sense strand comprises the nucleotide sequence of SEQ ID NO: 984 and the antisense strand comprises the nucleotides sequence of SEQ ID NO: 1020.
   E64. A pharmaceutical composition comprising the oligonucleotide of any one of embodiments E1-E63, and a pharmaceutically acceptable carrier, delivery agent or excipient.
   E65. A method of treating cancer in a subject, the method comprising administering to the subject an effective amount of the oligonucleotide of any one of embodiments E1-E63 or the pharmaceutical composition of embodiment E64.
   E66. A method of treating a disease, disorder or condition associated with activated CD274 expression, comprising administering to a subject in need thereof the oligonucleotide of any one of embodiments E1-E63 or the pharmaceutical composition of embodiment E64.
   E67. A method of treating cancer in a subject, the method comprising administering to the subject an effective amount of the oligonucleotide of any one of embodiments E1-E63 or the pharmaceutical composition of embodiment E64, in combination with a CTLA4 inhibitor.
   E68. A method of treating a treating a disease, disorder or condition associated with activated CD274 expression, comprising administering to a subject in need thereof the oligonucleotide of any one of embodiments E1-E63 or the pharmaceutical composition of embodiment E64, in combination with a CTLA4 inhibitor.
   E69. The method of embodiment E66 or E68, wherein the disease, disorder, or condition associated with activated CD274 expression is a cancer.
   E70. The method of any one of embodiments E65, E66, and E69, wherein the cancer is selected from carcinoma, sarcoma, melanoma, lymphoma, and leukemia, prostate cancer, breast cancer, hepatocellular carcinoma (HCC), colorectal cancer, pancreatic cancer and glioblastoma.
   E71. The method of any one of embodiments E65, E66, and E69-E70, wherein the cancer comprises an immunosuppressive tumor microenvironment.
   E72. The method of any one of embodiments E65, E66, and E69-E71, wherein the cancer comprises an inflamed tumor microenvironment.
   E73. The method of embodiment E72, wherein the inflamed tumor microenvironment comprises infiltrating T cells.
   E74. The method of any one of embodiments E67-E73, wherein the CTLA-4 inhibitor is an antibody.
   E75. The method of embodiment E74, wherein the antibody is an anti-CTLA-4 antibody.
   E76. The method of embodiment E75, wherein the anti-CTLA-4 antibody is selected from Ipilimumab and Tremelimumab.
   E77. Use of the oligonucleotide of any one of embodiments E1-E63, or the pharmaceutical composition of embodiment E64, in the manufacture of a medicament for the treatment of a disease, disorder, or condition associated with CD274 expression, optionally for the treatment of cancer.
   E78. The oligonucleotide of any one of embodiments E1-E63, or the pharmaceutical composition of embodiment E64, for use, or adaptable for use, in the treatment of a disease, disorder or condition associated with CD274 expression, optionally for the treatment of cancer.
   E79. A kit comprising the oligonucleotide of any one of embodiments E1-E63, an optional pharmaceutically acceptable carrier, and a package insert comprising instructions for administration to a subject having a disease, disorder or condition associated with CD274 expression.
   E80. The use of embodiment E77, the oligonucleotide or pharmaceutical composition for use, or adaptable for use, of embodiment E78, or the kit of embodiment E79, wherein the disease, disorder or condition associated with CD274 expression is cancer.
   E81. Use of the oligonucleotide of any one of embodiments E1-E63, or the pharmaceutical composition of embodiment E64, in the manufacture of a medicament for the treatment of a disease, disorder, or condition associated with CD274 expression, in combination with a CTLA4 inhibitor.
   E82. The oligonucleotide of any one of embodiments E1-E63, or the pharmaceutical composition of embodiment E64, for use, or adaptable for use, in the treatment of a disease, disorder or condition associated with CD274 expression, in combination with a CTLA4 inhibitor.
   E83. A kit comprising the oligonucleotide of any one of embodiments E1-E63, an optional pharmaceutically acceptable carrier, and a package insert comprising instructions for administration of the RNAi oligonucleotide in combination with a CTLA4 inhibitor to a subject having a disease, disorder or condition associated with CD274 expression.
   E84. The use of embodiment E81, the oligonucleotide or pharmaceutical composition for use, or adaptable for use, of embodiment E82, or the kit of embodiment E83, wherein the disease, disorder or condition associated with CD274 expression is cancer.

### EXAMPLES

### Example 1: Preparation of Double-Stranded RNAi Oligonucleotides

### General Synthetic Methods

The following examples are intended to illustrate the disclosure and are not to be construed as being limitations thereon. Temperatures are given in degrees centigrade (C). If not mentioned otherwise, all evaporations are performed under reduced pressure, preferably between about 15 mm Hg and 100 mm Hg (=20-133 mbar). The structure of final products, intermediates and starting materials was confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the nucleic acid or analogues thereof of the present disclosure are either commercially available or can be produced by organic synthesis methods known to one of ordinary skill in the art (METHODS OF ORGANIC SYNTHESIS, Thieme, Volume 21 (Houben-Weyl 4th Ed. 1952)). Further, the nucleic acid or analogues thereof of the present disclosure can be produced by organic synthesis methods known to one of ordinary skill in the art as shown in the following examples.

All reactions are carried out under nitrogen or argon unless otherwise stated.

Proton NMR (¹H NMR) was conducted in deuterated solvent. In certain nucleic acid or analogues thereof disclosed herein, one or more ¹H shifts overlap with residual proteo solvent signals; these signals have not been reported in the experimental provided hereinafter.

As depicted in the Examples below, in certain exemplary embodiments, the nucleic acid or analogues thereof were prepared according to the following general procedures. It will be appreciated that, although the general methods depict the synthesis of certain nucleic acid or analogues thereof of the present disclosure, the following general methods, and other methods known to one of ordinary skill in the art, can be applied to all nucleic acid or analogues thereof and subclasses and species of each of these nucleic acid or analogues thereof, as described herein.

### Example 1a: Synthesis of 2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy) ethan-1-ammonium formate (1-6)

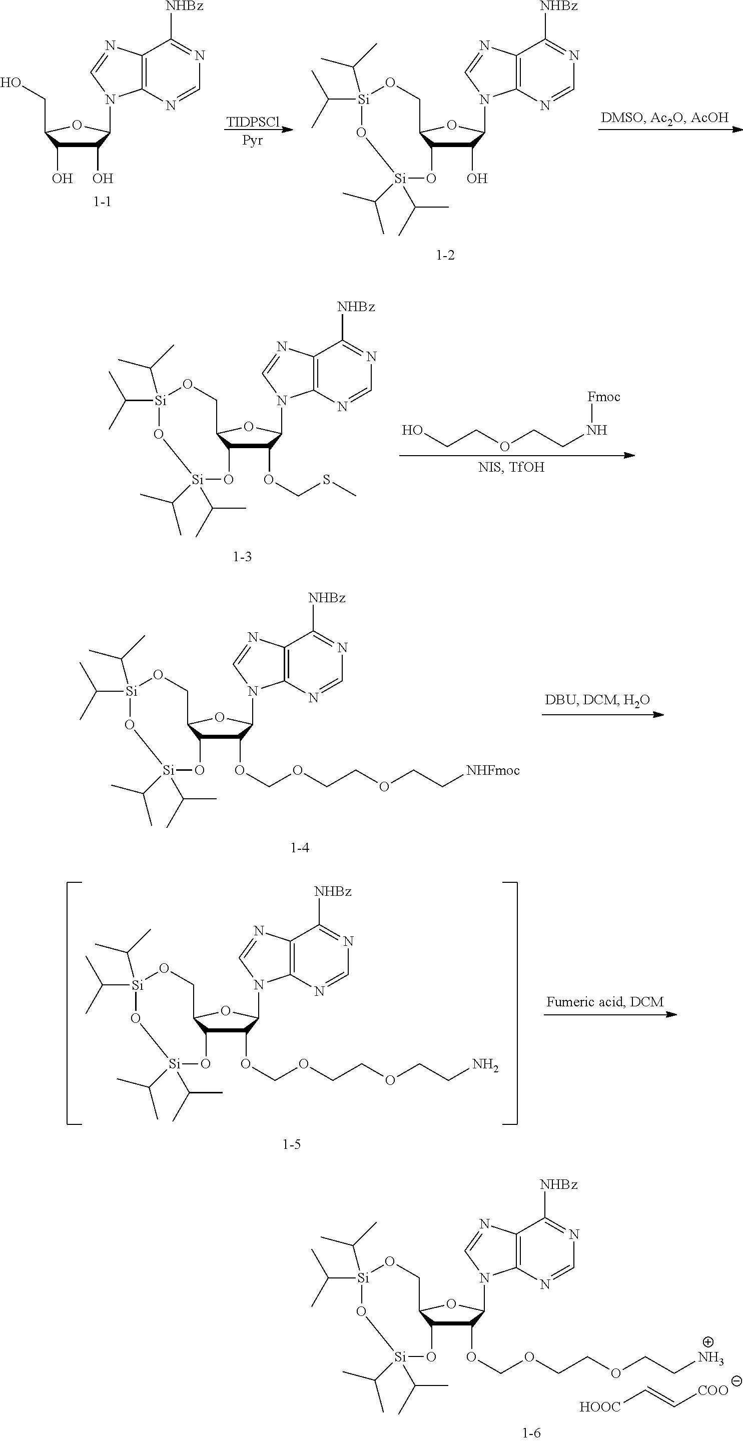

A solution of compound 1-1 (25.00 g, 67.38 mmol) in 20 mL of DMF was treated with pyridine (11 mL, 134.67 mmol) and tetraisopropyldisiloxane dichloride (22.63 mL, 70.75 mmol) at 10° C. The resulting mixture was stirred at 25° C. for 3 h and quenched with 20% citric acid (50 mL). The aqueous layer was extracted with EtOAc (3×50 mL) and the combined organic layers were concentrated in vacuo. The crude residue was recrystallized from a mixture of MTBE and n-heptane (1:15, 320 mL) to afford compound 1-2 (37.20 g, 90%) as a white oily solid.

A solution of compound 1-2 (37.00 g, 60.33 mmol) in 20 mL of DMSO was treated with AcOH (20 mL, 317.20 mmol) and Ac₂O (15 mL, 156.68 mmol). The mixture was stirred at 25° C. for 15 h. The reaction was diluted with EtOAc (100 mL) and quenched with sat. K₂CO₃ (50 mL). The aqueous layer was extracted with EtOAc (3×50 mL). The combined organic layers were concentrated and recrystallized with ACN (30 mL) to afford compound 1-3 (15.65 g, 38.4%) as a white solid.

A solution of compound 1-3 (20.00 g, 29.72 mmol) in 120 mL of DCM was treated with Fmoc-amino-ethoxy ethanol (11.67 g, 35.66 mmol) at 25° C. The mixture was stirred to afford a clear solution and then treated with 4 Å molecular sieves (20.0 g), N-iodosuccinimide (8.02 g, 35.66 mmol), and TfOH (5.25 mL, 59.44 mmol). The mixture was stirred at 30° C. until the HPLC analysis indicated >95% consumption of compound 1-3. The reaction was quenched with TEA (6 mL) and filtered. The filtrate was diluted with EtOAc, washed with sat. NaHCO₃ (2×100 mL), sat. Na₂SO₃ (2×100 mL), and water (2×100 mL) and concentrated in vacuo to afford crude compound 1-4 (26.34 g, 93.9%) as a yellow solid, which was used directly for the next step without further purification.

A solution of compound 1-4 (26.34 g, 27.62 mmol) in a mixture of DCM/water (10:7, 170 mL) was treated with DBU (7.00 mL, 45.08 mmol) at 5° C. The mixture was stirred at 5-25° C. for 1 h. The organic layer was then separated, washed with water (100 mL), and diluted with DCM (130 mL). The solution was treated with fumaric acid (7.05 g, 60.76 mmol) and 4 Å molecular sieves (26.34 g) in four portions. The mixture was stirred for 1 h, concentrated, and recrystallized from a mixture of MTBE and DCM (5:1) to afford compound 1-6 (14.74 g, 62.9%) as a white solid: ¹H NMR (400 MHz, d₆-DMSO) 8.73 (s, 1H), 8.58 (s, 1H), 8.15-8.02 (m, 2H), 7.65-7.60 (m, 1H), 7.59-7.51 (m, 2H), 6.52 (s, 2H), 6.15 (s, 1H), 5.08-4.90 (m, 3H), 4.83-4.78 (m, 1H), 4.15-3.90 (m, 3H), 3.79-3.65 (m, 2H), 2.98-2.85 (m, 6H), 1.20-0.95 (m, 28H).

### Example 1b: Synthesis of (2R,3R,4R,5R)-5-(6-benzamido-9H-purin-9-yl)-2-((bis(4-methoxyphenyl)(phenyl)methoxy)methyl)-4-((2-(2-[lipid]-amidoethoxy)ethoxy)methoxy) tetrahydrofuran-3-yl (2-cyanoethyl) diisopropylphosphoramidite (2-4a to 2-4e)

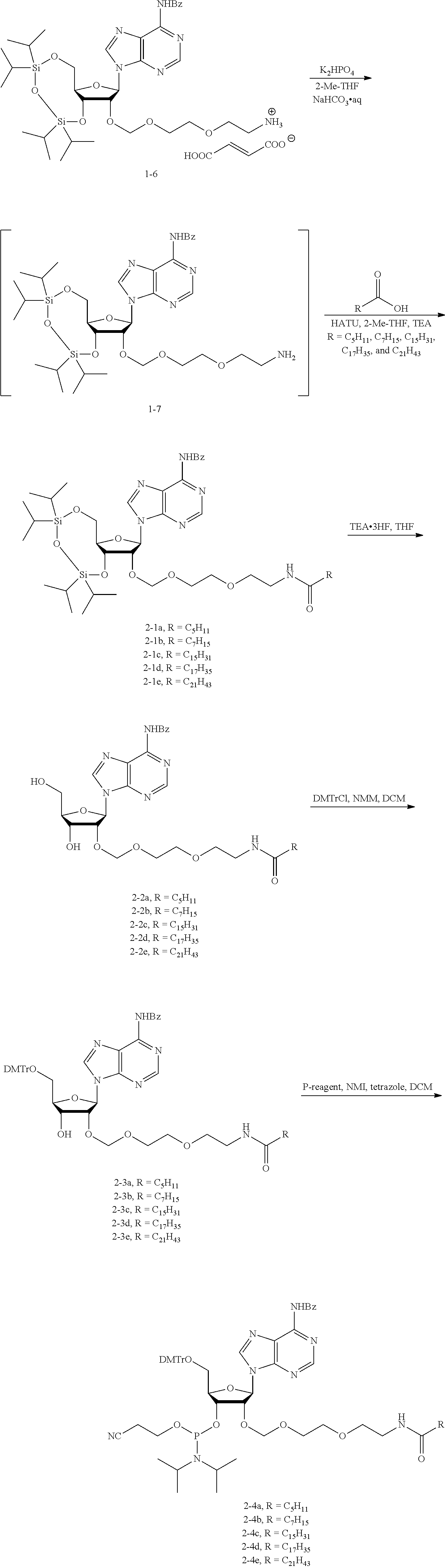

A solution of compound 1-6 (50.00 g, 59.01 mmol) in 150 mL of 2-methyltetrahydrofuran was washed with ice cold aqueous K₂HPO₄ (6%, 100 mL) and brine (20%, 2×100 mL). The organic layer was separated and treated with hexanoic acid (10.33 mL, 82.61 mmol), HATU (33.66 g, 88.52 mmol), and DMAP (10.81 g, 147.52 mmol) at 0° C. The resulting mixture was warmed to 25° C. and stirred for 1 h. The solution was washed with water (2×100 mL), brine (100 mL), and concentrated in vacuo to afford a crude residue. Flash chromatography on silica gel (1:1 hexanes/acetone) gave compound 2-1a (34.95 g, 71.5%) as a white solid.

A mixture of compound 2-1a (34.95 g, 42.19 mmol) and TEA (9.28 mL, 126.58 mmol) in 80 mL of THF was treated with triethylamine trihydrofluoride (20.61 mL, 126.58 mmol) dropwise at 10° C. The mixture was warmed to 25° C. and stirred for 2 h. The reaction was concentrated, dissolved in DCM (100 mL), and washed with sat. NaHCO₃ (5×20 mL) and brine (50 mL). The organic layer was concentrated in vacuo to afford crude compound 2-2a (24.72 g, 99%), which was used directly for the next step without further purification.

A solution of compound 2-2a (24.72 g, 42.18 mmol) in 50 mL of DCM was treated with N-methylmorpholine (18.54 mL, 168.67 mmol) and DMTr-Cl (15.69 g, 46.38 mmol). The mixture was stirred at 25° C. for 2 h and quenched with sat. NaHCO₃ (50 mL). The organic layer was separated, washed with water, concentrated to afford a slurry crude. Flash chromatography on silica gel (1:1 hexanes/acetone) gave compound 2-3a (30.05 g, 33.8 mmol, 79.9%) as a white solid.

A solution of compound 2-3a (25.00 g, 28.17 mmol) in 50 mL of DCM was treated with N-methylmorpholine (3.10 mL, 28.17 mmol) and tetrazole (0.67 mL, 14.09 mmol) under nitrogen atmosphere. Bis(diisopropylamino) chlorophosphine (9.02 g, 33.80 mmol) was added to the solution dropwise and the resulting mixture was stirred at 25° C. for 4 h. The reaction was quenched with water (15 mL), and the aqueous layer was extracted with DCM (3×50 mL). The combined organic layers were washed with sat. NaHCO₃ (50 mL), concentrated to afford a crude solid that was recrystallized from a mixture of DCM/MTBE/n-hexane (1:4:40) to afford compound 2-4a (25.52 g, 83.4%) as a white solid: ¹H NMR (400 MHz, d₆-DMSO) 11.25 (s, 1H), 8.65-8.60 (m, 2H), 8.09-8.02 (m, 2H), 7.71 (s, 1H), 7.67-7.60 (m, 1H), 7.59-7.51 (m, 2H), 7.38-7.34 (m, 2H), 7.30-7.25 (m, 7H), 6.85-6.79 (m, 4H), 6.23-6.20 (m, 1H), 5.23-5.14 (m, 1H), 4.80-4.69 (m, 3H), 4.33-4.23 (m, 2H), 3.90-3.78 (m, 1H), 3.75 (s, 6H), 3.74-3.52 (m, 3H), 3.50-3.20 (m, 6H), 3.14-3.09 (m, 2H), 3.09 (s, 1H), 2.82-2.80 (m, 1H), 2.65-2.60 (m, 1H), 2.05-1.96 (m, 2H), 1.50-1.39 (m, 2H), 1.31-1.10 (m, 14H), 1.08-1.05 (m, 2H), 0.85-0.79 (m, 3H); ³¹P NMR (162 MHz, d₆-DMSO) 149.43, 149.18.

Compound 2-4b, 2-4c, 2-4d, and 2-4e were prepared using similar procedures described above for compound 2-4a. Compound 2-4b was obtained (25.50 g, 85.4%) as a white solid: ¹H NMR (400 MHz, d₆-DMSO) 11.23 (s, 1H), 8.65-8.60 (m, 2H), 8.05-8.02 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.97 (m, 2H), 1.50-1.38 (m, 2H), 1.31-1.10 (m, 18H), 1.08-1.05 (m, 2H), 0.85-0.78 (m, 3H); ³¹P NMR (162 MHz, d₆-DMSO) 149.43, 149.19.

Compound 2-4c was obtained (36.60 g, 66.3%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.25-5.17 (m, 1H), 4.80-4.69 (m, 3H), 4.40-4.21 (m, 2H), 3.91-3.80 (m, 1H), 3.74 (s, 6H), 3.74-3.50 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.33-1.12 (m, 38H), 1.08-1.05 (m, 2H), 0.86-0.80 (m, 3H); ³¹P NMR (162 MHz, d₆-DMSO) 149.42, 149.17.

Compound 2-4d was obtained (26.60 g, 72.9%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.33 (m, 2H), 7.30-7.25 (m, 7H), 6.89-6.80 (m, 4H), 6.21-6.15 (m, 1H), 5.22-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.08 (m, 38H), 1.08-1.05 (m, 2H), 0.85-0.79 (m, 3H); ³¹P NMR (162 MHz, d₆-DMSO) 149.47, 149.22.

Compound 2-4e was obtained (38.10 g, 54.0%) as a white solid: ¹H NMR (400 MHz, d₆-DMSO) 11.21 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.73 (s, 6H), 3.74-3.52 (m, 3H), 3.47-3.22 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.06 (m, 46H), 1.08-1.06 (m, 2H), 0.85-0.77 (m, 3H); ³¹P NMR (162 MHz, d₆-DMSO) 149.41, 149.15.

### Example 2. Synthesis of GalXC RNAi Oligonucleotide-Lipid Conjugates

Scheme 1. Synthesis of GalXC RNAi oligonucleotide-lipid conjugates with mono-lipid (linear and branched) conjugated to the tetraloop. Post-synthetic conjugation was realized through amide coupling reactions.

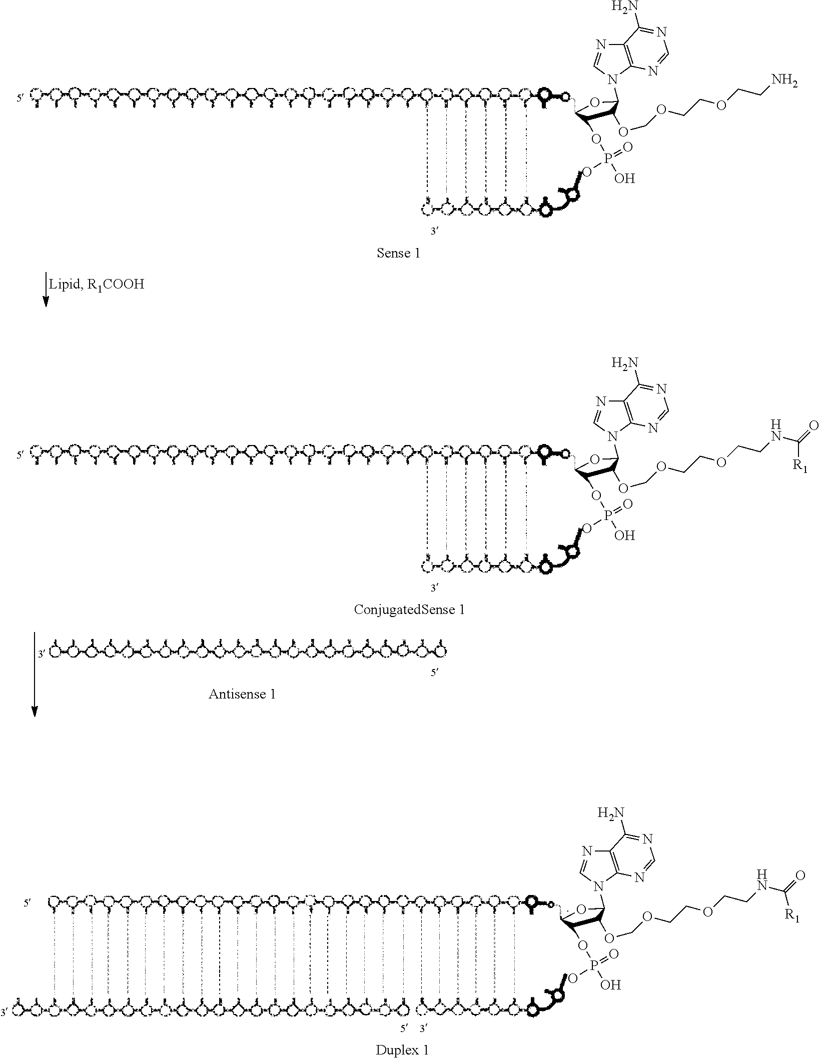

R₁COOH group represents fatty acid C8:0, C10:0, C11:0, C12:0, C14:0, C16:0, C17:0, C18:0, C18:1, C18:2, C22:5, C22:0, C24:0, C26:0, C22:6, C24:1, diacyl C16:0 or diacyl C18:1

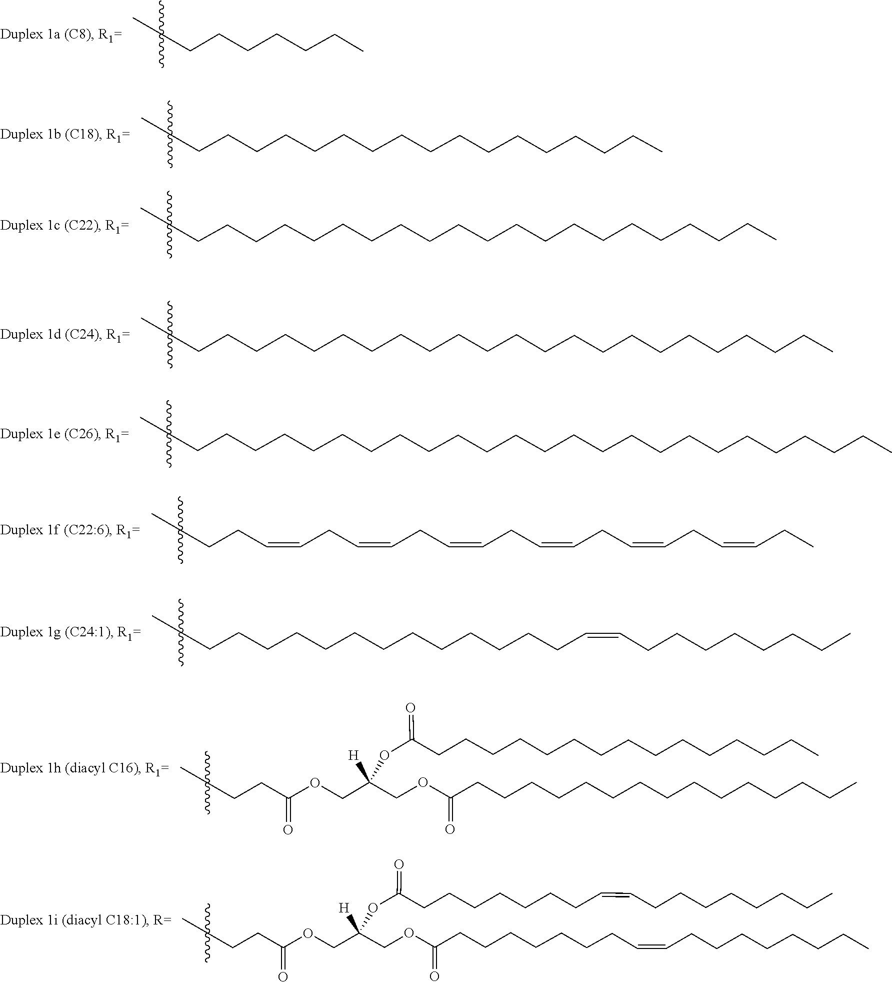

Synthesis Sense 1 and Antisense 1 were prepared by solid-phase synthesis.

Synthesis of Conjugated Sense 1a-1i.

Conjugated Sense 1a was synthesized through post-syntenic conjugation approach. In Eppendorf tube 1, a solution of octanoic acid (0.58 mg, 4 umol) in DMA (0.75 mL) was treated with HATU (1.52 mg, 4 umol) at rt. In Eppendorf tube 2, a solution of oligo Sense 1 (10.00 mg, 0.8 umol) in H₂O (0.25 mL) was treated with DIPEA (1.39 uL, 8 umol). The solution in Eppendorf tube 1 was added to the Eppendorf tube 2 and mixed using Thermomixer at rt. After the reaction was completed indicated by LC-MS analysis, the reaction mixture was diluted with 5 mL of water and purified by revers phase XBridge C18 column using a 5-95% gradient of 100 mM TEAA in ACN and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were then lyophilized to afford an amorphous white solid of Conjugated Sense 1a (6.43 mg, 64% yield).

Conjugated Sense 1b-1i were prepared using similar procedures as described for the synthesis of Conjugated Sense 1a and obtained in 42%-69% yields.

Annealing of Duplex 1a-1j.

Conjugated Sense 1a (10 mg, measured by weight) was dissolved in 0.5 mL deionized water to prepare a 20 mg/mL solution. Antisense 1 (10 mg, measured by OD) was dissolved in 0.5 mL deionized water to prepare a 20 mg/mL solution, which was used for the titration of the conjugated sense and quantification of the duplex amount. Based on the calculation of molar amounts of both conjugated sense and antisense, a proportion of required Antisense 1 was added to the Conjugated Sense 1a solution. The resulting mixture was stirred at 95° C. for 5 min and allowed to cool down to rt. The annealing progress was monitored by ion-exchange HPLC. Based on the annealing progress, several proportions of Antisense 1 were further added to complete the annealing with >95% purity. The solution was lyophilized to afford Duplex 1a (C8) and its amount was calculated based on the molar amount of the antisense consumed in the annealing.

Duplex 1b-1i were prepared using the same procedures as described for the annealing of Duplex 1a (C8).

The following Scheme 1-2 depicts the synthesis of Nicked tetraloop GalXC conjugates with mono-lipid on the loop. Post-synthetic conjugation was realized through Cu-catalyzed alkyne-azide cycloaddition reaction.

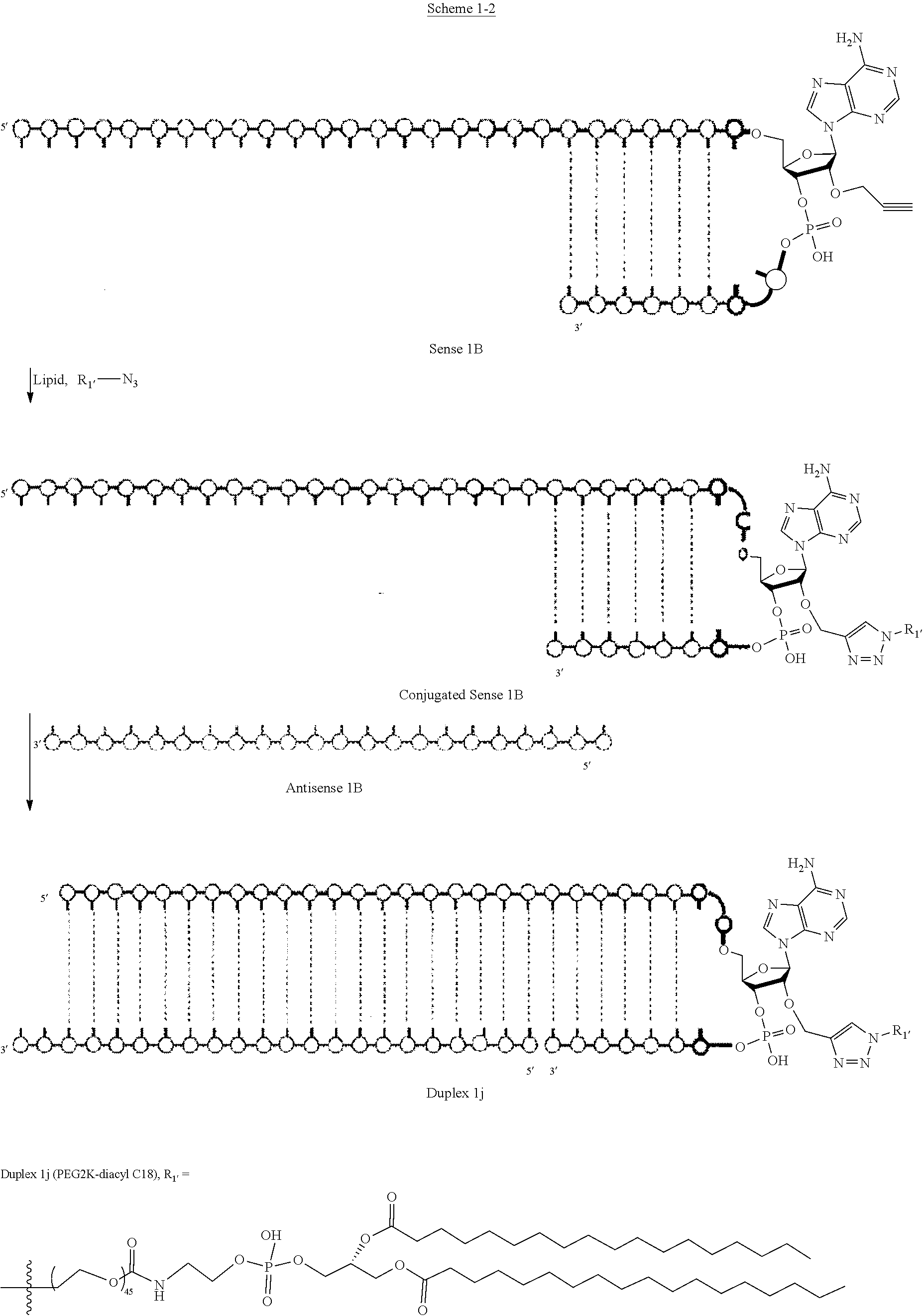

Sense 1B and Antisense 1B were prepared by solid-phase synthesis.

### Synthesis of Conjugated Sense 1j.

In Eppendorf tube 1, a solution of oligo (10.00 mg, 0.8 umol) in a 3:1 mixture of DMA/H₂O (0.5 mL) was treated with the lipid linker azide (11.26 mg, 4 umol). In Eppendorf tube 2, CuBr dimethyl sulfide (1.64 mg, 8 umol) was dissolved in ACN (0.5 mL). Both solutions were degassed for 10 min by bubbling N₂ through them. The ACN solution of CuBrSMe₂ was then added into tube 1 and the resulting mixture was stirred at 40° C. After the reaction was completed indicated by LC-MS analysis, the reaction mixture was diluted with 0.5 M EDTA (2 mL) and dialyzed against water (2×) using a Amicon® Ultra-15 Centrifugal (3K). The reaction crude was purified by revers phase XBridge C18 column using a 5-95% gradient of 100 mM TEAA in ACN (with 30% IPA spiked in) and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were lyophilized to afford an amorphous white solid of Conjugated Sense 1j (6.90 mg, 57% yield).

Duplex 1j (PEG2K-diacyl C18) was prepared using the same procedures as described for the annealing of Duplex 1a (C8).

The following Scheme 1-3 depicts the synthesis of Nicked tetraloop GalXC conjugates with di-lipid on the loop using post-synthetic conjugation approach.

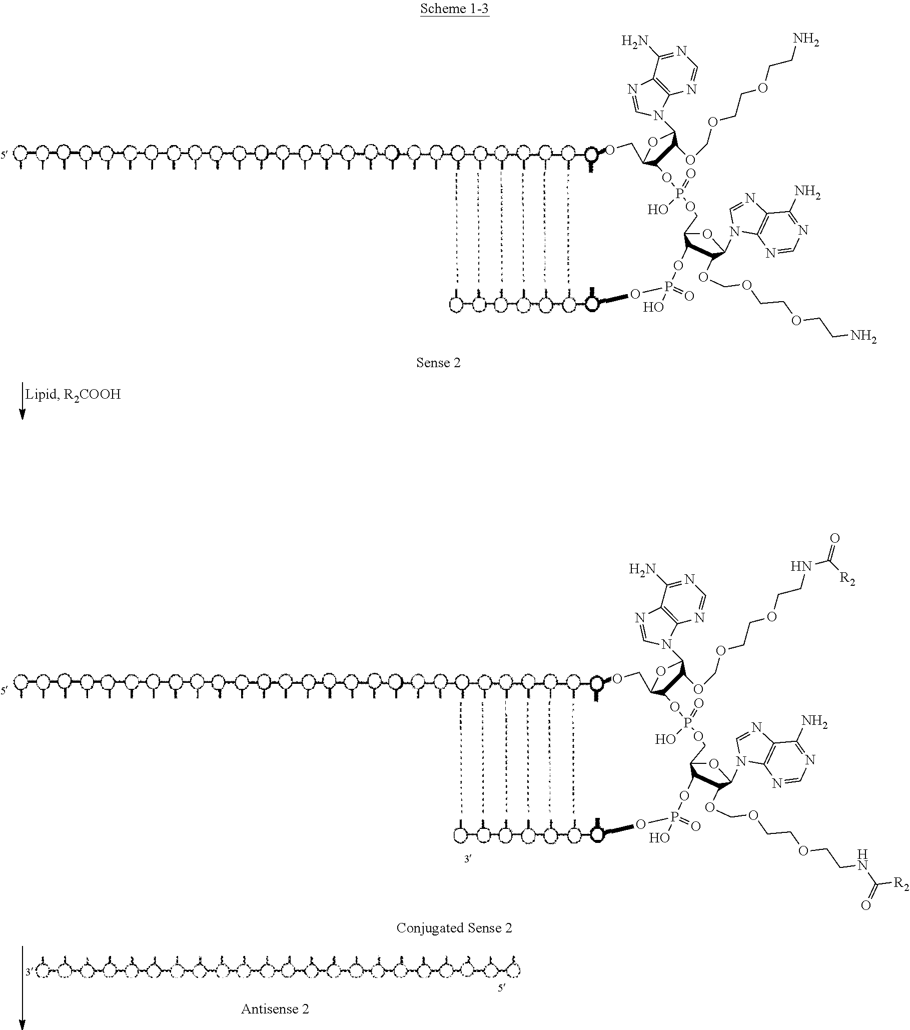

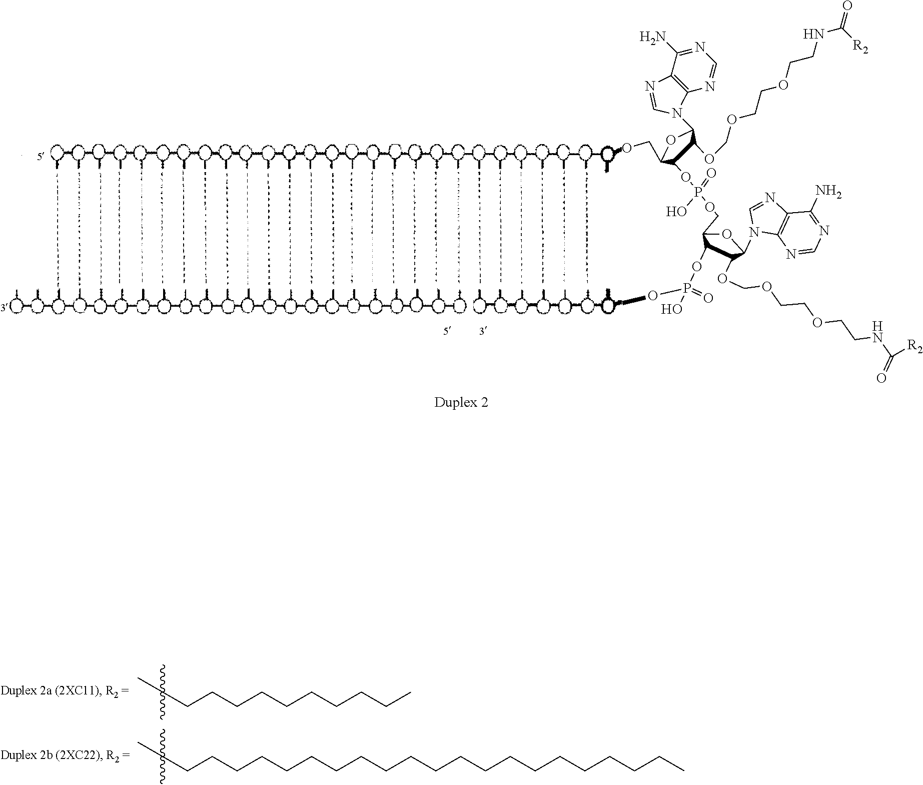

Sense 2 and Antisense 2 were prepared by solid-phase synthesis.

Conjugated Sense 2a and 2b were prepared using similar procedures as described for the synthesis of Conjugated Sense 1a but with 10 eq of lipid, 10 eq of HATU, and 20 eq of DIPEA.

Duplex 2a (2XC11) and 2b (2XC22) were prepared using the same procedures as described for the annealing of Duplex 1a (C8).

The following Scheme 1-4 depicts the synthesis of GalXC of fully phosphorothioated stem-loop conjugated with mono-lipid using post-synthetic conjugation approach.

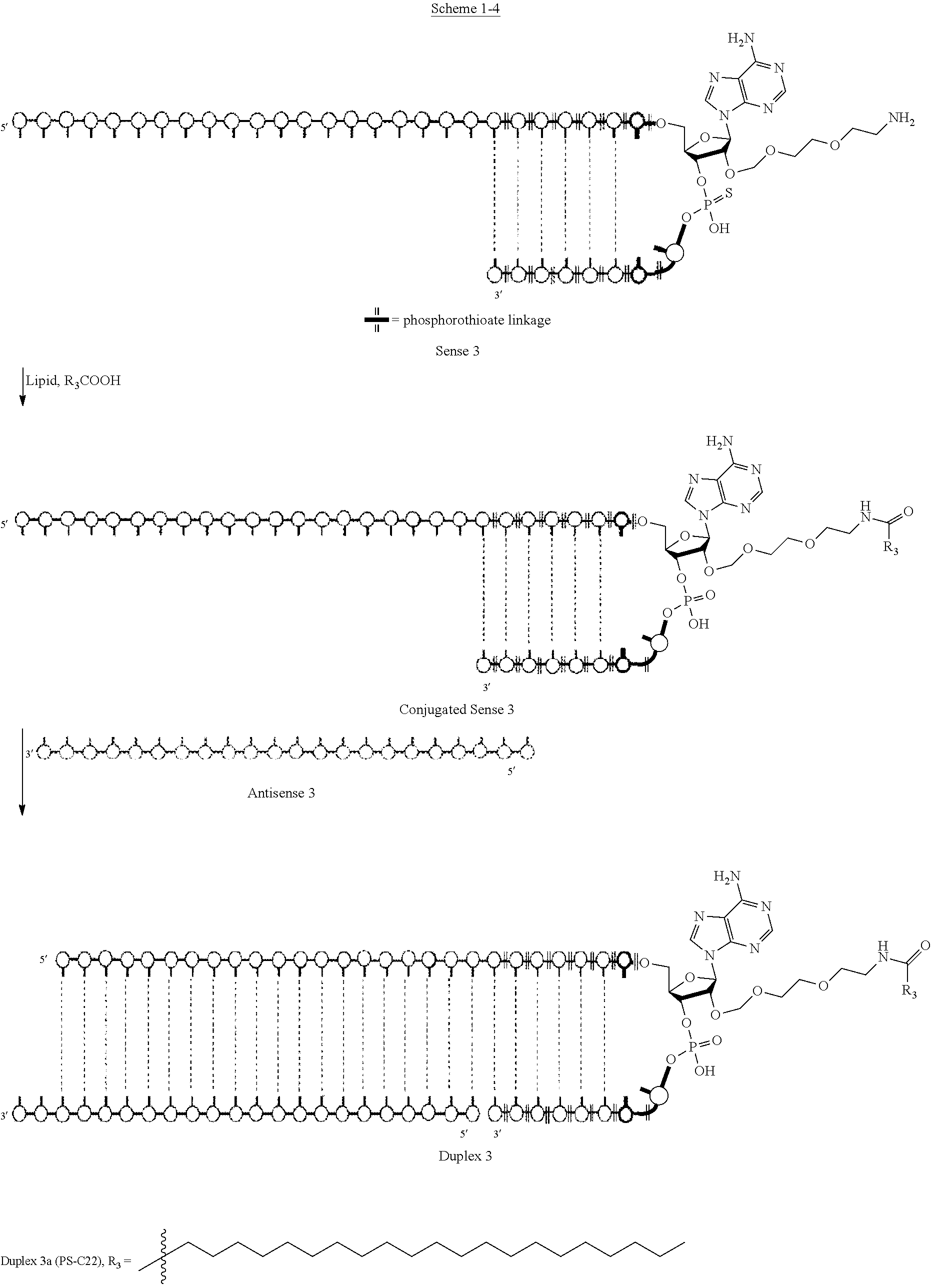

Sense 3 and Antisense 3 were prepared by solid-phase synthesis.

Conjugated Sense 3a was prepared using similar procedures as described for the synthesis of Conjugated Sense 1a and obtained in a 65% yield.

Duplex 3a (PS-C22) was prepared using the same procedures as described for the annealing of Duplex 1a (C8).

The following Scheme 1-5 depicts the synthesis of GalXC of short sense conjugated with mono-lipid using post-synthetic conjugation approach.

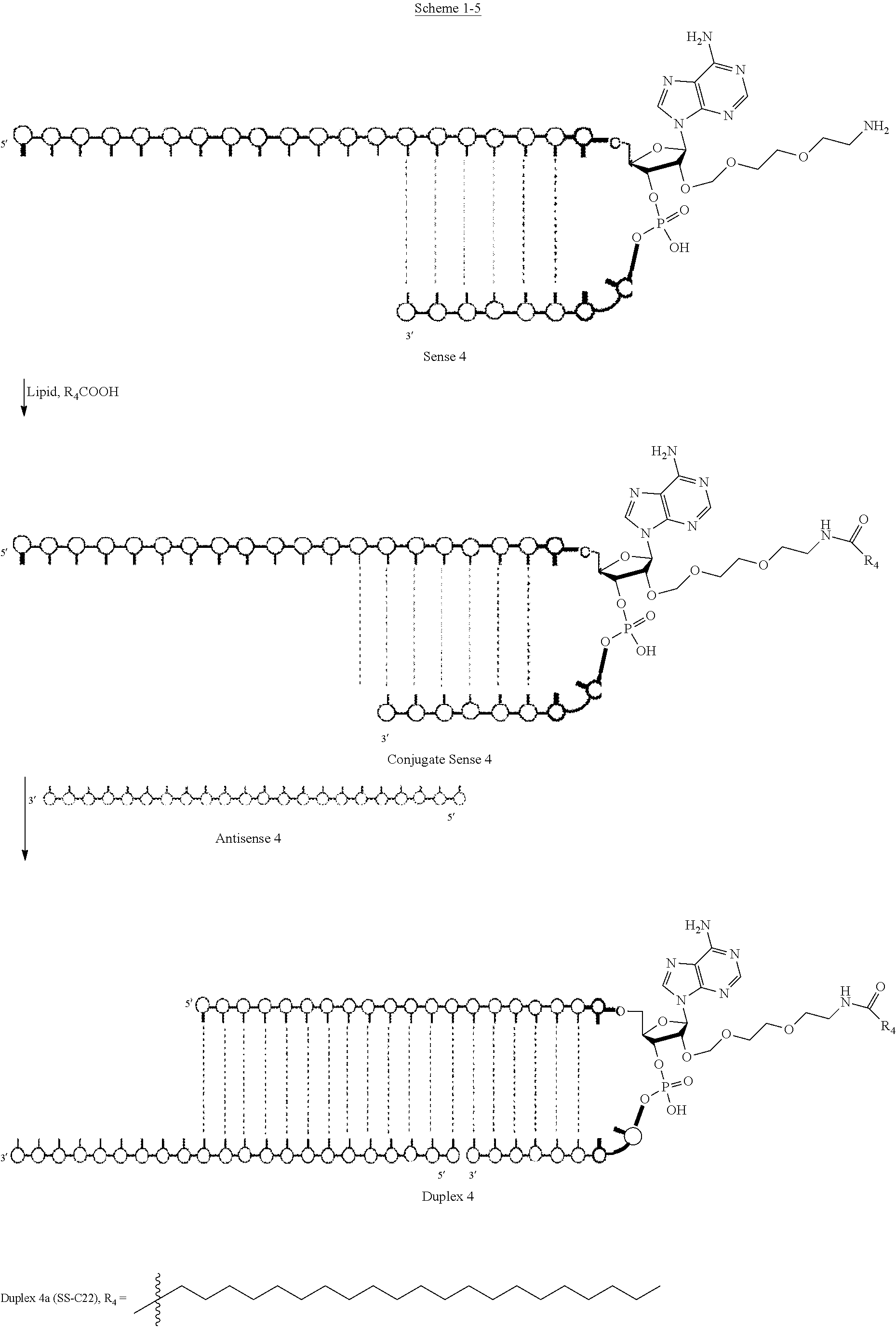

Sense 4 and Antisense 4 were prepared by solid-phase synthesis.

Conjugated Sense 4a was prepared using similar procedures as described for the synthesis of Conjugated Sense 1a and obtained in a 74% yield.

Duplex 4a (SS-C22) was prepared using the same procedures as described for the annealing of Duplex 1a (C8).

The following Scheme 1-6 depicts the synthesis of Nicked tetraloop GalXC conjugated with tri-adamantane moiety on the loop using post-synthetic conjugation approach.

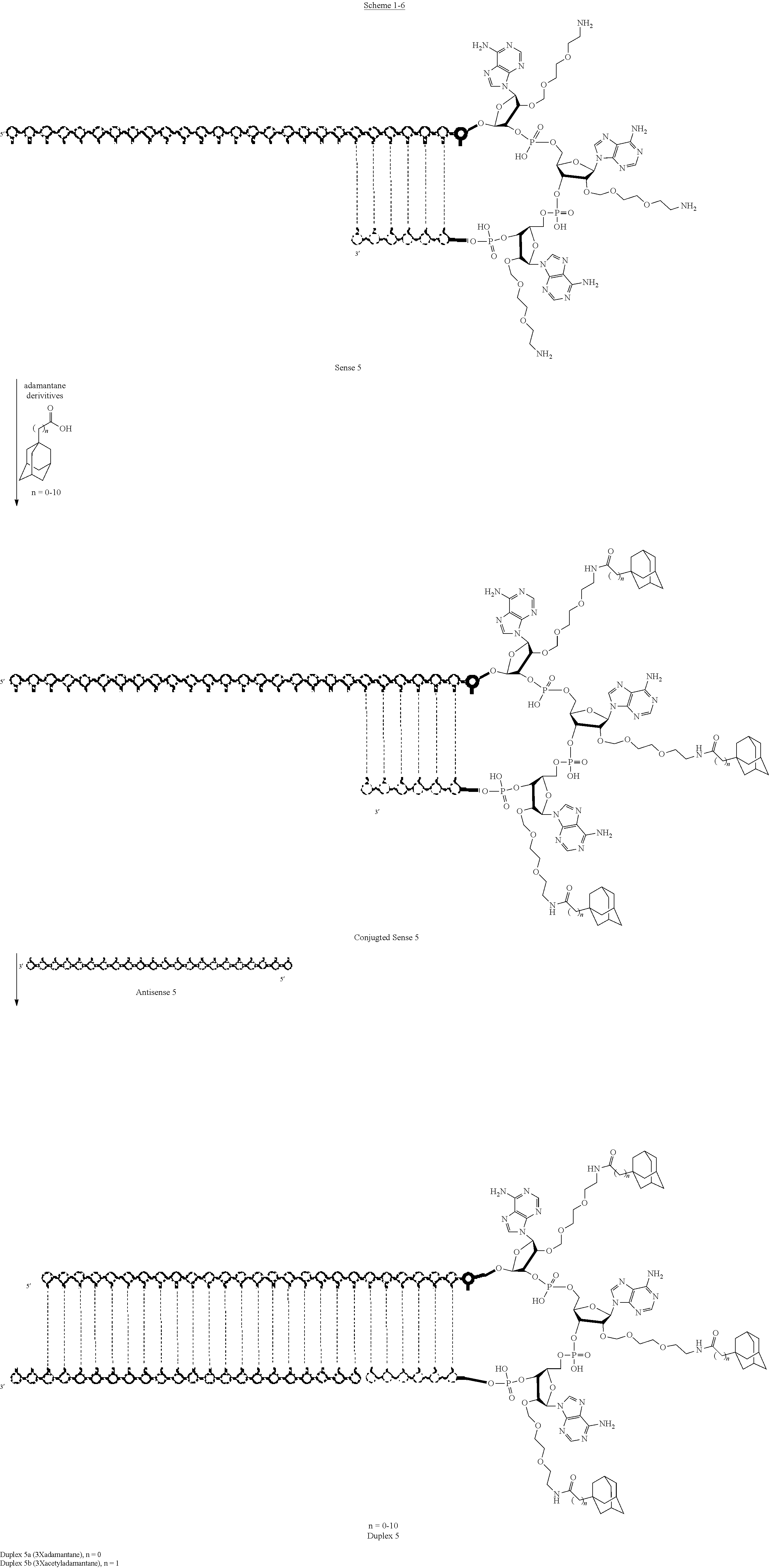

Sense 5 and Antisense 5 were prepared by solid-phase synthesis.

Conjugated Sense 5a and 5b were prepared using similar procedures as described for the synthesis of Conjugated Sense 1a and obtained in 42%-73% yields.

Duplex 5a (3×adamantane) and Duplex 5b (3×acetyladamantane) were prepared using the same procedures as described for the annealing of Duplex 1a (C8).

The following scheme 1-7 depicts an example of solid phase synthesis of Nicked tetraloop GalXC conjugated with lipid(s) on the loop.

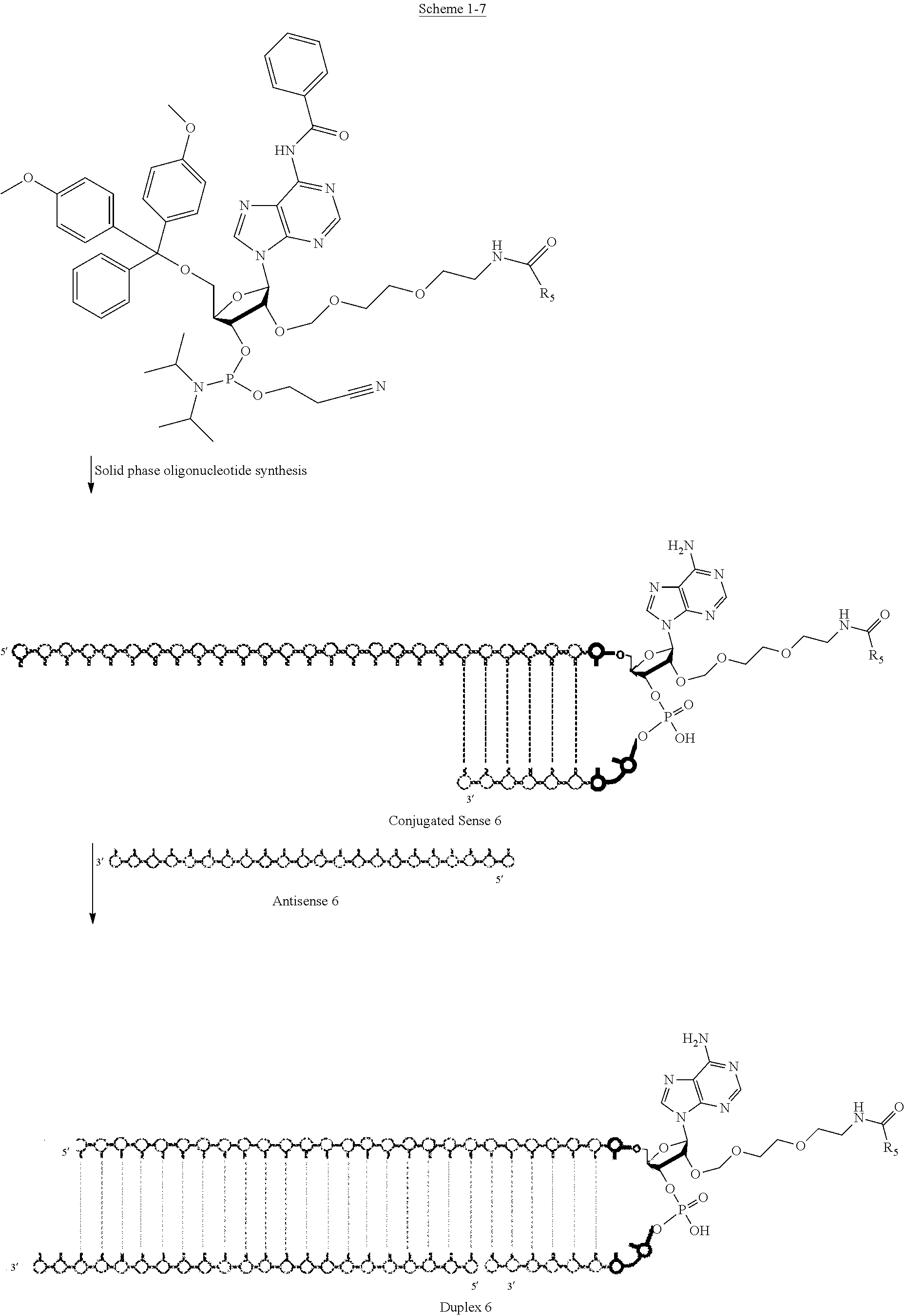

### Synthesis of Conjugated Sense 6.

Conjugated Sense 6 was prepared by solid-phase synthesis using a commercial oligo synthesizer. The oligonucleotides were synthesized using 2′-modified nucleoside phosphoramidites, such as 2′-F or 2′-OMe, and 2′-diethoxymethanol linked fatty acid amide nucleoside phosphoramidites. Oligonucleotide synthesis was conducted on a solid support in the 3′ to 5′direction using a standard oligonucleotide synthesis protocol. In these efforts, 5-ethylthio-1H-tetrazole (ETT) was used as an activator for the coupling reaction. Iodine solution was used for phosphite triester oxidation. 3-(Dimethylaminomethylidene)amino-3H-1,2,4-dithiazole-3-thione (DDTT) was used for the formation of phosphorothioate linkages. Synthesized oligonucleotides were treated with concentrated aqueous ammonium for 10 h. The ammonia was removed from the suspension and the solid support residues were removed by filtration. The crude oligonucleotide was treated with TEAA, analyzed, and purified by strong anion exchange high performance liquid chromatography (SAX-HPLC). The fractions were combined and dialyzed against water (3×), saline (1×), and water (3×) using Amicon® Ultra-15 Centrifugal (3K). The remaining solvent was then lyophilized to afford the desired Conjugated Sense 6.

Duplex 6 was prepared using the same procedures as described for the annealing of Duplex 1a (C8).

Scheme 8. Synthesis of Nicked tetraloop GalXC conjugated with one adamantane unit on the loop via a post-synthetic conjugation approach.

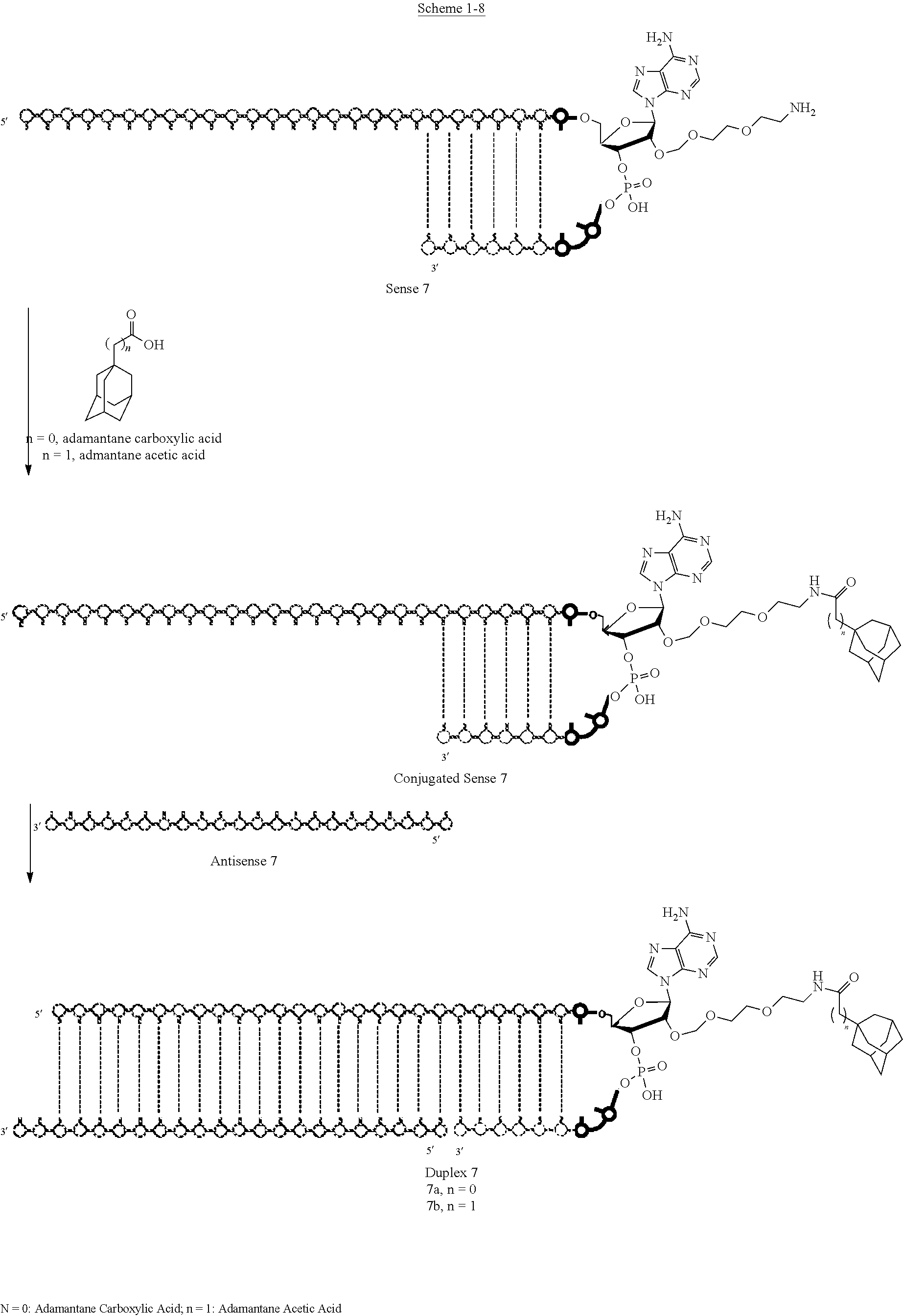

### Synthesis of Conjugated Sense 7a and 7b

Conjugated Sense 7a and Sense 7b were obtained using the same method or a substantially similar method to the synthesis of Conjugated Sense 5.

### Synthesis Example of Duplex 7a and 7b

Duplex 7a and Duplex 7b were obtained using the same method or a substantially similar method to the synthesis of Duplex 5.

Scheme 9. Synthesis of nicked tetraloop GalXC conjugated with two adamantane units on the loop via a post-synthetic conjugation approach.

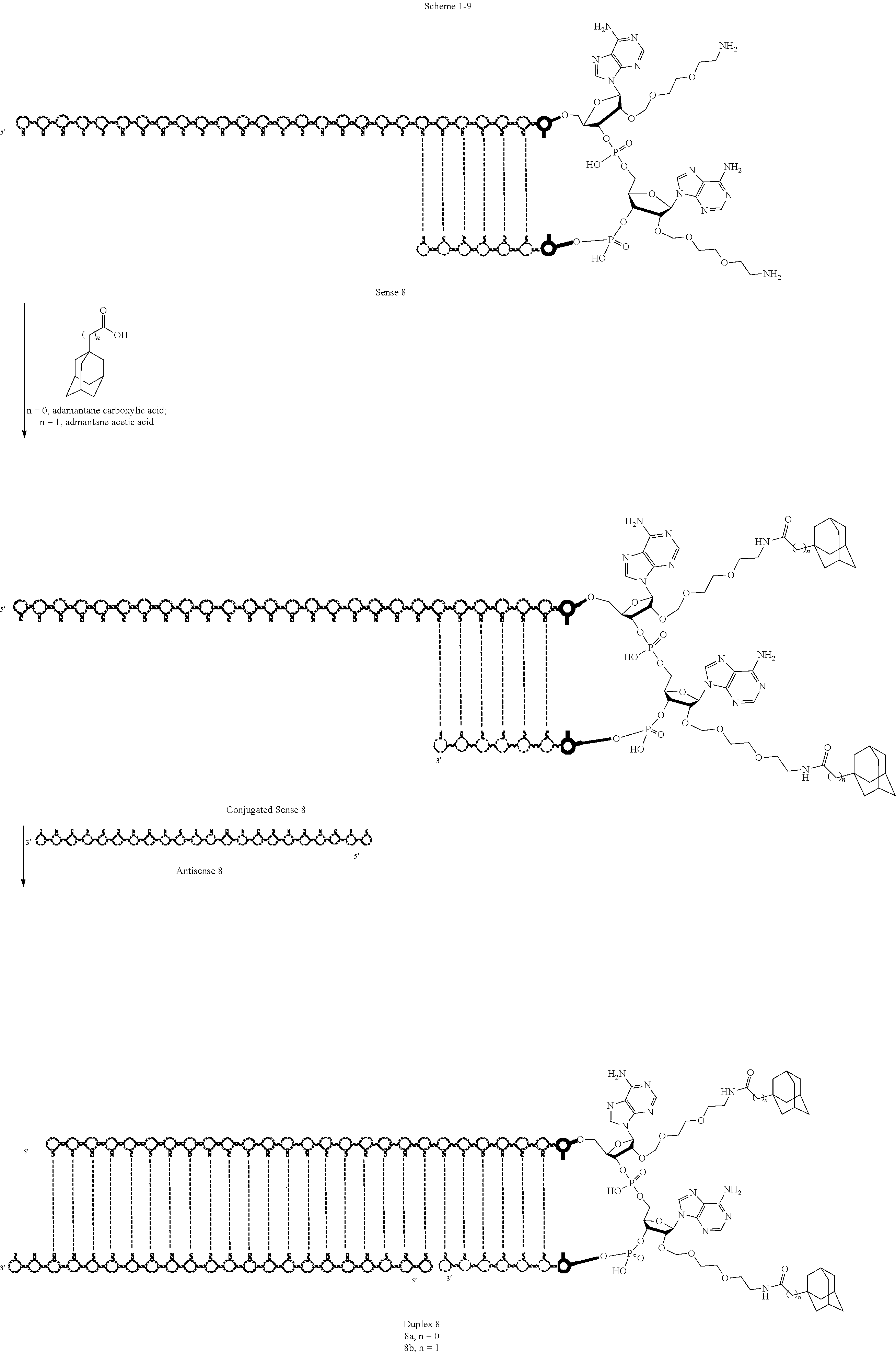

### Synthesis of Conjugated Sense 8a and 8b

Conjugated Sense 8a and Sense 8b were obtained using the same method or a substantially similar method to the synthesis of Conjugated Sense 5.

### Synthesis Example of Duplex 8a and 8b

Duplex 8a and Duplex 8b were obtained using the same method or a substantially similar method to the synthesis of Duplex 5.

The following Scheme1-10 depicts the synthesis of GalXC of short sense and short stem loop conjugated with mono-lipid using post-synthetic conjugation approach.

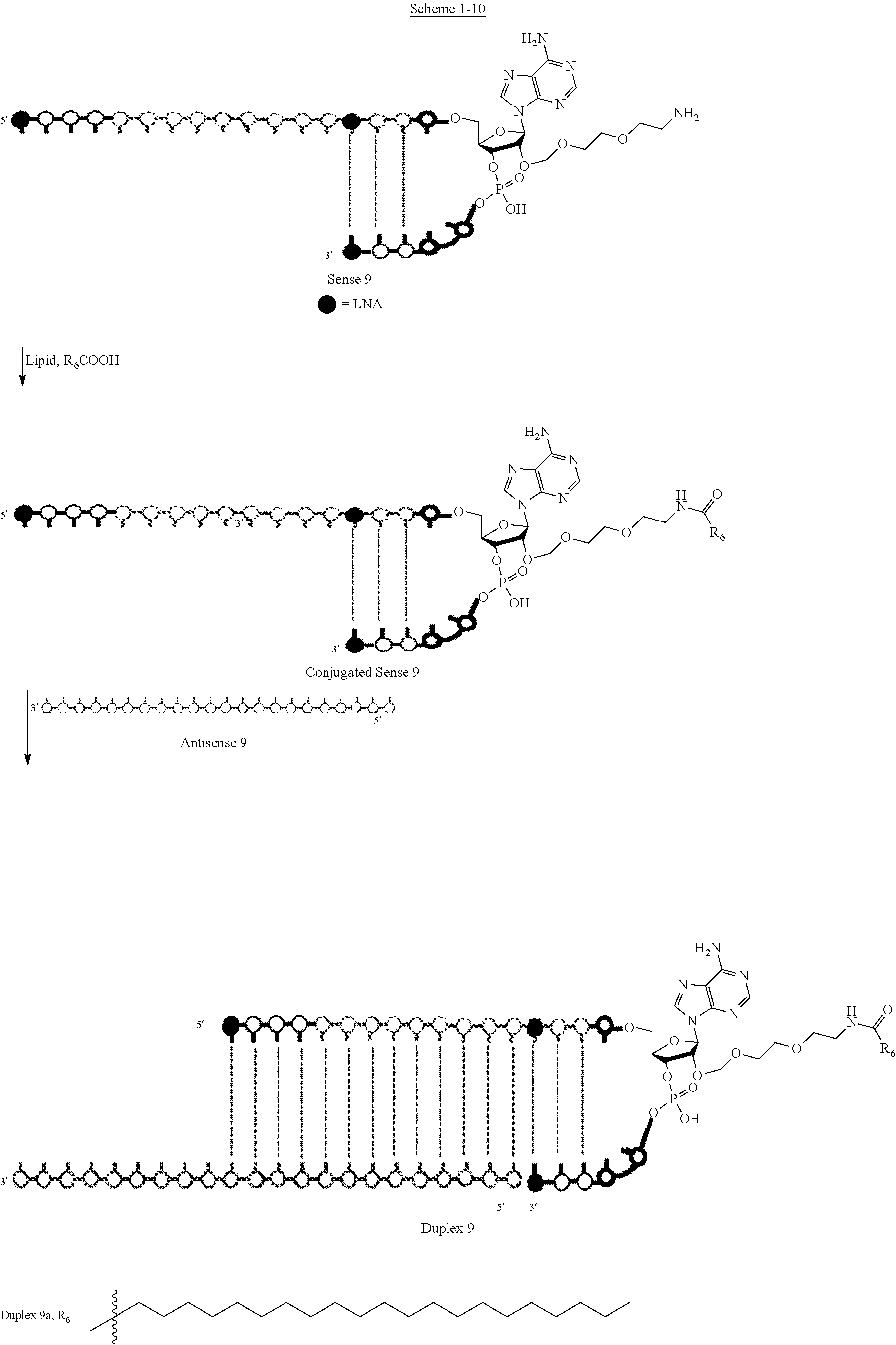

### Synthesis of Sense 9a

Conjugated Sense 9a was obtained using the same method or a substantially similar method to the synthesis of Conjugated Sense 5.

### Synthesis Example of Duplex 9a

Duplex 9a was obtained using the same method or a substantially similar method to the synthesis of Duplex 5.

The following Scheme1-11 depicts the synthesis of GalXC conjugated with mono-lipid at 5′-end using post-synthetic conjugation approach.

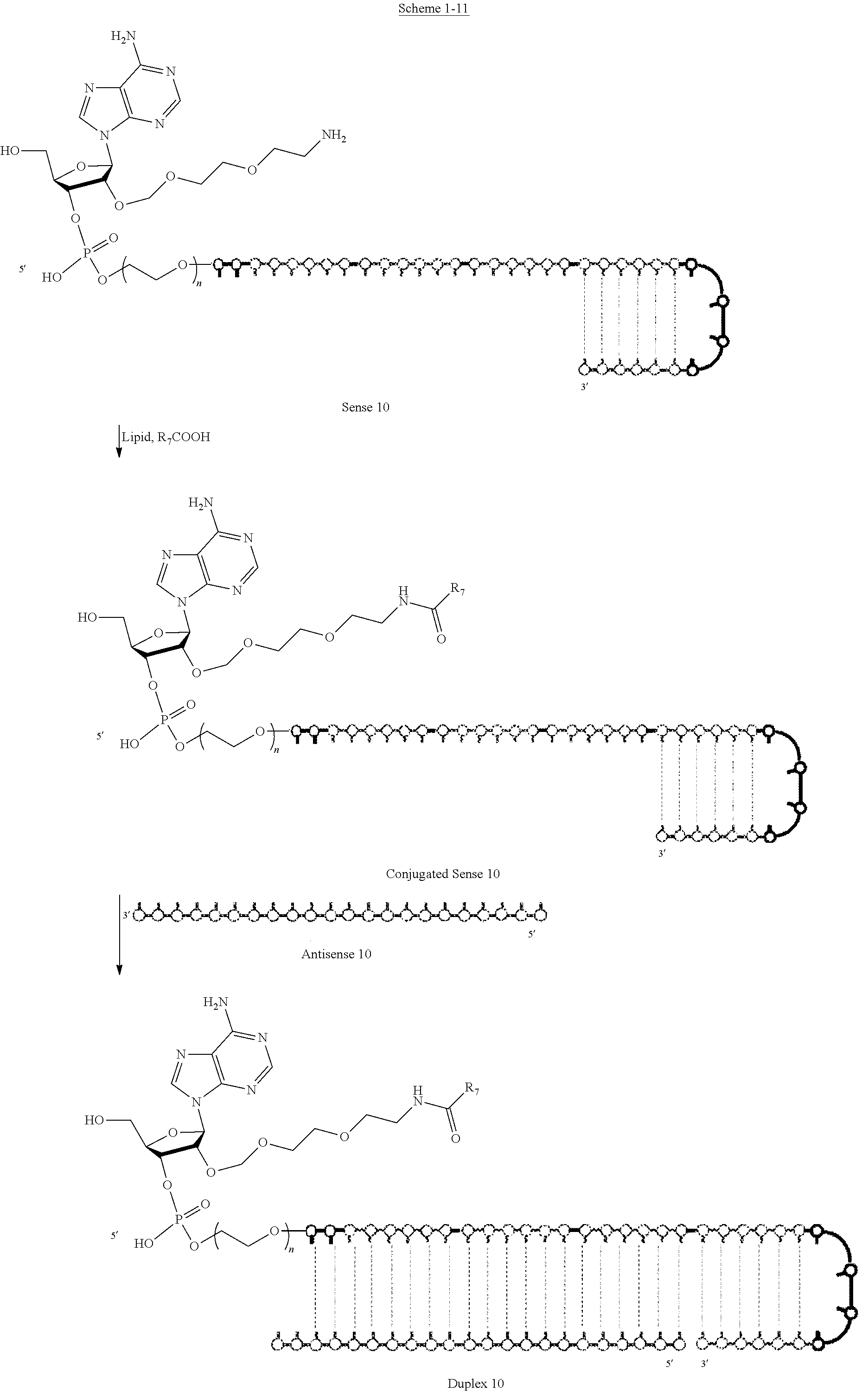

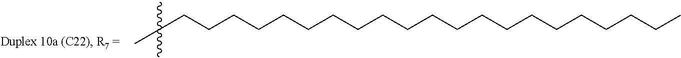 [SMILES: *CCCCCCCCCCCCCCCCCCCCC]

### Synthesis of Conjugated Sense 10a

Conjugated Sense 10a was obtained using the same method or a substantially similar method to the synthesis of Conjugated Sense 5.

### Synthesis Example of Duplex 10a

Duplex 10a was obtained using the same method or a substantially similar method to the synthesis of Duplex 5.

The following Scheme1-12a and 1-12b depict the synthesis of GalXC with blunt end conjugated with mono-lipid at 3′-end or 5′-end using post-synthetic conjugation approach.

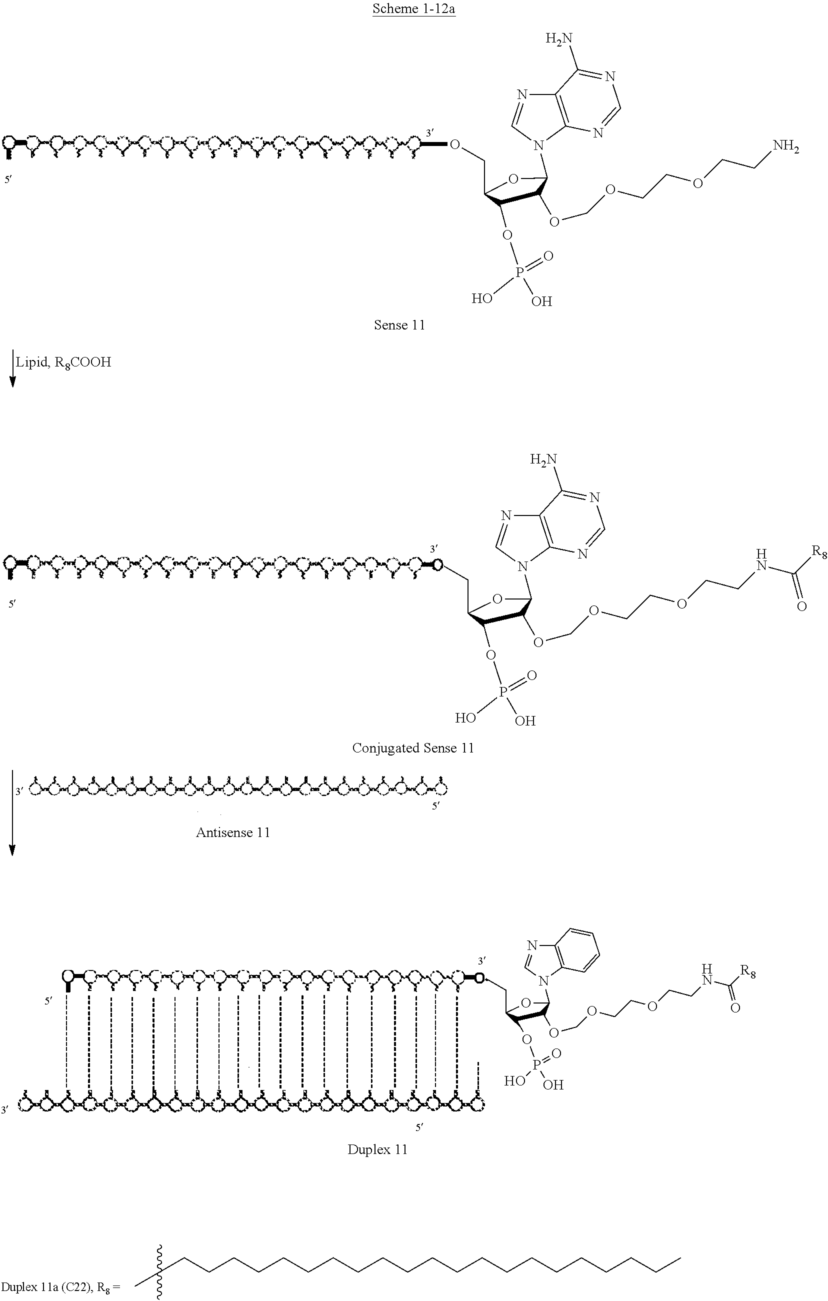

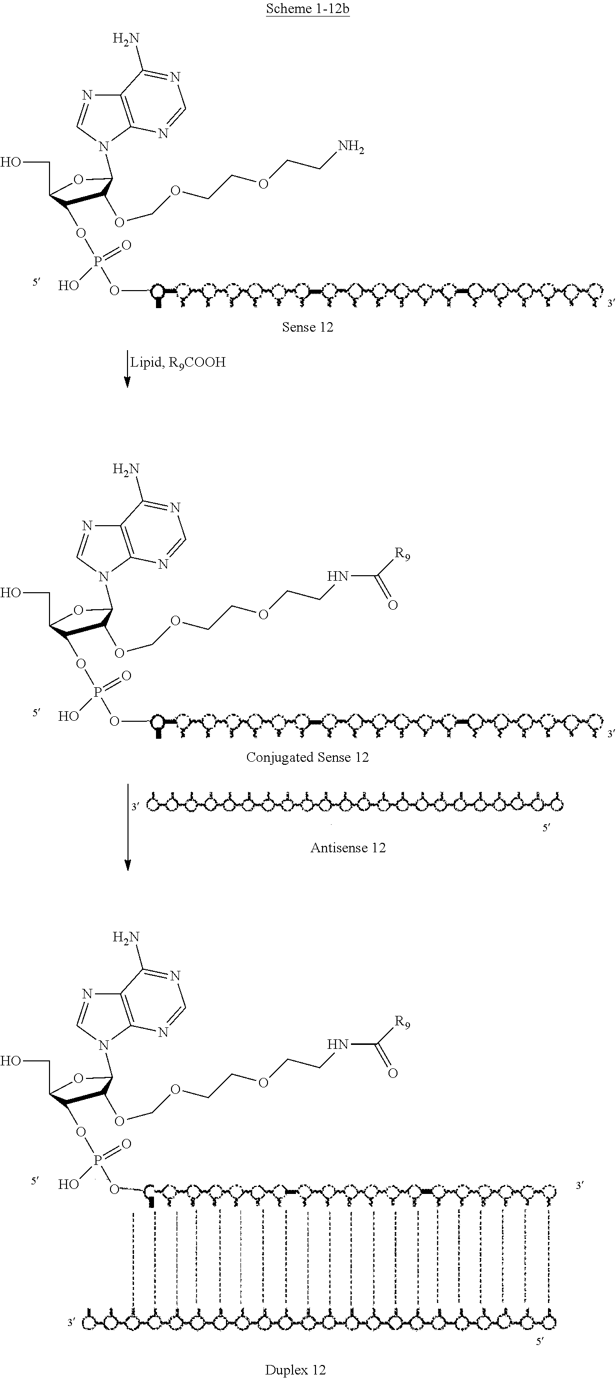

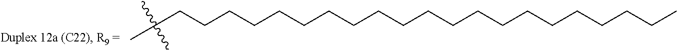 [SMILES: *CCCCCCCCCCCCCCCCCCCCC]

### Synthesis of Conjugated Sense 11a and 12a

Conjugated Sense 11a and 12a were obtained using the same method or a substantially similar method to the synthesis of Conjugated Sense 5.

### Synthesis Example of Duplex 11a and 12a

Duplex 11a and 12a were obtained using the same method or a substantially similar method to the synthesis of Duplex 5.

Conjugates Duplex 8D and Duplex 9D were obtained using the same method or a substantially similar method to the synthesis of Duplex 5.

### Example 3: In Vivo Tumor Models

Programmed death-ligand 1, or CD274 is an immune inhibitory receptor ligand expressed by cells to prevent immune cells from attaching nonharmful cells in the body while targeting foreign cells such as cancer cells or infectious cells. Tumor models were generated to evaluate the anti-tumor efficacy of lipid-conjugated oligonucleotides targeting CD274. Briefly, 6-8-week-old immunocompromised (Nude) or immunocompetent (C57BL/6 or Balbc) mice were injected subcutaneously with 4-5×10⁶ Pan02 cells (mouse pancreatic cancer cell line with matrigel), 2×10⁶ 4T1 cells (mouse triple negative breast), 5×10⁶ MC-38 cells (mouse colon), 2×10⁶ Hepa1-6 cells (hepatocellular carcinoma) under the right shoulder. When the tumors reached a volume of 300-500 mm³, mice were randomized into different cohorts and subjected to dosing with lipid conjugates (generated using the methods described in Examples 1 and 2). Each lipid conjugate was dosed subcutaneously at a total volume of 10 mL/kg. The mouse pancreatic cell line Pan02 was obtained from NCI; mouse breast, colon, and hepatocellular carcinoma cell lines (4T1, MC-38, and Hepa1-6 cell lines, respectively) were obtained from ATCC (Manassas, VA). All cells were grown in RPMI/DMEM medium supplemented with 10% FBS. Pan02 and 4T1 tumors are known to maintain very suppressive, or cold, tumor microenvironments whereas MC-38 and Hepa1-6 are known to be partially sensitive or sensitive to checkpoint inhibitors

### Example 4: Determining Delivery of Lipid Conjugates to Different Components of the Tumor Microenvironment

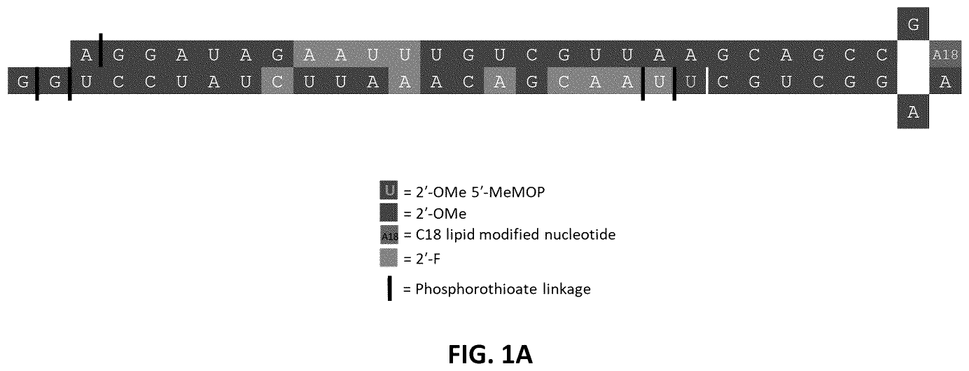

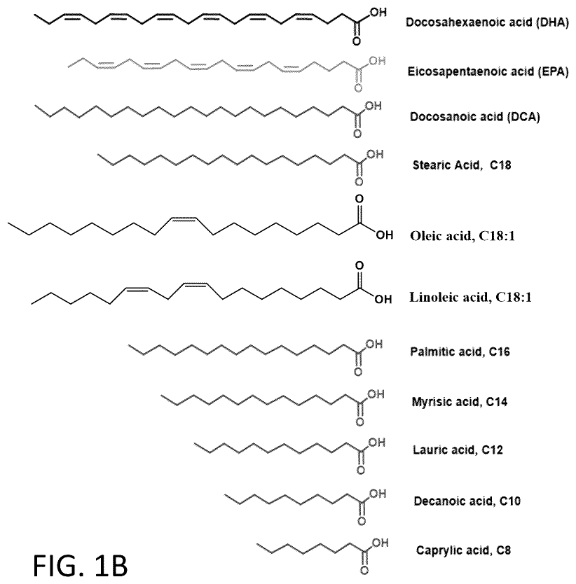

To elucidate delivery of oligonucleotide lipid conjugates, Pan02, 4T1, Hepa 1-6, and MC-38 cells were implanted in C57BL/6, Balb/c, and C57BL/6 mice, respectively as described in Example 3. At about two weeks post implant, when tumor volume reached ˜300-400 mm³, mice were randomized into 2 groups (n=3-4) and treated with either Phosphate Buffered Saline (PBS) or a C18 lipid-conjugated oligonucleotide targeting mouse Cd274 referred to as GalXC-mCD274-C18 (depicted in FIG. 1A with corresponding unmodified sense and antisense sequences of SEQ ID NOs: 1041 and 1042, respectively) at 25 mg/kg. The modification pattern of which is illustrated below:

Sense Strand:
5′ mX-S-mX-mX-mX-mX-mX-mX-fX-fX-fX-fX[-mX-]₁₇-
[ademX-L]-[mX]₈ 3′.

### Hybridized to:

Antisense Strand:
5′ [MePhosphonate-4O-mX]-S-fX-S-fX-fX-fX-mX-fX-
mX-mX-fX-mX-mX-mX-fX-mX-mX-mX-mX-mX-mX-S-mX-S-mX
3′.

Or, represented as:

Sense Strand:
[mXs][mX][mX][mX][mX][mX][mX][fX][fX][fX][fX][mX]
[mX][mX][mX][mX][mX][mX][mX][mX][mX][mX][mX][mX]
[mX][mX][mX][ademX-C18][mX][mX][mX][mX][mX][mX]
[mX][mX]

### Hybridized to:

Antisense Strand:
[MePhosphonate-4O-mXs][fXs][fX][fX][fX][mX][fX][mX]
[mX][fX][mX][mX][mX][fX][mX][mX][mX][mX][mX][mXs]
[mXs][mX]

(Modification key: Table 1).

| | |
|---|---|
| Symbol | Modification/linkage |

Key 1

| | |
|---|---|
| mX | 2′-O-methyl modified nucleotide |
| fX | 2′-fluoro modified nucleotide |
| —S— | phosphorothioate linkage |
| — | phosphodiester linkage |
| [MePhosphonate-4O-mX] | 5′-methoxyphosphonate-4′-oxy modified nucleotide |
| ademA-GalNAc | GalNAc attached to an adenine nucleotide |
| ademX-L | Lipid molecule (e.g., C18) attached to a nucleotide |

Key 2

| | |
|---|---|
| [mXs] | 2′-O-methyl modified nucleotide with a phosphorothioate |
| | linkage to the neighboring nucleotide |
| [fXs] | 2′-fluoro modified nucleotide with a phosphorothioate linkage |
| | to the neighboring nucleotide |
| [mX] | 2′-O-methyl modified nucleotide with phosphodiester linkages |
| | to neighboring nucleotides |
| [fX] | 2′-fluoro modified nucleotide with phosphodiester linkages to |
| | neighboring nucleotides |

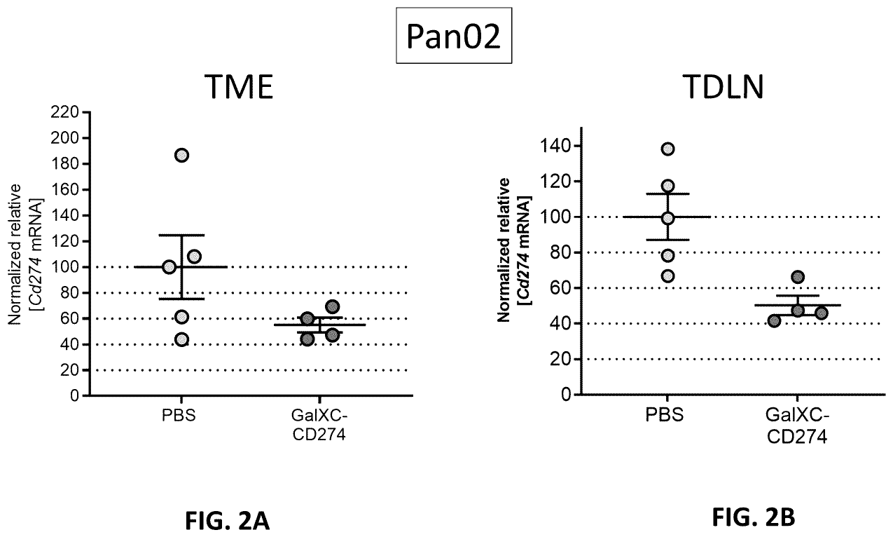

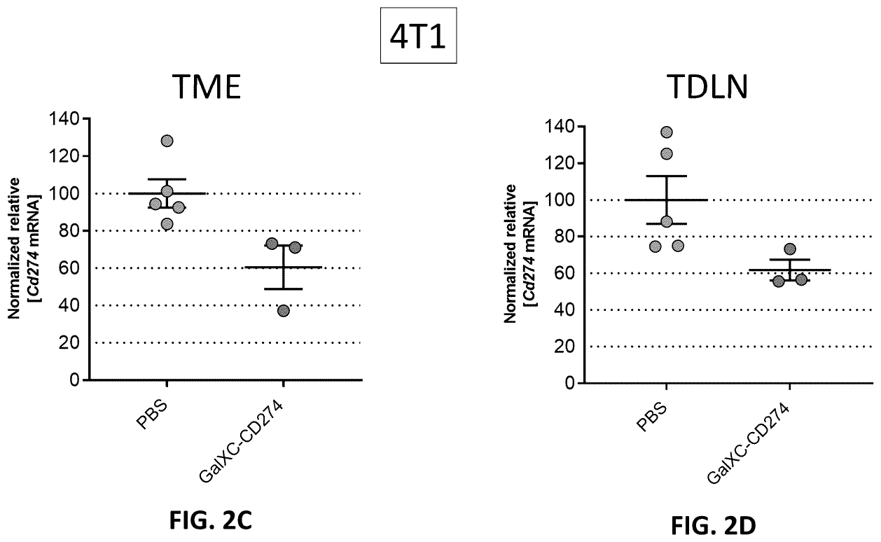

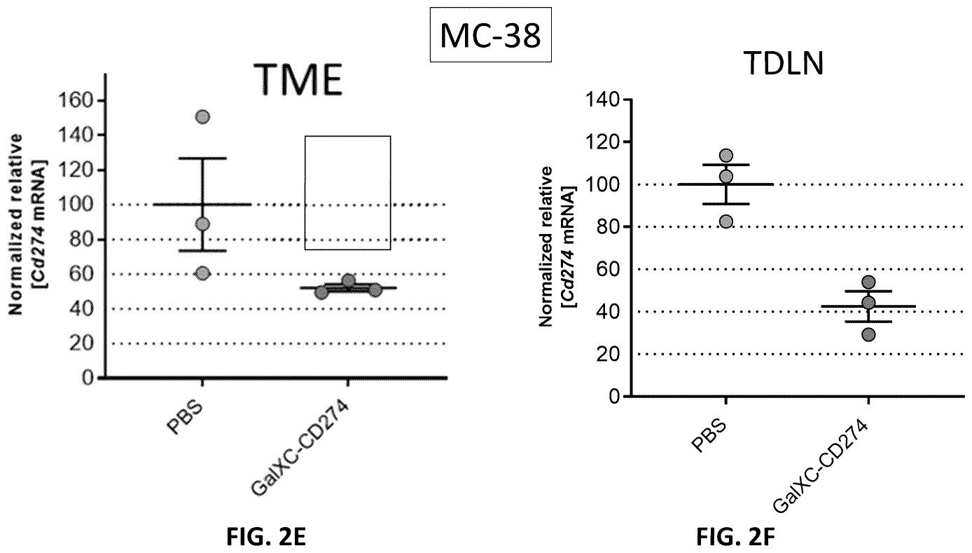

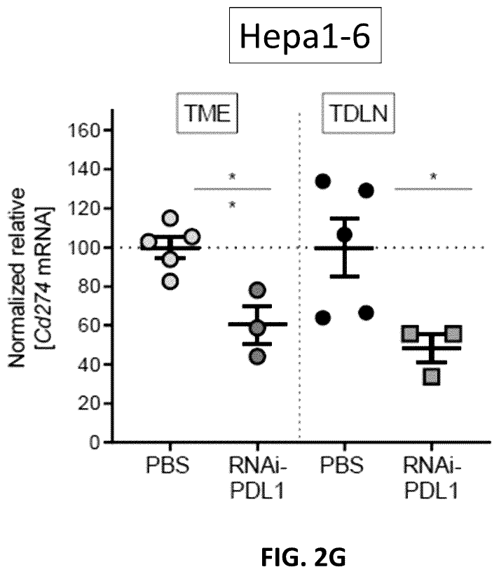

Mice were dosed subcutaneously once every three days (q3dx2; i.e., on Day 1 and Day 4). Seven days post the last subcutaneous injection, tumors and tumor draining lymph nodes (TDLN) were collected and analyzed by qPCR to determine mRNA levels of mouse Cd274. As shown in FIGS. 2A-2G, the mouse Cd274 mRNA levels were decreased by -40-50% in both Tumor microenvironment (TME) and Tumor draining lymph nodes (TDLN) of all 3 tumor types treated with GalXC-mCD274-C18 lipid conjugate. These data suggest that the GalXC-mCD274-C18 lipid conjugate mediates similar oligonucleotide delivery to components in tumor microenvironment and TDLN to facilitate target knockdown independent of the tumor type.

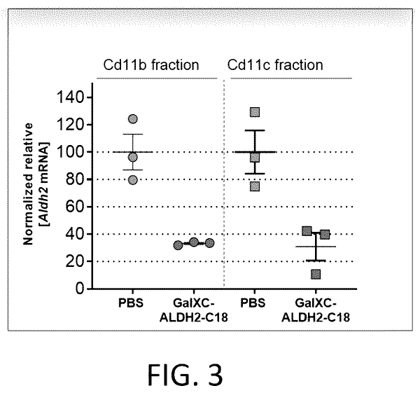

As Cd11c expressing cells in TDLN are known to be the major antigen presenting cells and the PD-L1 expressed by these cells is identified to play key role in antigen presentation machinery and mediating immune responses, first the delivery to these cells with lipid conjugates was evaluated. To do that, myeloid cell types present in the TDLN (Cd11c and Cd11b) were isolated and evaluated after treating the tumor mice with GalXC-ALDH2, a surrogate conjugate as ALDH2 is a protein ubiquitously expressed in the body. Pan02 cells were implanted in C57BL/6 mice as described in Example 3 and when the tumors reached the size of 300-400 mm³, mice (n=3/group) were treated with PBS or GalXC-ALDH2 (modified sense strand of SEQ ID NO: 1045 and modified antisense strand of SEQ ID NO: 1046) at 25 mg/kg. Three days post injection, both inguinal and axillary lymph nodes from 3 mice were pooled in one tube for isolation process. Single cell suspensions were made from pooled lymph nodes and were processed through Gentle MACs system to isolate the Cd11b and Cd11c enriched cell populations. To confirm knockdown in specific cell types such as Cd11b and Cd11c in TDLN, RNA was isolated from these enriched fractions and analyzed by qPCR for mRNA levels of mouse Aldh2. As demonstrated in FIG. 3, the target knockdown was about 70% in Cd11b cells and slightly higher in Cd11c cells. This suggests that the GalXC-ALDH2 conjugate efficiently delivers the payload to the major antigen presenting cells (Cd11c) in TDLN.

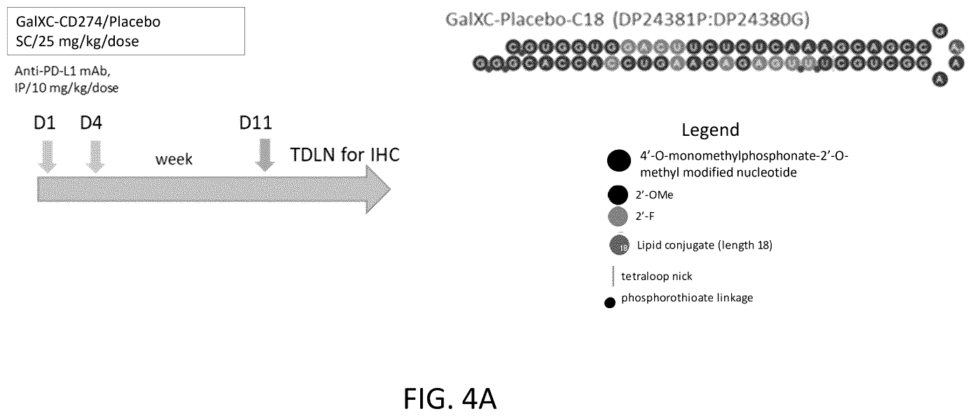

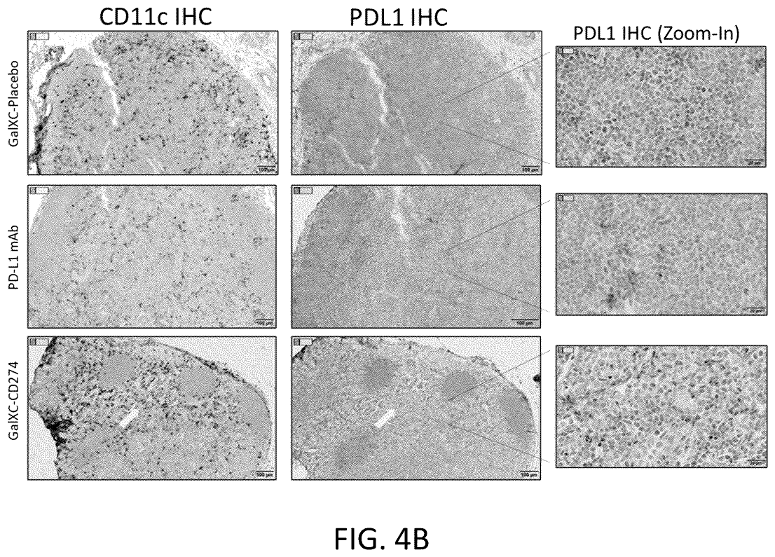

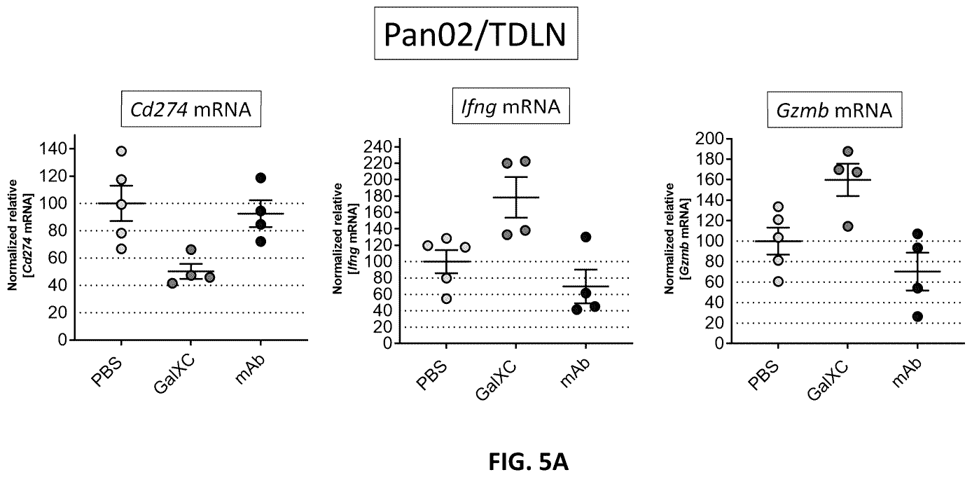

As the PD-L1, especially intracellular format expressed by these Cd11c⁺ DCs cells are identified to play a key role in initiating antigen presentation and mediating immune responses in resistant tumor phenotypes, it was investigated if GalXC-CD274 could silence the intracellular Cd274 in CD11c+DCs present in TDLN of resistant tumor type. To verify this observation with a GalXC-mCD274-C18 conjugate, another resistant tumor 4T1 was utilized. Tumors were grown as described in Example 3 and mice were sorted into 3 groups (n=3) and treated subcutaneously with either GalXC-Placebo-C18 or GalXC-mCD274-C18 at 25 mg/kg or intraperitoneally with an anti-PDL1 mAb (Clone #10G.9G2; Cat #BP0101) at 10 mg/kg. Mice were administered 2 doses of the oligonucleotide on Day 1 and 4 (q3dx2) (FIG. 4A). Seven days post last dose, TDLNs were collected (both inguinal and axillary in one cassette) and processed for immunohistochemistry and sections were stained for Cd11c and PD-L1. As demonstrated in FIG. 4B, the Cd11c levels were fairly similar in tumors from all 3 treatment groups. PD-L1 levels aligned with Cd11c levels in samples that had GalXC-Placebo-C18 or mAb treatment. However, the PD-L1 levels were significantly reduced in samples that had GalXC-mCD274-C18 treatment suggesting that GalXC-mCD274-C18 efficiently silenced the intracellular PD-L1 expressed by Cd11c cells in lymph nodes.

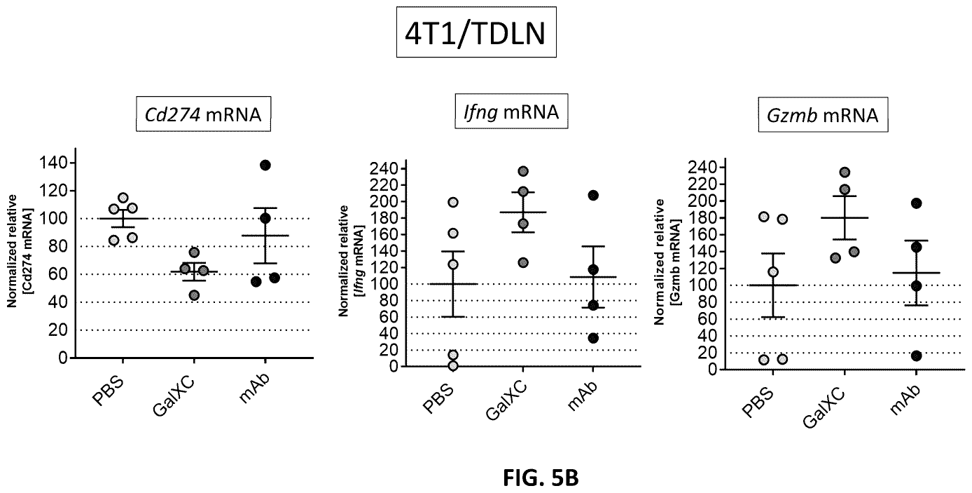

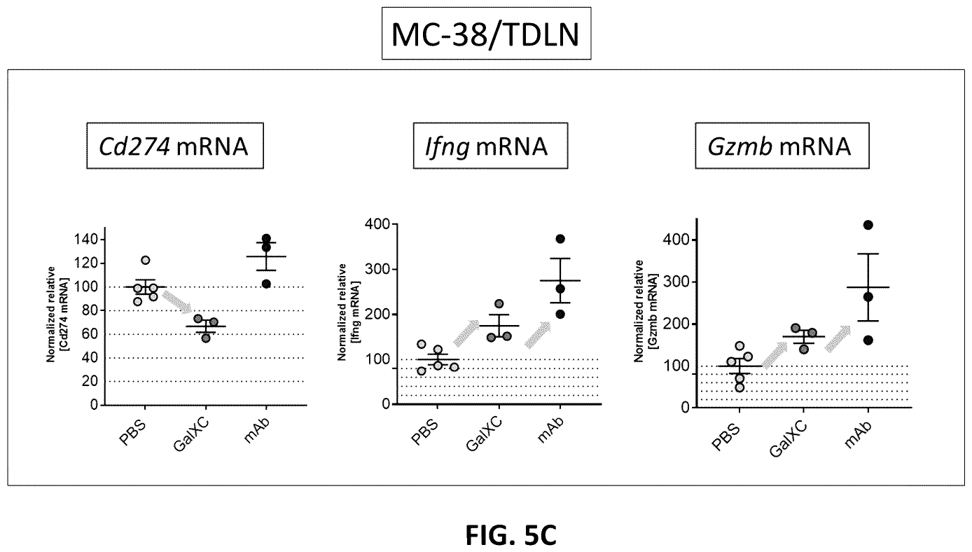

Anti-PD-L1 mAb typically acts to unleash the break between extracellular PD-1 and PD-L1 engagement, thus it is possible that mAb treatment may not reduce protein levels. However, one would expect to see immune activation in TDLN once the Cd274 gene is silenced by RNAi or the PD-1/PD-L1 bond is unleashed by mAb treatment. To evaluate immune activation in TDLN after silencing of Cd274 using RNAi or a mAb, lymph nodes collected from another similar study d were processed and analyzed for mRNA levels of Ifng and Gzmb. As shown in FIGS. 5A-5B, the activation markers were increased in TDLN that had GalXC-mCD274-C18 treatment but not the ones that had anti-PD-L1 mAb treatment suggesting that GalXC-mCD274-C18 mediated silencing of PD-L1 and enhances the antigen presentation machinery and priming of T-cells in a resistant tumor type whereas the anti-PD-L1 mAb was unable to increase T-cell activation. In contrast, when the same process was repeated in an inflamed phenotype, MC-38 tumor model, in which the antigen presentation, T-cell priming, and T-cell trafficking to TME happens fairly well, the activation markers increased to same extent by both anti-PD-L1 mAb and GalXC-mCD274-C18 (FIG. 5C). This indicates that the inhibition of PD-L1 on host or both host and tumor cells mediate similar immune responses in tumor types with intact immunity. However, RNAi-PDL1 mediated functions could be differentiated from mAb in resistant tumor phenotypes where anti-tumor immunity is suboptimal or dampened. Thus, RNAi-PDL1 mediated intracellular Cd274 silencing seems critical for rejuvenating antigen presentation to initiate T-cell activation.

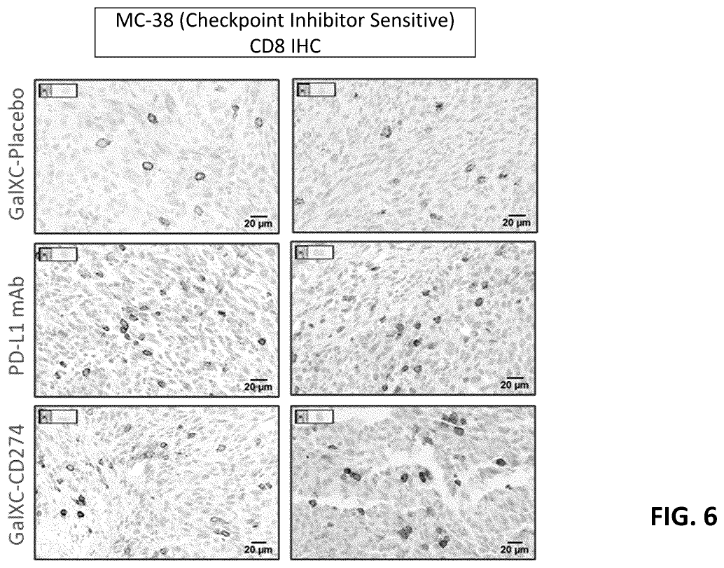

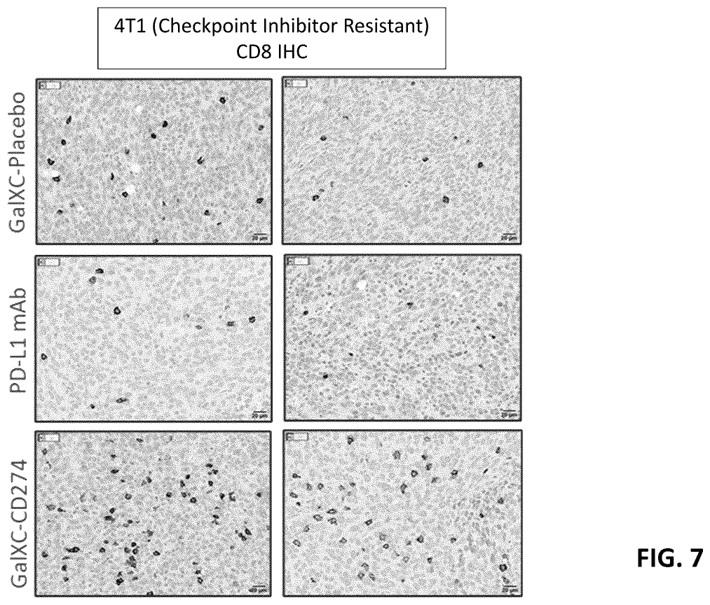

To see if the activated T-cells migrate into TME, the T-cell marker was measured using immunohistochemistry analysis of CD8 expression in 4T1 (checkpoint inhibitor resistant) and MC-38 (checkpoint inhibitor sensitive) tumors. Tumors from mice treated as described above were prepared for CD8 immunohistochemical analysis. Upon treatment with the anti-PD-L1 antibody or GalXC-mCD274-C18 RNAi oligonucleotide, similar increase in CD8 positive stain, indicating similar T-cell infiltration was observed in the checkpoint inhibitor sensitive tumors (FIG. 6). However, T-cell infiltration only increased in the checkpoint resistant tumors upon treatment with GalXC-mCD274-C18 (FIG. 7).

The data collectively demonstrate that both molecules act similarly in sensitive tumors but the CD274 targeting lipid-conjugated oligonucleotide mediated functions could be differentiated from mAb mainly in resistant tumor phenotypes as mAb might not have access to the other formats of PD-L1, especially the intracellular PD-L1 that is known to be in involved in mediating resistance whereas a CD274 targeting lipid-conjugated oligonucleotide could.

### Example 5: CD274 Inhibition Increases T-Cell Infiltration in TME and Mediates Acute Tumor Effects in Resistant Tumors

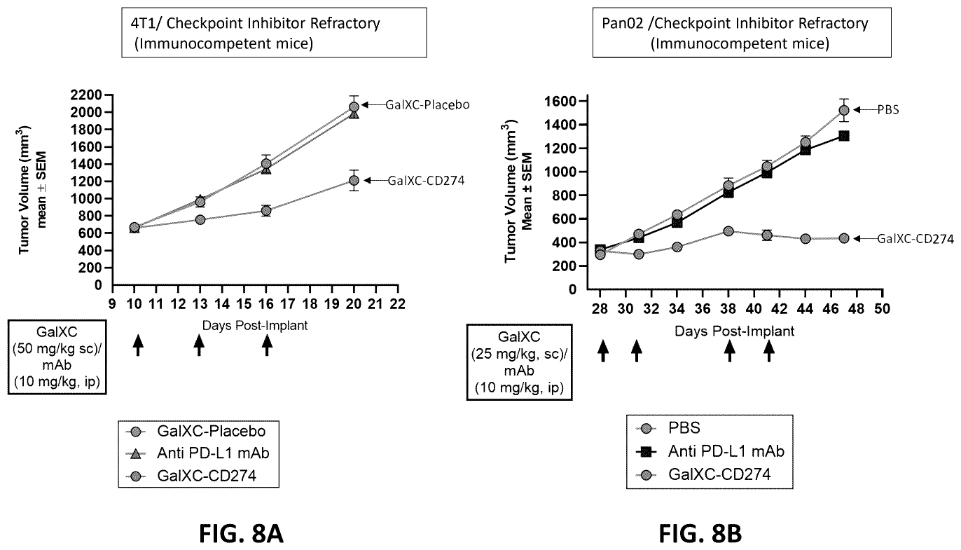

To evaluate if the infiltrated T-cells in the TME have any effect on tumor growth, studies were run in two resistant tumor models. In the first study, 4T1 tumor bearing Balb/c mice (n=5 per group) were treated subcutaneously with i) GalXC-placebo-C18; or ii) anti-PDL1 mAb; or, iii) GalXC-mCD274-C18. GalXC-mCD274-C18 and GalXC-Placebo-C18 were administered at a dose of 50 mg/kg, whereas the anti-PD-L1 mAb was administered at a dose of 10 mg/kg on days 10, 13, and 16 after tumor cell injection. Tumors treated with GalXC-mCD274-C18 demonstrated significant tumor growth inhibition whereas the tumors that had mAb or GalXC-Placebo-C18 treatment had no effect at all (FIG. 8A). Necrotic areas identified in tumors that had GalXC-mCD274-C18 treatment suggest tumor killing (data not shown). In a separate study, Pan02 tumor bearing mice were sorted and treated with the same agents at 25 mg/kg at q3dx2 per week for 2 weeks. Significant growth inhibition in tumors that had GalXC-mCD274-C18 treatment and not in tumors that had either mAb or GalXC-Placebo-C18 treatments confirming that the GalXC-mCD274-C18, by silencing PD-L1 in TME and TDLN a resistant phenotype enhance T-cell infiltration in TME to kill tumors (FIG. 8B).

### Example 6: CD274 Targeting Lipid-Conjugated Oligonucleotide Mediated Anti-Tumor Activity is CD8⁺ T-Cell Mediated

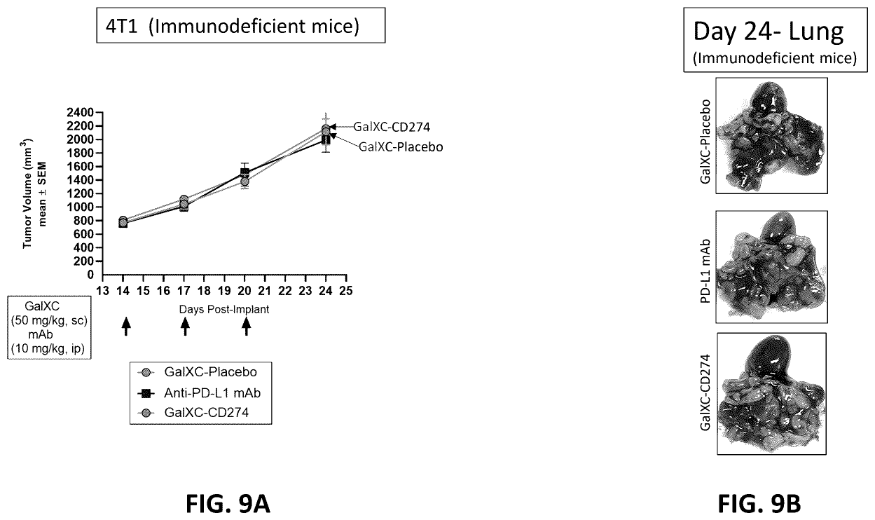

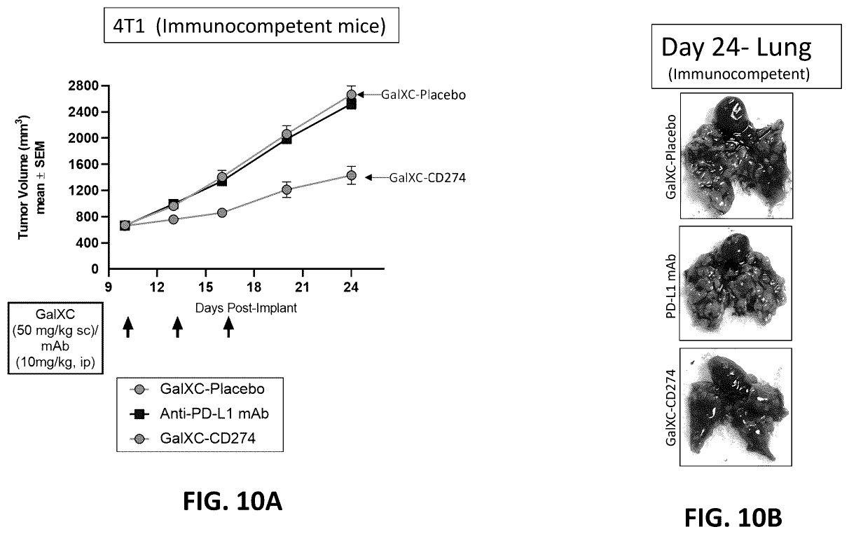

To evaluate if the anti-tumor efficacy observed with CD274 targeting lipid-conjugated oligonucleotide treatment is T-cell mediated, the same study was repeated in an immunodeficient mice (nude mice) which have no functional T-cells. 4T1 tumors were grown in nude mice and sorted and treated with i) GalXC-placebo-C18; or ii) anti-PDL1 mAb; or, iii) GalXC-mCD274-C18. GalXC-mCD274-C18 and GalXC-Placebo-C18 were administered at a dose of 50 mg/kg, whereas the anti-PD-L1 mAb was administered at a dose of 10 mg/kg on days 14, 17, and 20 as described in Example 5. No growth inhibition was observed in any of the groups suggesting that T-cells play a role in mediating tumor cell killing (FIG. 9A). For 4T1 tumors that were implanted in an immunocompetent mouse, tumor growth inhibition was significant after treatment with single agent GalXC-mCD274-C18 at 50 mg/kg as shown in FIGS. 8A and 10A. 4T1 tumors are known to spontaneously metastasize to lungs in about 3 weeks after the subcutaneous implantation, thus, in the same experiment that was run in Balb/c mice, lung tissue was collected to evaluate the presence of metastatic lesions. As shown in FIG. 10B, the lungs collected from GalXC-Placebo-C18 and mAb treatment groups had metastatic lesions whereas the lungs from GalXC-mCD274-C18 treatment group did not show any visual lesions in the lung. However, lungs collected from the study repeated in nude mice had metastatic lesions with all treatments suggesting that the GalXC-mCD274-C18 mediated anti-tumor activity requires activated T-cells (FIG. 9B). These studies also reveal that these activated T-cells generated in mice with intact immune system, beyond treating the primary tumors also limit the spontaneous metastasis to lung whereas the anti-PD-L1 mAb in the resistant tumor settings was unable to do any of the above.

### Example 7: CD274 Targeting Lipid-Conjugated Oligonucleotide and Anti-PD-L1 mAb are Active in Tumors with Inflamed Phenotype

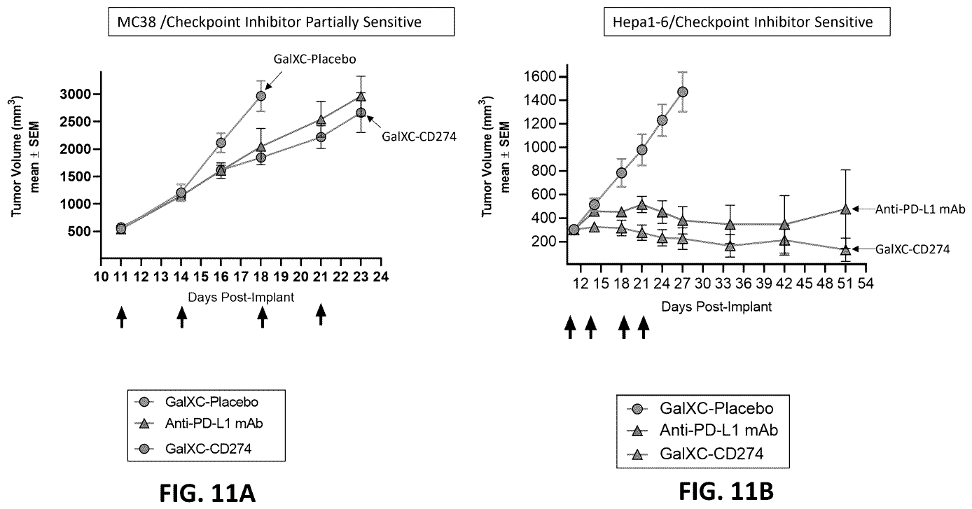

To evaluate how a CD274 targeting lipid-conjugated oligonucleotide and anti-PD-L1 mAb behave in an inflamed tumor phenotype, MC-38 and Hepa1-6 tumors were evaluated in mice. Specifically, 5×10⁶ MC-38 cells were first implanted in C57BL/6 mice and when the tumors reached the size of 300-500 mm³, they were sorted and treated subcutaneously with either GalXC-Placebo-C18, GalXC-mCD274-C18 at 25 mg/kg or intraperitoneally with anti-PD-L1 mAb at 10 mg/kg. During the study, the tumors were measured twice a week. As expected, mAb demonstrated some anti-tumor efficacy in this inflamed tumor model FIG. 11A. GalXC-mCD274-C18 demonstrated almost identical growth inhibition as mAb suggesting that both behave similarly in this type of tumor model. In another study, Hepa1-6 tumors, another inflamed tumor model, were grown in C57BL/6 mice and treated with the same compounds. The anti-PD-L1 mAb demonstrated more tumor growth inhibition compared to the growth inhibition observed in MC-38 tumors (FIG. 11B). GalXC-mCD274-C18 was as efficient as the anti-PD-L1 mAb in this inflamed tumor as well suggesting that both compounds behave similarly in tumors with inflamed phenotype where the immunity cycle works effectively.

### Example 8: Combination of CD274 Targeting Lipid-Conjugated Oligonucleotide with Immune Checkpoint Antibodies Improves the Efficacy of Single Agents in Resistant and Inflamed Tumors

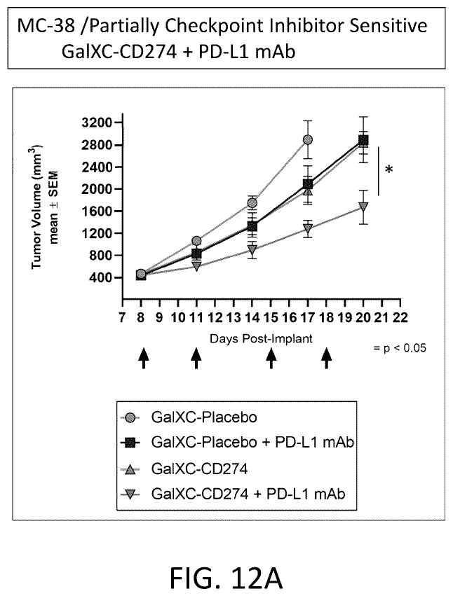

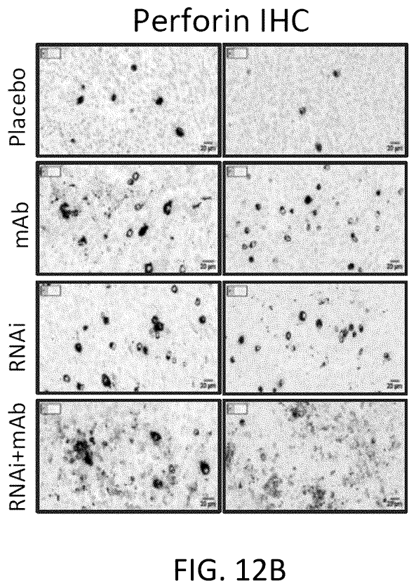

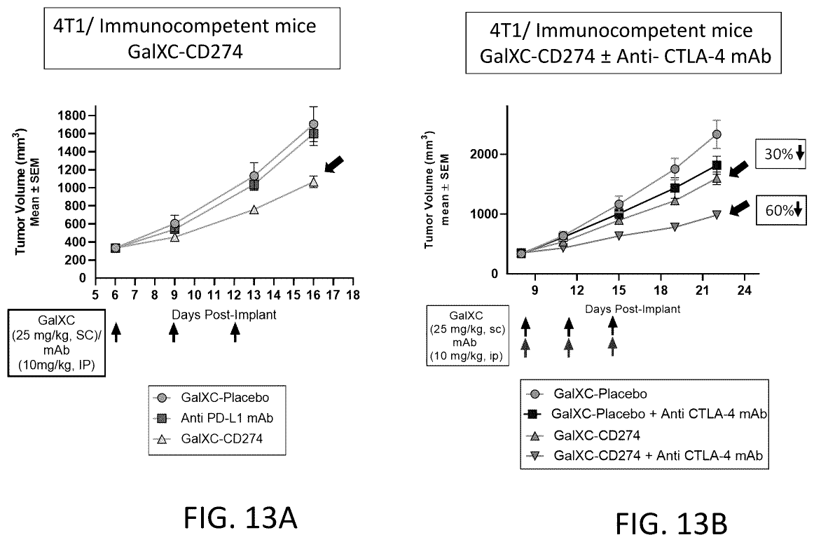

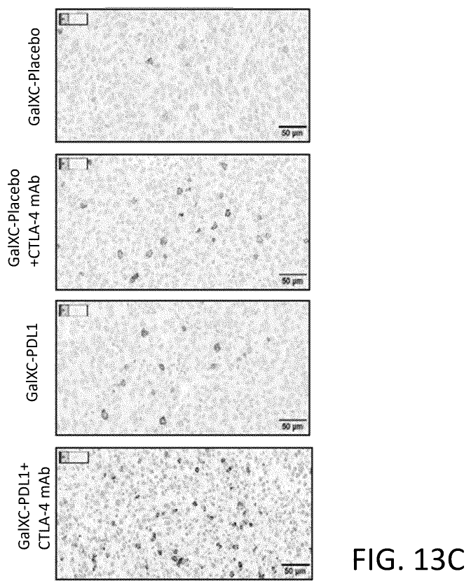

Based on the results presented here and literature, it is evident that PD-L1 expression in both host immune cells and tumor cells contribute to immune suppression, and that expression in both cell types could be predictive of sensitivity to therapeutic agents targeting the PD-L1/PD-1 axis in inflamed or partially inflamed tumor types (Lau et al, Nat. Commun., 8:14572, 2017). Since mAb targets both tumor and host extracellular PD-L1 and RNAi-PDL1 targets host extracellular and intracellular PD-L1 and demonstrate similar efficacy as single agents, it was investigated whether targeting PD-L1 using two different modalities could improve the efficacy of either of the single agents in these tumor types. To evaluate if combination of an immune checkpoint antibody with an anti-PD-L1 oligonucleotide would improve anti-tumor efficacy, studies were performed in the inflamed MC-38 tumor model and 4T1 checkpoint resistant model. MC-38 tumors were grown as described previously in C57BL/6 mice. Once the tumors reached the size of 300-500 mm³, mice were sorted into 4 groups (n=5) and treated with GalXC-Placebo-C18 or GalXC-mCD274-C18 with and without anti-PD-L1 mAb. GalXC compounds were dosed subcutaneously at 25 mg/kg and mAb was dosed intraperitoneally at 10 mg/kg at q3d×2. Tumor sizes were measured during the study period. As in FIG. 12, the combination of GalXC-mCD274-C18 and anti-PD-L1 mAb demonstrated improved efficacy compared to either of the single agents. Perforin staining was done of tumor tissue samples from the mice and increased perforin positive cells in tumors from mice treated with the combination was observed relative to control groups (FIG. 12B). This data supports the previous findings that the addition of PD-L1 mAb to RNAi-PDL1 treatment may increase the chances of targeting PD-L1 on both APCs and tumors and possibly other formats of PD-L1 that are not accessed by mAb in this tumor type. In addition, another study was conducted in 4T1 resistant model. 4T1 tumors were implanted in Balb/c mice and when the tumors reached the size of 300 mm³, they were sorted into 4 groups and treated with either i) GalXC-placebo-C18; or ii) anti-PD-L1 mAb; or, iii) GalXC-mCD274-C18 as single agents. GalXC compounds were dosed subcutaneously at 25 mg/kg and mAb was dosed intraperitoneally at 10 mg/kg on Days 6, 9, and 12. Treatment with GalXC-mCD274-C18 had improved anti-tumor efficacy compared to the anti-PD-L1 antibody (FIG. 13A). Since immunotherapy, especially blockade of the PD-1/PD-L1 and CTLA-4 axes, has resulted in durable responses in a range of cancer types with pre-existing immunity in patients, it was investigated whether combination could improve the activity of GalXC-CD274 in resistant tumors in preclinical mouse models. CTLA-4 mAb is thought to regulate T-cell proliferation early in an immune response, primarily in lymph nodes and thereby enhances the antigen presentation machinery (Seidel et al, Front. Oncol., 8:86, 2018). In contrast, PD-1 mAb suppresses T cells later in an immune response, primarily in peripheral tissues (Seidel et al, Front. Oncol., 8:86, 2018; Waldman et al, Nat. Rev. Immunol., Vol. 20: 651-668, 2020). Thus, CTLA-4 mAb may have a better chance of demonstrating some activity in resistant tumors compared to PD-1 or PD-L1 mAb treatments. Thus, it was evaluated whether combination therapy with the GalXC-mCD274-C18 oligonucleotide in combination with an anti-CTLA-4 immune checkpoint inhibitor would improve anti-tumor efficacy. Mice were administered GalXC-Placebo-C18 or GalXC-mCD274-C18 with and without an anti-CTLA-4 mAb (anti-mouse CTLA-4 mAb, clone #9H10, Cat #BP0131). As seen in FIG. 13B, the combination with anti-CTLA-4 mAb was able to increase the tumor growth inhibition by almost twice compared to anti-CTLA-4 mAb or GalXC-mCD274-C18 alone, and increase the presence of CD8+ cytotoxic T-cells in the tumor tissue (FIG. 13C), suggesting that this combination could be a potential combination therapy in clinic for resistant tumor types.

### Example 9: Generation of CD274-Targeting Double-Stranded RNAi Oligonucleotides

Identification of CD274 mRNA Target Sequences

To generate RNAi oligonucleotide inhibitors of human CD274 expression, a computer-based algorithm was used to computationally identify CD274 mRNA target sequences suitable for assaying inhibition of CD274 expression by the RNAi pathway. The algorithm provided RNAi oligonucleotide guide (antisense) strand sequences each having a region of complementarity to a suitable CD274 target sequence of human CD274 mRNA (Table 2). Some of the guide strand sequences identified by the algorithm were also complementary to the corresponding CD274 target sequence of monkey CD274 mRNA (Table 2) and/or mouse CD274 mRNA (Table 2) and/or rat CD274 mRNA (Table 2). CD274 RNAi oligonucleotides comprising a region of complementarity to homologous CD274 mRNA target sequences with nucleotide sequence similarity are predicted to have the ability to target homologous CD274 mRNAs.

TABLE 2
Sequences of Human, Monkey, Mouse, and Rat CD274 mRNA

| | | SEQ |
|---|---|---|
| Species | Ref Seq # | ID NO |
| Human (Hs) | NM_014143.4 | 1037 |
| Cynomolgus monkey (Mf) | XM_005581779.2 | 1038 |
| Mus Musculus (Mm) | NM_021893.3 | 1039 |
| Rattus norvegicus (Rn) | NM_001191954.1 | 1040 |

DsiRNAs identified in Table 3 were used to generate corresponding double-stranded RNAi oligonucleotides comprising a nicked tetraloop GalNAc-conjugated structure (referred to herein as “GalNAc-conjugated CD274 oligonucleotides” or “GalNAc-CD274 oligonucleotides”) having a 36-mer passenger strand and a 22-mer guide strand for evaluation in vitro. Further, the nucleotide sequences comprising the passenger strand and guide strand have a distinct pattern of modified nucleotides and phosphorothioate linkages.

TABLE 3
Distribution of dsiRNA across CD274
Sequence selection strategy: 240 sequences

| 108 Sequences from Exon 1-4 | | | | 132 Sequences from Exon 5-7 | | | |
|---|---|---|---|---|---|---|---|
| | | Hs | | | | Hs | |
| Location | Hs/Mf | Unique | Hs/Mf/Rat | Location | Hs/Mf | Unique | Hs/Mf/Rat |
| Exon 2 | 9 | 0 | 0 | Exon 5 | 5 | 0 | 0 |
| (CDS) | | | | (CDS) | | | |
| Exon 3 | 52 | 0 | 0 | Exon 6 | 0 | 1 | 0 |
| (CDS) | | | | (CDS) | | | |
| Exon 4 | 22 | 24 | 1 | Exon 7 | 96 | 29 | 1 |
| (CDS) | | | | (3′UTR) | | | |
| Total | 83 | 24 | 1 | Total | 101 | 30 | 1 |

| | |
|---|---|
| 108 | 132 |

Each GalNAc-CD274 oligonucleotide was generated with the same modification pattern, and each with a unique guide strand having a region of complementarity to a CD274 target sequence identified by SEQ ID NO: 1037. Modifications for the sense and anti-sense strand included the following:

Sense Strand:
5′ mX-S-mX-mX-mX-mX-mX-mX-fX-fX-fX-fX[-mX-]₁₆-
[ademX-GalNAc]-[ademX-GalNAc]-[ademX-GalNAc]-mX-
mX-mX-mX-mX-mX 3′.

### Hybridized to:

Antisense Strand:
5′ [MePhosphonate-4O-mX]-S-fX-S-fX-fX-fX-mX-fX-mX-
mX-fX-mX-mX-mX-fX-mX-mX-mX-mX-mX-mX-S-mX-S-mX 3′.

Or, represented as:

Sense Strand:
[mXs][mX][mX][mX][mX[mX][mX][fX][fX][fX][fX][mX]
[mX][mX][mX][mX][mX][mX][mX][mX][mX][mX][mX][mX]
[mX][mX][mX][ademA-GalNAc][ademA-GalNAc][ademA-
GalNAc][mX][mX[mX][mX][mX][mX]

### Hybridized to:

Antisense Strand:
[MePhosphonate-4O-mXs][fXs][fX][fX][fX][mX][fX]
[mX][mX][fX][mX][mX][mX][fX][mX][mX][mX][mX][mX]
[mXs][mXs][mX]

### (Modification key: Table 1).

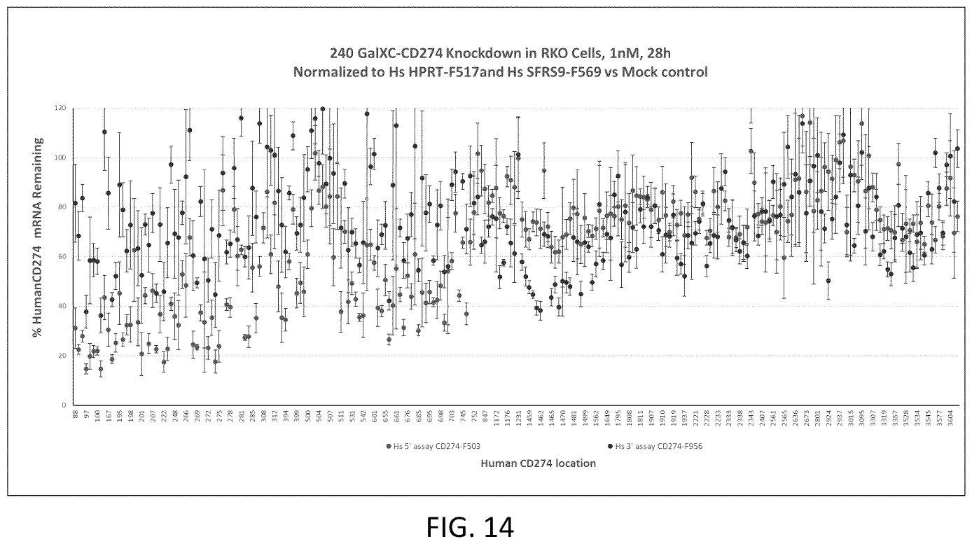

The ability of each of the GalNAc-CD274 oligonucleotides in Table 4 to reduce CD274 mRNA was measured using in vitro cell-based assays. Briefly, colon carcinoma (RKO) cells expressing endogenous human CD274 gene were transfected with each of the GalNAc-CD274 oligonucleotides listed in Table 4 and FIG. 14 at 1 nM in separate wells of a multi-well cell-culture plate. Cells were maintained for 28 hours following transfection with the GalNAc-CD274 oligonucleotides, and then the amount of remaining CD274 mRNA from the transfected cells was determined using TAQMAN®-based qPCR assays. Two qPCR assays, a 3′ assay (Hs01125301_m1; Assay location: 956, Exon Boundary: 6-7, Amplicon Length: 89) and a 5′ assay (Hs00204257_m1; Assay location: 503, Exon Boundary: 3-4, Amplicon Length: 77) were used to determine CD274 mRNA levels as measured using PCR probes conjugated to FAM-MGB. Each primer pair was assayed for % remaining RNA as shown in Table 4 and FIG. 14. The RKO cell-based assay evaluating the ability of the GalNAc-CD274 oligonucleotides listed in Table 4 and FIG. 14 to inhibit CD274 expression identified several candidate oligonucleotides.

TABLE 4
Analysis of CD274 mRNA in RKO cells

| | SED ID | | | | | |
|---|---|---|---|---|---|---|
| SED ID | NO | | | | | |
| NO | (Anti- | | | | | |
| (Sense | sense | | CD274-5′ Assay | | CD274-3′ Assay | |
| Strand) | Strand) | DsiRNA name | % remaining | SEM | % remaining | SEM |
| 483 | 724 | CD274-0088 | 31.131 | 8.166 | 81.52 | 15.779 |
| 484 | 725 | CD274-0094 | 22.467 | 1.982 | 68.275 | 9.8 |
| 519 | 760 | CD274-0095 | 27.836 | 2.463 | 83.562 | 5.402 |
| 486 | 727 | CD274-0097 | 14.624 | 1.989 | 37.777 | 6.621 |
| 487 | 728 | CD274-0098 | 19.688 | 5.22 | 58.411 | 17.505 |
| 520 | 761 | CD274-0099 | 21.654 | 2.444 | 58.524 | 6.653 |
| 488 | 729 | CD274-0100 | 21.955 | 1.686 | 58.043 | 5.27 |
| 489 | 730 | CD274-0102 | 14.706 | 3.206 | 36.176 | 7.098 |
| 521 | 762 | CD274-0103 | 43.544 | 8.938 | 110.249 | 15.649 |
| 490 | 731 | CD274-0167 | 30.462 | 6.748 | 85.565 | 14.554 |
| 491 | 732 | CD274-0193 | 18.532 | 1.77 | 42.613 | 2.838 |
| 492 | 733 | CD274-0194 | 25.08 | 3.734 | 52.139 | 5.447 |
| 522 | 763 | CD274-0195 | 45.2 | 12.572 | 88.839 | 21.094 |
| 523 | 764 | CD274-0196 | 26.453 | 2.662 | 78.79 | 10.877 |
| 493 | 734 | CD274-0197 | 32.419 | 4.17 | 62.235 | 8.532 |
| 524 | 765 | CD274-0198 | 32.598 | 7.179 | 72.723 | 17.723 |
| 525 | 766 | CD274-0199 | 62.634 | 20.672 | 147.834 | 27.428 |
| 494 | 735 | CD274-0200 | 33.61 | 9.742 | 63.268 | 15.099 |
| 526 | 767 | CD274-0201 | 20.67 | 8.82 | 52.382 | 19.352 |
| 527 | 768 | CD274-0202 | 44.248 | 3.273 | 72.803 | 4.238 |
| 495 | 736 | CD274-0204 | 24.794 | 3.963 | 64.526 | 10.869 |
| 528 | 769 | CD274-0207 | 46.253 | 6.873 | 77.443 | 7.824 |
| 496 | 737 | CD274-0219 | 22.568 | 1.5 | 45.206 | 3.705 |
| 529 | 770 | CD274-0221 | 36.692 | 7.56 | 72.824 | 14.824 |
| 497 | 738 | CD274-0222 | 17.408 | 4.065 | 45.821 | 11.643 |
| 498 | 739 | CD274-0225 | 22.667 | 4.679 | 65.495 | 14.048 |
| 530 | 771 | CD274-0245 | 40.961 | 2.67 | 97.103 | 7.481 |
| 531 | 772 | CD274-0248 | 35.884 | 9.454 | 69.114 | 17.944 |
| 532 | 773 | CD274-0249 | 32.318 | 9.969 | 67.66 | 23.129 |
| 533 | 774 | CD274-0265 | 52.712 | 5.609 | 77.545 | 4.677 |
| 534 | 775 | CD274-0266 | 48.364 | 12.889 | 92.158 | 27.092 |
| 535 | 776 | CD274-0267 | 67.687 | 7.444 | 111.065 | 12.415 |
| 499 | 740 | CD274-0268 | 24.417 | 5.5 | 60.403 | 14.082 |
| 500 | 741 | CD274-0269 | 23.346 | 1.106 | 49.42 | 2.061 |
| 536 | 777 | CD274-0270 | 37.356 | 3.264 | 82.157 | 6.125 |
| 501 | 742 | CD274-0271 | 33.57 | 20.274 | 59.016 | 35.992 |
| 537 | 778 | CD274-0272 | 23.079 | 4.531 | 50.465 | 8.805 |
| 538 | 779 | CD274-0273 | 35.336 | 7.161 | 71.047 | 11.705 |
| 502 | 743 | CD274-0274 | 17.571 | 4.631 | 44.591 | 13.132 |
| 503 | 744 | CD274-0275 | 23.769 | 6.433 | 68.437 | 18.304 |
| 539 | 780 | CD274-0276 | 86.685 | 14.294 | 93.539 | 14.98 |
| 504 | 745 | CD274-0277 | 40.665 | 2.339 | 61.793 | 4.828 |
| 540 | 781 | CD274-0278 | 39.561 | 3.986 | 65.163 | 5.506 |
| 541 | 782 | CD274-0279 | 78.972 | 9.244 | 95.57 | 12.252 |
| 542 | 783 | CD274-0280 | 60.112 | 11.875 | 66.845 | 12.462 |
| 543 | 784 | CD274-0281 | 62.654 | 1.914 | 115.961 | 7.28 |
| 505 | 746 | CD274-0282 | 27.321 | 1.139 | 59.937 | 4.127 |
| 506 | 747 | CD274-0283 | 27.658 | 4.375 | 63.588 | 12.755 |
| 544 | 785 | CD274-0285 | 55.463 | 9.866 | 87.534 | 19.056 |
| 545 | 786 | CD274-0303 | 35.186 | 5.987 | 75.895 | 10.403 |
| 546 | 787 | CD274-0305 | 56.025 | 3.939 | 113.756 | 8.055 |
| 547 | 788 | CD274-0308 | 71.524 | 6.064 | 142.48 | 11.081 |
| 548 | 789 | CD274-0309 | 85.974 | 18.56 | 104.235 | 20.277 |
| 549 | 790 | CD274-0310 | 60.856 | 7.784 | 102.91 | 14.216 |
| 550 | 791 | CD274-0312 | 81.697 | 18.488 | 101.073 | 24.339 |
| 551 | 792 | CD274-0313 | 47.952 | 10.222 | 86.381 | 17.656 |
| 552 | 793 | CD274-0392 | 35.313 | 10.073 | 72.643 | 19.061 |
| 507 | 748 | CD274-0394 | 34.533 | 4.527 | 61.795 | 10.376 |
| 553 | 794 | CD274-0395 | 57.943 | 3.249 | 85.57 | 2.828 |
| 554 | 795 | CD274-0398 | 78.961 | 5.981 | 108.755 | 5.647 |
| 508 | 749 | CD274-0399 | 45.247 | 5.935 | 69.271 | 6.786 |
| 555 | 796 | CD274-0497 | 49.399 | 8.31 | 72.717 | 11.033 |
| 556 | 797 | CD274-0499 | 45.77 | 10.107 | 83.657 | 17.594 |
| 557 | 798 | CD274-0500 | 60.879 | 5.775 | 95.202 | 9.23 |
| 558 | 799 | CD274-0501 | 79.425 | 7.344 | 110.765 | 11.3 |
| 559 | 800 | CD274-0502 | 101.777 | 11.072 | 115.645 | 10.26 |
| 560 | 801 | CD274-0504 | 86.588 | 5.657 | 97.582 | 5.378 |
| 561 | 802 | CD274-0505 | 88.084 | 8.852 | 119.591 | 18.275 |
| 562 | 803 | CD274-0506 | 80.134 | 8.34 | 89.148 | 11.903 |
| 563 | 804 | CD274-0507 | 84.236 | 19.13 | 99.615 | 27.193 |
| 564 | 805 | CD274-0509 | 59.654 | 11.452 | 93.381 | 20.147 |
| 565 | 806 | CD274-0510 | 84.182 | 13.358 | 121.09 | 23.181 |
| 566 | 807 | CD274-0511 | 37.722 | 8.209 | 71.435 | 16.151 |
| 567 | 808 | CD274-0513 | 69.923 | 4.642 | 89.348 | 5.818 |
| 509 | 750 | CD274-0530 | 41.726 | 8.837 | 62.528 | 16.987 |
| 510 | 751 | CD274-0531 | 49.269 | 4.524 | 69.733 | 5.656 |
| 568 | 809 | CD274-0535 | 42.833 | 2.855 | 65.22 | 5.915 |
| 569 | 810 | CD274-0537 | 35.5 | 1.623 | 56.41 | 2.935 |
| 570 | 811 | CD274-0542 | 36.307 | 9.025 | 65.621 | 16.508 |
| 571 | 812 | CD274-0583 | 64.631 | 18.243 | 117.563 | 34.03 |
| 572 | 813 | CD274-0594 | 64.738 | 6.236 | 96.315 | 7.453 |
| 573 | 814 | CD274-0601 | 57.424 | 2.985 | 101.399 | 6.459 |
| 511 | 752 | CD274-0653 | 39.347 | 7.458 | 63.368 | 11.409 |
| 574 | 815 | CD274-0654 | 38.035 | 2.244 | 68.814 | 4.15 |
| 575 | 816 | CD274-0655 | 50.502 | 6.281 | 72.479 | 9.581 |
| 576 | 817 | CD274-0656 | 26.434 | 2.197 | 42.131 | 3.669 |
| 577 | 818 | CD274-0658 | 40.229 | 11.877 | 88.739 | 30.141 |
| 578 | 819 | CD274-0661 | 55.074 | 24.269 | 112.789 | 56.263 |
| 579 | 820 | CD274-0667 | 44.652 | 2.824 | 71.567 | 3.542 |
| 512 | 753 | CD274-0673 | 31.408 | 3.369 | 58.398 | 7.122 |
| 580 | 821 | CD274-0676 | 52.316 | 5.006 | 67.287 | 9.849 |
| 581 | 822 | CD274-0679 | 43.883 | 5.096 | 76.975 | 8.949 |
| 582 | 823 | CD274-0681 | 60.948 | 20.529 | 104.591 | 37.457 |
| 583 | 824 | CD274-0685 | 30.2 | 2.29 | 54.492 | 4.559 |
| 584 | 825 | CD274-0692 | 45.56 | 13.81 | 91.513 | 27.21 |
| 513 | 754 | CD274-0693 | 41.287 | 8.011 | 77.58 | 15.297 |
| 585 | 826 | CD274-0695 | 45.63 | 4.714 | 81.152 | 7.448 |
| 586 | 827 | CD274-0696 | 41.525 | 1.814 | 58.329 | 1.93 |
| 514 | 755 | CD274-0697 | 42.473 | 7.504 | 72.755 | 14.161 |
| 587 | 828 | CD274-0698 | 48.288 | 5.829 | 80.46 | 9.816 |
| 515 | 756 | CD274-0700 | 33.352 | 3.445 | 53.838 | 6.534 |
| 516 | 757 | CD274-0701 | 54.142 | 21.568 | 55.952 | 27.121 |
| 588 | 829 | CD274-0703 | 58.221 | 3.36 | 88.83 | 6.412 |
| 589 | 830 | CD274-0709 | 77.371 | 7.558 | 94.049 | 8.223 |
| 517 | 758 | CD274-0743 | 44.282 | 2.319 | 126.324 | 5.388 |
| 590 | 831 | CD274-0745 | 65.521 | 2.253 | 90.277 | 3.219 |
| 518 | 759 | CD274-0746 | 36.95 | 4.351 | 71.04 | 10.009 |
| 591 | 832 | CD274-0749 | 65.698 | 9.266 | 92.359 | 12.22 |
| 592 | 833 | CD274-0752 | 77.711 | 14.747 | 81.458 | 12.595 |
| 593 | 834 | CD274-0800 | 101.614 | 12.187 | 84.061 | 7.365 |
| 594 | 835 | CD274-0804 | 94.618 | 10.291 | 64.501 | 3.344 |
| 595 | 836 | CD274-0847 | 87.273 | 10.051 | 66.012 | 8.744 |
| 596 | 837 | CD274-0852 | 81.629 | 6.848 | 71.983 | 4.279 |
| 597 | 838 | CD274-0889 | 84.449 | 2.629 | 76.008 | 3.317 |
| 598 | 839 | CD274-1172 | 87.554 | 20.066 | 75.283 | 21.587 |
| 599 | 840 | CD274-1173 | 77.381 | 6.273 | 51.721 | 4.623 |
| 600 | 841 | CD274-1174 | 76.232 | 2.502 | 57.527 | 1.389 |
| 601 | 842 | CD274-1176 | 92.286 | 4.181 | 72.036 | 3.177 |
| 602 | 843 | CD274-1178 | 90.703 | 9.822 | 65.447 | 9.592 |
| 603 | 844 | CD274-1227 | 87.843 | 14.618 | 61.184 | 11.576 |
| 604 | 845 | CD274-1231 | 99.647 | 16.42 | 101.278 | 15.079 |
| 605 | 846 | CD274-1423 | 74.846 | 7.088 | 57.747 | 3.354 |
| 606 | 847 | CD274-1425 | 70.873 | 7.145 | 51.942 | 3.924 |
| 607 | 848 | CD274-1459 | 67.012 | 4.231 | 47.607 | 2.626 |
| 608 | 849 | CD274-1460 | 73.956 | 3.971 | 44.648 | 1.563 |
| 609 | 850 | CD274-1461 | 73.688 | 5.333 | 39.205 | 2.882 |
| 610 | 851 | CD274-1462 | 71.131 | 7.851 | 38.196 | 3.75 |
| 611 | 852 | CD274-1463 | 94.585 | 11.296 | 68.95 | 4.86 |
| 612 | 853 | CD274-1464 | 71.931 | 5.855 | 68.114 | 4.49 |
| 613 | 854 | CD274-1465 | 63.807 | 6.413 | 43.567 | 3.485 |
| 614 | 855 | CD274-1467 | 61.71 | 6.34 | 48.726 | 3.334 |
| 615 | 856 | CD274-1469 | 61.962 | 4.694 | 39.678 | 3.636 |
| 616 | 857 | CD274-1470 | 67.735 | 20.113 | 50.039 | 13.925 |
| 617 | 858 | CD274-1473 | 68.872 | 5.99 | 49.518 | 4.118 |
| 618 | 859 | CD274-1474 | 75.242 | 6.376 | 47.828 | 3.511 |
| 619 | 860 | CD274-1481 | 79.557 | 11.572 | 67.562 | 9.505 |
| 620 | 861 | CD274-1484 | 76.996 | 17.926 | 66.005 | 13.328 |
| 621 | 862 | CD274-1486 | 64.851 | 7.712 | 44.903 | 5.084 |
| 622 | 863 | CD274-1499 | 75.557 | 9.817 | 65.643 | 9.631 |
| 623 | 864 | CD274-1500 | 70.205 | 2.619 | 63.891 | 2.108 |
| 624 | 865 | CD274-1541 | 68.299 | 4.42 | 49.619 | 3.3 |
| 625 | 866 | CD274-1562 | 71.195 | 4.333 | 57.049 | 5.217 |
| 626 | 867 | CD274-1606 | 78.485 | 18.402 | 80.868 | 17.544 |
| 627 | 868 | CD274-1607 | 71.565 | 3.599 | 58.279 | 3.459 |
| 628 | 869 | CD274-1649 | 76.356 | 9.594 | 68.991 | 7.379 |
| 629 | 870 | CD274-1728 | 77.22 | 18.743 | 67.444 | 17.035 |
| 630 | 871 | CD274-1794 | 76.237 | 4.033 | 84.87 | 5.324 |
| 631 | 872 | CD274-1795 | 80.053 | 11.775 | 92.368 | 10.395 |
| 632 | 873 | CD274-1806 | 74.8 | 8.029 | 56.655 | 9.198 |
| 633 | 874 | CD274-1807 | 80.433 | 16.631 | 77.905 | 20.096 |
| 634 | 875 | CD274-1808 | 73.574 | 11.705 | 59.667 | 5.989 |
| 635 | 876 | CD274-1809 | 86.54 | 14.407 | 71.61 | 10.012 |
| 636 | 877 | CD274-1810 | 84.532 | 11.837 | 62.905 | 7.538 |
| 637 | 878 | CD274-1811 | 84.074 | 3.107 | 78.893 | 5.342 |
| 638 | 879 | CD274-1812 | 83.763 | 8.447 | 72.064 | 6.848 |
| 639 | 880 | CD274-1906 | 82.981 | 2.043 | 83.87 | 4.354 |
| 640 | 881 | CD274-1907 | 78.78 | 11.148 | 71.935 | 8.162 |
| 641 | 882 | CD274-1908 | 80.371 | 6.974 | 80.41 | 7.182 |
| 642 | 883 | CD274-1909 | 74.66 | 5.311 | 70.485 | 4.094 |
| 643 | 884 | CD274-1910 | 85.884 | 10.611 | 60.787 | 7.087 |
| 644 | 885 | CD274-1913 | 76.59 | 12.573 | 68.56 | 11.281 |
| 645 | 886 | CD274-1915 | 69.771 | 3.545 | 68.054 | 4.276 |
| 646 | 887 | CD274-1919 | 82.047 | 6.525 | 76.726 | 8.448 |
| 647 | 888 | CD274-1931 | 72.74 | 6.142 | 59.325 | 6.893 |
| 648 | 889 | CD274-1935 | 77.483 | 5.082 | 56.988 | 4.098 |
| 649 | 890 | CD274-1937 | 68.404 | 11.544 | 52.111 | 8.172 |
| 650 | 891 | CD274-2014 | 76.822 | 4.852 | 68.445 | 5.364 |
| 651 | 892 | CD274-2088 | 91.722 | 14.303 | 65.355 | 14.579 |
| 652 | 893 | CD274-2221 | 86.013 | 6.484 | 69.271 | 4.694 |
| 653 | 894 | CD274-2225 | 75.122 | 4.509 | 74.009 | 4.759 |
| 654 | 895 | CD274-2227 | 81.197 | 4.121 | 81.293 | 5.032 |
| 655 | 896 | CD274-2228 | 67.462 | 2.955 | 56.161 | 4.178 |
| 656 | 897 | CD274-2229 | 70.281 | 4.973 | 65.174 | 3.579 |
| 657 | 898 | CD274-2231 | 86.398 | 9.572 | 68.494 | 9.207 |
| 658 | 899 | CD274-2233 | 79.982 | 10.124 | 67.927 | 8.868 |
| 659 | 900 | CD274-2263 | | | 87.346 | 14.266 |
| 660 | 901 | CD274-2266 | 82.392 | 3.97 | 94.145 | 5.632 |
| 661 | 902 | CD274-2333 | 67.739 | 3.609 | 74.549 | 4.779 |
| 662 | 903 | CD274-2335 | 71.904 | 10.781 | 71.563 | 11.226 |
| 663 | 904 | CD274-2337 | 67.641 | 4.013 | 66.638 | 4.701 |
| 664 | 905 | CD274-2338 | 68.988 | 7.212 | 62.032 | 4.997 |
| 665 | 906 | CD274-2339 | 65.341 | 7.397 | 65.622 | 6.824 |
| 666 | 907 | CD274-2341 | 71.849 | 7.242 | 60.174 | 5.111 |
| 667 | 908 | CD274-2343 | 102.538 | 9.126 | 123.408 | 9.475 |
| 668 | 909 | CD274-2362 | 89.652 | 12.296 | 76.304 | 10.019 |
| 669 | 910 | CD274-2363 | 77.02 | 4.291 | 68.242 | 3.643 |
| 670 | 911 | CD274-2407 | 74.086 | 5.292 | 78.084 | 5.515 |
| 671 | 912 | CD274-2556 | 73.666 | 19.942 | 78.02 | 17.788 |
| 672 | 913 | CD274-2557 | 74.91 | 7.804 | 74.17 | 8.223 |
| 673 | 914 | CD274-2561 | 76.481 | 4.198 | 90.053 | 6.788 |
| 674 | 915 | CD274-2562 | 69.872 | 16.664 | 76.055 | 17.434 |
| 675 | 916 | CD274-2563 | 80.051 | 11.551 | 76.803 | 10.262 |
| 676 | 917 | CD274-2565 | 59.49 | 16.262 | 89.121 | 21.009 |
| 677 | 918 | CD274-2568 | 74.185 | 8.521 | 104.189 | 12.759 |
| 678 | 919 | CD274-2634 | 76.654 | 16.677 | 83.99 | 15.417 |
| 679 | 920 | CD274-2636 | 90.954 | 26.89 | 93.168 | 28.42 |
| 680 | 921 | CD274-2637 | 91.338 | 25.739 | 85.813 | 21.959 |
| 681 | 922 | CD274-2640 | 116.761 | 12.198 | 113.669 | 11.745 |
| 682 | 923 | CD274-2673 | 86.028 | 24.44 | 77.461 | 19.531 |
| 683 | 924 | CD274-2678 | 114.031 | 17.007 | 90.335 | 10.099 |
| 684 | 925 | CD274-2679 | 78.274 | 29.426 | 96.469 | 26.668 |
| 685 | 926 | CD274-2801 | 82.569 | 11.526 | 100.836 | 15.161 |
| 686 | 927 | CD274-2815 | 86.338 | 23.745 | 78.125 | 21.969 |
| 687 | 928 | CD274-2819 | 96.07 | 9.877 | 71.233 | 5.842 |
| 688 | 929 | CD274-2924 | 94.156 | 15.497 | 50.28 | 7.591 |
| 689 | 930 | CD274-2935 | 91.167 | 9.762 | 74.965 | 7.967 |
| 690 | 931 | CD274-2936 | 99.059 | 17.897 | 84.048 | 14.829 |
| 691 | 932 | CD274-2937 | 106.083 | 10.705 | 97.768 | 11.766 |
| 692 | 933 | CD274-2939 | 106.806 | 17.393 | 109.05 | 17.098 |
| 693 | 934 | CD274-2994 | 69.771 | 9.976 | 72.625 | 11.959 |
| 694 | 935 | CD274-3015 | 96.283 | 38.168 | 92.756 | 32.238 |
| 695 | 936 | CD274-3019 | 92.724 | 6.653 | 64.357 | 2.981 |
| 696 | 937 | CD274-3094 | 90.184 | 16.514 | 80.36 | 11.63 |
| 697 | 938 | CD274-3095 | 113.735 | 16.856 | 102.098 | 11.162 |
| 698 | 939 | CD274-3305 | 86.273 | 22.8 | 70.121 | 13.416 |
| 699 | 940 | CD274-3306 | 100.649 | 21.391 | 87.516 | 16.825 |
| 700 | 941 | CD274-3307 | 87.853 | 5.819 | 67.927 | 5.719 |
| 701 | 942 | CD274-3308 | 78.768 | 8.254 | 84.03 | 9.488 |
| 702 | 943 | CD274-3310 | 74.787 | 5.185 | 60.7 | 4.36 |
| 703 | 944 | CD274-3319 | 70.915 | 5.742 | 62.091 | 3.153 |
| 704 | 945 | CD274-3325 | 71.261 | 4.837 | 54.784 | 3.032 |
| 705 | 946 | CD274-3355 | 70.529 | 7.469 | 52.859 | 4.601 |
| 706 | 947 | CD274-3357 | 69.587 | 10.871 | 67.389 | 9.487 |
| 707 | 948 | CD274-3441 | 97.36 | 8.347 | 80.562 | 8.503 |
| 708 | 949 | CD274-3522 | 66.524 | 4.472 | 71.288 | 6.057 |
| 709 | 950 | CD274-3528 | 73.115 | 19.545 | 68.012 | 18.797 |
| 710 | 951 | CD274-3531 | 70.512 | 4.608 | 61.545 | 7.75 |
| 711 | 952 | CD274-3532 | 73.266 | 3.514 | 55.408 | 5.591 |
| 712 | 953 | CD274-3534 | 66.004 | 10.803 | 68.839 | 13.256 |
| 713 | 954 | CD274-3541 | 73.463 | 10.63 | 69.396 | 10.755 |
| 714 | 955 | CD274-3542 | 64.873 | 3.182 | 60.533 | 3.872 |
| 715 | 956 | CD274-3545 | 80.501 | 4.498 | 85.456 | 4.598 |
| 716 | 957 | CD274-3546 | 73.681 | 11.453 | 62.886 | 5.905 |
| 717 | 958 | CD274-3569 | 66.64 | 5.295 | 101.888 | 5.931 |
| 718 | 959 | CD274-3577 | 80.081 | 5.329 | 87.497 | 5.765 |
| 719 | 960 | CD274-3578 | 68.184 | 5.522 | 69.505 | 4.931 |
| 720 | 961 | CD274-3580 | 87.343 | 6.656 | 97.01 | 4.904 |
| 721 | 962 | CD274-3604 | 91.519 | 15.223 | 100.502 | 17.138 |
| 722 | 963 | CD274-3610 | 69.406 | 18.142 | 82.185 | 20.548 |
| 723 | 964 | CD274-3622 | 76.133 | 6.237 | 103.52 | 7.601 |

Taken together, these results show that GalNAc-CD274 oligonucleotides designed to target human CD274 mRNA inhibit CD274 expression in cells, as determined by a reduced amount of CD274 mRNA in the oligo-transfected cells relative to control cells. These results demonstrate that multiple CD274 mRNA target sequences are suitable for the RNAi-mediated inhibition of CD274 expression.

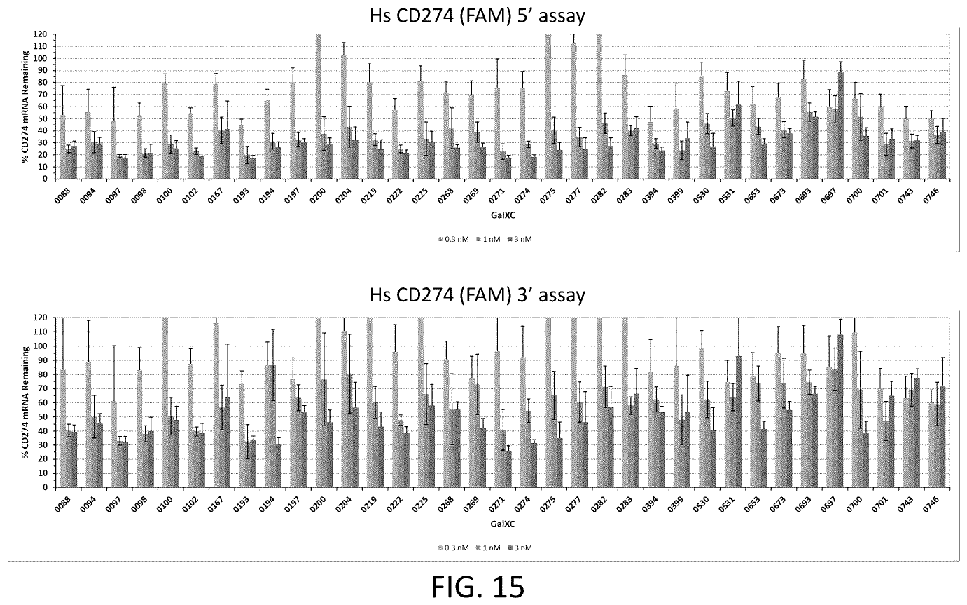

Following the initial in vitro screen, 35 constructs were selected for dosing studies. RKO cells were treated for 28 hours with 0.3 nM, 1 nM, and 3 nM of oligonucleotide. mRNA was isolated and measured to determine a potent dose (Table 5 and FIG. 15). These 35 sequences were selected for further testing in vivo (Table 5).

TABLE 5
Analysis of CD274 mRNA in Dose-Response Study in RKO cells

| | SED | | | | | | | |
|---|---|---|---|---|---|---|---|---|
| SED | ID NO | | | | | | 3 nM | |
| ID NO | (Anti- | | 0.3 nM | | 1 nM | | % | |
| (Sense | sense | DsiRNA | % | | % | | remain- | |
| Strand) | Strand) | name | remaining | SEM | remaining | SEM | ning | SEM |
| 965 | 1001 | CD274- | 68.1955 | 31.557 | 32.646 | 3.926 | 33.502 | 4.3365 |
| | | 0088 | | | | | | |
| 966 | 1002 | CD274- | 72.109 | 24.156 | 40.2615 | 12.015 | 37.8635 | 5.6855 |
| | | 0094 | | | | | | |
| 968 | 1004 | CD274- | 54.846 | 33.252 | 25.9785 | 2.167 | 24.9755 | 3.211 |
| | | 0097 | | | | | | |
| 969 | 1005 | CD274- | 67.9855 | 12.935 | 29.752 | 4.634 | 30.931 | 8.1975 |
| | | 0098 | | | | | | |
| 970 | 1006 | CD274- | 100.6175 | 8.997 | 39.67 | 10.359 | 36.75 | 7.879 |
| | | 0100 | | | | | | |
| 971 | 1007 | CD274- | 71.2525 | 7.3865 | 31.3205 | 2.9865 | 28.8135 | 7.1 |
| | | 0102 | | | | | | |
| 972 | 1008 | CD274- | 97.637 | 10.468 | 48.4765 | 13.39 | 52.562 | 30.5185 |
| | | 0167 | | | | | | |
| 973 | 1009 | CD274- | 59.014 | 6.947 | 26.147 | 9.728 | 25.486 | 2.387 |
| | | 0193 | | | | | | |
| 974 | 1010 | CD274- | 75.9815 | 12.73 | 58.997 | 15.777 | 28.4995 | 4.571 |
| | | 0194 | | | | | | |
| 975 | 1011 | CD274- | 78.4815 | 13.49 | 48.243 | 7.3815 | 42.379 | 3.358 |
| | | 0197 | | | | | | |
| 976 | 1012 | CD274- | 156.4925 | 47.47 | 57.041 | 23.257 | 37.7115 | 6.934 |
| | | 0200 | | | | | | |
| 977 | 1013 | CD274- | 106.8325 | 9.817 | 61.8775 | 22.394 | 44.528 | 14.2665 |
| | | 0204 | | | | | | |
| 978 | 1014 | CD274- | 106.3655 | 21.452 | 46.4935 | 8.115 | 34.067 | 8.9845 |
| | | 0219 | | | | | | |
| 979 | 1015 | CD274- | 76.658 | 14.271 | 36.3755 | 3.496 | 30.22 | 3.363 |
| | | 0222 | | | | | | |
| 980 | 1016 | CD274- | 105.7635 | 15.12 | 49.802 | 17.709 | 44.4205 | 11.9215 |
| | | 0225 | | | | | | |
| 981 | 1017 | CD274- | 81.539 | 10.697 | 48.6485 | 21.03 | 40.641 | 3.9295 |
| | | 0268 | | | | | | |
| 982 | 1018 | CD274- | 73.5275 | 13.628 | 55.9515 | 14.881 | 34.307 | 4.9905 |
| | | 0269 | | | | | | |
| 983 | 1019 | CD274- | 86.224 | 30.539 | 31.723 | 10.452 | 21.7565 | 2.667 |
| | | 0271 | | | | | | |
| 984 | 1020 | CD274- | 83.681 | 17.969 | 41.605 | 5.468 | 24.918 | 1.8835 |
| | | 0274 | | | | | | |
| 985 | 1021 | CD274- | 157.842 | 33.757 | 52.7885 | 13.872 | 29.4355 | 8.9055 |
| | | 0275 | | | | | | |
| 986 | 1022 | CD274- | 120.044 | 28.594 | 47.641 | 11.204 | 35.6665 | 15.304 |
| | | 0277 | | | | | | |
| 987 | 1023 | CD274- | 165.271 | 96.96 | 58.836 | 11.438 | 42.219 | 10.556 |
| | | 0282 | | | | | | |
| 988 | 1024 | CD274- | 112.219 | 19.616 | 48.9925 | 5.1515 | 54.382 | 13.5605 |
| | | 0283 | | | | | | |
| 989 | 1025 | CD274- | 64.8195 | 17.67 | 45.841 | 6.484 | 38.6535 | 3.2605 |
| | | 0394 | | | | | | |
| 990 | 1026 | CD274- | 72.1765 | 27.798 | 35.8075 | 12.719 | 43.658 | 19.6795 |
| | | 0399 | | | | | | |
| 991 | 1027 | CD274- | 91.8045 | 12.211 | 54.1095 | 10.602 | 33.9605 | 13.324 |
| | | 0530 | | | | | | |
| 992 | 1028 | CD274- | 73.99 | 15.171 | 57.32 | 8.2855 | 77.3445 | 23.5255 |
| | | 0531 | | | | | | |
| 993 | 1029 | CD274- | 70.4845 | 15.655 | 58.6105 | 9.457 | 35.415 | 4.795 |
| | | 0653 | | | | | | |
| 994 | 1030 | CD274- | 81.868 | 14.69 | 57.382 | 12.075 | 46.3645 | 5.087 |
| | | 0673 | | | | | | |
| 995 | 1031 | CD274- | 88.8905 | 17.812 | 65.039 | 8.101 | 58.942 | 4.6735 |
| | | 0693 | | | | | | |
| 996 | 1032 | CD274- | 72.578 | 18.137 | 70.689 | 13.116 | 98.576 | 9.4475 |
| | | 0697 | | | | | | |
| 997 | 1033 | CD274- | 88.039 | 17.009 | 60.3425 | 23.253 | 37.3435 | 7.3575 |
| | | 0700 | | | | | | |
| 998 | 1034 | CD274- | 64.731 | 12.59 | 37.932 | 11.519 | 49.3725 | 8.95 |
| | | 0701 | | | | | | |
| 999 | 1035 | CD274- | 56.5995 | 13.036 | 50.3755 | 8.645 | 54.8205 | 5.1925 |
| | | 0743 | | | | | | |
| 1000 | 1036 | CD274- | 55.083 | 7.7535 | 47.8085 | 11.22 | 54.9725 | 16.1295 |
| | | 0746 | | | | | | |

### Example 10: RNAi Oligonucleotide Inhibition of CD274 In Vivo

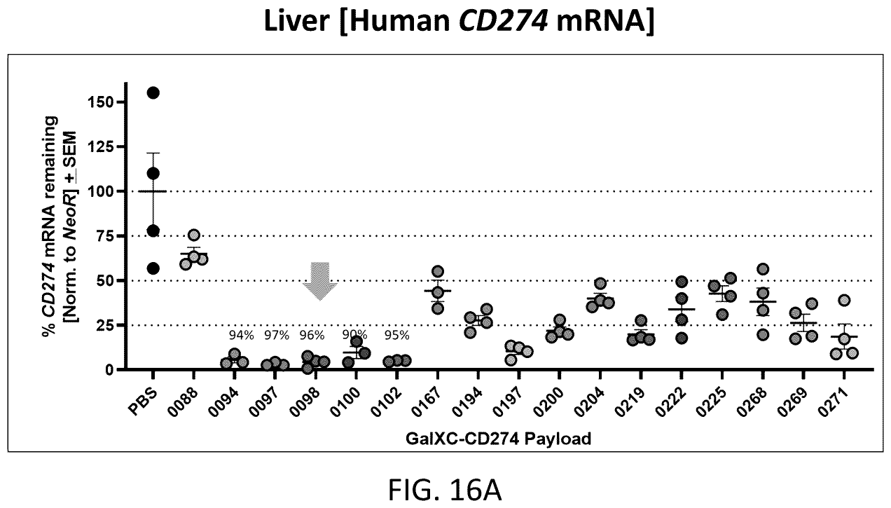

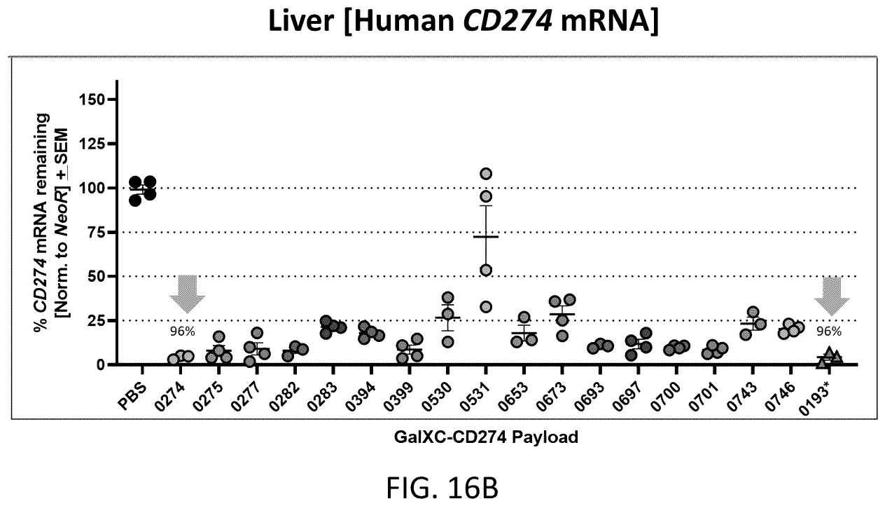

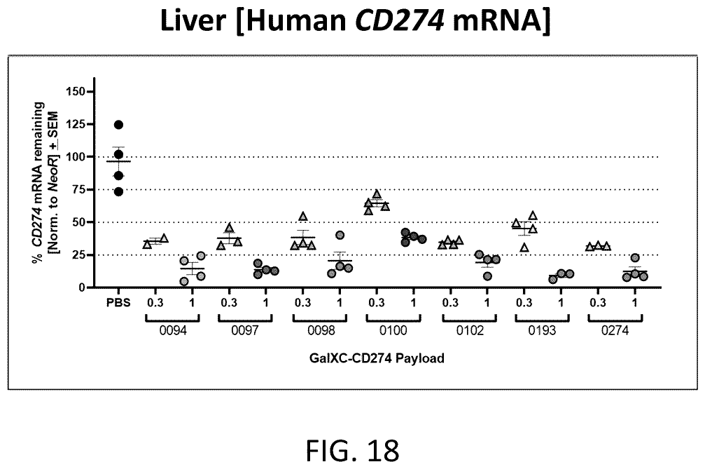

The in vitro screening assay in Example 9 validated the ability of CD274-targeting oligonucleotides to knock-down target mRNA. To confirm the ability of the RNAi oligonucleotides to knockdown CD274 in vivo, an HDI mouse model was used. Thirty-five GalNAc conjugated oligonucleotides selected from the in vitro screen were evaluated in mice engineered to transiently express human CD274 mRNA in hepatocytes of the mouse liver in two studies (FIGS. 16A and 16B). Briefly, 6-8-week-old female CD-1 mice (n=4) were subcutaneously administered the indicated GalNAc-conjugated CD274 oligonucleotides (17 in FIG. 16A and 18 in FIG. 16B; listed in Table 6) at a dose of 2 mg/kg formulated in PBS. A control group of mice (n=4) were administered only PBS. Three days later (72 hours), the mice were hydrodynamically injected (HDI) with a DNA plasmid encoding the full human CD274 gene (50 lag) under control of a ubiquitous cytomegalovirus (CMV) promoter sequence. About 20 hours after introduction of the DNA plasmid, liver samples from HDI mice were collected. Total RNA derived from these HDI mice were subjected to qRT-PCR analysis to determine CD274 mRNA levels as described in Example 11. mRNA levels were measured for human mRNA. The values were normalized for transfection efficiency using the NeoR gene included on the DNA plasmid.

TABLE 6
GalNAc-Conjugated Human CD274 RNAi
Oligonucleotides for HDI screen.

| | Unmodified | Unmodified | Modified | Modified |
|---|---|---|---|---|
| | Sense | Antisense | Sense | Antisense |
| | Strand | strand | Strand | strand |
| CD274-0088 | 483 | 724 | 965 | 1001 |
| CD274-0094 | 484 | 725 | 966 | 1002 |
| CD274-0097 | 486 | 727 | 968 | 1004 |
| CD274-0098 | 487 | 728 | 969 | 1005 |
| CD274-0100 | 488 | 729 | 970 | 1006 |
| CD274-0102 | 489 | 730 | 971 | 1007 |
| CD274-0167 | 490 | 731 | 972 | 1008 |
| CD274-0194 | 492 | 733 | 974 | 1010 |
| CD274-0197 | 493 | 734 | 975 | 1011 |
| CD274-0200 | 494 | 735 | 976 | 1012 |
| CD274-0204 | 495 | 736 | 977 | 1013 |
| CD274-0219 | 496 | 737 | 978 | 1014 |
| CD274-0222 | 497 | 738 | 979 | 1015 |
| CD274-0225 | 498 | 739 | 980 | 1016 |
| CD274-0268 | 499 | 740 | 981 | 1017 |
| CD274-0269 | 500 | 741 | 982 | 1018 |
| CD274-0271 | 501 | 742 | 983 | 1019 |
| CD274-0274 | 502 | 743 | 984 | 1020 |
| CD274-0275 | 503 | 744 | 985 | 1021 |
| CD274-0277 | 504 | 745 | 986 | 1022 |
| CD274-0282 | 505 | 746 | 987 | 1023 |
| CD274-0283 | 506 | 747 | 988 | 1024 |
| CD274-0394 | 507 | 748 | 989 | 1025 |
| CD274-0399 | 508 | 749 | 990 | 1026 |
| CD274-0530 | 509 | 750 | 991 | 1027 |
| CD274-0531 | 510 | 751 | 992 | 1028 |
| CD274-0653 | 511 | 752 | 993 | 1029 |
| CD274-0673 | 512 | 753 | 994 | 1030 |
| CD274-0693 | 513 | 754 | 995 | 1031 |
| CD274-0697 | 514 | 755 | 996 | 1032 |
| CD274-0700 | 515 | 756 | 997 | 1033 |
| CD274-0701 | 516 | 757 | 998 | 1034 |
| CD274-0743 | 517 | 758 | 999 | 1035 |
| CD274-0746 | 518 | 759 | 1000 | 1036 |
| CD274-0193 | 491 | 732 | 973 | 1009 |

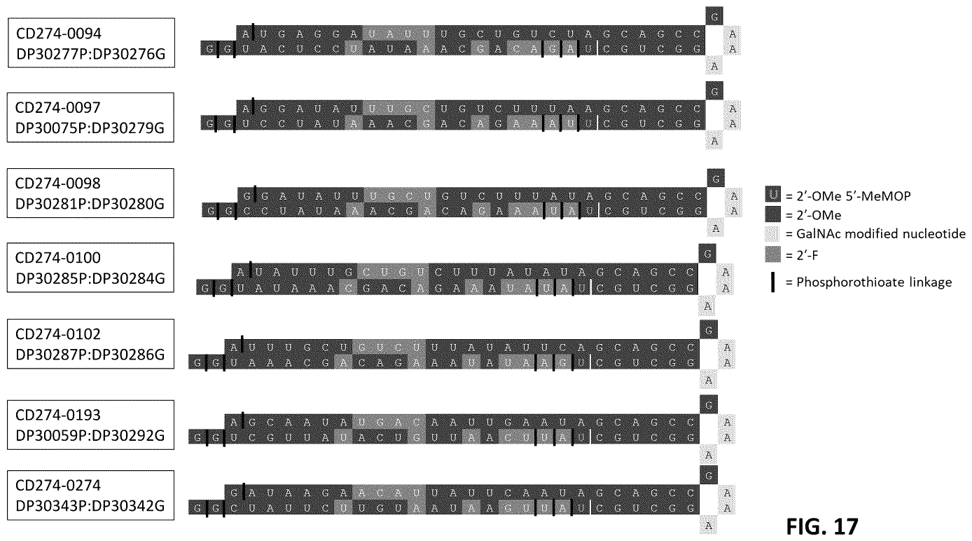

The results in FIGS. 16A and 16B demonstrate that GalNAc-conjugated CD274 oligonucleotides designed to target human CD274 mRNA inhibited human CD274 mRNA expression in HDI mice, as determined by a reduction in the amount of human CD274 mRNA expression in liver samples from HDI mice treated with GalNAc-conjugated CD274 oligonucleotides relative to control HDI mice treated with only PBS. Based on the mRNA knockdown, the best five from FIG. 16A and best two from FIG. 16B were selected for further dose response studies (Table 7 and depicted in FIG. 17).

TABLE 7
GalNAc-Conjugated Human CD274 RNAi
Oligonucleotides for Dose Study

| | Unmodified | Unmodified | Modified | Modified |
|---|---|---|---|---|
| | Sense | Antisense | Sense | Antisense |
| | Strand | strand | Strand | strand |
| CD274-0094 | 484 | 725 | 966 | 1002 |
| CD274-0097 | 486 | 727 | 968 | 1004 |
| CD274-0098 | 487 | 728 | 969 | 1005 |
| CD274-0100 | 488 | 729 | 970 | 1006 |
| CD274-0102 | 489 | 730 | 971 | 1007 |
| CD274-0193 | 491 | 732 | 973 | 1009 |
| CD274-0274 | 502 | 743 | 984 | 1020 |

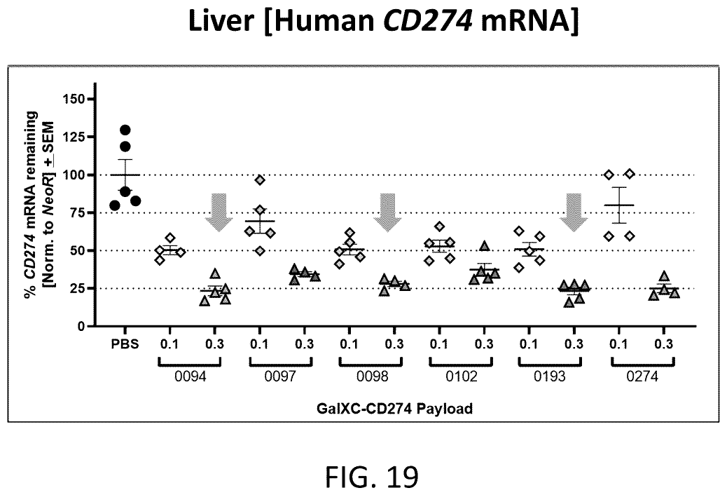

These subsets of GalNAc-conjugated CD274 oligonucleotides (CD274-094, 097, 098, 100, 102, 193 and 274) were further validated in two dose response studies (FIGS. 18 and 19). In the first study, seven GalNAc-conjugated CD274 oligonucleotides (CD274-094, 097, 098, 100, 102, 193 and 274) were tested. Mice were hydrodynamically injected as described above and treated with 0.3 mg/kg or 1 mg/kg of oligonucleotide. Livers were collected after 20 hours, and human CD274 expression was measured to determine a potent dose (FIG. 18). Then a second dose response study was run using six oligonucleotides (CD274-094, 097, 098, 102, 193 and 274) at 0.3 and 0.1 mg/kg dose levels (FIG. 19). A dose of 0.1 mg/kg was capable of reducing CD274 mRNA by about 50%, thereby identifying 3 potential GalNAc-conjugated CD274 oligonucleotides for inhibiting CD274 expression in liver. The best two sequences from FIG. 19 were further evaluated in in vitro cell line/immune cell culture settings to evaluate efficacy.

### Example 11: Evaluating the Intrinsic Potency of Two Selected CD274 Oligonucleotides

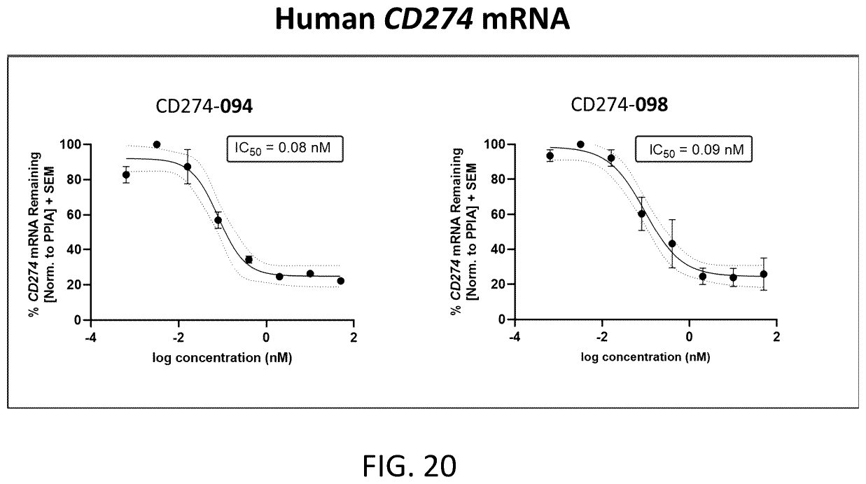

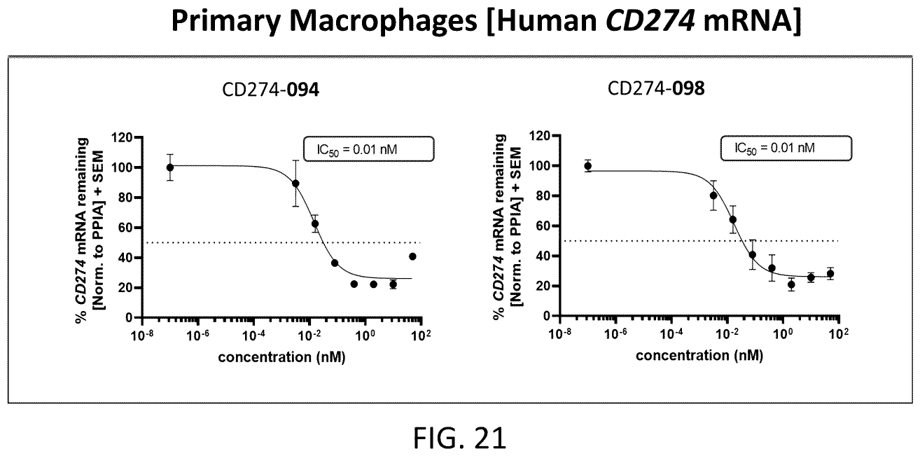

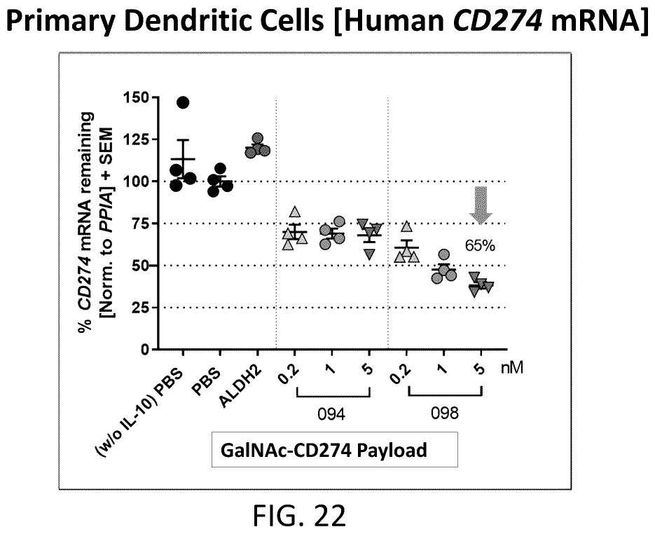

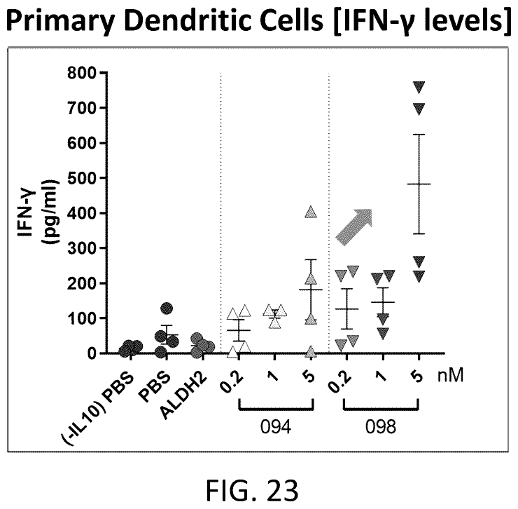

To evaluate the intrinsic potency of GalNAc-CD274-094 and GalNAc-CD274-098, the oligonucleotides were tested in the non-small cell lung cancer H460 cell line. H460 cells were treated for 24 hours with a series of concentrations of the GalNAc-conjugated CD274 oligonucleotides using lipofectamine as transfection agent. The percent (%) remaining mRNA was normalized using an endogenously expressed control gene PP1ACD274 oligonucleotides demonstrated similar IC₅₀ suggesting that the potencies are very similar (FIG. 20). These two oligonucleotides were then tested in human primary immune cell culture as these cells are the target cells in human patients Monocytes were first treated with M-CSF for 6 days for differentiation followed by polarization with IL-10 cytokine for 1 day to convert them into suppressive macrophages (M2 phenotype) and treated with both GalNAc-conjugated CD274 oligonucleotides for 72 hours with a series of dose levels (0.0032 nM, 0.16 nM, 0.08 nM, 0.04 nM, 2 nM, 10 nM, and 50 nM). The IC₅₀s were almost identical although they are slightly better than what were found in H460 cell line (FIG. 21). The monocytes were also converted into DCs after 7-day incubation in media with GM-CSF and IL-4 cytokines. Since CD274 expressed by the DCs identified as the key component that play a role in mediating immune response, the selected sequences were tested in these differentiated dendritic cells (FIG. 22). GalNAc-CD274-098 demonstrated dose-dependent knockdown while CD274-094 did not (FIG. 22) in these cells. To evaluate if the CD274 knockdown mediates any immune activation which is usually the case when DCs get activated in TDLN, cytokine levels were measured in the supernatant collected from the in vitro culture plates using MSD V-plex assay kit. GalNAc-CD274-098 mediated slightly higher levels of IFN-7 compared to GalNac-CD274-094 upon CD274 inhibition (FIG. 23), correlating to RNAi potency rank order observed in FIG. 22. Demonstration of IFN-7 release in human dendritic cells after treatment confirms targeting of the endogenous gene in the target cell type. Together this data demonstrates that the intrinsic potency of the CD274 oligonucleotides could be confirmed by measuring the efficiency of the sequence to reduce CD274 expression and the activating immune responses upon CD274 inhibition in the target cell type.

### Example 12: Using SAR to Identify a GalXC Lipid Conjugate Favorable for Delivery of siRNA to the Tumor Microenvironment and Tumor Draining Lymph Nodes

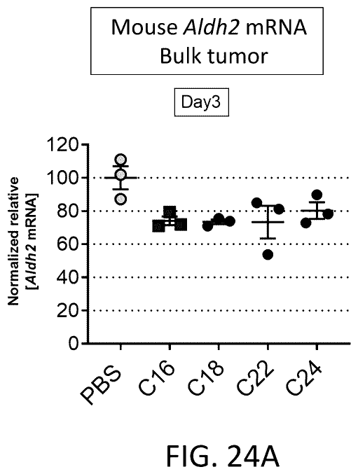

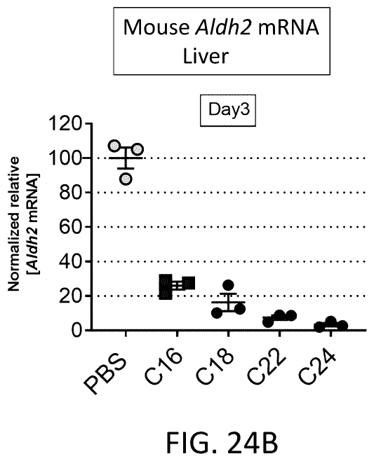

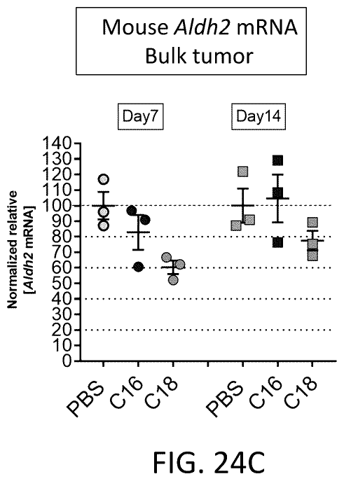

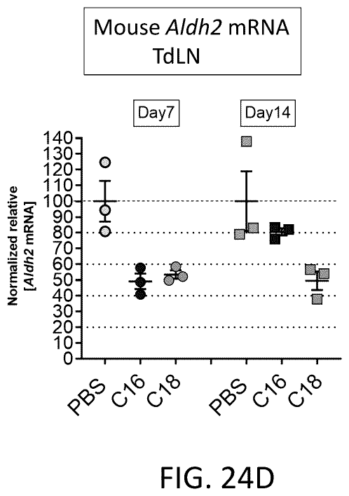

To identify a lipid conjugate with the most favorable properties to deliver payload and mediate target knockdown with the highest selectivity to myeloid cells in TME, a series of lipid conjugated oligonucleotides (C16, C18, C22 and C24) were generated (Table 8). To investigate these test articles, Pan02 murine pancreatic tumor cells were implanted in nude mice. When the tumors reached a volume of 300-400 mm³, the mice were randomized into groups and treated with either a single dose of PBS or a GalXC lipid conjugate (C16, C18, C22 and C24) at 25 mg/kg. Target knockdown was assessed on day 3 in bulk tumor and in liver (FIGS. 24A and 24B) to identify a GalXC lipid a conjugate with selectivity towards the target tissue (MDSCs) as compared to normal liver tissue. On day 3 post dose, Aldh2 mRNA levels in the tumors of all the treatment groups were decreased to a similar degree. There was a trend observed in the Aldh2 levels in livers of GalXC-ALDH2-lipid conjugate groups of a correlation of higher lipid acyl chain length with lower target knockdown (C24>C22>C18>C16), suggesting that these conjugates may use different mechanisms to traffic to TME versus Liver. Since the shorter lipid acyl chain conjugates C16 and C18 seem to be more liver sparing without compromising TME activity, as compared to longer acyl chain conjugates C22 and C24, the C16/C18 lipid conjugates were further explored in a separate study to further characterize their activity. Pan02 tumor bearing mice were treated with a single subcutaneous dose of GalXC-ALDH2-C16 or GalXC-ALDH-C18 at 25 mg/kg, or PBS and activity was monitored in bulk tumor tissue and TdLN on days 7 and 14. As shown in FIGS. 24C and 24D, the C18 conjugate outperformed C16 in target knockdown in bulk tumor at both time points. Although both test articles showed similar activity in TdLN on day 7, the C16 conjugate mediated activity was significantly reduced on day14 while C18 mediated activity was maintained.

TABLE 8
GalXC-lipid conjugate ALDH2 Tool Molecules

| | | Sense | Antisense | |
|---|---|---|---|---|
| | | Strand | strand | |
| | Sequence | SEQ | SEQ | |
| Oligo | Type | ID NO | ID NO | Conjugate |
| GalXC-ALDH2-C16 | Unmodified | 1043 | 1044 | C16 |
| | Modified | 1045 | 1046 | C16 |
| GalXC-ALDH2-C18 | Unmodified | 1043 | 1044 | C18 |
| | Modified | 1047 | 1046 | C18 |
| GalXC-ALDH2-C22 | Unmodified | 1043 | 1044 | C22 |
| | Modified | 1048 | 1046 | C22 |
| GalXC-ALDH2-C24 | Unmodified | 1043 | 1044 | C24 |
| | Modified | 1049 | 1046 | C24 |

### Example 13: CD274 Oligonucleotides for Treatment of Disease

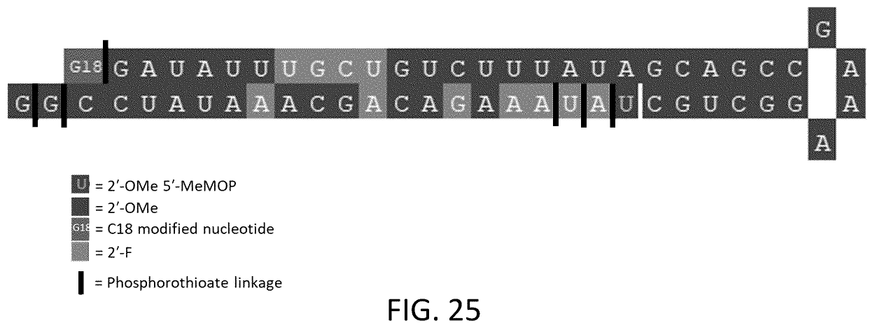

To investigate efficacy of CD274 RNAi oligonucleotides with ligand conjugation, subjects are administered a CD274 RNAi oligonucleotide conjugated to a hydrocarbon chain (e.g., a hydrocarbon chain depicted in FIG. 1B). Specifically, subjects are administered a CD274 RNAi oligonucleotide (depicted in FIG. 25) wherein the sense strand comprises SEQ ID NO: 1050 and the antisense strand comprises SEQ ID NO: 1005.

SEQUENCE LISTING

| | | | SEQ ID |
|---|---|---|---|
| Construct | Description | Sequence | NO |
| CD274- | 19-mer Sense | AGAAAGAUGAGGAUAUUUG | 1 |
| 0088 | Strand | | |
| CD274- | 19-mer Sense | AUGAGGAUAUUUGCUGUCU | 2 |
| 0094 | Strand | | |
| CD274- | 19-mer Sense | AGGAUAUUUGCUGUCUUUA | 4 |
| 0097 | Strand | | |
| CD274- | 19-mer Sense | GGAUAUUUGCUGUCUUUAU | 5 |
| 0098 | Strand | | |
| CD274- | 19-mer Sense | AUAUUUGCUGUCUUUAUAU | 6 |
| 0100 | Strand | | |
| CD274- | 19-mer Sense | AUUUGCUGUCUUUAUAUUC | 7 |
| 0102 | Strand | | |
| CD274- | 19-mer Sense | AGGACCUAUAUGUGGUAGA | 8 |
| 0167 | Strand | | |
| CD274- | 19-mer Sense | AGCAAUAUGACAAUUGAAU | 9 |
| 0193 | Strand | | |
| CD274- | 19-mer Sense | GCAAUAUGACAAUUGAAUG | 10 |
| 0194 | Strand | | |
| CD274- | 19-mer Sense | AUAUGACAAUUGAAUGCAA | 11 |
| 0197 | Strand | | |
| CD274- | 19-mer Sense | UGACAAUUGAAUGCAAAUU | 12 |
| 0200 | Strand | | |
| CD274- | 19-mer Sense | AAUUGAAUGCAAAUUCCCA | 13 |
| 0204 | Strand | | |
| CD274- | 19-mer Sense | CCCAGUAGAAAAACAAUUA | 14 |
| 0219 | Strand | | |
| CD274- | 19-mer Sense | AGUAGAAAAACAAUUAGAC | 15 |
| 0222 | Strand | | |
| CD274- | 19-mer Sense | AGAAAAACAAUUAGACCUG | 16 |
| 0225 | Strand | | |
| CD274- | 19-mer Sense | AUGGAGGAUAAGAACAUUA | 17 |
| 0268 | Strand | | |
| CD274- | 19-mer Sense | UGGAGGAUAAGAACAUUAU | 18 |
| 0269 | Strand | | |
| CD274- | 19-mer Sense | GAGGAUAAGAACAUUAUUC | 19 |
| 0271 | Strand | | |
| CD274- | 19-mer Sense | GAUAAGAACAUUAUUCAAU | 20 |
| 0274 | Strand | | |
| CD274- | 19-mer Sense | AUAAGAACAUUAUUCAAUU | 21 |
| 0275 | Strand | | |
| CD274- | 19-mer Sense | AAGAACAUUAUUCAAUUUG | 22 |
| 0277 | Strand | | |
| CD274- | 19-mer Sense | CAUUAUUCAAUUUGUGCAU | 23 |
| 0282 | Strand | | |
| CD274- | 19-mer Sense | AUUAUUCAAUUUGUGCAUG | 24 |
| 0283 | Strand | | |
| CD274- | 19-mer Sense | AUCACAGAUGUGAAAUUGC | 25 |
| 0394 | Strand | | |
| CD274- | 19-mer Sense | AGAUGUGAAAUUGCAGGAU | 26 |
| 0399 | Strand | | |
| CD274- | 19-mer Sense | CAGUCACCUCUGAACAUGA | 27 |
| 0530 | Strand | | |
| CD274- | 19-mer Sense | AGUCACCUCUGAACAUGAA | 28 |
| 0531 | Strand | | |
| CD274- | 19-mer Sense | AGGAGAAGCUUUUCAAUGU | 29 |
| 0653 | Strand | | |
| CD274- | 19-mer Sense | ACCAGCACACUGAGAAUCA | 30 |
| 0673 | Strand | | |
| CD274- | 19-mer Sense | CACAACAACUAAUGAGAUU | 31 |
| 0693 | Strand | | |
| CD274- | 19-mer Sense | ACAACUAAUGAGAUUUUCU | 32 |
| 0697 | Strand | | |
| CD274- | 19-mer Sense | ACUAAUGAGAUUUUCUACU | 33 |
| 0700 | Strand | | |
| CD274- | 19-mer Sense | CUAAUGAGAUUUUCUACUG | 34 |
| 0701 | Strand | | |
| CD274- | 19-mer Sense | AGGAAAACCAUACAGCUGA | 35 |
| 0743 | Strand | | |
| CD274- | 19-mer Sense | AAAACCAUACAGCUGAAUU | 36 |
| 0746 | Strand | | |
| CD274- | 19-mer Sense | UGAGGAUAUUUGCUGUCUU | 37 |
| 0095 | Strand | | |
| CD274- | 19-mer Sense | GAUAUUUGCUGUCUUUAUA | 38 |
| 0099 | Strand | | |
| CD274- | 19-mer Sense | UUUGCUGUCUUUAUAUUCA | 39 |
| 0103 | Strand | | |
| CD274- | 19-mer Sense | CAAUAUGACAAUUGAAUGC | 40 |
| 0195 | Strand | | |
| CD274- | 19-mer Sense | AAUAUGACAAUUGAAUGCA | 41 |
| 0196 | Strand | | |
| CD274- | 19-mer Sense | UAUGACAAUUGAAUGCAAA | 42 |
| 0198 | Strand | | |
| CD274- | 19-mer Sense | AUGACAAUUGAAUGCAAAU | 43 |
| 0199 | Strand | | |
| CD274- | 19-mer Sense | GACAAUUGAAUGCAAAUUC | 44 |
| 0201 | Strand | | |
| CD274- | 19-mer Sense | ACAAUUGAAUGCAAAUUCC | 45 |
| 0202 | Strand | | |
| CD274- | 19-mer Sense | UGAAUGCAAAUUCCCAGUA | 46 |
| 0207 | Strand | | |
| CD274- | 19-mer Sense | CAGUAGAAAAACAAUUAGA | 47 |
| 0221 | Strand | | |
| CD274- | 19-mer Sense | CUGCACUAAUUGUCUAUUG | 48 |
| 0245 | Strand | | |
| CD274- | 19-mer Sense | CACUAAUUGUCUAUUGGGA | 49 |
| 0248 | Strand | | |
| CD274- | 19-mer Sense | ACUAAUUGUCUAUUGGGAA | 50 |
| 0249 | Strand | | |
| CD274- | 19-mer Sense | GAAAUGGAGGAUAAGAACA | 51 |
| 0265 | Strand | | |
| CD274- | 19-mer Sense | AAAUGGAGGAUAAGAACAU | 52 |
| 0266 | Strand | | |
| CD274- | 19-mer Sense | AAUGGAGGAUAAGAACAUU | 53 |
| 0267 | Strand | | |
| CD274- | 19-mer Sense | GGAGGAUAAGAACAUUAUU | 54 |
| 0270 | Strand | | |
| CD274- | 19-mer Sense | AGGAUAAGAACAUUAUUCA | 55 |
| 0272 | Strand | | |
| CD274- | 19-mer Sense | GGAUAAGAACAUUAUUCAA | 56 |
| 0273 | Strand | | |
| CD274- | 19-mer Sense | UAAGAACAUUAUUCAAUUU | 57 |
| 0276 | Strand | | |
| CD274- | 19-mer Sense | AGAACAUUAUUCAAUUUGU | 58 |
| 0278 | Strand | | |
| CD274- | 19-mer Sense | GAACAUUAUUCAAUUUGUG | 59 |
| 0279 | Strand | | |
| CD274- | 19-mer Sense | AACAUUAUUCAAUUUGUGC | 60 |
| 0280 | Strand | | |
| CD274- | 19-mer Sense | ACAUUAUUCAAUUUGUGCA | 61 |
| 0281 | Strand | | |
| CD274- | 19-mer Sense | UAUUCAAUUUGUGCAUGGA | 62 |
| 0285 | Strand | | |
| CD274- | 19-mer Sense | AGAGGAAGACCUGAAGGUU | 63 |
| 0303 | Strand | | |
| CD274- | 19-mer Sense | AGGAAGACCUGAAGGUUCA | 64 |
| 0305 | Strand | | |
| CD274- | 19-mer Sense | AAGACCUGAAGGUUCAGCA | 65 |
| 0308 | Strand | | |
| CD274- | 19-mer Sense | AGACCUGAAGGUUCAGCAU | 66 |
| 0309 | Strand | | |
| CD274- | 19-mer Sense | GACCUGAAGGUUCAGCAUA | 67 |
| 0310 | Strand | | |
| CD274- | 19-mer Sense | CCUGAAGGUUCAGCAUAGU | 68 |
| 0312 | Strand | | |
| CD274- | 19-mer Sense | CUGAAGGUUCAGCAUAGUA | 69 |
| 0313 | Strand | | |
| CD274- | 19-mer Sense | AGAUCACAGAUGUGAAAUU | 70 |
| 0392 | Strand | | |
| CD274- | 19-mer Sense | UCACAGAUGUGAAAUUGCA | 71 |
| 0395 | Strand | | |
| CD274- | 19-mer Sense | CAGAUGUGAAAUUGCAGGA | 72 |
| 0398 | Strand | | |
| CD274- | 19-mer Sense | ACAAAAUCAACCAAAGAAU | 73 |
| 0497 | Strand | | |
| CD274- | 19-mer Sense | AAAAUCAACCAAAGAAUUU | 74 |
| 0499 | Strand | | |
| CD274- | 19-mer Sense | AAAUCAACCAAAGAAUUUU | 75 |
| 0500 | Strand | | |
| CD274- | 19-mer Sense | AAUCAACCAAAGAAUUUUG | 76 |
| 0501 | Strand | | |
| CD274- | 19-mer Sense | AUCAACCAAAGAAUUUUGG | 77 |
| 0502 | Strand | | |
| CD274- | 19-mer Sense | CAACCAAAGAAUUUUGGUU | 78 |
| 0504 | Strand | | |
| CD274- | 19-mer Sense | AACCAAAGAAUUUUGGUUG | 79 |
| 0505 | Strand | | |
| CD274- | 19-mer Sense | ACCAAAGAAUUUUGGUUGU | 80 |
| 0506 | Strand | | |
| CD274- | 19-mer Sense | CCAAAGAAUUUUGGUUGUG | 81 |
| 0507 | Strand | | |
| CD274- | 19-mer Sense | AAAGAAUUUUGGUUGUGGA | 82 |
| 0509 | Strand | | |
| CD274- | 19-mer Sense | AAGAAUUUUGGUUGUGGAU | 83 |
| 0510 | Strand | | |
| CD274- | 19-mer Sense | AGAAUUUUGGUUGUGGAUC | 84 |
| 0511 | Strand | | |
| CD274- | 19-mer Sense | AAUUUUGGUUGUGGAUCCA | 85 |
| 0513 | Strand | | |
| CD274- | 19-mer Sense | ACCUCUGAACAUGAACUGA | 86 |
| 0535 | Strand | | |
| CD274- | 19-mer Sense | CUCUGAACAUGAACUGACA | 87 |
| 0537 | Strand | | |
| CD274- | 19-mer Sense | AACAUGAACUGACAUGUCA | 88 |
| 0542 | Strand | | |
| CD274- | 19-mer Sense | GAAGUCAUCUGGACAAGCA | 89 |
| 0583 | Strand | | |
| CD274- | 19-mer Sense | GACAAGCAGUGACCAUCAA | 90 |
| 0594 | Strand | | |
| CD274- | 19-mer Sense | AGUGACCAUCAAGUCCUGA | 91 |
| 0601 | Strand | | |
| CD274- | 19-mer Sense | GGAGAAGCUUUUCAAUGUG | 92 |
| 0654 | Strand | | |
| CD274- | 19-mer Sense | GAGAAGCUUUUCAAUGUGA | 93 |
| 0655 | Strand | | |
| CD274- | 19-mer Sense | AGAAGCUUUUCAAUGUGAC | 94 |
| 0656 | Strand | | |
| CD274- | 19-mer Sense | AAGCUUUUCAAUGUGACCA | 95 |
| 0658 | Strand | | |
| CD274- | 19-mer Sense | CUUUUCAAUGUGACCAGCA | 96 |
| 0661 | Strand | | |
| CD274- | 19-mer Sense | AAUGUGACCAGCACACUGA | 97 |
| 0667 | Strand | | |
| CD274- | 19-mer Sense | AGCACACUGAGAAUCAACA | 98 |
| 0676 | Strand | | |
| CD274- | 19-mer Sense | ACACUGAGAAUCAACACAA | 99 |
| 0679 | Strand | | |
| CD274- | 19-mer Sense | ACUGAGAAUCAACACAACA | 100 |
| 0681 | Strand | | |
| CD274- | 19-mer Sense | AGAAUCAACACAACAACUA | 101 |
| 0685 | Strand | | |
| CD274- | 19-mer Sense | ACACAACAACUAAUGAGAU | 102 |
| 0692 | Strand | | |
| CD274- | 19-mer Sense | CAACAACUAAUGAGAUUUU | 103 |
| 0695 | Strand | | |
| CD274- | 19-mer Sense | AACAACUAAUGAGAUUUUC | 104 |
| 0696 | Strand | | |
| CD274- | 19-mer Sense | CAACUAAUGAGAUUUUCUA | 105 |
| 0698 | Strand | | |
| CD274- | 19-mer Sense | AAUGAGAUUUUCUACUGCA | 106 |
| 0703 | Strand | | |
| CD274- | 19-mer Sense | AUUUUCUACUGCACUUUUA | 107 |
| 0709 | Strand | | |
| CD274- | 19-mer Sense | GAAAACCAUACAGCUGAAU | 108 |
| 0745 | Strand | | |
| CD274- | 19-mer Sense | ACCAUACAGCUGAAUUGGU | 109 |
| 0749 | Strand | | |
| CD274- | 19-mer Sense | AUACAGCUGAAUUGGUCAU | 110 |
| 0752 | Strand | | |
| CD274- | 19-mer Sense | AUGAAAGGACUCACUUGGU | 111 |
| 0800 | Strand | | |
| CD274- | 19-mer Sense | AAGGACUCACUUGGUAAUU | 112 |
| 0804 | Strand | | |
| CD274- | 19-mer Sense | GGUGUAGCACUGACAUUCA | 113 |
| 0847 | Strand | | |
| CD274- | 19-mer Sense | AGCACUGACAUUCAUCUUC | 114 |
| 0852 | Strand | | |
| CD274- | 19-mer Sense | AUGAUGGAUGUGAAAAAAU | 115 |
| 0889 | Strand | | |
| CD274- | 19-mer Sense | GGAGACCUUGAUACUUUCA | 116 |
| 1172 | Strand | | |
| CD274- | 19-mer Sense | GAGACCUUGAUACUUUCAA | 117 |
| 1173 | Strand | | |
| CD274- | 19-mer Sense | AGACCUUGAUACUUUCAAA | 118 |
| 1174 | Strand | | |
| CD274- | 19-mer Sense | ACCUUGAUACUUUCAAAUG | 119 |
| 1176 | Strand | | |
| CD274- | 19-mer Sense | CUUGAUACUUUCAAAUGCC | 120 |
| 1178 | Strand | | |
| CD274- | 19-mer Sense | GAAAGGAUACUUCUGAACA | 121 |
| 1227 | Strand | | |
| CD274- | 19-mer Sense | GGAUACUUCUGAACAAGGA | 122 |
| 1231 | Strand | | |
| CD274- | 19-mer Sense | CUAUUUAUUUUGAGUCUGU | 123 |
| 1423 | Strand | | |
| CD274- | 19-mer Sense | AUUUAUUUUGAGUCUGUGA | 124 |
| 1425 | Strand | | |
| CD274- | 19-mer Sense | UGAGUGUGGUUGUGAAUGA | 125 |
| 1459 | Strand | | |
| CD274- | 19-mer Sense | GAGUGUGGUUGUGAAUGAU | 126 |
| 1460 | Strand | | |
| CD274- | 19-mer Sense | AGUGUGGUUGUGAAUGAUU | 127 |
| 1461 | Strand | | |
| CD274- | 19-mer Sense | GUGUGGUUGUGAAUGAUUU | 128 |
| 1462 | Strand | | |
| CD274- | 19-mer Sense | UGUGGUUGUGAAUGAUUUC | 129 |
| 1463 | Strand | | |
| CD274- | 19-mer Sense | GUGGUUGUGAAUGAUUUCU | 130 |
| 1464 | Strand | | |
| CD274- | 19-mer Sense | UGGUUGUGAAUGAUUUCUU | 131 |
| 1465 | Strand | | |
| CD274- | 19-mer Sense | GUUGUGAAUGAUUUCUUUU | 132 |
| 1467 | Strand | | |
| CD274- | 19-mer Sense | UGUGAAUGAUUUCUUUUGA | 133 |
| 1469 | Strand | | |
| CD274- | 19-mer Sense | GUGAAUGAUUUCUUUUGAA | 134 |
| 1470 | Strand | | |
| CD274- | 19-mer Sense | AAUGAUUUCUUUUGAAGAU | 135 |
| 1473 | Strand | | |
| CD274- | 19-mer Sense | AUGAUUUCUUUUGAAGAUA | 136 |
| 1474 | Strand | | |
| CD274- | 19-mer Sense | CUUUUGAAGAUAUAUUGUA | 137 |
| 1481 | Strand | | |
| CD274- | 19-mer Sense | UUGAAGAUAUAUUGUAGUA | 138 |
| 1484 | Strand | | |
| CD274- | 19-mer Sense | GAAGAUAUAUUGUAGUAGA | 139 |
| 1486 | Strand | | |
| CD274- | 19-mer Sense | AGUAGAUGUUACAAUUUUG | 140 |
| 1499 | Strand | | |
| CD274- | 19-mer Sense | GUAGAUGUUACAAUUUUGU | 141 |
| 1500 | Strand | | |
| CD274- | 19-mer Sense | AAUGAUUUGCUCACAUCUA | 142 |
| 1541 | Strand | | |
| CD274- | 19-mer Sense | AAAACAUGGAGUAUUUGUA | 143 |
| 1562 | Strand | | |
| CD274- | 19-mer Sense | CAAGUAUACAUUGGAAGCA | 144 |
| 1606 | Strand | | |
| CD274- | 19-mer Sense | AAGUAUACAUUGGAAGCAU | 145 |
| 1607 | Strand | | |
| CD274- | 19-mer Sense | AGGAUGUCACCUUUAUUUA | 146 |
| 1649 | Strand | | |
| CD274- | 19-mer Sense | CUGUUCCAUUUAAAUAUCA | 147 |
| 1728 | Strand | | |
| CD274- | 19-mer Sense | UGUAACCACCCUGUUGUGA | 148 |
| 1794 | Strand | | |
| CD274- | 19-mer Sense | GUAACCACCCUGUUGUGAU | 149 |
| 1795 | Strand | | |
| CD274- | 19-mer Sense | GUUGUGAUAACCACUAUUA | 150 |
| 1806 | Strand | | |
| CD274- | 19-mer Sense | UUGUGAUAACCACUAUUAU | 151 |
| 1807 | Strand | | |
| CD274- | 19-mer Sense | UGUGAUAACCACUAUUAUU | 152 |
| 1808 | Strand | | |
| CD274- | 19-mer Sense | GUGAUAACCACUAUUAUUU | 153 |
| 1809 | Strand | | |
| CD274- | 19-mer Sense | UGAUAACCACUAUUAUUUU | 154 |
| 1810 | Strand | | |
| CD274- | 19-mer Sense | GAUAACCACUAUUAUUUUA | 155 |
| 1811 | Strand | | |
| CD274- | 19-mer Sense | AUAACCACUAUUAUUUUAC | 156 |
| 1812 | Strand | | |
| CD274- | 19-mer Sense | CCACUGUCCUUUUAUAAUA | 157 |
| 1906 | Strand | | |
| CD274- | 19-mer Sense | CACUGUCCUUUUAUAAUAC | 158 |
| 1907 | Strand | | |
| CD274- | 19-mer Sense | ACUGUCCUUUUAUAAUACA | 159 |
| 1908 | Strand | | |
| CD274- | 19-mer Sense | CUGUCCUUUUAUAAUACAA | 160 |
| 1909 | Strand | | |
| CD274- | 19-mer Sense | UGUCCUUUUAUAAUACAAU | 161 |
| 1910 | Strand | | |
| CD274- | 19-mer Sense | CCUUUUAUAAUACAAUUUA | 162 |
| 1913 | Strand | | |
| CD274- | 19-mer Sense | UUUUAUAAUACAAUUUACA | 163 |
| 1915 | Strand | | |
| CD274- | 19-mer Sense | AUAAUACAAUUUACAGCUA | 164 |
| 1919 | Strand | | |
| CD274- | 19-mer Sense | ACAGCUAUAUUUUACUUUA | 165 |
| 1931 | Strand | | |
| CD274- | 19-mer Sense | CUAUAUUUUACUUUAAGCA | 166 |
| 1935 | Strand | | |
| CD274- | 19-mer Sense | AUAUUUUACUUUAAGCAAU | 167 |
| 1937 | Strand | | |
| CD274- | 19-mer Sense | AUUGAAUCUACAGAUGUGA | 168 |
| 2014 | Strand | | |
| CD274- | 19-mer Sense | GAAAAUGUAUUAUUACAAU | 169 |
| 2088 | Strand | | |
| CD274- | 19-mer Sense | AUAAUCAGAGUAAUUUUCA | 170 |
| 2221 | Strand | | |
| CD274- | 19-mer Sense | UCAGAGUAAUUUUCAUUUA | 171 |
| 2225 | Strand | | |
| CD274- | 19-mer Sense | AGAGUAAUUUUCAUUUACA | 172 |
| 2227 | Strand | | |
| CD274- | 19-mer Sense | GAGUAAUUUUCAUUUACAA | 173 |
| 2228 | Strand | | |
| CD274- | 19-mer Sense | AGUAAUUUUCAUUUACAAA | 174 |
| 2229 | Strand | | |
| CD274- | 19-mer Sense | UAAUUUUCAUUUACAAAGA | 175 |
| 2231 | Strand | | |
| CD274- | 19-mer Sense | AUUUUCAUUUACAAAGAGA | 176 |
| 2233 | Strand | | |
| CD274- | 19-mer Sense | AAAAUAACCCUGAAAAAUA | 177 |
| 2263 | Strand | | |
| CD274- | 19-mer Sense | AUAACCCUGAAAAAUAACA | 178 |
| 2266 | Strand | | |
| CD274- | 19-mer Sense | AUAUAAUCUAAUGCUUGUU | 179 |
| 2333 | Strand | | |
| CD274- | 19-mer Sense | AUAAUCUAAUGCUUGUUUA | 180 |
| 2335 | Strand | | |
| CD274- | 19-mer Sense | AAUCUAAUGCUUGUUUAUA | 181 |
| 2337 | Strand | | |
| CD274- | 19-mer Sense | AUCUAAUGCUUGUUUAUAU | 182 |
| 2338 | Strand | | |
| CD274- | 19-mer Sense | UCUAAUGCUUGUUUAUAUA | 183 |
| 2339 | Strand | | |
| CD274- | 19-mer Sense | UAAUGCUUGUUUAUAUAGU | 184 |
| 2341 | Strand | | |
| CD274- | 19-mer Sense | AUGCUUGUUUAUAUAGUGU | 185 |
| 2343 | Strand | | |
| CD274- | 19-mer Sense | CUGGUAUUGUUUAACAGUU | 186 |
| 2362 | Strand | | |
| CD274- | 19-mer Sense | UGGUAUUGUUUAACAGUUC | 187 |
| 2363 | Strand | | |
| CD274- | 19-mer Sense | AAUUUUAAAUUCAUACCUU | 188 |
| 2407 | Strand | | |
| CD274- | 19-mer Sense | AGUCUACAUUUGGAAAUGU | 189 |
| 2556 | Strand | | |
| CD274- | 19-mer Sense | GUCUACAUUUGGAAAUGUA | 190 |
| 2557 | Strand | | |
| CD274- | 19-mer Sense | ACAUUUGGAAAUGUAUGUU | 191 |
| 2561 | Strand | | |
| CD274- | 19-mer Sense | CAUUUGGAAAUGUAUGUUA | 192 |
| 2562 | Strand | | |
| CD274- | 19-mer Sense | AUUUGGAAAUGUAUGUUAA | 193 |
| 2563 | Strand | | |
| CD274- | 19-mer Sense | UUGGAAAUGUAUGUUAAAA | 194 |
| 2565 | Strand | | |
| CD274- | 19-mer Sense | GAAAUGUAUGUUAAAAGCA | 195 |
| 2568 | Strand | | |
| CD274- | 19-mer Sense | CUGUGUACUUUGCUAUUUU | 196 |
| 2634 | Strand | | |
| CD274- | 19-mer Sense | GUGUACUUUGCUAUUUUUA | 197 |
| 2636 | Strand | | |
| CD274- | 19-mer Sense | UGUACUUUGCUAUUUUUAU | 198 |
| 2637 | Strand | | |
| CD274- | 19-mer Sense | ACUUUGCUAUUUUUAUUUA | 199 |
| 2640 | Strand | | |
| CD274- | 19-mer Sense | AUAUAGCAGAUGGAAUGAA | 200 |
| 2673 | Strand | | |
| CD274- | 19-mer Sense | GCAGAUGGAAUGAAUUUGA | 201 |
| 2678 | Strand | | |
| CD274- | 19-mer Sense | CAGAUGGAAUGAAUUUGAA | 202 |
| 2679 | Strand | | |
| CD274- | 19-mer Sense | AUAUACAUUUAGACAACCA | 203 |
| 2801 | Strand | | |
| CD274- | 19-mer Sense | AACCACCAUUUGUUAAGUA | 204 |
| 2815 | Strand | | |
| CD274- | 19-mer Sense | ACCAUUUGUUAAGUAUUUG | 205 |
| 2819 | Strand | | |
| CD274- | 19-mer Sense | AAAAGCAAUCUUAUUAUUA | 206 |
| 2924 | Strand | | |
| CD274- | 19-mer Sense | UAUUAUUAACUCUGUAUGA | 207 |
| 2935 | Strand | | |
| CD274- | 19-mer Sense | AUUAUUAACUCUGUAUGAC | 208 |
| 2936 | Strand | | |
| CD274- | 19-mer Sense | UUAUUAACUCUGUAUGACA | 209 |
| 2937 | Strand | | |
| CD274- | 19-mer Sense | AUUAACUCUGUAUGACAGA | 210 |
| 2939 | Strand | | |
| CD274- | 19-mer Sense | AGUAUAAACUUCACUUUGA | 211 |
| 2994 | Strand | | |
| CD274- | 19-mer Sense | CUGUACUUGCAAAAUCACA | 212 |
| 3015 | Strand | | |
| CD274- | 19-mer Sense | ACUUGCAAAAUCACAUUUU | 213 |
| 3019 | Strand | | |
| CD274- | 19-mer Sense | CCUCAUUCGUUGUGCUUGA | 214 |
| 3094 | Strand | | |
| CD274- | 19-mer Sense | CUCAUUCGUUGUGCUUGAA | 215 |
| 3095 | Strand | | |
| CD274- | 19-mer Sense | CUUGUGUUAUCUGUUUGUA | 216 |
| 3305 | Strand | | |
| CD274- | 19-mer Sense | UUGUGUUAUCUGUUUGUAC | 217 |
| 3306 | Strand | | |
| CD274- | 19-mer Sense | UGUGUUAUCUGUUUGUACA | 218 |
| 3307 | Strand | | |
| CD274- | 19-mer Sense | GUGUUAUCUGUUUGUACAU | 219 |
| 3308 | Strand | | |
| CD274- | 19-mer Sense | GUUAUCUGUUUGUACAUGU | 220 |
| 3310 | Strand | | |
| CD274- | 19-mer Sense | UUGUACAUGUGCAUUUGUA | 221 |
| 3319 | Strand | | |
| CD274- | 19-mer Sense | AUGUGCAUUUGUACAGUAA | 222 |
| 3325 | Strand | | |
| CD274- | 19-mer Sense | GUGUUCUUUGUGUGAAUUA | 223 |
| 3355 | Strand | | |
| CD274- | 19-mer Sense | GUUCUUUGUGUGAAUUACA | 224 |
| 3357 | Strand | | |
| CD274- | 19-mer Sense | UUGUGGUGUUGGAUUUGUA | 225 |
| 3441 | Strand | | |
| CD274- | 19-mer Sense | AUUCUGCAUUUGAUUGUCA | 226 |
| 3522 | Strand | | |
| CD274- | 19-mer Sense | CAUUUGAUUGUCACUUUUU | 227 |
| 3528 | Strand | | |
| CD274- | 19-mer Sense | UUGAUUGUCACUUUUUGUA | 228 |
| 3531 | Strand | | |
| CD274- | 19-mer Sense | UGAUUGUCACUUUUUGUAC | 229 |
| 3532 | Strand | | |
| CD274- | 19-mer Sense | AUUGUCACUUUUUGUACCU | 230 |
| 3534 | Strand | | |
| CD274- | 19-mer Sense | CUUUUUGUACCUGCAUUAA | 231 |
| 3541 | Strand | | |
| CD274- | 19-mer Sense | UUUUUGUACCUGCAUUAAU | 232 |
| 3542 | Strand | | |
| CD274- | 19-mer Sense | UUGUACCUGCAUUAAUUUA | 233 |
| 3545 | Strand | | |
| CD274- | 19-mer Sense | UGUACCUGCAUUAAUUUAA | 234 |
| 3546 | Strand | | |
| CD274- | 19-mer Sense | AUAUUCUUAUUUAUUUUGU | 235 |
| 3569 | Strand | | |
| CD274- | 19-mer Sense | AUUUAUUUUGUUACUUGGU | 236 |
| 3577 | Strand | | |
| CD274- | 19-mer Sense | UUUAUUUUGUUACUUGGUA | 237 |
| 3578 | Strand | | |
| CD274- | 19-mer Sense | UAUUUUGUUACUUGGUACA | 238 |
| 3580 | Strand | | |
| CD274- | 19-mer Sense | AUGUCCAUUUUCUUGUUUA | 239 |
| 3604 | Strand | | |
| CD274- | 19-mer Sense | AUUUUCUUGUUUAUUUUGU | 240 |
| 3610 | Strand | | |
| CD274- | 19-mer Sense | AUUUUGUGUUUAAUAAAAU | 241 |
| 3622 | Strand | | |
| CD274- | 19-mer Anti- | UCAAAUAUCCUCAUCUUUC | 242 |
| 0088 | sense Strand | | |
| CD274- | 19-mer Anti- | UAGACAGCAAAUAUCCUCA | 243 |
| 0094 | sense Strand | | |
| CD274- | 19-mer Anti- | UUAAAGACAGCAAAUAUCC | 245 |
| 0097 | sense Strand | | |
| CD274- | 19-mer Anti- | UAUAAAGACAGCAAAUAUC | 246 |
| 0098 | sense Strand | | |
| CD274- | 19-mer Anti- | UAUAUAAAGACAGCAAAUA | 247 |
| 0100 | sense Strand | | |
| CD274- | 19-mer Anti- | UGAAUAUAAAGACAGCAAA | 248 |
| 0102 | sense Strand | | |
| CD274- | 19-mer Anti- | UUCUACCACAUAUAGGUCC | 249 |
| 0167 | sense Strand | | |
| CD274- | 19-mer Anti- | UAUUCAAUUGUCAUAUUGC | 250 |
| 0193 | sense Strand | | |
| CD274- | 19-mer Anti- | UCAUUCAAUUGUCAUAUUG | 251 |
| 0194 | sense Strand | | |
| CD274- | 19-mer Anti- | UUUGCAUUCAAUUGUCAUA | 252 |
| 0197 | sense Strand | | |
| CD274- | 19-mer Anti- | UAAUUUGCAUUCAAUUGUC | 253 |
| 0200 | sense Strand | | |
| CD274- | 19-mer Anti- | UUGGGAAUUUGCAUUCAAU | 254 |
| 0204 | sense Strand | | |
| CD274- | 19-mer Anti- | UUAAUUGUUUUUCUACUGG | 255 |
| 0219 | sense Strand | | |
| CD274- | 19-mer Anti- | UGUCUAAUUGUUUUUCUAC | 256 |
| 0222 | sense Strand | | |
| CD274- | 19-mer Anti- | UCAGGUCUAAUUGUUUUUC | 257 |
| 0225 | sense Strand | | |
| CD274- | 19-mer Anti- | UUAAUGUUCUUAUCCUCCA | 258 |
| 0268 | sense Strand | | |
| CD274- | 19-mer Anti- | UAUAAUGUUCUUAUCCUCC | 259 |
| 0269 | sense Strand | | |
| CD274- | 19-mer Anti- | UGAAUAAUGUUCUUAUCCU | 260 |
| 0271 | sense Strand | | |
| CD274- | 19-mer Anti- | UAUUGAAUAAUGUUCUUAU | 261 |
| 0274 | sense Strand | | |
| CD274- | 19-mer Anti- | UAAUUGAAUAAUGUUCUUA | 262 |
| 0275 | sense Strand | | |
| CD274- | 19-mer Anti- | UCAAAUUGAAUAAUGUUCU | 263 |
| 0277 | sense Strand | | |
| CD274- | 19-mer Anti- | UAUGCACAAAUUGAAUAAU | 264 |
| 0282 | sense Strand | | |
| CD274- | 19-mer Anti- | UCAUGCACAAAUUGAAUAA | 265 |
| 0283 | sense Strand | | |
| CD274- | 19-mer Anti- | UGCAAUUUCACAUCUGUGA | 266 |
| 0394 | sense Strand | | |
| CD274- | 19-mer Anti- | UAUCCUGCAAUUUCACAUC | 267 |
| 0399 | sense Strand | | |
| CD274- | 19-mer Anti- | UUCAUGUUCAGAGGUGACU | 268 |
| 0530 | sense Strand | | |
| CD274- | 19-mer Anti- | UUUCAUGUUCAGAGGUGAC | 269 |
| 0531 | sense Strand | | |
| CD274- | 19-mer Anti- | UACAUUGAAAAGCUUCUCC | 270 |
| 0653 | sense Strand | | |
| CD274- | 19-mer Anti- | UUGAUUCUCAGUGUGCUGG | 271 |
| 0673 | sense Strand | | |
| CD274- | 19-mer Anti- | UAAUCUCAUUAGUUGUUGU | 272 |
| 0693 | sense Strand | | |
| CD274- | 19-mer Anti- | UAGAAAAUCUCAUUAGUUG | 273 |
| 0697 | sense Strand | | |
| CD274- | 19-mer Anti- | UAGUAGAAAAUCUCAUUAG | 274 |
| 0700 | sense Strand | | |
| CD274- | 19-mer Anti- | UCAGUAGAAAAUCUCAUUA | 275 |
| 0701 | sense Strand | | |
| CD274- | 19-mer Anti- | UUCAGCUGUAUGGUUUUCC | 276 |
| 0743 | sense Strand | | |
| CD274- | 19-mer Anti- | UAAUUCAGCUGUAUGGUUU | 277 |
| 0746 | sense Strand | | |
| CD274- | 19-mer Anti- | UAAGACAGCAAAUAUCCUC | 278 |
| 0095 | sense Strand | | |
| CD274- | 19-mer Anti- | UUAUAAAGACAGCAAAUAU | 279 |
| 0099 | sense Strand | | |
| CD274- | 19-mer Anti- | UUGAAUAUAAAGACAGCAA | 280 |
| 0103 | sense Strand | | |
| CD274- | 19-mer Anti- | UGCAUUCAAUUGUCAUAUU | 281 |
| 0195 | sense Strand | | |
| CD274- | 19-mer Anti- | UUGCAUUCAAUUGUCAUAU | 282 |
| 0196 | sense Strand | | |
| CD274- | 19-mer Anti- | UUUUGCAUUCAAUUGUCAU | 283 |
| 0198 | sense Strand | | |
| CD274- | 19-mer Anti- | UAUUUGCAUUCAAUUGUCA | 284 |
| 0199 | sense Strand | | |
| CD274- | 19-mer Anti- | UGAAUUUGCAUUCAAUUGU | 285 |
| 0201 | sense Strand | | |
| CD274- | 19-mer Anti- | UGGAAUUUGCAUUCAAUUG | 286 |
| 0202 | sense Strand | | |
| CD274- | 19-mer Anti- | UUACUGGGAAUUUGCAUUC | 287 |
| 0207 | sense Strand | | |
| CD274- | 19-mer Anti- | UUCUAAUUGUUUUUCUACU | 288 |
| 0221 | sense Strand | | |
| CD274- | 19-mer Anti- | UCAAUAGACAAUUAGUGCA | 289 |
| 0245 | sense Strand | | |
| CD274- | 19-mer Anti- | UUCCCAAUAGACAAUUAGU | 290 |
| 0248 | sense Strand | | |
| CD274- | 19-mer Anti- | UUUCCCAAUAGACAAUUAG | 291 |
| 0249 | sense Strand | | |
| CD274- | 19-mer Anti- | UUGUUCUUAUCCUCCAUUU | 292 |
| 0265 | sense Strand | | |
| CD274- | 19-mer Anti- | UAUGUUCUUAUCCUCCAUU | 293 |
| 0266 | sense Strand | | |
| CD274- | 19-mer Anti- | UAAUGUUCUUAUCCUCCAU | 294 |
| 0267 | sense Strand | | |
| CD274- | 19-mer Anti- | UAAUAAUGUUCUUAUCCUC | 295 |
| 0270 | sense Strand | | |
| CD274- | 19-mer Anti- | UUGAAUAAUGUUCUUAUCC | 296 |
| 0272 | sense Strand | | |
| CD274- | 19-mer Anti- | UUUGAAUAAUGUUCUUAUC | 297 |
| 0273 | sense Strand | | |
| CD274- | 19-mer Anti- | UAAAUUGAAUAAUGUUCUU | 298 |
| 0276 | sense Strand | | |
| CD274- | 19-mer Anti- | UACAAAUUGAAUAAUGUUC | 299 |
| 0278 | sense Strand | | |
| CD274- | 19-mer Anti- | UCACAAAUUGAAUAAUGUU | 300 |
| 0279 | sense Strand | | |
| CD274- | 19-mer Anti- | UGCACAAAUUGAAUAAUGU | 301 |
| 0280 | sense Strand | | |
| CD274- | 19-mer Anti- | UUGCACAAAUUGAAUAAUG | 302 |
| 0281 | sense Strand | | |
| CD274- | 19-mer Anti- | UUCCAUGCACAAAUUGAAU | 303 |
| 0285 | sense Strand | | |
| CD274- | 19-mer Anti- | UAACCUUCAGGUCUUCCUC | 304 |
| 0303 | sense Strand | | |
| CD274- | 19-mer Anti- | UUGAACCUUCAGGUCUUCC | 305 |
| 0305 | sense Strand | | |
| CD274- | 19-mer Anti- | UUGCUGAACCUUCAGGUCU | 306 |
| 0308 | sense Strand | | |
| CD274- | 19-mer Anti- | UAUGCUGAACCUUCAGGUC | 307 |
| 0309 | sense Strand | | |
| CD274- | 19-mer Anti- | UUAUGCUGAACCUUCAGGU | 308 |
| 0310 | sense Strand | | |
| CD274- | 19-mer Anti- | UACUAUGCUGAACCUUCAG | 309 |
| 0312 | sense Strand | | |
| CD274- | 19-mer Anti- | UUACUAUGCUGAACCUUCA | 310 |
| 0313 | sense Strand | | |
| CD274- | 19-mer Anti- | UAAUUUCACAUCUGUGAUC | 311 |
| 0392 | sense Strand | | |
| CD274- | 19-mer Anti- | UUGCAAUUUCACAUCUGUG | 312 |
| 0395 | sense Strand | | |
| CD274- | 19-mer Anti- | UUCCUGCAAUUUCACAUCU | 313 |
| 0398 | sense Strand | | |
| CD274- | 19-mer Anti- | UAUUCUUUGGUUGAUUUUG | 314 |
| 0497 | sense Strand | | |
| CD274- | 19-mer Anti- | UAAAUUCUUUGGUUGAUUU | 315 |
| 0499 | sense Strand | | |
| CD274- | 19-mer Anti- | UAAAAUUCUUUGGUUGAUU | 316 |
| 0500 | sense Strand | | |
| CD274- | 19-mer Anti- | UCAAAAUUCUUUGGUUGAU | 317 |
| 0501 | sense Strand | | |
| CD274- | 19-mer Anti- | UCCAAAAUUCUUUGGUUGA | 318 |
| 0502 | sense Strand | | |
| CD274- | 19-mer Anti- | UAACCAAAAUUCUUUGGUU | 319 |
| 0504 | sense Strand | | |
| CD274- | 19-mer Anti- | UCAACCAAAAUUCUUUGGU | 320 |
| 0505 | sense Strand | | |
| CD274- | 19-mer Anti- | UACAACCAAAAUUCUUUGG | 321 |
| 0506 | sense Strand | | |
| CD274- | 19-mer Anti- | UCACAACCAAAAUUCUUUG | 322 |
| 0507 | sense Strand | | |
| CD274- | 19-mer Anti- | UUCCACAACCAAAAUUCUU | 323 |
| 0509 | sense Strand | | |
| CD274- | 19-mer Anti- | UAUCCACAACCAAAAUUCU | 324 |
| 0510 | sense Strand | | |
| CD274- | 19-mer Anti- | UGAUCCACAACCAAAAUUC | 325 |
| 0511 | sense Strand | | |
| CD274- | 19-mer Anti- | UUGGAUCCACAACCAAAAU | 326 |
| 0513 | sense Strand | | |
| CD274- | 19-mer Anti- | UUCAGUUCAUGUUCAGAGG | 327 |
| 0535 | sense Strand | | |
| CD274- | 19-mer Anti- | UUGUCAGUUCAUGUUCAGA | 328 |
| 0537 | sense Strand | | |
| CD274- | 19-mer Anti- | UUGACAUGUCAGUUCAUGU | 329 |
| 0542 | sense Strand | | |
| CD274- | 19-mer Anti- | UUGCUUGUCCAGAUGACUU | 330 |
| 0583 | sense Strand | | |
| CD274- | 19-mer Anti- | UUUGAUGGUCACUGCUUGU | 331 |
| 0594 | sense Strand | | |
| CD274- | 19-mer Anti- | UUCAGGACUUGAUGGUCAC | 332 |
| 0601 | sense Strand | | |
| CD274- | 19-mer Anti- | UCACAUUGAAAAGCUUCUC | 333 |
| 0654 | sense Strand | | |
| CD274- | 19-mer Anti- | UUCACAUUGAAAAGCUUCU | 334 |
| 0655 | sense Strand | | |
| CD274- | 19-mer Anti- | UGUCACAUUGAAAAGCUUC | 335 |
| 0656 | sense Strand | | |
| CD274- | 19-mer Anti- | UUGGUCACAUUGAAAAGCU | 336 |
| 0658 | sense Strand | | |
| CD274- | 19-mer Anti- | UUGCUGGUCACAUUGAAAA | 337 |
| 0661 | sense Strand | | |
| CD274- | 19-mer Anti- | UUCAGUGUGCUGGUCACAU | 338 |
| 0667 | sense Strand | | |
| CD274- | 19-mer Anti- | UUGUUGAUUCUCAGUGUGC | 339 |
| 0676 | sense Strand | | |
| CD274- | 19-mer Anti- | UUUGUGUUGAUUCUCAGUG | 340 |
| 0679 | sense Strand | | |
| CD274- | 19-mer Anti- | UUGUUGUGUUGAUUCUCAG | 341 |
| 0681 | sense Strand | | |
| CD274- | 19-mer Anti- | UUAGUUGUUGUGUUGAUUC | 342 |
| 0685 | sense Strand | | |
| CD274- | 19-mer Anti- | UAUCUCAUUAGUUGUUGUG | 343 |
| 0692 | sense Strand | | |
| CD274- | 19-mer Anti- | UAAAAUCUCAUUAGUUGUU | 344 |
| 0695 | sense Strand | | |
| CD274- | 19-mer Anti- | UGAAAAUCUCAUUAGUUGU | 345 |
| 0696 | sense Strand | | |
| CD274- | 19-mer Anti- | UUAGAAAAUCUCAUUAGUU | 346 |
| 0698 | sense Strand | | |
| CD274- | 19-mer Anti- | UUGCAGUAGAAAAUCUCAU | 347 |
| 0703 | sense Strand | | |
| CD274- | 19-mer Anti- | UUAAAAGUGCAGUAGAAAA | 348 |
| 0709 | sense Strand | | |
| CD274- | 19-mer Anti- | UAUUCAGCUGUAUGGUUUU | 349 |
| 0745 | sense Strand | | |
| CD274- | 19-mer Anti- | UACCAAUUCAGCUGUAUGG | 350 |
| 0749 | sense Strand | | |
| CD274- | 19-mer Anti- | UAUGACCAAUUCAGCUGUA | 351 |
| 0752 | sense Strand | | |
| CD274- | 19-mer Anti- | UACCAAGUGAGUCCUUUCA | 352 |
| 0800 | sense Strand | | |
| CD274- | 19-mer Anti- | UAAUUACCAAGUGAGUCCU | 353 |
| 0804 | sense Strand | | |
| CD274- | 19-mer Anti- | UUGAAUGUCAGUGCUACAC | 354 |
| 0847 | sense Strand | | |
| CD274- | 19-mer Anti- | UGAAGAUGAAUGUCAGUGC | 355 |
| 0852 | sense Strand | | |
| CD274- | 19-mer Anti- | UAUUUUUUCACAUCCAUCA | 356 |
| 0889 | sense Strand | | |
| CD274- | 19-mer Anti- | UUGAAAGUAUCAAGGUCUC | 357 |
| 1172 | sense Strand | | |
| CD274- | 19-mer Anti- | UUUGAAAGUAUCAAGGUCU | 358 |
| 1173 | sense Strand | | |
| CD274- | 19-mer Anti- | UUUUGAAAGUAUCAAGGUC | 359 |
| 1174 | sense Strand | | |
| CD274- | 19-mer Anti- | UCAUUUGAAAGUAUCAAGG | 360 |
| 1176 | sense Strand | | |
| CD274- | 19-mer Anti- | UGGCAUUUGAAAGUAUCAA | 361 |
| 1178 | sense Strand | | |
| CD274- | 19-mer Anti- | UUGUUCAGAAGUAUCCUUU | 362 |
| 1227 | sense Strand | | |
| CD274- | 19-mer Anti- | UUCCUUGUUCAGAAGUAUC | 363 |
| 1231 | sense Strand | | |
| CD274- | 19-mer Anti- | UACAGACUCAAAAUAAAUA | 364 |
| 1423 | sense Strand | | |
| CD274- | 19-mer Anti- | UUCACAGACUCAAAAUAAA | 365 |
| 1425 | sense Strand | | |
| CD274- | 19-mer Anti- | UUCAUUCACAACCACACUC | 366 |
| 1459 | sense Strand | | |
| CD274- | 19-mer Anti- | UAUCAUUCACAACCACACU | 367 |
| 1460 | sense Strand | | |
| CD274- | 19-mer Anti- | UAAUCAUUCACAACCACAC | 368 |
| 1461 | sense Strand | | |
| CD274- | 19-mer Anti- | UAAAUCAUUCACAACCACA | 369 |
| 1462 | sense Strand | | |
| CD274- | 19-mer Anti- | UGAAAUCAUUCACAACCAC | 370 |
| 1463 | sense Strand | | |
| CD274- | 19-mer Anti- | UAGAAAUCAUUCACAACCA | 371 |
| 1464 | sense Strand | | |
| CD274- | 19-mer Anti- | UAAGAAAUCAUUCACAACC | 372 |
| 1465 | sense Strand | | |
| CD274- | 19-mer Anti- | UAAAAGAAAUCAUUCACAA | 373 |
| 1467 | sense Strand | | |
| CD274- | 19-mer Anti- | UUCAAAAGAAAUCAUUCAC | 374 |
| 1469 | sense Strand | | |
| CD274- | 19-mer Anti- | UUUCAAAAGAAAUCAUUCA | 375 |
| 1470 | sense Strand | | |
| CD274- | 19-mer Anti- | UAUCUUCAAAAGAAAUCAU | 376 |
| 1473 | sense Strand | | |
| CD274- | 19-mer Anti- | UUAUCUUCAAAAGAAAUCA | 377 |
| 1474 | sense Strand | | |
| CD274- | 19-mer Anti- | UUACAAUAUAUCUUCAAAA | 378 |
| 1481 | sense Strand | | |
| CD274- | 19-mer Anti- | UUACUACAAUAUAUCUUCA | 379 |
| 1484 | sense Strand | | |
| CD274- | 19-mer Anti- | UUCUACUACAAUAUAUCUU | 380 |
| 1486 | sense Strand | | |
| CD274- | 19-mer Anti- | UCAAAAUUGUAACAUCUAC | 381 |
| 1499 | sense Strand | | |
| CD274- | 19-mer Anti- | UACAAAAUUGUAACAUCUA | 382 |
| 1500 | sense Strand | | |
| CD274- | 19-mer Anti- | UUAGAUGUGAGCAAAUCAU | 383 |
| 1541 | sense Strand | | |
| CD274- | 19-mer Anti- | UUACAAAUACUCCAUGUUU | 384 |
| 1562 | sense Strand | | |
| CD274- | 19-mer Anti- | UUGCUUCCAAUGUAUACUU | 385 |
| 1606 | sense Strand | | |
| CD274- | 19-mer Anti- | UAUGCUUCCAAUGUAUACU | 386 |
| 1607 | sense Strand | | |
| CD274- | 19-mer Anti- | UUAAAUAAAGGUGACAUCC | 387 |
| 1649 | sense Strand | | |
| CD274- | 19-mer Anti- | UUGAUAUUUAAAUGGAACA | 388 |
| 1728 | sense Strand | | |
| CD274- | 19-mer Anti- | UUCACAACAGGGUGGUUAC | 389 |
| 1794 | sense Strand | | |
| CD274- | 19-mer Anti- | UAUCACAACAGGGUGGUUA | 390 |
| 1795 | sense Strand | | |
| CD274- | 19-mer Anti- | UUAAUAGUGGUUAUCACAA | 391 |
| 1806 | sense Strand | | |
| CD274- | 19-mer Anti- | UAUAAUAGUGGUUAUCACA | 392 |
| 1807 | sense Strand | | |
| CD274- | 19-mer Anti- | UAAUAAUAGUGGUUAUCAC | 393 |
| 1808 | sense Strand | | |
| CD274- | 19-mer Anti- | UAAAUAAUAGUGGUUAUCA | 394 |
| 1809 | sense Strand | | |
| CD274- | 19-mer Anti- | UAAAAUAAUAGUGGUUAUC | 395 |
| 1810 | sense Strand | | |
| CD274- | 19-mer Anti- | UUAAAAUAAUAGUGGUUAU | 396 |
| 1811 | sense Strand | | |
| CD274- | 19-mer Anti- | UGUAAAAUAAUAGUGGUUA | 397 |
| 1812 | sense Strand | | |
| CD274- | 19-mer Anti- | UUAUUAUAAAAGGACAGUG | 398 |
| 1906 | sense Strand | | |
| CD274- | 19-mer Anti- | UGUAUUAUAAAAGGACAGU | 399 |
| 1907 | sense Strand | | |
| CD274- | 19-mer Anti- | UUGUAUUAUAAAAGGACAG | 400 |
| 1908 | sense Strand | | |
| CD274- | 19-mer Anti- | UUUGUAUUAUAAAAGGACA | 401 |
| 1909 | sense Strand | | |
| CD274- | 19-mer Anti- | UAUUGUAUUAUAAAAGGAC | 402 |
| 1910 | sense Strand | | |
| CD274- | 19-mer Anti- | UUAAAUUGUAUUAUAAAAG | 403 |
| 1913 | sense Strand | | |
| CD274- | 19-mer Anti- | UUGUAAAUUGUAUUAUAAA | 404 |
| 1915 | sense Strand | | |
| CD274- | 19-mer Anti- | UUAGCUGUAAAUUGUAUUA | 405 |
| 1919 | sense Strand | | |
| CD274- | 19-mer Anti- | UUAAAGUAAAAUAUAGCUG | 406 |
| 1931 | sense Strand | | |
| CD274- | 19-mer Anti- | UUGCUUAAAGUAAAAUAUA | 407 |
| 1935 | sense Strand | | |
| CD274- | 19-mer Anti- | UAUUGCUUAAAGUAAAAUA | 408 |
| 1937 | sense Strand | | |
| CD274- | 19-mer Anti- | UUCACAUCUGUAGAUUCAA | 409 |
| 2014 | sense Strand | | |
| CD274- | 19-mer Anti- | UAUUGUAAUAAUACAUUUU | 410 |
| 2088 | sense Strand | | |
| CD274- | 19-mer Anti- | UUGAAAAUUACUCUGAUUA | 411 |
| 2221 | sense Strand | | |
| CD274- | 19-mer Anti- | UUAAAUGAAAAUUACUCUG | 412 |
| 2225 | sense Strand | | |
| CD274- | 19-mer Anti- | UUGUAAAUGAAAAUUACUC | 413 |
| 2227 | sense Strand | | |
| CD274- | 19-mer Anti- | UUUGUAAAUGAAAAUUACU | 414 |
| 2228 | sense Strand | | |
| CD274- | 19-mer Anti- | UUUUGUAAAUGAAAAUUAC | 415 |
| 2229 | sense Strand | | |
| CD274- | 19-mer Anti- | UUCUUUGUAAAUGAAAAUU | 416 |
| 2231 | sense Strand | | |
| CD274- | 19-mer Anti- | UUCUCUUUGUAAAUGAAAA | 417 |
| 2233 | sense Strand | | |
| CD274- | 19-mer Anti- | UUAUUUUUCAGGGUUAUUU | 418 |
| 2263 | sense Strand | | |
| CD274- | 19-mer Anti- | UUGUUAUUUUUCAGGGUUA | 419 |
| 2266 | sense Strand | | |
| CD274- | 19-mer Anti- | UAACAAGCAUUAGAUUAUA | 420 |
| 2333 | sense Strand | | |
| CD274- | 19-mer Anti- | UUAAACAAGCAUUAGAUUA | 421 |
| 2335 | sense Strand | | |
| CD274- | 19-mer Anti- | UUAUAAACAAGCAUUAGAU | 422 |
| 2337 | sense Strand | | |
| CD274- | 19-mer Anti- | UAUAUAAACAAGCAUUAGA | 423 |
| 2338 | sense Strand | | |
| CD274- | 19-mer Anti- | UUAUAUAAACAAGCAUUAG | 424 |
| 2339 | sense Strand | | |
| CD274- | 19-mer Anti- | UACUAUAUAAACAAGCAUU | 425 |
| 2341 | sense Strand | | |
| CD274- | 19-mer Anti- | UACACUAUAUAAACAAGCA | 426 |
| 2343 | sense Strand | | |
| CD274- | 19-mer Anti- | UAACUGUUAAACAAUACCA | 427 |
| 2362 | sense Strand | | |
| CD274- | 19-mer Anti- | UGAACUGUUAAACAAUACC | 428 |
| 2363 | sense Strand | | |
| CD274- | 19-mer Anti- | UAAGGUAUGAAUUUAAAAU | 429 |
| 2407 | sense Strand | | |
| CD274- | 19-mer Anti- | UACAUUUCCAAAUGUAGAC | 430 |
| 2556 | sense Strand | | |
| CD274- | 19-mer Anti- | UUACAUUUCCAAAUGUAGA | 431 |
| 2557 | sense Strand | | |
| CD274- | 19-mer Anti- | UAACAUACAUUUCCAAAUG | 432 |
| 2561 | sense Strand | | |
| CD274- | 19-mer Anti- | UUAACAUACAUUUCCAAAU | 433 |
| 2562 | sense Strand | | |
| CD274- | 19-mer Anti- | UUUAACAUACAUUUCCAAA | 434 |
| 2563 | sense Strand | | |
| CD274- | 19-mer Anti- | UUUUUAACAUACAUUUCCA | 435 |
| 2565 | sense Strand | | |
| CD274- | 19-mer Anti- | UUGCUUUUAACAUACAUUU | 436 |
| 2568 | sense Strand | | |
| CD274- | 19-mer Anti- | UAAAAUAGCAAAGUACACA | 437 |
| 2634 | sense Strand | | |
| CD274- | 19-mer Anti- | UUAAAAAUAGCAAAGUACA | 438 |
| 2636 | sense Strand | | |
| CD274- | 19-mer Anti- | UAUAAAAAUAGCAAAGUAC | 439 |
| 2637 | sense Strand | | |
| CD274- | 19-mer Anti- | UUAAAUAAAAAUAGCAAAG | 440 |
| 2640 | sense Strand | | |
| CD274- | 19-mer Anti- | UUUCAUUCCAUCUGCUAUA | 441 |
| 2673 | sense Strand | | |
| CD274- | 19-mer Anti- | UUCAAAUUCAUUCCAUCUG | 442 |
| 2678 | sense Strand | | |
| CD274- | 19-mer Anti- | UUUCAAAUUCAUUCCAUCU | 443 |
| 2679 | sense Strand | | |
| CD274- | 19-mer Anti- | UUGGUUGUCUAAAUGUAUA | 444 |
| 2801 | sense Strand | | |
| CD274- | 19-mer Anti- | UUACUUAACAAAUGGUGGU | 445 |
| 2815 | sense Strand | | |
| CD274- | 19-mer Anti- | UCAAAUACUUAACAAAUGG | 446 |
| 2819 | sense Strand | | |
| CD274- | 19-mer Anti- | UUAAUAAUAAGAUUGCUUU | 447 |
| 2924 | sense Strand | | |
| CD274- | 19-mer Anti- | UUCAUACAGAGUUAAUAAU | 448 |
| 2935 | sense Strand | | |
| CD274- | 19-mer Anti- | UGUCAUACAGAGUUAAUAA | 449 |
| 2936 | sense Strand | | |
| CD274- | 19-mer Anti- | UUGUCAUACAGAGUUAAUA | 450 |
| 2937 | sense Strand | | |
| CD274- | 19-mer Anti- | UUCUGUCAUACAGAGUUAA | 451 |
| 2939 | sense Strand | | |
| CD274- | 19-mer Anti- | UUCAAAGUGAAGUUUAUAC | 452 |
| 2994 | sense Strand | | |
| CD274- | 19-mer Anti- | UUGUGAUUUUGCAAGUACA | 453 |
| 3015 | sense Strand | | |
| CD274- | 19-mer Anti- | UAAAAUGUGAUUUUGCAAG | 454 |
| 3019 | sense Strand | | |
| CD274- | 19-mer Anti- | UUCAAGCACAACGAAUGAG | 455 |
| 3094 | sense Strand | | |
| CD274- | 19-mer Anti- | UUUCAAGCACAACGAAUGA | 456 |
| 3095 | sense Strand | | |
| CD274- | 19-mer Anti- | UUACAAACAGAUAACACAA | 457 |
| 3305 | sense Strand | | |
| CD274- | 19-mer Anti- | UGUACAAACAGAUAACACA | 458 |
| 3306 | sense Strand | | |
| CD274- | 19-mer Anti- | UUGUACAAACAGAUAACAC | 459 |
| 3307 | sense Strand | | |
| CD274- | 19-mer Anti- | UAUGUACAAACAGAUAACA | 460 |
| 3308 | sense Strand | | |
| CD274- | 19-mer Anti- | UACAUGUACAAACAGAUAA | 461 |
| 3310 | sense Strand | | |
| CD274- | 19-mer Anti- | UUACAAAUGCACAUGUACA | 462 |
| 3319 | sense Strand | | |
| CD274- | 19-mer Anti- | UUUACUGUACAAAUGCACA | 463 |
| 3325 | sense Strand | | |
| CD274- | 19-mer Anti- | UUAAUUCACACAAAGAACA | 464 |
| 3355 | sense Strand | | |
| CD274- | 19-mer Anti- | UUGUAAUUCACACAAAGAA | 465 |
| 3357 | sense Strand | | |
| CD274- | 19-mer Anti- | UUACAAAUCCAACACCACA | 466 |
| 3441 | sense Strand | | |
| CD274- | 19-mer Anti- | UUGACAAUCAAAUGCAGAA | 467 |
| 3522 | sense Strand | | |
| CD274- | 19-mer Anti- | UAAAAAGUGACAAUCAAAU | 468 |
| 3528 | sense Strand | | |
| CD274- | 19-mer Anti- | UUACAAAAAGUGACAAUCA | 469 |
| 3531 | sense Strand | | |
| CD274- | 19-mer Anti- | UGUACAAAAAGUGACAAUC | 470 |
| 3532 | sense Strand | | |
| CD274- | 19-mer Anti- | UAGGUACAAAAAGUGACAA | 471 |
| 3534 | sense Strand | | |
| CD274- | 19-mer Anti- | UUUAAUGCAGGUACAAAAA | 472 |
| 3541 | sense Strand | | |
| CD274- | 19-mer Anti- | UAUUAAUGCAGGUACAAAA | 473 |
| 3542 | sense Strand | | |
| CD274- | 19-mer Anti- | UUAAAUUAAUGCAGGUACA | 474 |
| 3545 | sense Strand | | |
| CD274- | 19-mer Anti- | UUUAAAUUAAUGCAGGUAC | 475 |
| 3546 | sense Strand | | |
| CD274- | 19-mer Anti- | UACAAAAUAAAUAAGAAUA | 476 |
| 3569 | sense Strand | | |
| CD274- | 19-mer Anti- | UACCAAGUAACAAAAUAAA | 477 |
| 3577 | sense Strand | | |
| CD274- | 19-mer Anti- | UUACCAAGUAACAAAAUAA | 478 |
| 3578 | sense Strand | | |
| CD274- | 19-mer Anti- | UUGUACCAAGUAACAAAAU | 479 |
| 3580 | sense Strand | | |
| CD274- | 19-mer Anti- | UUAAACAAGAAAAUGGACA | 480 |
| 3604 | sense Strand | | |
| CD274- | 19-mer Anti- | UACAAAAUAAACAAGAAAA | 481 |
| 3610 | sense Strand | | |
| CD274- | 19-mer Anti- | UAUUUUAUUAAACACAAAA | 482 |
| 3622 | sense Strand | | |
| CD274- | Unmodified | AGAAAGAUGAGGAUAUUUGAGCAGCCGAAAGG | 483 |
| 0088 | 36-mer | CUGC | |
| CD274- | Unmodified | AUGAGGAUAUUUGCUGUCUAGCAGCCGAAAGG | 484 |
| 0094 | 36-mer | CUGC | |
| CD274- | Unmodified | AGGAUAUUUGCUGUCUUUAAGCAGCCGAAAGG | 486 |
| 0097 | 36-mer | CUGC | |
| CD274- | Unmodified | GGAUAUUUGCUGUCUUUAUAGCAGCCGAAAGG | 487 |
| 0098 | 36-mer | CUGC | |
| CD274- | Unmodified | AUAUUUGCUGUCUUUAUAUAGCAGCCGAAAGG | 488 |
| 0100 | 36-mer | CUGC | |
| CD274- | Unmodified | AUUUGCUGUCUUUAUAUUCAGCAGCCGAAAGGC | 489 |
| 0102 | 36-mer | UGC | |
| CD274- | Unmodified | AGGACCUAUAUGUGGUAGAAGCAGCCGAAAGG | 490 |
| 0167 | 36-mer | CUGC | |
| CD274- | Unmodified | AGCAAUAUGACAAUUGAAUAGCAGCCGAAAGG | 491 |
| 0193 | 36-mer | CUGC | |
| CD274- | Unmodified | GCAAUAUGACAAUUGAAUGAGCAGCCGAAAGG | 492 |
| 0194 | 36-mer | CUGC | |
| CD274- | Unmodified | AUAUGACAAUUGAAUGCAAAGCAGCCGAAAGG | 493 |
| 0197 | 36-mer | CUGC | |
| CD274- | Unmodified | UGACAAUUGAAUGCAAAUUAGCAGCCGAAAGG | 494 |
| 0200 | 36-mer | CUGC | |
| CD274- | Unmodified | AAUUGAAUGCAAAUUCCCAAGCAGCCGAAAGGC | 495 |
| 0204 | 36-mer | UGC | |
| CD274- | Unmodified | CCCAGUAGAAAAACAAUUAAGCAGCCGAAAGGC | 496 |
| 0219 | 36-mer | UGC | |
| CD274- | Unmodified | AGUAGAAAAACAAUUAGACAGCAGCCGAAAGG | 497 |
| 0222 | 36-mer | CUGC | |
| CD274- | Unmodified | AGAAAAACAAUUAGACCUGAGCAGCCGAAAGGC | 498 |
| 0225 | 36-mer | UGC | |
| CD274- | Unmodified | AUGGAGGAUAAGAACAUUAAGCAGCCGAAAGG | 499 |
| 0268 | 36-mer | CUGC | |
| CD274- | Unmodified | UGGAGGAUAAGAACAUUAUAGCAGCCGAAAGG | 500 |
| 0269 | 36-mer | CUGC | |
| CD274- | Unmodified | GAGGAUAAGAACAUUAUUCAGCAGCCGAAAGG | 501 |
| 0271 | 36-mer | CUGC | |
| CD274- | Unmodified | GAUAAGAACAUUAUUCAAUAGCAGCCGAAAGG | 502 |
| 0274 | 36-mer | CUGC | |
| CD274- | Unmodified | AUAAGAACAUUAUUCAAUUAGCAGCCGAAAGG | 503 |
| 0275 | 36-mer | CUGC | |
| CD274- | Unmodified | AAGAACAUUAUUCAAUUUGAGCAGCCGAAAGG | 504 |
| 0277 | 36-mer | CUGC | |
| CD274- | Unmodified | CAUUAUUCAAUUUGUGCAUAGCAGCCGAAAGGC | 505 |
| 0282 | 36-mer | UGC | |
| CD274- | Unmodified | AUUAUUCAAUUUGUGCAUGAGCAGCCGAAAGG | 506 |
| 0283 | 36-mer | CUGC | |
| CD274- | Unmodified | AUCACAGAUGUGAAAUUGCAGCAGCCGAAAGGC | 507 |
| 0394 | 36-mer | UGC | |
| CD274- | Unmodified | AGAUGUGAAAUUGCAGGAUAGCAGCCGAAAGG | 508 |
| 0399 | 36-mer | CUGC | |
| CD274- | Unmodified | CAGUCACCUCUGAACAUGAAGCAGCCGAAAGGC | 509 |
| 0530 | 36-mer | UGC | |
| CD274- | Unmodified | AGUCACCUCUGAACAUGAAAGCAGCCGAAAGGC | 510 |
| 0531 | 36-mer | UGC | |
| CD274- | Unmodified | AGGAGAAGCUUUUCAAUGUAGCAGCCGAAAGG | 511 |
| 0653 | 36-mer | CUGC | |
| CD274- | Unmodified | ACCAGCACACUGAGAAUCAAGCAGCCGAAAGGC | 512 |
| 0673 | 36-mer | UGC | |
| CD274- | Unmodified | CACAACAACUAAUGAGAUUAGCAGCCGAAAGGC | 513 |
| 0693 | 36-mer | UGC | |
| CD274- | Unmodified | ACAACUAAUGAGAUUUUCUAGCAGCCGAAAGGC | 514 |
| 0697 | 36-mer | UGC | |
| CD274- | Unmodified | ACUAAUGAGAUUUUCUACUAGCAGCCGAAAGGC | 515 |
| 0700 | 36-mer | UGC | |
| CD274- | Unmodified | CUAAUGAGAUUUUCUACUGAGCAGCCGAAAGGC | 516 |
| 0701 | 36-mer | UGC | |
| CD274- | Unmodified | AGGAAAACCAUACAGCUGAAGCAGCCGAAAGGC | 517 |
| 0743 | 36-mer | UGC | |
| CD274- | Unmodified | AAAACCAUACAGCUGAAUUAGCAGCCGAAAGGC | 518 |
| 0746 | 36-mer | UGC | |
| CD274- | Unmodified | UGAGGAUAUUUGCUGUCUUAGCAGCCGAAAGG | 519 |
| 0095 | 36-mer | CUGC | |
| CD274- | Unmodified | GAUAUUUGCUGUCUUUAUAAGCAGCCGAAAGG | 520 |
| 0099 | 36-mer | CUGC | |
| CD274- | Unmodified | UUUGCUGUCUUUAUAUUCAAGCAGCCGAAAGGC | 521 |
| 0103 | 36-mer | UGC | |
| CD274- | Unmodified | CAAUAUGACAAUUGAAUGCAGCAGCCGAAAGGC | 522 |
| 0195 | 36-mer | UGC | |
| CD274- | Unmodified | AAUAUGACAAUUGAAUGCAAGCAGCCGAAAGG | 523 |
| 0196 | 36-mer | CUGC | |
| CD274- | Unmodified | UAUGACAAUUGAAUGCAAAAGCAGCCGAAAGG | 524 |
| 0198 | 36-mer | CUGC | |
| CD274- | Unmodified | AUGACAAUUGAAUGCAAAUAGCAGCCGAAAGG | 525 |
| 0199 | 36-mer | CUGC | |
| CD274- | Unmodified | GACAAUUGAAUGCAAAUUCAGCAGCCGAAAGGC | 526 |
| 0201 | 36-mer | UGC | |
| CD274- | Unmodified | ACAAUUGAAUGCAAAUUCCAGCAGCCGAAAGGC | 527 |
| 0202 | 36-mer | UGC | |
| CD274- | Unmodified | UGAAUGCAAAUUCCCAGUAAGCAGCCGAAAGGC | 528 |
| 0207 | 36-mer | UGC | |
| CD274- | Unmodified | CAGUAGAAAAACAAUUAGAAGCAGCCGAAAGG | 529 |
| 0221 | 36-mer | CUGC | |
| CD274- | Unmodified | CUGCACUAAUUGUCUAUUGAGCAGCCGAAAGGC | 530 |
| 0245 | 36-mer | UGC | |
| CD274- | Unmodified | CACUAAUUGUCUAUUGGGAAGCAGCCGAAAGGC | 531 |
| 0248 | 36-mer | UGC | |
| CD274- | Unmodified | ACUAAUUGUCUAUUGGGAAAGCAGCCGAAAGG | 532 |
| 0249 | 36-mer | CUGC | |
| CD274- | Unmodified | GAAAUGGAGGAUAAGAACAAGCAGCCGAAAGG | 533 |
| 0265 | 36-mer | CUGC | |
| CD274- | Unmodified | AAAUGGAGGAUAAGAACAUAGCAGCCGAAAGG | 534 |
| 0266 | 36-mer | CUGC | |
| CD274- | Unmodified | AAUGGAGGAUAAGAACAUUAGCAGCCGAAAGG | 535 |
| 0267 | 36-mer | CUGC | |
| CD274- | Unmodified | GGAGGAUAAGAACAUUAUUAGCAGCCGAAAGG | 536 |
| 0270 | 36-mer | CUGC | |
| CD274- | Unmodified | AGGAUAAGAACAUUAUUCAAGCAGCCGAAAGG | 537 |
| 0272 | 36-mer | CUGC | |
| CD274- | Unmodified | GGAUAAGAACAUUAUUCAAAGCAGCCGAAAGG | 538 |
| 0273 | 36-mer | CUGC | |
| CD274- | Unmodified | UAAGAACAUUAUUCAAUUUAGCAGCCGAAAGG | 539 |
| 0276 | 36-mer | CUGC | |
| CD274- | Unmodified | AGAACAUUAUUCAAUUUGUAGCAGCCGAAAGG | 540 |
| 0278 | 36-mer | CUGC | |
| CD274- | Unmodified | GAACAUUAUUCAAUUUGUGAGCAGCCGAAAGG | 541 |
| 0279 | 36-mer | CUGC | |
| CD274- | Unmodified | AACAUUAUUCAAUUUGUGCAGCAGCCGAAAGGC | 542 |
| 0280 | 36-mer | UGC | |
| CD274- | Unmodified | ACAUUAUUCAAUUUGUGCAAGCAGCCGAAAGGC | 543 |
| 0281 | 36-mer | UGC | |
| CD274- | Unmodified | UAUUCAAUUUGUGCAUGGAAGCAGCCGAAAGG | 544 |
| 0285 | 36-mer | CUGC | |
| CD274- | Unmodified | AGAGGAAGACCUGAAGGUUAGCAGCCGAAAGG | 545 |
| 0303 | 36-mer | CUGC | |
| CD274- | Unmodified | AGGAAGACCUGAAGGUUCAAGCAGCCGAAAGGC | 546 |
| 0305 | 36-mer | UGC | |
| CD274- | Unmodified | AAGACCUGAAGGUUCAGCAAGCAGCCGAAAGGC | 547 |
| 0308 | 36-mer | UGC | |
| CD274- | Unmodified | AGACCUGAAGGUUCAGCAUAGCAGCCGAAAGGC | 548 |
| 0309 | 36-mer | UGC | |
| CD274- | Unmodified | GACCUGAAGGUUCAGCAUAAGCAGCCGAAAGGC | 549 |
| 0310 | 36-mer | UGC | |
| CD274- | Unmodified | CCUGAAGGUUCAGCAUAGUAGCAGCCGAAAGGC | 550 |
| 0312 | 36-mer | UGC | |
| CD274- | Unmodified | CUGAAGGUUCAGCAUAGUAAGCAGCCGAAAGGC | 551 |
| 0313 | 36-mer | UGC | |
| CD274- | Unmodified | AGAUCACAGAUGUGAAAUUAGCAGCCGAAAGG | 552 |
| 0392 | 36-mer | CUGC | |
| CD274- | Unmodified | UCACAGAUGUGAAAUUGCAAGCAGCCGAAAGGC | 553 |
| 0395 | 36-mer | UGC | |
| CD274- | Unmodified | CAGAUGUGAAAUUGCAGGAAGCAGCCGAAAGG | 554 |
| 0398 | 36-mer | CUGC | |
| CD274- | Unmodified | ACAAAAUCAACCAAAGAAUAGCAGCCGAAAGGC | 555 |
| 0497 | 36-mer | UGC | |
| CD274- | Unmodified | AAAAUCAACCAAAGAAUUUAGCAGCCGAAAGGC | 556 |
| 0499 | 36-mer | UGC | |
| CD274- | Unmodified | AAAUCAACCAAAGAAUUUUAGCAGCCGAAAGGC | 557 |
| 0500 | 36-mer | UGC | |
| CD274- | Unmodified | AAUCAACCAAAGAAUUUUGAGCAGCCGAAAGGC | 558 |
| 0501 | 36-mer | UGC | |
| CD274- | Unmodified | AUCAACCAAAGAAUUUUGGAGCAGCCGAAAGGC | 559 |
| 0502 | 36-mer | UGC | |
| CD274- | Unmodified | CAACCAAAGAAUUUUGGUUAGCAGCCGAAAGGC | 560 |
| 0504 | 36-mer | UGC | |
| CD274- | Unmodified | AACCAAAGAAUUUUGGUUGAGCAGCCGAAAGG | 561 |
| 0505 | 36-mer | CUGC | |
| CD274- | Unmodified | ACCAAAGAAUUUUGGUUGUAGCAGCCGAAAGG | 562 |
| 0506 | 36-mer | CUGC | |
| CD274- | Unmodified | CCAAAGAAUUUUGGUUGUGAGCAGCCGAAAGG | 563 |
| 0507 | 36-mer | CUGC | |
| CD274- | Unmodified | AAAGAAUUUUGGUUGUGGAAGCAGCCGAAAGG | 564 |
| 0509 | 36-mer | CUGC | |
| CD274- | Unmodified | AAGAAUUUUGGUUGUGGAUAGCAGCCGAAAGG | 565 |
| 0510 | 36-mer | CUGC | |
| CD274- | Unmodified | AGAAUUUUGGUUGUGGAUCAGCAGCCGAAAGG | 566 |
| 0511 | 36-mer | CUGC | |
| CD274- | Unmodified | AAUUUUGGUUGUGGAUCCAAGCAGCCGAAAGG | 567 |
| 0513 | 36-mer | CUGC | |
| CD274- | Unmodified | ACCUCUGAACAUGAACUGAAGCAGCCGAAAGGC | 568 |
| 0535 | 36-mer | UGC | |
| CD274- | Unmodified | CUCUGAACAUGAACUGACAAGCAGCCGAAAGGC | 569 |
| 0537 | 36-mer | UGC | |
| CD274- | Unmodified | AACAUGAACUGACAUGUCAAGCAGCCGAAAGGC | 570 |
| 0542 | 36-mer | UGC | |
| CD274- | Unmodified | GAAGUCAUCUGGACAAGCAAGCAGCCGAAAGGC | 571 |
| 0583 | 36-mer | UGC | |
| CD274- | Unmodified | GACAAGCAGUGACCAUCAAAGCAGCCGAAAGGC | 572 |
| 0594 | 36-mer | UGC | |
| CD274- | Unmodified | AGUGACCAUCAAGUCCUGAAGCAGCCGAAAGGC | 573 |
| 0601 | 36-mer | UGC | |
| CD274- | Unmodified | GGAGAAGCUUUUCAAUGUGAGCAGCCGAAAGG | 574 |
| 0654 | 36-mer | CUGC | |
| CD274- | Unmodified | GAGAAGCUUUUCAAUGUGAAGCAGCCGAAAGG | 575 |
| 0655 | 36-mer | CUGC | |
| CD274- | Unmodified | AGAAGCUUUUCAAUGUGACAGCAGCCGAAAGGC | 576 |
| 0656 | 36-mer | UGC | |
| CD274- | Unmodified | AAGCUUUUCAAUGUGACCAAGCAGCCGAAAGGC | 577 |
| 0658 | 36-mer | UGC | |
| CD274- | Unmodified | CUUUUCAAUGUGACCAGCAAGCAGCCGAAAGGC | 578 |
| 0661 | 36-mer | UGC | |
| CD274- | Unmodified | AAUGUGACCAGCACACUGAAGCAGCCGAAAGGC | 579 |
| 0667 | 36-mer | UGC | |
| CD274- | Unmodified | AGCACACUGAGAAUCAACAAGCAGCCGAAAGGC | 580 |
| 0676 | 36-mer | UGC | |
| CD274- | Unmodified | ACACUGAGAAUCAACACAAAGCAGCCGAAAGGC | 581 |
| 0679 | 36-mer | UGC | |
| CD274- | Unmodified | ACUGAGAAUCAACACAACAAGCAGCCGAAAGGC | 582 |
| 0681 | 36-mer | UGC | |
| CD274- | Unmodified | AGAAUCAACACAACAACUAAGCAGCCGAAAGGC | 583 |
| 0685 | 36-mer | UGC | |
| CD274- | Unmodified | ACACAACAACUAAUGAGAUAGCAGCCGAAAGGC | 584 |
| 0692 | 36-mer | UGC | |
| CD274- | Unmodified | CAACAACUAAUGAGAUUUUAGCAGCCGAAAGGC | 585 |
| 0695 | 36-mer | UGC | |
| CD274- | Unmodified | AACAACUAAUGAGAUUUUCAGCAGCCGAAAGGC | 586 |
| 0696 | 36-mer | UGC | |
| CD274- | Unmodified | CAACUAAUGAGAUUUUCUAAGCAGCCGAAAGGC | 587 |
| 0698 | 36-mer | UGC | |
| CD274- | Unmodified | AAUGAGAUUUUCUACUGCAAGCAGCCGAAAGGC | 588 |
| 0703 | 36-mer | UGC | |
| CD274- | Unmodified | AUUUUCUACUGCACUUUUAAGCAGCCGAAAGGC | 589 |
| 0709 | 36-mer | UGC | |
| CD274- | Unmodified | GAAAACCAUACAGCUGAAUAGCAGCCGAAAGGC | 590 |
| 0745 | 36-mer | UGC | |
| CD274- | Unmodified | ACCAUACAGCUGAAUUGGUAGCAGCCGAAAGGC | 591 |
| 0749 | 36-mer | UGC | |
| CD274- | Unmodified | AUACAGCUGAAUUGGUCAUAGCAGCCGAAAGGC | 592 |
| 0752 | 36-mer | UGC | |
| CD274- | Unmodified | AUGAAAGGACUCACUUGGUAGCAGCCGAAAGGC | 593 |
| 0800 | 36-mer | UGC | |
| CD274- | Unmodified | AAGGACUCACUUGGUAAUUAGCAGCCGAAAGGC | 594 |
| 0804 | 36-mer | UGC | |
| CD274- | Unmodified | GGUGUAGCACUGACAUUCAAGCAGCCGAAAGGC | 595 |
| 0847 | 36-mer | UGC | |
| CD274- | Unmodified | AGCACUGACAUUCAUCUUCAGCAGCCGAAAGGC | 596 |
| 0852 | 36-mer | UGC | |
| CD274- | Unmodified | AUGAUGGAUGUGAAAAAAUAGCAGCCGAAAGG | 597 |
| 0889 | 36-mer | CUGC | |
| CD274- | Unmodified | GGAGACCUUGAUACUUUCAAGCAGCCGAAAGGC | 598 |
| 1172 | 36-mer | UGC | |
| CD274- | Unmodified | GAGACCUUGAUACUUUCAAAGCAGCCGAAAGGC | 599 |
| 1173 | 36-mer | UGC | |
| CD274- | Unmodified | AGACCUUGAUACUUUCAAAAGCAGCCGAAAGGC | 600 |
| 1174 | 36-mer | UGC | |
| CD274- | Unmodified | ACCUUGAUACUUUCAAAUGAGCAGCCGAAAGGC | 601 |
| 1176 | 36-mer | UGC | |
| CD274- | Unmodified | CUUGAUACUUUCAAAUGCCAGCAGCCGAAAGGC | 602 |
| 1178 | 36-mer | UGC | |
| CD274- | Unmodified | GAAAGGAUACUUCUGAACAAGCAGCCGAAAGGC | 603 |
| 1227 | 36-mer | UGC | |
| CD274- | Unmodified | GGAUACUUCUGAACAAGGAAGCAGCCGAAAGGC | 604 |
| 1231 | 36-mer | UGC | |
| CD274- | Unmodified | CUAUUUAUUUUGAGUCUGUAGCAGCCGAAAGG | 605 |
| 1423 | 36-mer | CUGC | |
| CD274- | Unmodified | AUUUAUUUUGAGUCUGUGAAGCAGCCGAAAGG | 606 |
| 1425 | 36-mer | CUGC | |
| CD274- | Unmodified | UGAGUGUGGUUGUGAAUGAAGCAGCCGAAAGG | 607 |
| 1459 | 36-mer | CUGC | |
| CD274- | Unmodified | GAGUGUGGUUGUGAAUGAUAGCAGCCGAAAGG | 608 |
| 1460 | 36-mer | CUGC | |
| CD274- | Unmodified | AGUGUGGUUGUGAAUGAUUAGCAGCCGAAAGG | 609 |
| 1461 | 36-mer | CUGC | |
| CD274- | Unmodified | GUGUGGUUGUGAAUGAUUUAGCAGCCGAAAGG | 610 |
| 1462 | 36-mer | CUGC | |
| CD274- | Unmodified | UGUGGUUGUGAAUGAUUUCAGCAGCCGAAAGG | 611 |
| 1463 | 36-mer | CUGC | |
| CD274- | Unmodified | GUGGUUGUGAAUGAUUUCUAGCAGCCGAAAGG | 612 |
| 1464 | 36-mer | CUGC | |
| CD274- | Unmodified | UGGUUGUGAAUGAUUUCUUAGCAGCCGAAAGG | 613 |
| 1465 | 36-mer | CUGC | |
| CD274- | Unmodified | GUUGUGAAUGAUUUCUUUUAGCAGCCGAAAGG | 614 |
| 1467 | 36-mer | CUGC | |
| CD274- | Unmodified | UGUGAAUGAUUUCUUUUGAAGCAGCCGAAAGG | 615 |
| 1469 | 36-mer | CUGC | |
| CD274- | Unmodified | GUGAAUGAUUUCUUUUGAAAGCAGCCGAAAGG | 616 |
| 1470 | 36-mer | CUGC | |
| CD274- | Unmodified | AAUGAUUUCUUUUGAAGAUAGCAGCCGAAAGG | 617 |
| 1473 | 36-mer | CUGC | |
| CD274- | Unmodified | AUGAUUUCUUUUGAAGAUAAGCAGCCGAAAGG | 618 |
| 1474 | 36-mer | CUGC | |
| CD274- | Unmodified | CUUUUGAAGAUAUAUUGUAAGCAGCCGAAAGG | 619 |
| 1481 | 36-mer | CUGC | |
| CD274- | Unmodified | UUGAAGAUAUAUUGUAGUAAGCAGCCGAAAGG | 620 |
| 1484 | 36-mer | CUGC | |
| CD274- | Unmodified | GAAGAUAUAUUGUAGUAGAAGCAGCCGAAAGG | 621 |
| 1486 | 36-mer | CUGC | |
| CD274- | Unmodified | AGUAGAUGUUACAAUUUUGAGCAGCCGAAAGG | 622 |
| 1499 | 36-mer | CUGC | |
| CD274- | Unmodified | GUAGAUGUUACAAUUUUGUAGCAGCCGAAAGG | 623 |
| 1500 | 36-mer | CUGC | |
| CD274- | Unmodified | AAUGAUUUGCUCACAUCUAAGCAGCCGAAAGGC | 624 |
| 1541 | 36-mer | UGC | |
| CD274- | Unmodified | AAAACAUGGAGUAUUUGUAAGCAGCCGAAAGG | 625 |
| 1562 | 36-mer | CUGC | |
| CD274- | Unmodified | CAAGUAUACAUUGGAAGCAAGCAGCCGAAAGGC | 626 |
| 1606 | 36-mer | UGC | |
| CD274- | Unmodified | AAGUAUACAUUGGAAGCAUAGCAGCCGAAAGG | 627 |
| 1607 | 36-mer | CUGC | |
| CD274- | Unmodified | AGGAUGUCACCUUUAUUUAAGCAGCCGAAAGGC | 628 |
| 1649 | 36-mer | UGC | |
| CD274- | Unmodified | CUGUUCCAUUUAAAUAUCAAGCAGCCGAAAGGC | 629 |
| 1728 | 36-mer | UGC | |
| CD274- | Unmodified | UGUAACCACCCUGUUGUGAAGCAGCCGAAAGGC | 630 |
| 1794 | 36-mer | UGC | |
| CD274- | Unmodified | GUAACCACCCUGUUGUGAUAGCAGCCGAAAGGC | 63 |
| 1795 | 36-mer | UGC | |
| CD274- | Unmodified | GUUGUGAUAACCACUAUUAAGCAGCCGAAAGGC | 632 |
| 1806 | 36-mer | UGC | |
| CD274- | Unmodified | UUGUGAUAACCACUAUUAUAGCAGCCGAAAGGC | 633 |
| 1807 | 36-mer | UGC | |
| CD274- | Unmodified | UGUGAUAACCACUAUUAUUAGCAGCCGAAAGGC | 634 |
| 1808 | 36-mer | UGC | |
| CD274- | Unmodified | GUGAUAACCACUAUUAUUUAGCAGCCGAAAGGC | 635 |
| 1809 | 36-mer | UGC | |
| CD274- | Unmodified | UGAUAACCACUAUUAUUUUAGCAGCCGAAAGGC | 636 |
| 1810 | 36-mer | UGC | |
| CD274- | Unmodified | GAUAACCACUAUUAUUUUAAGCAGCCGAAAGGC | 637 |
| 1811 | 36-mer | UGC | |
| CD274- | Unmodified | AUAACCACUAUUAUUUUACAGCAGCCGAAAGGC | 638 |
| 1812 | 36-mer | UGC | |
| CD274- | Unmodified | CCACUGUCCUUUUAUAAUAAGCAGCCGAAAGGC | 639 |
| 1906 | 36-mer | UGC | |
| CD274- | Unmodified | CACUGUCCUUUUAUAAUACAGCAGCCGAAAGGC | 640 |
| 1907 | 36-mer | UGC | |
| CD274- | Unmodified | ACUGUCCUUUUAUAAUACAAGCAGCCGAAAGGC | 641 |
| 1908 | 36-mer | UGC | |
| CD274- | Unmodified | CUGUCCUUUUAUAAUACAAAGCAGCCGAAAGGC | 642 |
| 1909 | 36-mer | UGC | |
| CD274- | Unmodified | UGUCCUUUUAUAAUACAAUAGCAGCCGAAAGGC | 643 |
| 1910 | 36-mer | UGC | |
| CD274- | Unmodified | CCUUUUAUAAUACAAUUUAAGCAGCCGAAAGGC | 644 |
| 1913 | 36-mer | UGC | |
| CD274- | Unmodified | UUUUAUAAUACAAUUUACAAGCAGCCGAAAGG | 645 |
| 1915 | 36-mer | CUGC | |
| CD274- | Unmodified | AUAAUACAAUUUACAGCUAAGCAGCCGAAAGGC | 646 |
| 1919 | 36-mer | UGC | |
| CD274- | Unmodified | ACAGCUAUAUUUUACUUUAAGCAGCCGAAAGGC | 647 |
| 1931 | 36-mer | UGC | |
| CD274- | Unmodified | CUAUAUUUUACUUUAAGCAAGCAGCCGAAAGGC | 648 |
| 1935 | 36-mer | UGC | |
| CD274- | Unmodified | AUAUUUUACUUUAAGCAAUAGCAGCCGAAAGG | 649 |
| 1937 | 36-mer | CUGC | |
| CD274- | Unmodified | AUUGAAUCUACAGAUGUGAAGCAGCCGAAAGG | 650 |
| 2014 | 36-mer | CUGC | |
| CD274- | Unmodified | GAAAAUGUAUUAUUACAAUAGCAGCCGAAAGG | 651 |
| 2088 | 36-mer | CUGC | |
| CD274- | Unmodified | AUAAUCAGAGUAAUUUUCAAGCAGCCGAAAGG | 652 |
| 2221 | 36-mer | CUGC | |
| CD274- | Unmodified | UCAGAGUAAUUUUCAUUUAAGCAGCCGAAAGG | 653 |
| 2225 | 36-mer | CUGC | |
| CD274- | Unmodified | AGAGUAAUUUUCAUUUACAAGCAGCCGAAAGG | 654 |
| 2227 | 36-mer | CUGC | |
| CD274- | Unmodified | GAGUAAUUUUCAUUUACAAAGCAGCCGAAAGG | 655 |
| 2228 | 36-mer | CUGC | |
| CD274- | Unmodified | AGUAAUUUUCAUUUACAAAAGCAGCCGAAAGG | 656 |
| 2229 | 36-mer | CUGC | |
| CD274- | Unmodified | UAAUUUUCAUUUACAAAGAAGCAGCCGAAAGG | 657 |
| 2231 | 36-mer | CUGC | |
| CD274- | Unmodified | AUUUUCAUUUACAAAGAGAAGCAGCCGAAAGG | 658 |
| 2233 | 36-mer | CUGC | |
| CD274- | Unmodified | AAAAUAACCCUGAAAAAUAAGCAGCCGAAAGGC | 659 |
| 2263 | 36-mer | UGC | |
| CD274- | Unmodified | AUAACCCUGAAAAAUAACAAGCAGCCGAAAGGC | 660 |
| 2266 | 36-mer | UGC | |
| CD274- | Unmodified | AUAUAAUCUAAUGCUUGUUAGCAGCCGAAAGG | 661 |
| 2333 | 36-mer | CUGC | |
| CD274- | Unmodified | AUAAUCUAAUGCUUGUUUAAGCAGCCGAAAGG | 662 |
| 2335 | 36-mer | CUGC | |
| CD274- | Unmodified | AAUCUAAUGCUUGUUUAUAAGCAGCCGAAAGG | 663 |
| 2337 | 36-mer | CUGC | |
| CD274- | Unmodified | AUCUAAUGCUUGUUUAUAUAGCAGCCGAAAGG | 664 |
| 2338 | 36-mer | CUGC | |
| CD274- | Unmodified | UCUAAUGCUUGUUUAUAUAAGCAGCCGAAAGG | 665 |
| 2339 | 36-mer | CUGC | |
| CD274- | Unmodified | UAAUGCUUGUUUAUAUAGUAGCAGCCGAAAGG | 666 |
| 2341 | 36-mer | CUGC | |
| CD274- | Unmodified | AUGCUUGUUUAUAUAGUGUAGCAGCCGAAAGG | 667 |
| 2343 | 36-mer | CUGC | |
| CD274- | Unmodified | CUGGUAUUGUUUAACAGUUAGCAGCCGAAAGG | 668 |
| 2362 | 36-mer | CUGC | |
| CD274- | Unmodified | UGGUAUUGUUUAACAGUUCAGCAGCCGAAAGG | 669 |
| 2363 | 36-mer | CUGC | |
| CD274- | Unmodified | AAUUUUAAAUUCAUACCUUAGCAGCCGAAAGGC | 670 |
| 2407 | 36-mer | UGC | |
| CD274- | Unmodified | AGUCUACAUUUGGAAAUGUAGCAGCCGAAAGG | 671 |
| 2556 | 36-mer | CUGC | |
| CD274- | Unmodified | GUCUACAUUUGGAAAUGUAAGCAGCCGAAAGG | 672 |
| 2557 | 36-mer | CUGC | |
| CD274- | Unmodified | ACAUUUGGAAAUGUAUGUUAGCAGCCGAAAGG | 673 |
| 2561 | 36-mer | CUGC | |
| CD274- | Unmodified | CAUUUGGAAAUGUAUGUUAAGCAGCCGAAAGG | 674 |
| 2562 | 36-mer | CUGC | |
| CD274- | Unmodified | AUUUGGAAAUGUAUGUUAAAGCAGCCGAAAGG | 675 |
| 2563 | 36-mer | CUGC | |
| CD274- | Unmodified | UUGGAAAUGUAUGUUAAAAAGCAGCCGAAAGG | 676 |
| 2565 | 36-mer | CUGC | |
| CD274- | Unmodified | GAAAUGUAUGUUAAAAGCAAGCAGCCGAAAGG | 677 |
| 2568 | 36-mer | CUGC | |
| CD274- | Unmodified | CUGUGUACUUUGCUAUUUUAGCAGCCGAAAGGC | 678 |
| 2634 | 36-mer | UGC | |
| CD274- | Unmodified | GUGUACUUUGCUAUUUUUAAGCAGCCGAAAGG | 679 |
| 2636 | 36-mer | CUGC | |
| CD274- | Unmodified | UGUACUUUGCUAUUUUUAUAGCAGCCGAAAGG | 680 |
| 2637 | 36-mer | CUGC | |
| CD274- | Unmodified | ACUUUGCUAUUUUUAUUUAAGCAGCCGAAAGG | 681 |
| 2640 | 36-mer | CUGC | |
| CD274- | Unmodified | AUAUAGCAGAUGGAAUGAAAGCAGCCGAAAGG | 682 |
| 2673 | 36-mer | CUGC | |
| CD274- | Unmodified | GCAGAUGGAAUGAAUUUGAAGCAGCCGAAAGG | 683 |
| 2678 | 36-mer | CUGC | |
| CD274- | Unmodified | CAGAUGGAAUGAAUUUGAAAGCAGCCGAAAGG | 684 |
| 2679 | 36-mer | CUGC | |
| CD274- | Unmodified | AUAUACAUUUAGACAACCAAGCAGCCGAAAGGC | 685 |
| 2801 | 36-mer | UGC | |
| CD274- | Unmodified | AACCACCAUUUGUUAAGUAAGCAGCCGAAAGGC | 686 |
| 2815 | 36-mer | UGC | |
| CD274- | Unmodified | ACCAUUUGUUAAGUAUUUGAGCAGCCGAAAGG | 687 |
| 2819 | 36-mer | CUGC | |
| CD274- | Unmodified | AAAAGCAAUCUUAUUAUUAAGCAGCCGAAAGG | 688 |
| 2924 | 36-mer | CUGC | |
| CD274- | Unmodified | UAUUAUUAACUCUGUAUGAAGCAGCCGAAAGG | 689 |
| 2935 | 36-mer | CUGC | |
| CD274- | Unmodified | AUUAUUAACUCUGUAUGACAGCAGCCGAAAGGC | 690 |
| 2936 | 36-mer | UGC | |
| CD274- | Unmodified | UUAUUAACUCUGUAUGACAAGCAGCCGAAAGGC | 691 |
| 2937 | 36-mer | UGC | |
| CD274- | Unmodified | AUUAACUCUGUAUGACAGAAGCAGCCGAAAGGC | 692 |
| 2939 | 36-mer | UGC | |
| CD274- | Unmodified | AGUAUAAACUUCACUUUGAAGCAGCCGAAAGGC | 693 |
| 2994 | 36-mer | UGC | |
| CD274- | Unmodified | CUGUACUUGCAAAAUCACAAGCAGCCGAAAGGC | 694 |
| 3015 | 36-mer | UGC | |
| CD274- | Unmodified | ACUUGCAAAAUCACAUUUUAGCAGCCGAAAGGC | 695 |
| 3019 | 36-mer | UGC | |
| CD274- | Unmodified | CCUCAUUCGUUGUGCUUGAAGCAGCCGAAAGGC | 696 |
| 3094 | 36-mer | UGC | |
| CD274- | Unmodified | CUCAUUCGUUGUGCUUGAAAGCAGCCGAAAGGC | 697 |
| 3095 | 36-mer | UGC | |
| CD274- | Unmodified | CUUGUGUUAUCUGUUUGUAAGCAGCCGAAAGG | 698 |
| 3305 | 36-mer | CUGC | |
| CD274- | Unmodified | UUGUGUUAUCUGUUUGUACAGCAGCCGAAAGG | 699 |
| 3306 | 36-mer | CUGC | |
| CD274- | Unmodified | UGUGUUAUCUGUUUGUACAAGCAGCCGAAAGG | 700 |
| 3307 | 36-mer | CUGC | |
| CD274- | Unmodified | GUGUUAUCUGUUUGUACAUAGCAGCCGAAAGG | 701 |
| 3308 | 36-mer | CUGC | |
| CD274- | Unmodified | GUUAUCUGUUUGUACAUGUAGCAGCCGAAAGG | 702 |
| 3310 | 36-mer | CUGC | |
| CD274- | Unmodified | UUGUACAUGUGCAUUUGUAAGCAGCCGAAAGG | 703 |
| 3319 | 36-mer | CUGC | |
| CD274- | Unmodified | AUGUGCAUUUGUACAGUAAAGCAGCCGAAAGG | 704 |
| 3325 | 36-mer | CUGC | |
| CD274- | Unmodified | GUGUUCUUUGUGUGAAUUAAGCAGCCGAAAGG | 705 |
| 3355 | 36-mer | CUGC | |
| CD274- | Unmodified | GUUCUUUGUGUGAAUUACAAGCAGCCGAAAGG | 706 |
| 3357 | 36-mer | CUGC | |
| CD274- | Unmodified | UUGUGGUGUUGGAUUUGUAAGCAGCCGAAAGG | 707 |
| 3441 | 36-mer | CUGC | |
| CD274- | Unmodified | AUUCUGCAUUUGAUUGUCAAGCAGCCGAAAGGC | 708 |
| 3522 | 36-mer | UGC | |
| CD274- | Unmodified | CAUUUGAUUGUCACUUUUUAGCAGCCGAAAGGC | 709 |
| 3528 | 36-mer | UGC | |
| CD274- | Unmodified | UUGAUUGUCACUUUUUGUAAGCAGCCGAAAGG | 710 |
| 3531 | 36-mer | CUGC | |
| CD274- | Unmodified | UGAUUGUCACUUUUUGUACAGCAGCCGAAAGGC | 711 |
| 3532 | 36-mer | UGC | |
| CD274- | Unmodified | AUUGUCACUUUUUGUACCUAGCAGCCGAAAGGC | 712 |
| 3534 | 36-mer | UGC | |
| CD274- | Unmodified | CUUUUUGUACCUGCAUUAAAGCAGCCGAAAGGC | 713 |
| 3541 | 36-mer | UGC | |
| CD274- | Unmodified | UUUUUGUACCUGCAUUAAUAGCAGCCGAAAGGC | 714 |
| 3542 | 36-mer | UGC | |
| CD274- | Unmodified | UUGUACCUGCAUUAAUUUAAGCAGCCGAAAGGC | 715 |
| 3545 | 36-mer | UGC | |
| CD274- | Unmodified | UGUACCUGCAUUAAUUUAAAGCAGCCGAAAGGC | 716 |
| 3546 | 36-mer | UGC | |
| CD274- | Unmodified | AUAUUCUUAUUUAUUUUGUAGCAGCCGAAAGG | 717 |
| 3569 | 36-mer | CUGC | |
| CD274- | Unmodified | AUUUAUUUUGUUACUUGGUAGCAGCCGAAAGG | 718 |
| 3577 | 36-mer | CUGC | |
| CD274- | Unmodified | UUUAUUUUGUUACUUGGUAAGCAGCCGAAAGG | 719 |
| 3578 | 36-mer | CUGC | |
| CD274- | Unmodified | UAUUUUGUUACUUGGUACAAGCAGCCGAAAGG | 720 |
| 3580 | 36-mer | CUGC | |
| CD274- | Unmodified | AUGUCCAUUUUCUUGUUUAAGCAGCCGAAAGGC | 721 |
| 3604 | 36-mer | UGC | |
| CD274- | Unmodified | AUUUUCUUGUUUAUUUUGUAGCAGCCGAAAGG | 722 |
| 3610 | 36-mer | CUGC | |
| CD274- | Unmodified | AUUUUGUGUUUAAUAAAAUAGCAGCCGAAAGG | 723 |
| 3622 | 36-mer | CUGC | |
| CD274- | Unmodified | UCAAAUAUCCUCAUCUUUCUGG | 724 |
| 0088 | 22-mer | | |
| CD274- | Unmodified | UAGACAGCAAAUAUCCUCAUGG | 725 |
| 0094 | 22-mer | | |
| CD274- | Unmodified | UUAAAGACAGCAAAUAUCCUGG | 727 |
| 0097 | 22-mer | | |
| CD274- | Unmodified | UAUAAAGACAGCAAAUAUCCGG | 728 |
| 0098 | 22-mer | | |
| CD274- | Unmodified | UAUAUAAAGACAGCAAAUAUGG | 729 |
| 0100 | 22-mer | | |
| CD274- | Unmodified | UGAAUAUAAAGACAGCAAAUGG | 730 |
| 0102 | 22-mer | | |
| CD274- | Unmodified | UUCUACCACAUAUAGGUCCUGG | 731 |
| 0167 | 22-mer | | |
| CD274- | Unmodified | UAUUCAAUUGUCAUAUUGCUGG | 732 |
| 0193 | 22-mer | | |
| CD274- | Unmodified | UCAUUCAAUUGUCAUAUUGCGG | 733 |
| 0194 | 22-mer | | |
| CD274- | Unmodified | UUUGCAUUCAAUUGUCAUAUGG | 734 |
| 0197 | 22-mer | | |
| CD274- | Unmodified | UAAUUUGCAUUCAAUUGUCAGG | 735 |
| 0200 | 22-mer | | |
| CD274- | Unmodified | UUGGGAAUUUGCAUUCAAUUGG | 736 |
| 0204 | 22-mer | | |
| CD274- | Unmodified | UUAAUUGUUUUUCUACUGGGGG | 737 |
| 0219 | 22-mer | | |
| CD274- | Unmodified | UGUCUAAUUGUUUUUCUACUGG | 738 |
| 0222 | 22-mer | | |
| CD274- | Unmodified | UCAGGUCUAAUUGUUUUUCUGG | 739 |
| 0225 | 22-mer | | |
| CD274- | Unmodified | UUAAUGUUCUUAUCCUCCAUGG | 740 |
| 0268 | 22-mer | | |
| CD274- | Unmodified | UAUAAUGUUCUUAUCCUCCAGG | 741 |
| 0269 | 22-mer | | |
| CD274- | Unmodified | UGAAUAAUGUUCUUAUCCUCGG | 742 |
| 0271 | 22-mer | | |
| CD274- | Unmodified | UAUUGAAUAAUGUUCUUAUCGG | 743 |
| 0274 | 22-mer | | |
| CD274- | Unmodified | UAAUUGAAUAAUGUUCUUAUGG | 744 |
| 0275 | 22-mer | | |
| CD274- | Unmodified | UCAAAUUGAAUAAUGUUCUUGG | 745 |
| 0277 | 22-mer | | |
| CD274- | Unmodified | UAUGCACAAAUUGAAUAAUGGG | 746 |
| 0282 | 22-mer | | |
| CD274- | Unmodified | UCAUGCACAAAUUGAAUAAUGG | 747 |
| 0283 | 22-mer | | |
| CD274- | Unmodified | UGCAAUUUCACAUCUGUGAUGG | 748 |
| 0394 | 22-mer | | |
| CD274- | Unmodified | UAUCCUGCAAUUUCACAUCUGG | 749 |
| 0399 | 22-mer | | |
| CD274- | Unmodified | UUCAUGUUCAGAGGUGACUGGG | 750 |
| 0530 | 22-mer | | |
| CD274- | Unmodified | UUUCAUGUUCAGAGGUGACUGG | 751 |
| 0531 | 22-mer | | |
| CD274- | Unmodified | UACAUUGAAAAGCUUCUCCUGG | 752 |
| 0653 | 22-mer | | |
| CD274- | Unmodified | UUGAUUCUCAGUGUGCUGGUGG | 753 |
| 0673 | 22-mer | | |
| CD274- | Unmodified | UAAUCUCAUUAGUUGUUGUGGG | 754 |
| 0693 | 22-mer | | |
| CD274- | Unmodified | UAGAAAAUCUCAUUAGUUGUGG | 755 |
| 0697 | 22-mer | | |
| CD274- | Unmodified | UAGUAGAAAAUCUCAUUAGUGG | 756 |
| 0700 | 22-mer | | |
| CD274- | Unmodified | UCAGUAGAAAAUCUCAUUAGGG | 757 |
| 0701 | 22-mer | | |
| CD274- | Unmodified | UUCAGCUGUAUGGUUUUCCUGG | 758 |
| 0743 | 22-mer | | |
| CD274- | Unmodified | UAAUUCAGCUGUAUGGUUUUGG | 759 |
| 0746 | 22-mer | | |
| CD274- | Unmodified | UAAGACAGCAAAUAUCCUCAGG | 760 |
| 0095 | 22-mer | | |
| CD274- | Unmodified | UUAUAAAGACAGCAAAUAUCGG | 761 |
| 0099 | 22-mer | | |
| CD274- | Unmodified | UUGAAUAUAAAGACAGCAAAGG | 762 |
| 0103 | 22-mer | | |
| CD274- | Unmodified | UGCAUUCAAUUGUCAUAUUGGG | 763 |
| 0195 | 22-mer | | |
| CD274- | Unmodified | UUGCAUUCAAUUGUCAUAUUGG | 764 |
| 0196 | 22-mer | | |
| CD274- | Unmodified | UUUUGCAUUCAAUUGUCAUAGG | 765 |
| 0198 | 22-mer | | |
| CD274- | Unmodified | UAUUUGCAUUCAAUUGUCAUGG | 766 |
| 0199 | 22-mer | | |
| CD274- | Unmodified | UGAAUUUGCAUUCAAUUGUCGG | 767 |
| 0201 | 22-mer | | |
| CD274- | Unmodified | UGGAAUUUGCAUUCAAUUGUGG | 768 |
| 0202 | 22-mer | | |
| CD274- | Unmodified | UUACUGGGAAUUUGCAUUCAGG | 769 |
| 0207 | 22-mer | | |
| CD274- | Unmodified | UUCUAAUUGUUUUUCUACUGGG | 770 |
| 0221 | 22-mer | | |
| CD274- | Unmodified | UCAAUAGACAAUUAGUGCAGGG | 771 |
| 0245 | 22-mer | | |
| CD274- | Unmodified | UUCCCAAUAGACAAUUAGUGGG | 772 |
| 0248 | 22-mer | | |
| CD274- | Unmodified | UUUCCCAAUAGACAAUUAGUGG | 773 |
| 0249 | 22-mer | | |
| CD274- | Unmodified | UUGUUCUUAUCCUCCAUUUCGG | 774 |
| 0265 | 22-mer | | |
| CD274- | Unmodified | UAUGUUCUUAUCCUCCAUUUGG | 775 |
| 0266 | 22-mer | | |
| CD274- | Unmodified | UAAUGUUCUUAUCCUCCAUUGG | 776 |
| 0267 | 22-mer | | |
| CD274- | Unmodified | UAAUAAUGUUCUUAUCCUCCGG | 777 |
| 0270 | 22-mer | | |
| CD274- | Unmodified | UUGAAUAAUGUUCUUAUCCUGG | 778 |
| 0272 | 22-mer | | |
| CD274- | Unmodified | UUUGAAUAAUGUUCUUAUCCGG | 779 |
| 0273 | 22-mer | | |
| CD274- | Unmodified | UAAAUUGAAUAAUGUUCUUAGG | 780 |
| 0276 | 22-mer | | |
| CD274- | Unmodified | UACAAAUUGAAUAAUGUUCUGG | 781 |
| 0278 | 22-mer | | |
| CD274- | Unmodified | UCACAAAUUGAAUAAUGUUCGG | 782 |
| 0279 | 22-mer | | |
| CD274- | Unmodified | UGCACAAAUUGAAUAAUGUUGG | 783 |
| 0280 | 22-mer | | |
| CD274- | Unmodified | UUGCACAAAUUGAAUAAUGUGG | 784 |
| 0281 | 22-mer | | |
| CD274- | Unmodified | UUCCAUGCACAAAUUGAAUAGG | 785 |
| 0285 | 22-mer | | |
| CD274- | Unmodified | UAACCUUCAGGUCUUCCUCUGG | 786 |
| 0303 | 22-mer | | |
| CD274- | Unmodified | UUGAACCUUCAGGUCUUCCUGG | 787 |
| 0305 | 22-mer | | |
| CD274- | Unmodified | UUGCUGAACCUUCAGGUCUUGG | 788 |
| 0308 | 22-mer | | |
| CD274- | Unmodified | UAUGCUGAACCUUCAGGUCUGG | 789 |
| 0309 | 22-mer | | |
| CD274- | Unmodified | UUAUGCUGAACCUUCAGGUCGG | 790 |
| 0310 | 22-mer | | |
| CD274- | Unmodified | UACUAUGCUGAACCUUCAGGGG | 791 |
| 0312 | 22-mer | | |
| CD274- | Unmodified | UUACUAUGCUGAACCUUCAGGG | 792 |
| 0313 | 22-mer | | |
| CD274- | Unmodified | UAAUUUCACAUCUGUGAUCUGG | 793 |
| 0392 | 22-mer | | |
| CD274- | Unmodified | UUGCAAUUUCACAUCUGUGAGG | 794 |
| 0395 | 22-mer | | |
| CD274- | Unmodified | UUCCUGCAAUUUCACAUCUGGG | 795 |
| 0398 | 22-mer | | |
| CD274- | Unmodified | UAUUCUUUGGUUGAUUUUGUGG | 796 |
| 0497 | 22-mer | | |
| CD274- | Unmodified | UAAAUUCUUUGGUUGAUUUUGG | 797 |
| 0499 | 22-mer | | |
| CD274- | Unmodified | UAAAAUUCUUUGGUUGAUUUGG | 798 |
| 0500 | 22-mer | | |
| CD274- | Unmodified | UCAAAAUUCUUUGGUUGAUUGG | 799 |
| 0501 | 22-mer | | |
| CD274- | Unmodified | UCCAAAAUUCUUUGGUUGAUGG | 800 |
| 0502 | 22-mer | | |
| CD274- | Unmodified | UAACCAAAAUUCUUUGGUUGGG | 801 |
| 0504 | 22-mer | | |
| CD274- | Unmodified | UCAACCAAAAUUCUUUGGUUGG | 802 |
| 0505 | 22-mer | | |
| CD274- | Unmodified | UACAACCAAAAUUCUUUGGUGG | 803 |
| 0506 | 22-mer | | |
| CD274- | Unmodified | UCACAACCAAAAUUCUUUGGGG | 804 |
| 0507 | 22-mer | | |
| CD274- | Unmodified | UUCCACAACCAAAAUUCUUUGG | 805 |
| 0509 | 22-mer | | |
| CD274- | Unmodified | UAUCCACAACCAAAAUUCUUGG | 806 |
| 0510 | 22-mer | | |
| CD274- | Unmodified | UGAUCCACAACCAAAAUUCUGG | 807 |
| 0511 | 22-mer | | |
| CD274- | Unmodified | UUGGAUCCACAACCAAAAUUGG | 808 |
| 0513 | 22-mer | | |
| CD274- | Unmodified | UUCAGUUCAUGUUCAGAGGUGG | 809 |
| 0535 | 22-mer | | |
| CD274- | Unmodified | UUGUCAGUUCAUGUUCAGAGGG | 810 |
| 0537 | 22-mer | | |
| CD274- | Unmodified | UUGACAUGUCAGUUCAUGUUGG | 811 |
| 0542 | 22-mer | | |
| CD274- | Unmodified | UUGCUUGUCCAGAUGACUUCGG | 812 |
| 0583 | 22-mer | | |
| CD274- | Unmodified | UUUGAUGGUCACUGCUUGUCGG | 813 |
| 0594 | 22-mer | | |
| CD274- | Unmodified | UUCAGGACUUGAUGGUCACUGG | 814 |
| 0601 | 22-mer | | |
| CD274- | Unmodified | UCACAUUGAAAAGCUUCUCCGG | 815 |
| 0654 | 22-mer | | |
| CD274- | Unmodified | UUCACAUUGAAAAGCUUCUCGG | 816 |
| 0655 | 22-mer | | |
| CD274- | Unmodified | UGUCACAUUGAAAAGCUUCUGG | 817 |
| 0656 | 22-mer | | |
| CD274- | Unmodified | UUGGUCACAUUGAAAAGCUUGG | 818 |
| 0658 | 22-mer | | |
| CD274- | Unmodified | UUGCUGGUCACAUUGAAAAGGG | 819 |
| 0661 | 22-mer | | |
| CD274- | Unmodified | UUCAGUGUGCUGGUCACAUUGG | 820 |
| 0667 | 22-mer | | |
| CD274- | Unmodified | UUGUUGAUUCUCAGUGUGCUGG | 821 |
| 0676 | 22-mer | | |
| CD274- | Unmodified | UUUGUGUUGAUUCUCAGUGUGG | 822 |
| 0679 | 22-mer | | |
| CD274- | Unmodified | UUGUUGUGUUGAUUCUCAGUGG | 823 |
| 0681 | 22-mer | | |
| CD274- | Unmodified | UUAGUUGUUGUGUUGAUUCUGG | 824 |
| 0685 | 22-mer | | |
| CD274- | Unmodified | UAUCUCAUUAGUUGUUGUGUGG | 825 |
| 0692 | 22-mer | | |
| CD274- | Unmodified | UAAAAUCUCAUUAGUUGUUGGG | 826 |
| 0695 | 22-mer | | |
| CD274- | Unmodified | UGAAAAUCUCAUUAGUUGUUGG | 827 |
| 0696 | 22-mer | | |
| CD274- | Unmodified | UUAGAAAAUCUCAUUAGUUGGG | 828 |
| 0698 | 22-mer | | |
| CD274- | Unmodified | UUGCAGUAGAAAAUCUCAUUGG | 829 |
| 0703 | 22-mer | | |
| CD274- | Unmodified | UUAAAAGUGCAGUAGAAAAUGG | 830 |
| 0709 | 22-mer | | |
| CD274- | Unmodified | UAUUCAGCUGUAUGGUUUUCGG | 831 |
| 0745 | 22-mer | | |
| CD274- | Unmodified | UACCAAUUCAGCUGUAUGGUGG | 832 |
| 0749 | 22-mer | | |
| CD274- | Unmodified | UAUGACCAAUUCAGCUGUAUGG | 833 |
| 0752 | 22-mer | | |
| CD274- | Unmodified | UACCAAGUGAGUCCUUUCAUGG | 834 |
| 0800 | 22-mer | | |
| CD274- | Unmodified | UAAUUACCAAGUGAGUCCUUGG | 835 |
| 0804 | 22-mer | | |
| CD274- | Unmodified | UUGAAUGUCAGUGCUACACCGG | 836 |
| 0847 | 22-mer | | |
| CD274- | Unmodified | UGAAGAUGAAUGUCAGUGCUGG | 837 |
| 0852 | 22-mer | | |
| CD274- | Unmodified | UAUUUUUUCACAUCCAUCAUGG | 838 |
| 0889 | 22-mer | | |
| CD274- | Unmodified | UUGAAAGUAUCAAGGUCUCCGG | 839 |
| 1172 | 22-mer | | |
| CD274- | Unmodified | UUUGAAAGUAUCAAGGUCUCGG | 840 |
| 1173 | 22-mer | | |
| CD274- | Unmodified | UUUUGAAAGUAUCAAGGUCUGG | 841 |
| 1174 | 22-mer | | |
| CD274- | Unmodified | UCAUUUGAAAGUAUCAAGGUGG | 842 |
| 1176 | 22-mer | | |
| CD274- | Unmodified | UGGCAUUUGAAAGUAUCAAGGG | 843 |
| 1178 | 22-mer | | |
| CD274- | Unmodified | UUGUUCAGAAGUAUCCUUUCGG | 844 |
| 1227 | 22-mer | | |
| CD274- | Unmodified | UUCCUUGUUCAGAAGUAUCCGG | 845 |
| 1231 | 22-mer | | |
| CD274- | Unmodified | UACAGACUCAAAAUAAAUAGGG | 846 |
| 1423 | 22-mer | | |
| CD274- | Unmodified | UUCACAGACUCAAAAUAAAUGG | 847 |
| 1425 | 22-mer | | |
| CD274- | Unmodified | UUCAUUCACAACCACACUCAGG | 848 |
| 1459 | 22-mer | | |
| CD274- | Unmodified | UAUCAUUCACAACCACACUCGG | 849 |
| 1460 | 22-mer | | |
| CD274- | Unmodified | UAAUCAUUCACAACCACACUGG | 850 |
| 1461 | 22-mer | | |
| CD274- | Unmodified | UAAAUCAUUCACAACCACACGG | 851 |
| 1462 | 22-mer | | |
| CD274- | Unmodified | UGAAAUCAUUCACAACCACAGG | 852 |
| 1463 | 22-mer | | |
| CD274- | Unmodified | UAGAAAUCAUUCACAACCACGG | 853 |
| 1464 | 22-mer | | |
| CD274- | Unmodified | UAAGAAAUCAUUCACAACCAGG | 854 |
| 1465 | 22-mer | | |
| CD274- | Unmodified | UAAAAGAAAUCAUUCACAACGG | 855 |
| 1467 | 22-mer | | |
| CD274- | Unmodified | UUCAAAAGAAAUCAUUCACAGG | 856 |
| 1469 | 22-mer | | |
| CD274- | Unmodified | UUUCAAAAGAAAUCAUUCACGG | 857 |
| 1470 | 22-mer | | |
| CD274- | Unmodified | UAUCUUCAAAAGAAAUCAUUGG | 858 |
| 1473 | 22-mer | | |
| CD274- | Unmodified | UUAUCUUCAAAAGAAAUCAUGG | 859 |
| 1474 | 22-mer | | |
| CD274- | Unmodified | UUACAAUAUAUCUUCAAAAGGG | 860 |
| 1481 | 22-mer | | |
| CD274- | Unmodified | UUACUACAAUAUAUCUUCAAGG | 861 |
| 1484 | 22-mer | | |
| CD274- | Unmodified | UUCUACUACAAUAUAUCUUCGG | 862 |
| 1486 | 22-mer | | |
| CD274- | Unmodified | UCAAAAUUGUAACAUCUACUGG | 863 |
| 1499 | 22-mer | | |
| CD274- | Unmodified | UACAAAAUUGUAACAUCUACGG | 864 |
| 1500 | 22-mer | | |
| CD274- | Unmodified | UUAGAUGUGAGCAAAUCAUUGG | 865 |
| 1541 | 22-mer | | |
| CD274- | Unmodified | UUACAAAUACUCCAUGUUUUGG | 866 |
| 1562 | 22-mer | | |
| CD274- | Unmodified | UUGCUUCCAAUGUAUACUUGGG | 867 |
| 1606 | 22-mer | | |
| CD274- | Unmodified | UAUGCUUCCAAUGUAUACUUGG | 868 |
| 1607 | 22-mer | | |
| CD274- | Unmodified | UUAAAUAAAGGUGACAUCCUGG | 869 |
| 1649 | 22-mer | | |
| CD274- | Unmodified | UUGAUAUUUAAAUGGAACAGGG | 870 |
| 1728 | 22-mer | | |
| CD274- | Unmodified | UUCACAACAGGGUGGUUACAGG | 871 |
| 1794 | 22-mer | | |
| CD274- | Unmodified | UAUCACAACAGGGUGGUUACGG | 872 |
| 1795 | 22-mer | | |
| CD274- | Unmodified | UUAAUAGUGGUUAUCACAACGG | 873 |
| 1806 | 22-mer | | |
| CD274- | Unmodified | UAUAAUAGUGGUUAUCACAAGG | 874 |
| 1807 | 22-mer | | |
| CD274- | Unmodified | UAAUAAUAGUGGUUAUCACAGG | 875 |
| 1808 | 22-mer | | |
| CD274- | Unmodified | UAAAUAAUAGUGGUUAUCACGG | 876 |
| 1809 | 22-mer | | |
| CD274- | Unmodified | UAAAAUAAUAGUGGUUAUCAGG | 877 |
| 1810 | 22-mer | | |
| CD274- | Unmodified | UUAAAAUAAUAGUGGUUAUCGG | 878 |
| 1811 | 22-mer | | |
| CD274- | Unmodified | UGUAAAAUAAUAGUGGUUAUGG | 879 |
| 1812 | 22-mer | | |
| CD274- | Unmodified | UUAUUAUAAAAGGACAGUGGGG | 880 |
| 1906 | 22-mer | | |
| CD274- | Unmodified | UGUAUUAUAAAAGGACAGUGGG | 881 |
| 1907 | 22-mer | | |
| CD274- | Unmodified | UUGUAUUAUAAAAGGACAGUGG | 882 |
| 1908 | 22-mer | | |
| CD274- | Unmodified | UUUGUAUUAUAAAAGGACAGGG | 883 |
| 1909 | 22-mer | | |
| CD274- | Unmodified | UAUUGUAUUAUAAAAGGACAGG | 884 |
| 1910 | 22-mer | | |
| CD274- | Unmodified | UUAAAUUGUAUUAUAAAAGGGG | 885 |
| 1913 | 22-mer | | |
| CD274- | Unmodified | UUGUAAAUUGUAUUAUAAAAGG | 886 |
| 1915 | 22-mer | | |
| CD274- | Unmodified | UUAGCUGUAAAUUGUAUUAUGG | 887 |
| 1919 | 22-mer | | |
| CD274- | Unmodified | UUAAAGUAAAAUAUAGCUGUGG | 888 |
| 1931 | 22-mer | | |
| CD274- | Unmodified | UUGCUUAAAGUAAAAUAUAGGG | 889 |
| 1935 | 22-mer | | |
| CD274- | Unmodified | UAUUGCUUAAAGUAAAAUAUGG | 890 |
| 1937 | 22-mer | | |
| CD274- | Unmodified | UUCACAUCUGUAGAUUCAAUGG | 891 |
| 2014 | 22-mer | | |
| CD274- | Unmodified | UAUUGUAAUAAUACAUUUUCGG | 892 |
| 2088 | 22-mer | | |
| CD274- | Unmodified | UUGAAAAUUACUCUGAUUAUGG | 893 |
| 2221 | 22-mer | | |
| CD274- | Unmodified | UUAAAUGAAAAUUACUCUGAGG | 894 |
| 2225 | 22-mer | | |
| CD274- | Unmodified | UUGUAAAUGAAAAUUACUCUGG | 895 |
| 2227 | 22-mer | | |
| CD274- | Unmodified | UUUGUAAAUGAAAAUUACUCGG | 896 |
| 2228 | 22-mer | | |
| CD274- | Unmodified | UUUUGUAAAUGAAAAUUACUGG | 897 |
| 2229 | 22-mer | | |
| CD274- | Unmodified | UUCUUUGUAAAUGAAAAUUAGG | 898 |
| 2231 | 22-mer | | |
| CD274- | Unmodified | UUCUCUUUGUAAAUGAAAAUGG | 899 |
| 2233 | 22-mer | | |
| CD274- | Unmodified | UUAUUUUUCAGGGUUAUUUUGG | 900 |
| 2263 | 22-mer | | |
| CD274- | Unmodified | UUGUUAUUUUUCAGGGUUAUGG | 901 |
| 2266 | 22-mer | | |
| CD274- | Unmodified | UAACAAGCAUUAGAUUAUAUGG | 902 |
| 2333 | 22-mer | | |
| CD274- | Unmodified | UUAAACAAGCAUUAGAUUAUGG | 903 |
| 2335 | 22-mer | | |
| CD274- | Unmodified | UUAUAAACAAGCAUUAGAUUGG | 904 |
| 2337 | 22-mer | | |
| CD274- | Unmodified | UAUAUAAACAAGCAUUAGAUGG | 905 |
| 2338 | 22-mer | | |
| CD274- | Unmodified | UUAUAUAAACAAGCAUUAGAGG | 906 |
| 2339 | 22-mer | | |
| CD274- | Unmodified | UACUAUAUAAACAAGCAUUAGG | 907 |
| 2341 | 22-mer | | |
| CD274- | Unmodified | UACACUAUAUAAACAAGCAUGG | 908 |
| 2343 | 22-mer | | |
| CD274- | Unmodified | UAACUGUUAAACAAUACCAGGG | 909 |
| 2362 | 22-mer | | |
| CD274- | Unmodified | UGAACUGUUAAACAAUACCAGG | 910 |
| 2363 | 22-mer | | |
| CD274- | Unmodified | UAAGGUAUGAAUUUAAAAUUGG | 911 |
| 2407 | 22-mer | | |
| CD274- | Unmodified | UACAUUUCCAAAUGUAGACUGG | 912 |
| 2556 | 22-mer | | |
| CD274- | Unmodified | UUACAUUUCCAAAUGUAGACGG | 913 |
| 2557 | 22-mer | | |
| CD274- | Unmodified | UAACAUACAUUUCCAAAUGUGG | 914 |
| 2561 | 22-mer | | |
| CD274- | Unmodified | UUAACAUACAUUUCCAAAUGGG | 915 |
| 2562 | 22-mer | | |
| CD274- | Unmodified | UUUAACAUACAUUUCCAAAUGG | 916 |
| 2563 | 22-mer | | |
| CD274- | Unmodified | UUUUUAACAUACAUUUCCAAGG | 917 |
| 2565 | 22-mer | | |
| CD274- | Unmodified | UUGCUUUUAACAUACAUUUCGG | 918 |
| 2568 | 22-mer | | |
| CD274- | Unmodified | UAAAAUAGCAAAGUACACAGGG | 919 |
| 2634 | 22-mer | | |
| CD274- | Unmodified | UUAAAAAUAGCAAAGUACACGG | 920 |
| 2636 | 22-mer | | |
| CD274- | Unmodified | UAUAAAAAUAGCAAAGUACAGG | 921 |
| 2637 | 22-mer | | |
| CD274- | Unmodified | UUAAAUAAAAAUAGCAAAGUGG | 922 |
| 2640 | 22-mer | | |
| CD274- | Unmodified | UUUCAUUCCAUCUGCUAUAUGG | 923 |
| 2673 | 22-mer | | |
| CD274- | Unmodified | UUCAAAUUCAUUCCAUCUGCGG | 924 |
| 2678 | 22-mer | | |
| CD274- | Unmodified | UUUCAAAUUCAUUCCAUCUGGG | 925 |
| 2679 | 22-mer | | |
| CD274- | Unmodified | UUGGUUGUCUAAAUGUAUAUGG | 926 |
| 2801 | 22-mer | | |
| CD274- | Unmodified | UUACUUAACAAAUGGUGGUUGG | 927 |
| 2815 | 22-mer | | |
| CD274- | Unmodified | UCAAAUACUUAACAAAUGGUGG | 928 |
| 2819 | 22-mer | | |
| CD274- | Unmodified | UUAAUAAUAAGAUUGCUUUUGG | 929 |
| 2924 | 22-mer | | |
| CD274- | Unmodified | UUCAUACAGAGUUAAUAAUAGG | 930 |
| 2935 | 22-mer | | |
| CD274- | Unmodified | UGUCAUACAGAGUUAAUAAUGG | 931 |
| 2936 | 22-mer | | |
| CD274- | Unmodified | UUGUCAUACAGAGUUAAUAAGG | 932 |
| 2937 | 22-mer | | |
| CD274- | Unmodified | UUCUGUCAUACAGAGUUAAUGG | 933 |
| 2939 | 22-mer | | |
| CD274- | Unmodified | UUCAAAGUGAAGUUUAUACUGG | 934 |
| 2994 | 22-mer | | |
| CD274- | Unmodified | UUGUGAUUUUGCAAGUACAGGG | 935 |
| 3015 | 22-mer | | |
| CD274- | Unmodified | UAAAAUGUGAUUUUGCAAGUGG | 936 |
| 3019 | 22-mer | | |
| CD274- | Unmodified | UUCAAGCACAACGAAUGAGGGG | 937 |
| 3094 | 22-mer | | |
| CD274- | Unmodified | UUUCAAGCACAACGAAUGAGGG | 938 |
| 3095 | 22-mer | | |
| CD274- | Unmodified | UUACAAACAGAUAACACAAGGG | 939 |
| 3305 | 22-mer | | |
| CD274- | Unmodified | UGUACAAACAGAUAACACAAGG | 940 |
| 3306 | 22-mer | | |
| CD274- | Unmodified | UUGUACAAACAGAUAACACAGG | 941 |
| 3307 | 22-mer | | |
| CD274- | Unmodified | UAUGUACAAACAGAUAACACGG | 942 |
| 3308 | 22-mer | | |
| CD274- | Unmodified | UACAUGUACAAACAGAUAACGG | 943 |
| 3310 | 22-mer | | |
| CD274- | Unmodified | UUACAAAUGCACAUGUACAAGG | 944 |
| 3319 | 22-mer | | |
| CD274- | Unmodified | UUUACUGUACAAAUGCACAUGG | 945 |
| 3325 | 22-mer | | |
| CD274- | Unmodified | UUAAUUCACACAAAGAACACGG | 946 |
| 3355 | 22-mer | | |
| CD274- | Unmodified | UUGUAAUUCACACAAAGAACGG | 947 |
| 3357 | 22-mer | | |
| CD274- | Unmodified | UUACAAAUCCAACACCACAAGG | 948 |
| 3441 | 22-mer | | |
| CD274- | Unmodified | UUGACAAUCAAAUGCAGAAUGG | 949 |
| 3522 | 22-mer | | |
| CD274- | Unmodified | UAAAAAGUGACAAUCAAAUGGG | 950 |
| 3528 | 22-mer | | |
| CD274- | Unmodified | UUACAAAAAGUGACAAUCAAGG | 951 |
| 3531 | 22-mer | | |
| CD274- | Unmodified | UGUACAAAAAGUGACAAUCAGG | 952 |
| 3532 | 22-mer | | |
| CD274- | Unmodified | UAGGUACAAAAAGUGACAAUGG | 953 |
| 3534 | 22-mer | | |
| CD274- | Unmodified | UUUAAUGCAGGUACAAAAAGGG | 954 |
| 3541 | 22-mer | | |
| CD274- | Unmodified | UAUUAAUGCAGGUACAAAAAGG | 955 |
| 3542 | 22-mer | | |
| CD274- | Unmodified | UUAAAUUAAUGCAGGUACAAGG | 956 |
| 3545 | 22-mer | | |
| CD274- | Unmodified | UUUAAAUUAAUGCAGGUACAGG | 957 |
| 3546 | 22-mer | | |
| CD274- | Unmodified | UACAAAAUAAAUAAGAAUAUGG | 958 |
| 3569 | 22-mer | | |
| CD274- | Unmodified | UACCAAGUAACAAAAUAAAUGG | 959 |
| 3577 | 22-mer | | |
| CD274- | Unmodified | UUACCAAGUAACAAAAUAAAGG | 960 |
| 3578 | 22-mer | | |
| CD274- | Unmodified | UUGUACCAAGUAACAAAAUAGG | 961 |
| 3580 | 22-mer | | |
| CD274- | Unmodified | UUAAACAAGAAAAUGGACAUGG | 962 |
| 3604 | 22-mer | | |
| CD274- | Unmodified | UACAAAAUAAACAAGAAAAUGG | 963 |
| 3610 | 22-mer | | |
| CD274- | Unmodified | UAUUUUAUUAAACACAAAAUGG | 964 |
| 3622 | 22-mer | | |
| CD274- | Modified 36- | [mAs][mG][mA][mA][mA][mG][mA][fU][fG][fA][fG] | 965 |
| 0088 | mer | [mG][mA][mU][mA][mU][mU][mU][mG][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mU][mG][mA][mG][mG][mA][fU][fA][fU][fU] | 966 |
| 0094 | mer | [mU][mG][mC][mU][mG][mU][mC][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mG][mG][mA][mU][mA][mU][fU][fU][fG][fC] | 968 |
| 0097 | mer | [mU][mG][mU][mC][mU][mU][mU][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mGs][mG][mA][mU][mA][mU][mU][fU][fG][fC][fU] | 969 |
| 0098 | mer | [mG][mU][mC][mU][mU][mU][mA][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mU][mA][mU][mU][mU][mG][fC][fU][fG][fU] | 970 |
| 0100 | mer | [mC][mU][mU][mU][mA][mU][mA][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mU][mU][mU][mG][mC][mU][fG][fU][fC][fU] | 971 |
| 0102 | mer | [mU][mU][mA][mU][mA][mU][mU][mC][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mG][mG][mA][mC][mC][mU][fA][fU][fA][fU] | 972 |
| 0167 | mer | [mG][mU][mG][mG][mU][mA][mG][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mGs][mC][mA][mA][mU][mA][mU][fG][fA][fC][fA] | 973 |
| 0193 | mer | [mA][mU][mU][mG][mA][mA][mU][mG][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mU][mA][mU][mG][mA][mC][fA][fA][fU][fU] | 974 |
| 0194 | mer | [mG][mA][mA][mU][mG][mC][mA][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mUs][mG][mA][mC][mA][mA][mU][fU][fG][fA][fA] | 975 |
| 0197 | mer | [mU][mG][mC][mA][mA][mA][mU][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mA][mU][mU][mG][mA][mA][fU][fG][fC][fA] | 976 |
| 0200 | mer | [mA][mA][mU][mU][mC][mC][mC][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mCs][mC][mC][mA][mG][mU][mA][fG][fA][fA][fA] | 977 |
| 0204 | mer | [mA][mA][mC][mA][mA][mU][mU][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mG][mU][mA][mG][mA][mA][fA][fA][fA][fC] | 978 |
| 0219 | mer | [mA][mA][mU][mU][mA][mG][mA][mC][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mG][mA][mA][mA][mA][mA][fC][fA][fA][fU] | 979 |
| 0222 | mer | [mU][mA][mG][mA][mC][mC][mU][mG][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mU][mG][mG][mA][mG][mG][fA][fU][fA][fA] | 980 |
| 0225 | mer | [mG][mA][mA][mC][mA][mU][mU][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mUs][mG][mG][mA][mG][mG][mA][fU][fA][fA][fG] | 981 |
| 0268 | mer | [mA][mA][mC][mA][mU[mU][mA][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mGs][mA][mG][mG][mA][mU][mA][fA][fG][fA][fA] | 982 |
| 0269 | mer | [mC][mA][mU][mU][mA][mU][mU][mC][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mG][mC][mA][mA][mU][mA][fU][fG][fA][fC] | 983 |
| 0271 | mer | [mA][mA][mU][mU][mG][mA][mA][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mGs][mA][mU][mA][mA][mG][mA][fA][fC][fA][fU] | 984 |
| 0274 | mer | [mU][mA][mU][mU][mC][mA][mA][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mU][mA][mA][mG][mA][mA][fC][fA][fU][fU] | 985 |
| 0275 | mer | [mA][mU][mU][mC][mA][mA][mU][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mA][mG][mA][mA][mC][mA][fU][fU][fA][fU] | 986 |
| 0277 | mer | [mU][mC][mA][mA][mU][mU][mU][mG][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mCs][mA][mU][mU][mA][mU][mU][fC][fA][fA][fU] | 987 |
| 0282 | mer | [mU][mU][mG][mU][mG][mC][mA][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mU][mU][mA][mU][mU][mC][fA][fA][fU][fU] | 988 |
| 0283 | mer | [mU][mG][mU][mG][mC][mA][mU][mG][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mU][mC][mA][mC][mA][mG][fA][fU][fG][fU] | 989 |
| 0394 | mer | [mG][mA][mA][mA][mU][mU][mG][mC][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mG][mA][mU][mG][mU][mG][fA][fA][fA][fU] | 990 |
| 0399 | mer | [mU][mG][mC][mA][mG][mG][mA][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mCs][mA][mG][mU][mC][mA][mC][fC][fU][fC][fU] | 991 |
| 0530 | mer | [mG][mA][mA][mC][mA][mU][mG][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mG][mU][mC][mA][mC][mC][fU][fC][fU][fG] | 992 |
| 0531 | mer | [mA][mA][mC][mA][mU][mG][mA][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mG][mG][mA][mG][mA][mA][fG][fC][fU][fU] | 993 |
| 0653 | mer | [mU][mU][mC][mA][mA][mU][mG][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mC][mC][mA][mG][mC][mA][fC][fA][fC][fU] | 994 |
| 0673 | mer | [mG][mA][mG][mA][mA][mU][mC][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mCs][mA][mC][mA][mA][mC][mA][fA][fC][fU][fA] | 995 |
| 0693 | mer | [mA][mU][mG][mA][mG][mA][mU][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mC][mA][mA][mC][mU][mA][fA][fU][fG][fA] | 996 |
| 0697 | mer | [mG][mA][mU][mU][mU][mU][mC][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mC][mU][mA][mA][mU][mG][fA][fG][fA][fU] | 997 |
| 0700 | mer | [mU][mU][mU][mC][mU][mA][mC][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mCs][mU][mA][mA][mU][mG][mA][fG][fA][fU][fU] | 998 |
| 0701 | mer | [mU][mU][mC][mU][mA][mC][mU][mG][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mG][mG][mA][mA][mA][mA][fC][fC][fA][fU] | 999 |
| 0743 | mer | [mA][mC][mA][mG][mC][mU][mG][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 36- | [mAs][mA][mA][mA][mC][mC][mA][fU][fA][fC][fA] | 1000 |
| 0746 | mer | [mG][mC][mU][mG][mA][mA][mU][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| CD274- | Modified 22- | [MePhosphonate-4O- | 1001 |
| 0088 | mer | mUs][fCs][fAs][fA][fA][mU][fA][mU][mC][fC][mU] | |
| | | [mC][mA][fU][mC][mU][mU][mU][mC][mUs][mGs][mG] | |
| CD274- | Modified 22- | [MePhosphonate-4O- | 1002 |
| 0094 | mer | mUs][fAs][fGs][fA][fC][mA][fG][mC][mA][fA][mA] | |
| | | [mU][mA][fU][mC][mC][mU][mC][mA][mUs][mGs][mG] | |
| CD274- | Modified 22- | [MePhosphonate-4O- | 1004 |
| 0097 | mer | mUs][fUs][fAs][fA][fA][mG][fA][mC][mA][fG][mC] | |
| | | [mA][mA][fA][mU][mA][mU][mC][mC][mUs][mGs][mG] | |
| CD274- | Modified 22- | [MePhosphonate-4O- | 1005 |
| 0098 | mer | mUs][fAs][fUs][fA][fA][mA][fG][mA][mC][fA][mG] | |
| | | [mC][mA][fA][mA][mU][mA][mU][mC][mCs][mGs][mG] | |
| CD274- | Modified 22- | [MePhosphonate-4O- | 1006 |
| 0100 | mer | mUs][fAs][fUs][fA][fU][mA][fA][mA][mG][fA][mC] | |
| | | [mA][mG][fC][mA][mA][mA][mU][mA][mUs][mGs][mG] | |
| CD274- | Modified 22- | [MePhosphonate-4O- | 1007 |
| 0102 | mer | mUs][fGs][fAs][fA][fU][mA][fU][mA][mA][fA][mG] | |
| | | [mA][mC][fA][mG][mC][mA][mA][mA][mUs][mGs][mG] | |
| CD274- | Modified 22- | [MePhosphonate-4O- | 1008 |
| 0167 | mer | mUs][fUs][fCs][fU][fA][mC][fC][mA][mC][fA][mU] | |
| | | [mA][mU][fA][mG][mG][mU][mC][mC][mUs][mGs][mG] | |
| CD274- | Modified 22- | [MePhosphonate-4O- | 1009 |
| 0193 | mer | mUs][fCs][fAs][fU][fU][mC][fA][mA][mU][fU][mG] | |
| | | [mU][mC][fA][mU][mA][mU][mU][mG][mCs][mGs][mG] | |
| CD274- | Modified 22- | [MePhosphonate-4O- | 1010 |
| 0194 | mer | mUs][fUs][fUs][fG][fC][mA][fU][mU][mC][fA][mA] | |
| | | [mU][mU][fG][mU][mC][mA][mU][mA][mUs][mGs][mG] | |
| CD274- | Modified 22- | [MePhosphonate-4O- | 1011 |
| 0197 | mer | mUs][fAs][fAs][fU][fU][mU][fG][mC][mA][fU][mU] | |
| | | [mC][mA][fA][mU][mU][mG][mU][mC][mAs][mGs][mG] | |
| CD274- | Modified 22- | [MePhosphonate-4O- | 1012 |
| 0200 | mer | mUs][fUs][fGs][fG][fG][mA][fA][mU][mU][fU][mG] | |
| | | [mC][mA][fU][mU][mC][mA][mA][mU][mUs][mGs][mG] | |
| CD274- | Modified 22- | [MePhosphonate-4O- | 1013 |
| 0204 | mer | mUs][fUs][fAs][fA][fU][mU][fG][mU][mU][fU][mU] | |
| | | [mU][mC][fU][mA][mC][mU][mG][mG][mGs][mGs][mG] | |
| CD274- | Modified 22- | [MePhosphonate-4O- | 1014 |
| 0219 | mer | mUs][fGs][fUs][fC][fU][mA][fA][mU][mU][fG][mU] | |
| | | U[m][mU][fU][mU][mC][mU][mA][mC][mUs][mGs][mG] | |
| CD274- | Modified 22- | [MePhosphonate-4O- | 1015 |
| 0222 | mer | mUs][fCs][fAs][fG][fG][mU][fC][mU][mA][fA][mU] | |
| | | [mU][mG][fU][mU][mU][mU][mU][mC][mUs][mGs][mG] | |
| CD274- | Modified 22- | [MePhosphonate-4O- | 1016 |
| 0225 | mer | mUs][fUs][fAs][fA][fU][mG][fU][mU][mC][fU][mU] | |
| | | A[m][mU][fC][mC][mU][mC][mC][mA][mUs][mGs][mG] | |
| CD274- | Modified 22- | [MePhosphonate-4O- | 1017 |
| 0268 | mer | mUs][fAs][fUs][fA][fA][mU][fG][mU][mU][fC][mU] | |
| | | [mU][mA][fU][mC][mC][mU][mC][mC][mAs][mGs][mG] | |
| CD274- | Modified 22- | [MePhosphonate-4O- | 1018 |
| 0269 | mer | mUs][fGs][fAs][fA][fU][mA][fA][mU][mG][fU][mU] | |
| | [m | C][mU][fU][mA][mU][mC][mC][mU][mCs][mGs][mG] | |
| CD274- | Modified 22- | [MePhosphonate-4O- | 1019 |
| 0271 | mer | mUs][fAs][fU][fU][fC][mA][fA][mU][mU][fG][mU] | |
| | | [mC][mA][fU][mA][mU][mU][mG][mC][mUs][mGs][mG] | |
| CD274- | Modified 22- | [MePhosphonate-4O- | 1020 |
| 0274 | mer | mUs][fAs][fUs][fU][fG][mA][fA][mU][mA][fA][mU] | |
| | | [mG][mU][fU][mC][mU][mU][mA][mU][mCs][mGs][mG] | |
| CD274- | Modified 22- | [MePhosphonate-4O- | 1021 |
| 0275 | mer | mUs][fAs][fAs][fU][fU][mG][fA][mA][mU][fA][mA] | |
| | | [mU][mG][fU][mU][mC][mU][mU][mA][mUs][mGs][mG] | |
| CD274- | Modified 22- | [MePhosphonate-4O- | 1022 |
| 0277 | mer | mUs][fCs][fAs][fA][fA][mU][fU][mG][mA][fA][mU] | |
| | | [mA][mA][fU][mG][mU][mU][mC][mU][mUs][mGs][mG] | |
| CD274- | Modified 22- | [MePhosphonate-4O- | 1023 |
| 0282 | mer | mUs][fAs][fUs][fG][fC][mA][fC][mA][mA][fA][mU] | |
| | | [mU][mG][fA][mA][mU][mA][mA][mU][mGs][mGs][mG] | |
| CD274- | Modified 22- | [MePhosphonate-4O- | 1024 |
| 0283 | mer | mUs][fCs][fAs][fU][fG][mC][fA][mC][mA][fA][mA] | |
| | | [mU][mU][fG][mA][mA][mU][mA][mA][mUs][mGs][mG] | |
| CD274- | Modified 22- | [MePhosphonate-4O- | 1025 |
| 0394 | mer | mUs][fGs][fCs][fA][fA][mU][fU][mU][mC][fA][mC] | |
| | | [mA][mU][fC][mU][mG][mU][mG][mA][mUs][mGs][mG] | |
| CD274- | Modified 22- | [MePhosphonate-4O- | 1026 |
| 0399 | mer | mUs][fAs][fUs][fC][fC][mU][fG][mC][mA][fA][mU] | |
| | | [mU][mU][fC][mA][mC][mA][mU][mC][mUs][mGs][mG] | |
| CD274- | Modified 22- | [MePhosphonate-4O- | 1027 |
| 0530 | mer | mUs][fUs][fCs][fA][fU][mG][fU][mU][mC][fA][mG] | |
| | | [mA][mG][fG][mU][mG][mA][mC][mU][mGs][mGs][mG] | |
| CD274- | Modified 22- | [MePhosphonate-4O- | 1028 |
| 0531 | mer | mUs][fUs][fUs][fC][fA][mU][fG][mU][mU][fC][mA] | |
| | | [mG][mA][fG][mG][mU][mG][mA][mC][mUs][mGs][mG] | |
| CD274- | Modified 22- | [MePhosphonate-4O- | 1029 |
| 0653 | mer | mUs][fAs][fCs][fA][fU][mU][fG][mA][mA][fA][mA] | |
| | | [mG][mC][fU][mU][mC][mU][mC][mC][mUs][mGs][mG] | |
| CD274- | Modified 22- | [MePhosphonate-4O- | 1030 |
| 0673 | mer | mUs][fUs][fGs][fA][fU][mU][fC][mU][mC][fA][mG] | |
| | | [mU][mG][fU][mG][mC][mU][mG][mG][mUs][mGs][mG] | |
| CD274- | Modified 22- | [MePhosphonate-4O- | 1031 |
| 0693 | mer | mUs][fAs][fAs][fU][fC][mU][fC][mA][mU][fU][mA] | |
| | | [mG][mU][fU][mG][mU][mU][mG][mU][mGs][mGs][mG] | |
| CD274- | Modified 22- | [MePhosphonate-4O- | 1032 |
| 0697 | mer | mUs][fAs][fGs][fA][fA][mA][fA][mU][mC][fU][mC] | |
| | | [mA][mU][fU][mA][mG][mU][mU][mG][mUs][mGs][mG] | |
| CD274- | Modified 22- | [MePhosphonate-4O- | 1033 |
| 0700 | mer | mUs][fAs][fGs][fU][fA][mG][fA][mA][mA][fA][mU] | |
| | | [mC][mU][fC][mA][mU][mU][mA][mG][mUs][mGs][mG] | |
| CD274- | Modified 22- | [MePhosphonate-4O- | 1034 |
| 0701 | mer | mUs][fCs][fAs][fG][fU][mA][fG][mA][mA][fA][mA] | |
| | | [mU][mC][fU][mC][mA][mU][mU][mA][mGs][mGs][mG] | |
| CD274- | Modified 22- | [MePhosphonate-4O- | 1035 |
| 0743 | mer | mUs][fUs][fCs][fA][fG][mC][fU][mG][mU][fA][mU] | |
| | | [mG][mG][fU][mU][mU][mU][mC][mC][mUs][mGs][mG] | |
| CD274- | Modified 22- | [MePhosphonate-4O- | 1036 |
| 0746 | mer | mUs][fAs][fAs][fU][fU][mC][fA][mG][mC][fU][mG] | |
| | | [mU][mA][fU][mG][mG][mU][mU][mU][mUs][mGs][mG] | |
| Human | RefSeq | AGTTCTGCGCAGCTTCCCGAGGCTCCGCACCAGC | 1037 |
| CD274 | NM_014143.4 | CGCGCTTCTGTCCGCCTGCAGGGCATTCCAGAAA | |
| mRNA | | GATGAGGATATTTGCTGTCTTTATATTCATGACCT | |
| | | ACTGGCATTTGCTGAACGCATTTACTGTCACGGT | |
| | | TCCCAAGGACCTATATGTGGTAGAGTATGGTAGC | |
| | | AATATGACAATTGAATGCAAATTCCCAGTAGAAA | |
| | | AACAATTAGACCTGGCTGCACTAATTGTCTATTG | |
| | | GGAAATGGAGGATAAGAACATTATTCAATTTGTG | |
| | | CATGGAGAGGAAGACCTGAAGGTTCAGCATAGT | |
| | | AGCTACAGACAGAGGGCCCGGCTGTTGAAGGAC | |
| | | CAGCTCTCCCTGGGAAATGCTGCACTTCAGATCA | |
| | | CAGATGTGAAATTGCAGGATGCAGGGGTGTACC | |
| | | GCTGCATGATCAGCTATGGTGGTGCCGACTACAA | |
| | | GCGAATTACTGTGAAAGTCAATGCCCCATACAAC | |
| | | AAAATCAACCAAAGAATTTTGGTTGTGGATCCAG | |
| | | TCACCTCTGAACATGAACTGACATGTCAGGCTGA | |
| | | GGGCTACCCCAAGGCCGAAGTCATCTGGACAAG | |
| | | CAGTGACCATCAAGTCCTGAGTGGTAAGACCACC | |
| | | ACCACCAATTCCAAGAGAGAGGAGAAGCTTTTCA | |
| | | ATGTGACCAGCACACTGAGAATCAACACAACAA | |
| | | CTAATGAGATTTTCTACTGCACTTTTAGGAGATTA | |
| | | GATCCTGAGGAAAACCATACAGCTGAATTGGTCA | |
| | | TCCCAGAACTACCTCTGGCACATCCTCCAAATGA | |
| | | AAGGACTCACTTGGTAATTCTGGGAGCCATCTTA | |
| | | TTATGCCTTGGTGTAGCACTGACATTCATCTTCCG | |
| | | TTTAAGAAAAGGGAGAATGATGGATGTGAAAAA | |
| | | ATGTGGCATCCAAGATACAAACTCAAAGAAGCA | |
| | | AAGTGATACACATTTGGAGGAGACGTAATCCAGC | |
| | | ATTGGAACTTCTGATCTTCAAGCAGGGATTCTCA | |
| | | ACCTGTGGTTTAGGGGTTCATCGGGGCTGAGCGT | |
| | | GACAAGAGGAAGGAATGGGCCCGTGGGATGCAG | |
| | | GCAATGTGGGACTTAAAAGGCCCAAGCACTGAA | |
| | | AATGGAACCTGGCGAAAGCAGAGGAGGAGAATG | |
| | | AAGAAAGATGGAGTCAAACAGGGAGCCTGGAGG | |
| | | GAGACCTTGATACTTTCAAATGCCTGAGGGGCTC | |
| | | ATCGACGCCTGTGACAGGGAGAAAGGATACTTCT | |
| | | GAACAAGGAGCCTCCAAGCAAATCATCCATTGCT | |
| | | CATCCTAGGAAGACGGGTTGAGAATCCCTAATTT | |
| | | GAGGGTCAGTTCCTGCAGAAGTGCCCTTTGCCTC | |
| | | CACTCAATGCCTCAATTTGTTTTCTGCATGACTGA | |
| | | GAGTCTCAGTGTTGGAACGGGACAGTATTTATGT | |
| | | ATGAGTTTTTCCTATTTATTTTGAGTCTGTGAGGT | |
| | | CTTCTTGTCATGTGAGTGTGGTTGTGAATGATTTC | |
| | | TTTTGAAGATATATTGTAGTAGATGTTACAATTTT | |
| | | GTCGCCAAACTAAACTTGCTGCTTAATGATTTGC | |
| | | TCACATCTAGTAAAACATGGAGTATTTGTAAGGT | |
| | | GCTTGGTCTCCTCTATAACTACAAGTATACATTG | |
| | | GAAGCATAAAGATCAAACCGTTGGTTGCATAGG | |
| | | ATGTCACCTTTATTTAACCCATTAATACTCTGGTT | |
| | | GACCTAATCTTATTCTCAGACCTCAAGTGTCTGTG | |
| | | CAGTATCTGTTCCATTTAAATATCAGCTTTACAAT | |
| | | TATGTGGTAGCCTACACACATAATCTCATTTCATC | |
| | | GCTGTAACCACCCTGTTGTGATAACCACTATTATT | |
| | | TTACCCATCGTACAGCTGAGGAAGCAAACAGATT | |
| | | AAGTAACTTGCCCAAACCAGTAAATAGCAGACCT | |
| | | CAGACTGCCACCCACTGTCCTTTTATAATACAATT | |
| | | TACAGCTATATTTTACTTTAAGCAATTCTTTTATT | |
| | | CAAAAACCATTTATTAAGTGCCCTTGCAATATCA | |
| | | ATCGCTGTGCCAGGCATTGAATCTACAGATGTGA | |
| | | GCAAGACAAAGTACCTGTCCTCAAGGAGCTCATA | |
| | | GTATAATGAGGAGATTAACAAGAAAATGTATTAT | |
| | | TACAATTTAGTCCAGTGTCATAGCATAAGGATGA | |
| | | TGCGAGGGGAAAACCCGAGCAGTGTTGCCAAGA | |
| | | GGAGGAAATAGGCCAATGTGGTCTGGGACGGTT | |
| | | GGATATACTTAAACATCTTAATAATCAGAGTAAT | |
| | | TTTCATTTACAAAGAGAGGTCGGTACTTAAAATA | |
| | | ACCCTGAAAAATAACACTGGAATTCCTTTTCTAG | |
| | | CATTATATTTATTCCTGATTTGCCTTTGCCATATA | |
| | | ATCTAATGCTTGTTTATATAGTGTCTGGTATTGTT | |
| | | TAACAGTTCTGTCTTTTCTATTTAAATGCCACTAA | |
| | | ATTTTAAATTCATACCTTTCCATGATTCAAAATTC | |
| | | AAAAGATCCCATGGGAGATGGTTGGAAAATCTCC | |
| | | ACTTCATCCTCCAAGCCATTCAAGTTTCCTTTCCA | |
| | | GAAGCAACTGCTACTGCCTTTCATTCATATGTTCT | |
| | | TCTAAAGATAGTCTACATTTGGAAATGTATGTTA | |
| | | AAAGCACGTATTTTTAAAATTTTTTTCCTAAATAG | |
| | | TAACACATTGTATGTCTGCTGTGTACTTTGCTATT | |
| | | TTTATTTATTTTAGTGTTTCTTATATAGCAGATGG | |
| | | AATGAATTTGAAGTTCCCAGGGCTGAGGATCCAT | |
| | | GCCTTCTTTGTTTCTAAGTTATCTTTCCCATAGCT | |
| | | TTTCATTATCTTTCATATGATCCAGTATATGTTAA | |
| | | ATATGTCCTACATATACATTTAGACAACCACCAT | |
| | | TTGTTAAGTATTTGCTCTAGGACAGAGTTTGGATT | |
| | | TGTTTATGTTTGCTCAAAAGGAGACCCATGGGCT | |
| | | CTCCAGGGTGCACTGAGTCAATCTAGTCCTAAAA | |
| | | AGCAATCTTATTATTAACTCTGTATGACAGAATC | |
| | | ATGTCTGGAACTTTTGTTTTCTGCTTTCTGTCAAG | |
| | | TATAAACTTCACTTTGATGCTGTACTTGCAAAATC | |
| | | ACATTTTCTTTCTGGAAATTCCGGCAGTGTACCTT | |
| | | GACTGCTAGCTACCCTGTGCCAGAAAAGCCTCAT | |
| | | TCGTTGTGCTTGAACCCTTGAATGCCACCAGCTG | |
| | | TCATCACTACACAGCCCTCCTAAGAGGCTTCCTG | |
| | | GAGGTTTCGAGATTCAGATGCCCTGGGAGATCCC | |
| | | AGAGTTTCCTTTCCCTCTTGGCCATATTCTGGTGT | |
| | | CAATGACAAGGAGTACCTTGGCTTTGCCACATGT | |
| | | CAAGGCTGAAGAAACAGTGTCTCCAACAGAGCT | |
| | | CCTTGTGTTATCTGTTTGTACATGTGCATTTGTAC | |
| | | AGTAATTGGTGTGACAGTGTTCTTTGTGTGAATT | |
| | | ACAGGCAAGAATTGTGGCTGAGCAAGGCACATA | |
| | | GTCTACTCAGTCTATTCCTAAGTCCTAACTCCTCC | |
| | | TTGTGGTGTTGGATTTGTAAGGCACTTTATCCCTT | |
| | | TTGTCTCATGTTTCATCGTAAATGGCATAGGCAG | |
| | | AGATGATACCTAATTCTGCATTTGATTGTCACTTT | |
| | | TTGTACCTGCATTAATTTAATAAAATATTCTTATT | |
| | | TATTTTGTTACTTGGTACACCAGCATGTCCATTTT | |
| | | CTTGTTTATTTTGTGTTTAATAAAATGTTCAGTTT | |
| | | AACATCCCA | |
| Cyno- | XM_ | CACACACACGCACACACCCCTACTTTCTAGAATA | 1038 |
| molgus | 005581779.2 | AAAACCAAAGCCATATGGGTCTGCTGTTGACTTT | |
| Monkey | | CTATATGTCGTAGAGTTATATCAAGTTAGGTCAA | |
| CD274 | | GATGTTCAGTCACCTTGAAGACGCTTTTATCAGA | |
| mRNA | | AAGGGGGAAACCTTTCTGATAAAGGTTAAGGGG | |
| | | TAACCTTAAGCTGTCACCCCTCTGAAGGTAAAAT | |
| | | CAAGGTGCGTTCAGATGTTGGCTTGTTGTAAATT | |
| | | TCTGTTTATATTAATAACATACCAAATGTGGATTT | |
| | | GTTTTAATCTTCGGAACTCTTTCCGGTGAAAACCT | |
| | | CATTTACAAGAAAACTGGACTGACAGGTTTCACT | |
| | | TTCTGTTTCATTTCTATACATAGCTTTATTCCTAG | |
| | | GACACCAACACCACTCGCTACCCAAACTGAAAGC | |
| | | TTCCCCGATTCCGCCGAAGGTCAGGAAAGTCCAA | |
| | | TGCCGGGCAAACTGGATTTGCTGCCTTGCGCAGA | |
| | | GGTGGGCGGGACCCCGCCTCCGGGCCGGGCGCC | |
| | | AAGTTGAGCAGCTGGCACGCCTCGCGAAGCCCCA | |
| | | GTCCTGAAGCCCCAGTCCTGCGCTGCTTCCCGAG | |
| | | GCTCCGCACCAGCCGCGCTTCTCTCTGCCTGCAG | |
| | | CACATTCCAGAAAGATGAGGATATTTGCTGTCTT | |
| | | TATATTCACGATCTACTGGCATTTGCTGAATGCAT | |
| | | TTACTGTCACGGTTCCCAAGGACCTATATGTGGT | |
| | | AGAGTATGGCAGCAATATGACAATTGAATGCAA | |
| | | ATTCCCAGTAGAAAAACAATTAGACCTGACTTCA | |
| | | CTAATTGTCTATTGGGAAATGGAGGATAAGAACA | |
| | | TTATTCAATTTGTGCATGGAGAGGAAGACCTGAA | |
| | | GGTTCAGCATAGTAACTACAGACAGAGGGCCCA | |
| | | GCTGTTGAAGGACCAGCTCTCCCTGGGAAATGCT | |
| | | GCACTTCGGATCACAGATGTGAAATTGCAGGATG | |
| | | CAGGGGTTTACCGCTGCATGATCAGCTATGGTGG | |
| | | TGCCGACTACAAGCGGATTACCGTGAAAGTCAAT | |
| | | GCTCCATACAACAAAATCAACCAAAGAATTTTGG | |
| | | TTGTCGATCCAGTCACCTCTGAACATGAACTAAC | |
| | | ATGTCAGGCTGAGGGCTACCCCAAGGCCGAAGTC | |
| | | ATTTGGACAAGCAGTGACCATCAAGTCCTGAGTG | |
| | | GTAAGACCACCACCACCAATTCCAAGAGAGAGG | |
| | | AGAAGCTTTTAAATGTGACCAGCACACTGAGAAT | |
| | | CAACACAACAGCTAATGAGATTTTCTACTGCATT | |
| | | TTTAGGAGATTAGATCCTGAGGAAAACCATACAG | |
| | | CTGAATTGGTCATCCCAGAACTACCTCTGGCGCT | |
| | | TCCTCCAAATGAAAGGACTCACTTGGTAATTCTG | |
| | | GGAGCCATCTTTTTACTCCTTGGTGTAGCACTGAC | |
| | | ATTCATCTTCTATTTAAGAAAAGGGAGAATGATG | |
| | | GATATGAAAAAATGTGGCATTCGAGTTACAAACT | |
| | | CAAAGAAGCAACGTGATACACAATTGGAGGAGA | |
| | | CGTAATCCAGCATTGGAACTTCTGATCTTCAAGC | |
| | | AGGGATTCTCAGCCTGTGGTTTGGGGGTTCGTCA | |
| | | GGGCTGAGCATGACCAGAGGAATGAATGGGCCC | |
| | | GTGGGATGCATGCAGTATGGGACTTAAAAGGCCC | |
| | | AAGCACTGAAAATGGAACCTGGCGAAAGCAGAG | |
| | | GAGGAGAATGAAGAAAAATGGAGTTGAACAGGG | |
| | | AGCGTGGAGGGAGACCTTGATACTTTCAAATGCC | |
| | | TGAGGGGCTCATCGGTGCATGTGACAGGGAGAA | |
| | | AGGATACTTCTGAACAAGGAGCCTCCAAGCAAAT | |
| | | CATCCACTGCTCATCTTAGGAAAACGGGTTGAGA | |
| | | ATCCCTAATTTGAGGGTCAGTTCCTGCAGAAGTG | |
| | | CCCTTTGCCTCCACTCAATGCCTCAATTTGTTTTC | |
| | | TGCGTGACTGAGGGTCCCAGTGTTGGAACAGTAT | |
| | | TTATGTATGAGATTTTCCTATTTATTTTGAGTCTG | |
| | | TGAGGTCTTCTTGTCATGGGAGTGTGGTTGTGAA | |
| | | TGATTTCTTTTGAAGATATATTGTAGTAGATGTTA | |
| | | CAATTTTGTCGCCAAACTAAACTTGATGCTTAAT | |
| | | GACTTGCTCACATCTAGTAAAACATGGAGTATTT | |
| | | GTAAGGTGCTTGGTCTCCTCTATAACTACAAGTA | |
| | | CACATTGGAAGCATAAAGATCAAACCGTTGATTT | |
| | | GTATAGGATGTCACCTTTATTTAACCCATTAATAC | |
| | | TCTGATTGACTTAATCTTATTCTCAGACCTCAAGT | |
| | | GTCTGTGCAGTATCTGTTCCATTTAAATATCAGCT | |
| | | TTATAATTATGTGGTACCATACACACATAATCTC | |
| | | CTTTCATCGCTGTAACCACCCTGTTGTGATGACCA | |
| | | CTATTATTTTACCCATTGTACAGCTGAGGAAGCA | |
| | | AACAGATTAAGTAACTTGCCAAAACCAGTAAATA | |
| | | GCAGAGCTCAGACTGCCACCCACTGTCCTTTTAT | |
| | | AATACAATTTACAGCTATATTTTACTTTAAGCAAT | |
| | | TCATTTATTCAAAACCCATTTATTAAGTGCCCTTG | |
| | | CAATATCAATCACTGTACCAGGCATTGAATCTAC | |
| | | AGATGTGAGCAAGAGAAAGTACCTGTCCTCAAG | |
| | | GAGCTTGGAGTATAATAAGGAGATTAATAAGAA | |
| | | AATATATTATTACAATCTAGTCCAGTGTCATAGC | |
| | | ATAAGGATGATGTGAGGAGAAAAGCTGAGCAGT | |
| | | GTTGCCAAGAGGAGGAAATAGGCCAATGTGGTC | |
| | | TGGGACAGTTGAATGTATTTAAACATCTTAATAA | |
| | | TCAAAGTAATTTTCATTTACAAAGAGAAGTCAGT | |
| | | ACTTAAAATAACCCTGAAAAATAACACTGGAATT | |
| | | CCTTTTCTAGCATTATATTTATCCCTGATTTGCCT | |
| | | TTGCCATACAATCTAATGCTTGTTTATATAGTGTC | |
| | | TGATATTGTTTAACAGTTCTGTCTTTTCTATTCAA | |
| | | ATGCTATTAAATTTTAAATTCATACCTTTCCATGA | |
| | | TTCAAAATTCAAAAGATCCCATGGGAGATGGTTT | |
| | | GAAAATCTCCACTTCATCCTCCAAGCCATTCAAG | |
| | | TTTCCTTTCCAGAAGCAACTGCTACTGCCTTTTAT | |
| | | TCATATGTTCTTCTAAAGATAGTCTACATTTGGAA | |
| | | ATGTATGTTAAAAGCATATATTTTTAAATTTTTTT | |
| | | CCCTAAATAGTAACACATTATATGTCTGCTGTGC | |
| | | ACTTTGCTATTTTTATTTATTGTAGTGTTTCTTATG | |
| | | TAGCAGATGGAATGAATTTGAAGCTCCCAAGGGT | |
| | | CAGGACACATGCCTTCTTTGTTTCTAAGTTATCTT | |
| | | TCCCATAGCTTTTCATAATCTTTCATATGATTTAG | |
| | | TACATGTTAAATATGTGCTACATATACATTTAGA | |
| | | CAACCAGCATTTGTTAAGTATTTGCTCTAGGACT | |
| | | GAGTTTGGATTTATGTTTGCTCAAAAGGAGACCC | |
| | | ATGGGCTCTCCAGGGTGCACTGAGTCAATCTAGT | |
| | | CCTAAAAAGCAATCTTATTATTAACTCTGTATGA | |
| | | CAGAATCATATCTGGAACTTTTGTTTTCTGCTTTC | |
| | | TGTCAAGTATAAACTTCACTTTGATGCTGTACTTG | |
| | | CAAAATCACATTTTCTTTCTGGAAATTCCAGTAGT | |
| | | GTACCTTGACTGCTAGTTACCCTGTGCCAGAAAA | |
| | | GCCTCATTCGTTGTGCTTGAACCCTTTAATGCCAC | |
| | | CAGCTGTCATCACTACACAGGCCTCCTAAGAGGC | |
| | | TTCCTGGAGGTTTTGAGATTCAGATGCCCTGAGA | |
| | | GATCCCAGAGTTTCCTTTCCCTCTTGGCCACATTC | |
| | | TGGTGTCAGTGACAAGGAATACCTTCGCTTTGCC | |
| | | ACCCGTCAAGGTTGAAGAAACAGCGTCTCCAACA | |
| | | GAGCTCCTTGTGTTATCTGTTTGTACATGTGCATT | |
| | | TGTACAGTAATTTGTGTGACAGTGTTCTTTGTGTG | |
| | | AATTACAGGCAAGAACTGTGGCTGAGCAAGGCA | |
| | | CATAGTCTACTCAGTCTATTCCTAACTCCTCCTTT | |
| | | TGGTGTTGGATTTGTAAGGCACTTTATCCCTTTTG | |
| | | TCTCATGTTTCATCGTAAATGGCATAGGCAGAGA | |
| | | TGATATCTAATTCTGCATTTGATTGTCACTTTTTG | |
| | | TACCTGCATTAATTTAATAAAATATCCTTATTTAT | |
| | | TTTGTTACTTGGTACACCAGCATGTCCATTTTCTT | |
| | | GTTTATTTTGTGTTTCATAAAATGCTTAGTTTAA | |
| Mus | NM_021893.3 | GAAATCGTGGTCCCCAAGCCTCATGCCAGGCTGC | 1039 |
| Musculus | | ACTTGCACGTCGCGGGCCAGTCTCCTCGCCTGCA | |
| CD274 | | GATAGTTCCCAAAACATGAGGATATTTGCTGGCA | |
| mRNA | | TTATATTCACAGCCTGCTGTCACTTGCTACGGGC | |
| | | GTTTACTATCACGGCTCCAAAGGACTTGTACGTG | |
| | | GTGGAGTATGGCAGCAACGTCACGATGGAGTGC | |
| | | AGATTCCCTGTAGAACGGGAGCTGGACCTGCTTG | |
| | | CGTTAGTGGTGTACTGGGAAAAGGAAGATGAGC | |
| | | AAGTGATTCAGTTTGTGGCAGGAGAGGAGGACCT | |
| | | TAAGCCTCAGCACAGCAACTTCAGGGGGAGAGC | |
| | | CTCGCTGCCAAAGGACCAGCTTTTGAAGGGAAAT | |
| | | GCTGCCCTTCAGATCACAGACGTCAAGCTGCAGG | |
| | | ACGCAGGCGTTTACTGCTGCATAATCAGCTACGG | |
| | | TGGTGCGGACTACAAGCGAATCACGCTGAAAGTC | |
| | | AATGCCCCATACCGCAAAATCAACCAGAGAATTT | |
| | | CCGTGGATCCAGCCACTTCTGAGCATGAACTAAT | |
| | | ATGTCAGGCCGAGGGTTATCCAGAAGCTGAGGTA | |
| | | ATCTGGACAAACAGTGACCACCAACCCGTGAGTG | |
| | | GGAAGAGAAGTGTCACCACTTCCCGGACAGAGG | |
| | | GGATGCTTCTCAATGTGACCAGCAGTCTGAGGGT | |
| | | CAACGCCACAGCGAATGATGTTTTCTACTGTACG | |
| | | TTTTGGAGATCACAGCCAGGGCAAAACCACACA | |
| | | GCGGAGCTGATCATCCCAGAACTGCCTGCAACAC | |
| | | ATCCTCCACAGAACAGGACTCACTGGGTGCTTCT | |
| | | GGGATCCATCCTGTTGTTCCTCATTGTAGTGTCCA | |
| | | CGGTCCTCCTCTTCTTGAGAAAACAAGTGAGAAT | |
| | | GCTAGATGTGGAGAAATGTGGCGTTGAAGATAC | |
| | | AAGCTCAAAAAACCGAAATGATACACAATTCGA | |
| | | GGAGACGTAAGCAGTGTTGAACCCTCTGATCGTC | |
| | | GATTGGCAGCTTGTGGTCTGTGAAAGAAAGGGCC | |
| | | CATGGGACATGAGTCCAAAGACTCAAGATGGAA | |
| | | CCTGAGGGAGAGAACCAAGAAAGTGTTGGGAGA | |
| | | GGAGCCTGGAACAACGGACATTTTTTCCAGGGAG | |
| | | ACACTGCTAAGCAAGTTGCCCATCAGTCGTCTTG | |
| | | GGAAATGGATTGAGGGTTCCTGGCTTAGCAGCTG | |
| | | GTCCTTGCACAGTGACCTTTTCCTCTGCTCAGTGC | |
| | | CGGGATGAGAGATGGAGTCATGAGTGTTGAAGA | |
| | | ATAAGTGCCTTCTATTTATTTTGAGTCTGTGTGTT | |
| | | CTCACTTTGGGCATGTAATTATGACTGGTGAATT | |
| | | CTGACGACATGATAGATCTTAAGATGTAGTCACC | |
| | | AAACTCAACTGCTGCTTAGCATCCTCCGTAACTA | |
| | | CTGATACAAGCAGGGAACACAGAGGTCACCTGC | |
| | | TTGGTTTGACAGGCTCTTGCTGTCTGACTCAAATA | |
| | | ATCTTTATTTTTCAGTCCTCAAGGCTCTTCGATAG | |
| | | CAGTTGTTCTGTATCAGCCTTATAGGTGTCAGGT | |
| | | ATAGCACTCAACATCTCATCTCATTACAATAGCA | |
| | | ACCCTCATCACCATAGCAACAGCTAACCTCTGTT | |
| | | ATCCTCACTTCATAGCCAGGAAGCTGAGCGACTA | |
| | | AGTCACTTGCCCACAGAGTATCAGCTCTCAGATT | |
| | | TCTGTTCTTCAGCCACTGTCCTTTCAGGATAGAAT | |
| | | TTGTCGTTAAGAAATTAATTTAAAAACTGATTAT | |
| | | TGAGTAGCATTGTATATCAATCACAACATGCCTT | |
| | | GTGCACTGTGCTGGCCTCTGAGCATAAAGATGTA | |
| | | CGCCGGAGTACCGGTCGGACATGTTTATGTGTGT | |
| | | TAAATACTCAGAGAAATGTTCATTAACAAGGAGC | |
| | | TTGCATTTTAGAGACACTGGAAAGTAACTCCAGT | |
| | | TCATTGTCTAGCATTACATTTACCTCATTTGCTAT | |
| | | CCTTGCCATACAGTCTCTTGTTCTCCATGAAGTGT | |
| | | CATGAATCTTGTTGAATAGTTCTTTTATTTTTTAA | |
| | | ATGTTTCTATTTAAATGATATTGACATCTGAGGC | |
| | | GATAGCTCAGTTGGTAAAACCCTTTCCTCACAAG | |
| | | TGTGAAACCCTGAGTCTTATCCCTAGAACCCACA | |
| | | TAAAAAACAGTTGCGTATGTTTGTGCATGCTTTT | |
| | | GATCCCAGCACTAGGGAGGCAGAGGCAGGCAGA | |
| | | TCCTGAGCTCTCATTGACCACCCAGCCTAGCCTA | |
| | | CATGGTTAGCTCCAGGCCTACAGGAGCTGGCAGA | |
| | | GCCTGAAAAACGATGCCTAGACACACACACACA | |
| | | CACACACACACACACACACACACACACACACCA | |
| | | TGTACTCATAGACCTAAGTGCACCCTCCTACACA | |
| | | TGCACACACATACAATTCAAACACAAATCAACAG | |
| | | GGAATTGTCTCAGAATGGTCCCCAAGACAAAGA | |
| | | AGAAGAAAAACACCAAACCAGCTCTATTCCCTCA | |
| | | GCCTATCCTCTCTACTCCTTCCTAGAAGCAACTAC | |
| | | TATTGTTTTTGTATATAAATTTACCCAACGACAGT | |
| | | TAATATGTAGAATATATATTAAAGTGTCTGTCAA | |
| | | TATATATTATCTCTTTCTTTCTTTCTTCCTTTCTTT | |
| | | CTTTCTTTCTTTCTTTCTTTCTTTCTTTCTTTCTTTC | |
| | | TTTCTTCCTTCCTTCCTTCCTTCCTTCCTTCCTTCC | |
| | | TTCCTTTCTTTCTTTCTTTCTTTTTTTCTGTCTATCT | |
| | | GTACCTAAATGGTTGCTCACTATGCATTTTCTGTG | |
| | | CTCTTCGCCCTTTTTATTTAATGTATGGATATTTA | |
| | | TGCTGCTTCCAGAATGGATCTAAAGCTCTTTGTTT | |
| | | CTAGGTTTTCTCCCCCATCCTTCTAGGCATCTCTC | |
| | | ACACTGTCTAGGCCAGACACCATGTCTGCTGCCT | |
| | | GAATCTGTAGACACCATTTATAAAGCACGTACTC | |
| | | ACCGAGTTTGTATTTGGCTTGTTCTGTGTCTGATT | |
| | | AAAGGGAGACCATGAGTCCCCAGGGTACACTGA | |
| | | GTTACCCCAGTACCAAGGGGGAGCCTTGTTTGTG | |
| | | TCTCCATGGCAGAAGCAGGCCTGGAGCCATTTTG | |
| | | GTTTCTTCCTTGACTTCTCTCAAACACAGACGCCT | |
| | | CACTTGCTCATTACAGGTTCTCCTTTGGGAATGTC | |
| | | AGCATTGCTCCTTGACTGCTGGCTGCCCTGGAAG | |
| | | GAGCCCATTAGCTCTGTGTGAGCCCTTGACAGCT | |
| | | ACTGCCTCTCCTTACCACAGGGGCCTCTAAGATA | |
| | | CTGTTACCTAGAGGTCTTGAGGATCTGTGTTCTCT | |
| | | GGGGGGAGGAAAGGAGGAGGAACCCAGAACTTT | |
| | | CTTACAGTTTTCCTTGTTCTGTCACATGTCAAGAC | |
| | | TGAAGGAACAGGCTGGGCTACGTAGTGAGATCCT | |
| | | GTCTCAAAGGAAAGACGAGCATAGCCGAACCCC | |
| | | CGGTGGAACCCCCTCTGTTACCTGTTCACACAAG | |
| | | CTTATTGATGAGTCTCATGTTAATGTCTTGTTTGT | |
| | | ATGAAGTTTAAGAAAATATCGGGTTGGGCAACAC | |
| | | ATTCTATTTATTCATTTTATTTGAAATCTTAATGC | |
| | | CATCTCATGGTGTTGGATTGGTGTGGCACTTTATT | |
| | | CTTTTGTGTTGTGTATAACCATAAATTTTATTTTG | |
| | | CATCAGATTGTCAATGTATTGCATTAATTTAATA | |
| | | AATATTTTTATTTATTAAAAAAAAAAAAAAAAA | |
| Rattus | NM_ | ATGAGGATATTTGCTGTCCTTATAGTCACAGCCT | 1040 |
| nor- | 001191954.1 | GCAGTCACGTGCTAGCGGCATTTACCATCACAGC | |
| vegicus | | TCCAAAGGACCTGTACGTGGTGGAGTATGGCAGC | |
| CD274 | | AATGTCACGATGGAATGCAGATTCCCAGTAGAAC | |
| mRNA | | AGAAATTGGACCTGCTTGCCTTAGTGGTGTACTG | |
| | | GGAAAAGGAAGACAAGGAAGTTATTCAGTTTGT | |
| | | GGAGGGAGAGGAGGACCTGAAGCCTCAACACAG | |
| | | CAGCTTCAGGGGGAGAGCCTTCTTGCCAAAGGAC | |
| | | CAGCTTTTGAAGGGGAACGCGGTGCTTCAGATCA | |
| | | CAGATGTCAAGCTGCAGGACGCAGGTGTCTACTG | |
| | | CTGCATGATCAGCTATGGTGGAGCGGACTACAAG | |
| | | CGAATCACATTGAAAGTCAACGCTCCATACCGCA | |
| | | AAATCAACCAAAGAATTTCCATGGATCCAGCCAC | |
| | | TTCTGAGCATGAACTAATGTGCCAGGCTGAGGGT | |
| | | TACCCAGAAGCCGAAGTGATCTGGACAAACAGT | |
| | | GACCACCAGTCCCTGAGTGGGGAAACAACTGTCA | |
| | | CCACTTCCCAGACTGAGGAGAAGCTTCTCAACGT | |
| | | GACCAGCGTTCTGAGGGTCAACGCAACAGCTAAT | |
| | | GATGTTTTCCACTGTACGTTCTGGAGAGTACACT | |
| | | CAGGGGAGAACCACACGGCTGAACTGATCATCC | |
| | | CAGAACTGCCTGTACCACGTCTCCCACATAACAG | |
| | | GACACACTGGGTACTCCTGGGATCCGTCCTTTTG | |
| | | TTCCTCATCGTGGGGTTCACCGTCTTCTTCTGCTT | |
| | | GAGAAAACAAGTGAGAATGCTAGATGTGGAAAA | |
| | | ATGCGGCTTCGAAGATAGAAATTCAAAGAACCG | |
| | | AAATGATACACAGTTCGAGGAGACGTAA | |
| GalXC- | Unmodified | AGGAUAGAAUUUGUCGUUAAGCAGCCGAAAGG | 1041 |
| mCD274 | 36 mer | CUGC | |
| GalXC- | Unmodified | UUAACGACAAAUUCUAUCCUGG | 1042 |
| mCD274 | 22 mer | | |
| GalXC- | Unmodified | GGUGGAUGAAACUCAGUUUAGCAGCCGAAAGG | 1043 |
| ALDH2- | 36 mer | CUGC | |
| C18 | | | |
| GalXC- | Unmodified | UAAACUGAGUUUCAUCCACCGG | 1044 |
| ALDH2- | 22 mer | | |
| C18 | | | |
| GalXC- | Modified 36 | [mGs][mG][fU][mG][fG][mA][mU][fG][mA][fA][mA] | 1045 |
| ALDH2- | mer | [fC][fU][mC][fA][mG][fU][mU][mU][mA][mG][mC][mA] | |
| C18 | | [mG][mC][mC][mG][ademA- | |
| | | C18][mA][mA][mG][mG][mC][mU][mG][mC] | |
| GalXC- | Modified 22 | [MePhosphonate-4O- | 1046 |
| ALDH2- | mer | mUs][fAs][fA][fA][fC][mU][fG][mA][mG][fU][mU] | |
| C18 | | [mU][mC][fA][mU][fC][mC][mA][fC][mCs][mGs][mG] | |
| GalXC- | Modified 36 | [mGs][mG][fU][mG][fG][mA][mU][fG][mA][fA][mA] | 1047 |
| ALDH2- | mer | [fC][fU][mC][fA][mG][fU][mU][mU][mA][mG][mC][mA] | |
| C16 | | [mG][mC][mC][mG][ademA- | |
| | | C16][mA][mA][mG][mG][mC][mU][mG][mC] | |
| GalXC- | Modified | [mGs][mG][fU][mG][fG][mA][mU][fG][mA][fA][mA] | 1048 |
| ALDH2- | 36 mer | [fC][fU][mC][fA][mG][fU][mU][mU][mA][mG][mC][mA] | |
| C22 | | [mG][mC][mC][mG][ademA- | |
| | | C18][mA][mA][mG][mG][mC] | |
| | | [mU][mG][mC] | |
| GalXC- | Modified | [mGs][mG][fU][mG][fG][mA][mU][fG][mA][fA][mA] | 1049 |
| ALDH2- | 36 mer | [fC][fU][mC][fA][mG][fU][mU][mU][mA][mG][mC][mA] | |
| C24 | | [mG][mC][mC][mG][ademA- | |
| | | C22][mA][mA][mG][mG][mC] | |
| | | [mU][mG][mC] | |
| CD274- | Modified | [ademGs- | 1050 |
| 0098 | 36 mer | C18][mG][mA][mU][mA][mU][mU][fU][fG][fC][fU] | |
| | | [mG][mU][mC][mU][mU][mU][mA][mU][mA][mG][mC] | |
| | | [mA][mG][mC][mC][mG][mA][mA][mA][mG][mG][mC] | |
| | | [mU][mG][mC] | |

1.-68. (canceled)

69. An oligonucleotide for reducing CD274 expression, the oligonucleotide comprising an antisense strand of 15 to 30 nucleotides in length and a sense strand of 15 to 40 nucleotides in length, wherein the sense and antisense strand form a duplex region, wherein the antisense strand has a region of complementarity to a target sequence of CD274 as set forth in SEQ ID NO: 5, wherein the sense strand comprises at least one lipid moiety conjugated to the 5′ terminal nucleotide of the sense strand.

70. The oligonucleotide of claim 69, wherein the antisense strand comprises a sequence as set forth in SEQ ID NO: 728.

71. The oligonucleotide of claim 69, wherein the sense strand comprises a sequence as set forth in SEQ ID NO: 487.

72. The oligonucleotide of claim 69, wherein the lipid moiety is a saturated fatty acid moiety that ranges in size from C8 to C30 in length.

73. The oligonucleotide of claim 72, wherein the lipid moiety is a C18 saturated fatty acid moiety.

74. The oligonucleotide of claim 69, wherein the lipid moiety is conjugated to the 2′ carbon of the ribose ring of the 5′ terminal nucleotide.

75. The oligonucleotide of claim 69, wherein the sense strand comprises the sequence set forth in SEQ ID NO: 1050.

76. The oligonucleotide of claim 69, wherein the antisense strand comprises the sequence set forth in SEQ ID NO: 1005.

77. The oligonucleotide of claim 69, wherein the sense strand comprises the sequence set forth in SEQ ID NO: 1050, and wherein the antisense strand comprises the sequence set forth in SEQ ID NO: 1005.

78. An oligonucleotide for reducing CD274 expression, wherein the oligonucleotide comprises a sense strand comprising the sequence set forth in SEQ ID NO: 1050 and the antisense strand comprises the sequence set forth in SEQ ID NO: 1005, wherein the sense strand and antisense strand form an asymmetric duplex region of 20 nucleotides in length and having an overhang of 2 nucleotides at the 3′ terminus of the antisense strand.

79. The oligonucleotide of claim 69, wherein the oligonucleotide reduces expression of CD274 mRNA in one or more immune cells associated with a tumor microenvironment.

80. A pharmaceutical composition comprising the oligonucleotide of claim 69, and a pharmaceutically acceptable carrier, delivery agent, or excipient.

81. The oligonucleotide of claim 78, wherein the oligonucleotide reduces expression of CD274 mRNA in one or more immune cells associated with a tumor microenvironment.

82. A pharmaceutical composition comprising the oligonucleotide of claim 78, and a pharmaceutically acceptable carrier, delivery agent, or excipient.
